(19) United States
(12) Patent Application Publication
(10) Pub. No.: US 2004/0077625 A1
(43) Pub. Date: Apr. 22, 2004

(54) Novel 1,4-benzothiazepine and 1,5-benzothiazepine compounds as inhibitors of apical sodium codependent bile acid transport abd taurocholate uptake

(76) Inventors: Samuel J. Tremont, St. Louis, MO (US); Kevin J. Koeller, Maryland Heights, MO (US); William L. Neumann, St. Louis, MO (US)

Correspondence Address: BANNER & WITCOFF, 1001 G STREET N W, SUITE 1100, WASHINGTON, DC, 20001, US

(21) Appl. No.: 10/333,842

(22) PCT Filed: Jul. 26, 2001
(86) PCT No.: PCT/US01/23533

(30) Foreign Application Priority Data
Jul. 25, 2001 (US) 09/912,233

(51) Int. Cl. A61K 31/554; C07D 267/02

(52) U.S. Cl. 514/211.09; 540/546

(57) ABSTRACT

Compounds, pharmaceutical compositions, and methods for the treatment of a hyperlipidemic condition in a subject. The compounds of the present invention are apical sodium co-dependent bile acid transport inhibitors and are 1,4-benzothiazepine and 1,5-benzothiazepine compounds corresponding to Formula I:

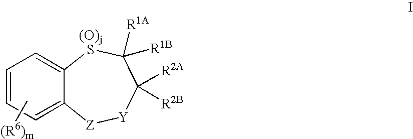

wherein j, m, Y, Z, R^{1A}, R^{1B}, R^{2A}, R^{2B} and R⁶ are as defined in the specification.

This application claims priority to provisional U.S. Application Ser. No. 60/220,966 filed Jul. 26, 2000, incorporated herein by reference in its entirety.

### FIELD OF THE INVENTION

The present invention relates to compounds, pharmaceutical compositions, and methods for the treatment of a hyperlipidemic condition in a subject. More particularly, the present invention relates to novel 1,4-benzothiazepine and 1,5-benzothiazepine compounds that are useful as apical sodium co-dependent bile acid transport inhibitors.

### BACKGROUND OF THE INVENTION

The major metabolic fate of cholesterol in the human body is in the hepatic synthesis of bile acids. Bile acids are both passively and actively reabsorbed from the small intestine and recycled via the enterohepatic circulation to conserve the total pool of bile acids. Dietschy, “Mechanisms for the intestinal absorption of bile acids”, J. Lipid Res., 9:297-309 (1968). Bile acids undergo passive absorption in the proximal small intestine and active transport in the terminal ileum. Love et al., “New insights into bile acid transport”, Curr. Opin. Lipidol., 9(3):225-229 (1998). Ileal active transport accounts for the majority of intestinal bile acid uptake and is the exclusive route for taurine-conjugated bile acids. Id. Ileal active transport is mediated by the apical sodium co-dependent bile acid transporter (“ASBT”, also known as the ileal bile acid transporter or “IBAT”) localized to the distal one-third of the ileum. Craddock et al., “Expression and transport properties of the human ileal and renal sodium-dependent bile acid transporter”, Am. J. Physiol., 274 (Gastrointest. Liver Physiol. 37):G157-G169 (1998).

An equilibrium generally exists between hepatic cholesterol and the bile acid pool. Interruption of the enterohepatic recirculation of bile acids (e.g., the binding of intestinal bile acids to a sequestering resin such as cholestyramine; the surgical removal of the ileum to physically eliminate ileal ASBT; or the specific inhibition of ileal ASBT) results in a decrease in the liver bile acid pool and stimulates increased hepatic synthesis of bile acids from cholesterol (i.e., an upregulation of cholesterol-7α-hydroxylase activity), eventually depleting the liver's pool of esterified cholesterol. In order to maintain liver cholesterol levels necessary to support bile acid synthesis, the de novo synthesis of cholesterol increases in the hepatocytes (i.e., an upregulation of 3-hydroxy-3-methylglutaryl coenzyme-A reductase activity) and also increases the uptake of serum cholesterol by upregulating the number of cell surface low density lipoprotein cholesterol receptors (“LDL receptors”). The number of hepatic LDL receptors directly impacts serum low density lipoprotein (“LDL”) cholesterol levels, with an increase in the number of LDL receptors resulting in a decrease in serum cholesterol. The net result, therefore, is that serum LDL cholesterol levels decrease when intestinal bile acid reabsorption is reduced. Stedronsky, in “Interaction of bile acids and cholesterol with nonsystemic agents having hypocholesterolemic properties,” Biochimica et Biophysica Acta, 1210 (1994) 255-287 discusses biochemistry, physiology, and known active agents surrounding bile acids and cholesterol. Agents that inhibit the transport of bile acids across the tissue of the ileum, therefore, can cause a decrease in the levels of cholesterol in blood serum.

A large number of adults have cholesterol levels that exceed recommended levels and can be considered as having hypercholesterolemia. If left untreated, such hypercholesteroleamia can result, for example, in atherosclerosis and complications of atherosclerosis such as myocardial infarction, stroke and peripheral vascular disease. Accordingly, the development of new therapeutic agents (such as ASBT inhibitors) that overcome the problems associated with, and/or show improved performance relative to, the therapeutic agents disclosed in the literature would be desirable. The present invention therefore comprises novel 1,4- and 1,5-benzothiazepines that represent an improvement over the therapeutic agents previously disclosed for use in the treatment of a hyperlipidemic condition, together with pharmaceutical compositions and methods of use thereof.

Those 1,4- and 1,5-benzothiepines that have been disclosed in the literature as agents for the treatment of a hyperlipidemic condition include the following:

WO93/16055 discloses selected 1,4-benzothiazepines as useful in the treatment of a hyperlipidemic condition.

WO94/18183 discloses selected 1,4-benzothiazepines as useful in the treatment of a hyperlipidemic condition.

WO94/18184 discloses selected 1,4-benzothiazepines as useful in the treatment of a hyperlipidemic condition.

WO96/05188 discloses selected 1,4-benzothiazepines as useful in the treatment of a hyperlipidemic condition.

WO98/05657 discloses selected 2,3-dihydro-1,4-benzothiazepines as therapeutic agents.

U.S. Pat. No. 5,910,494 discloses selected 1,4-benzothiazepines as useful in the treatment of a hyperlipidemic condition.

U.S. Pat. No. 6,020,330 discloses selected 1,4-benzothiazepines as useful in the treatment of a hyperlipidemic condition.

WO96/16051 discloses selected 1,5-benzothiazepines as useful in the treatment of a hyperlipidemic condition.

WO99/35135 discloses selected 1,5-benzothiazepines as useful in the treatment of a hyperlipidemic condition.

M. Booker et al., “Ileal Bile Acid Transport Inhibitors As Potential Hypocholesterolemic Agents,” Curr. Opin. In Cardiovascular, Pulmonary & Renal Invest. Drugs, Vol. 2, No. 3, pp. 208-215 (2000), discloses 1,4- and 1,5-benzothiepines (including Glaxo Wellcome compounds 2164U90 and 264W94) as useful for the treatment of a hyperlipidemic condition.

In addition, selected benzothiepines have been disclosed in the literature as agents for the treatment of a hyperlipidemic condition. For example, U.S. Pat. No. 5,994,391 discloses substituted benzothiepine compounds (including 5-(substituted phenyl)-benzothiepine compounds) for use as ASBT inhibitors. WO99/64409 discloses similar 5-(substituted phenyl)-benzothiepine compounds wherein the phenyl substituent comprises a mono-, di-, tri- or tetrasaccharide moiety as useful for the treatment of a hyperlipidemic condition.

Further, other classes of compounds have been disclosed in the literature as agents for the treatment of a hyperlipidemic condition. For example, PCT Patent Application No. WO94/24087 discloses a group of substituted naphthalene compounds as useful for the treatment of a hyperlipidemic condition.

### BRIEF SUMMARY OF THE INVENTION

A first aspect of the invention comprises novel 1,4- and 1,5-benzothiazepine compounds corresponding to Formula I (as later defined in the Detailed Description) that are effective agents for the treatment of a hyperlipidemic condition or conditions.

Another aspect of the invention comprises pharmaceutical compositions comprising one or more of the novel 1,4- and 1,5-benzothiazepine compounds corresponding to Formula I that are suitable for use in treating a hyperlipidemic condition or conditions.

Still another aspect of the invention comprises methods for the treatment of a hyperlipidemic condition or conditions comprising administering to a subject a therapeutically effective amount of one or more of the novel 1,4- and 1,5-benzothiazepine compounds corresponding to Formula I.

Still another aspect of the invention comprises methods of making the novel 1,4- and 1,5-benzothiazepine compounds corresponding to Formula I.

Still another aspect of the invention comprises novel 1,4- and 1,5-benzothiazepine compounds corresponding to Formula VII (as later defined in the Detailed Description) that are effective agents for the treatment of a hyperlipidemic condition or conditions.

Still another aspect of the invention comprises pharmaceutical compositions comprising one or more of the novel 1,4- and 1,5-benzothiazepine compounds corresponding to Formula VII that are suitable for use in treating a hyperlipidemic condition or conditions.

Still another aspect of the invention comprises methods for the treatment of a hyperlipidemic condition or conditions comprising administering to a subject a therapeutically effective amount of one or more of the novel 1,4- and 1,5-benzothiazepine compounds corresponding to Formula VII.

Still another aspect of the invention comprises methods of making the novel 1,4- and 1,5-benzothiazepine compounds corresponding to Formula VII.

Other aspects of the invention will be in part apparent and in part pointed out hereinafter.

### DETAILED DESCRIPTION OF THE PREFERRED EMBODIMENTS OF THE INVENTION

The present invention comprises novel 1,4- and 1,5-benzothiazepine compounds that are safe and effective anti-hyperlipidemic agents. These compounds generally exhibit one or more superior characteristics relative to conventional 1,4- and 1,5-benzothiazepine compounds previously disclosed in the literature as therapeutic agents. These characteristics can include, but are not limited to, for example: (a) improved potency, (b) an improved solubility profile, (c) improved compatibility with conventional routes of oral administration, (d) an improved safety profile, and (e) elimination of a chiral center at the 3-position carbon ring atom without a significant loss in potency relative to the corresponding conventional 1,4- and 1,5-benzothiazepine compounds having a chiral center at the 3-position carbon ring atom and lacking the novel substituent(s) present in the claimed compounds.

The compounds of the present invention are useful for, but not limited to, the treatment of a hyperlipidemic condition or conditions in a subject, including the prophylactic or preventative treatment of a hyperlipidemic condition or conditions in a subject. The methods, combinations, compositions and kits of the present invention also are useful for the prophylaxis and/or treatment of gallstones. Besides being useful for human treatment, these methods and compounds are also useful for veterinary treatment of companion animals, exotic animals and farm animals, including mammals, rodents, and the like. More preferred animals include horses, dogs, and cats.

More specifically, the present invention comprises a class of compounds useful in treating a hyperlipidemic condition that is defined by Formula I:

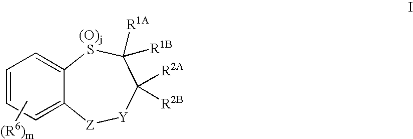

wherein:

j is 0, 1 or 2; and

m is 0, 1, 2, 3 or 4; and

R^{1A} and R^{1B} are independently selected from the group consisting of hydrogen and hydrocarbyl, wherein said hydrocarbyl may be optionally substituted with one or more groups comprising one or more heteroatoms, and

wherein said hydrocarbyl optionally may have one or more carbon atoms replaced by one or more heteroatoms independently selected from the group consisting of oxygen, nitrogen, sulfur and phosphorus;

R^{2A} and R^{2B} are independently selected from the group consisting of hydrogen and hydrocarbyl, wherein said hydrocarbyl may be optionally substituted with one or more groups comprising one or more heteroatoms, and

wherein said hydrocarbyl optionally may have one or more carbon atoms replaced by one or more heteroatoms independently selected from the group consisting of oxygen, nitrogen, sulfur and phosphorus;

one of Z and Y is NR³ and the other of Z and Y is CHR⁴;

wherein R³ and R⁴ are independently selected from the group consisting of hydrogen, oxo, hydrocarbyl; —R⁵; —OR⁹; —NR⁹R¹⁰; —SR⁹; —S(O)R⁹; —SO₂R⁹; and —SO₃R⁹; wherein said hydrocarbyl may be optionally substituted with one or more groups comprising one or more heteroatoms, and wherein said hydrocarbyl optionally may have one or more carbon atoms replaced by one or more heteroatoms independently selected from the group consisting of oxygen, nitrogen, sulfur and phosphorus;

wherein R⁹ and R¹⁰ are independently selected from the group consisting of hydrogen; hydrocarbyl; amino; and hydrocarbylamino; wherein said hydrocarbyl moeities may be optionally substituted with one or more groups comprising one or more heteroatoms, and wherein said hydrocarbyl moieties optionally may have one or more carbon atoms replaced by one or more heteroatoms independently selected from the group consisting of oxygen, nitrogen, sulfur and phosphorus; and

R⁵ is selected from the group consisting of hydrogen; alkyl; cycloalkyl; alkenyl; alkynyl; aryl; heterocyclyl; quaternary heterocyclyl; —OR⁹; —SR⁹; —S(O)R⁹; —SO₂R⁹; and —SO₃R⁹;

wherein R⁵ group optionally may be substituted with one or more radicals independently selected from the group consisting of halogen; —NO₂; —CN; oxo; hydrocarbyl; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P⁺R¹³R¹⁴; —P⁺R¹⁴R¹⁵A⁻; ——P(OR¹³)OR¹⁴; —S⁺R¹³R¹⁴A⁻; and —N⁺R¹³R¹⁴R¹⁵A⁻; wherein said hydrocarbyl may be optionally substituted with one or more groups comprising one or more heteroatoms, and wherein said hydrocarbyl optionally may have one or more carbon atoms replaced by one or more heteroatoms independently selected from the group consisting of oxygen, nitrogen, sulfur and phosphorus;

wherein R¹³, R¹⁴, and R¹⁵ are independently selected from the group consisting of hydrogen or hydrocarbyl, wherein said hydrocarbyl may be optionally substituted with one or more groups comprising one or more heteroatoms, and wherein said hydrocarbyl optionally may have one or more carbon atoms replaced by one or more heteroatoms independently selected from the group consisting of oxygen, nitrogen, sulfur and phosphorus; or

wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or

wherein R¹⁴ and R¹⁵ together with the nitrogen atom to which they are attached form a cyclic ring; and

wherein A⁻ is a pharmaceutically acceptable anion, and M is a pharmaceutically acceptable cation; and

wherein R⁹ is as defined above; and

one or more R⁶ radicals are independently selected from the group consisting of hydrogen; halogen; —CN; —NO₂; hydrocarbyl; —R⁵; —OR¹³; —NR¹³R¹⁴; —SR¹³; —S(O)R¹³; —S(O)₂R¹³; —SO₃R¹³; —S⁺R¹³R¹⁴A⁻; —NR¹³OR¹⁴; —NR¹³NR¹⁴R¹⁵; —OM; —SO₂OM; —SO₂NR¹³R¹⁴; —NR¹⁴C(O)R¹³; —C(O)OM; —S(O)NR¹³R¹⁴; —N⁺R¹³R¹⁴R¹⁵A⁻; —PR¹³R¹⁴; —P(O)R¹³R¹⁴; —P⁺R¹³R¹⁴R¹⁵A⁻; amino acid residue; peptide residue; polypeptide residue; and carbohydrate residue, wherein said hydrocarbyl may be optionally substituted with one or more groups comprising one or more heteroatoms, and wherein said hydrocarbyl optionally may have one or more carbon atoms replaced by one or more heteroatoms independently selected from the group consisting of oxygen, nitrogen, sulfur and phosphorus; and

wherein R¹³, R¹⁴, R¹⁵, A⁻, and M are as defined above; or

a pharmaceutically acceptable salt, solvate, or prodrug thereof; and

provided that at least one of R³, R⁴ and R⁶ is R⁵; and

provided that at least one of the following conditions is satisfied:

(a) the R⁵ moiety possesses an overall positive charge; and/or

(b) the R⁵ moiety comprises a quaternary ammonium group or a quaternary amine salt; and/or

(c) the R⁵ moiety comprises a phosphonic acid group or at least two carboxyl groups; and/or

(d) the R⁵ moiety comprises a polyethylene glycol group having a molecular weight of at least 1000.

Preferably, the class of compounds is defined by Formula I wherein:

j is 0, 1 or 2; and

m is 0, 1, 2, 3 or 4; and

R^{1A} and R^{1B} are independently selected from hydrogen and alkyl; and

R^{2A} and R^{2B} are independently selected from hydrogen, alkyl, alkenyl, alkynyl, cycloalkyl, cycloalkylalkyl, aryl and aralkyl; or

R^{2A} and R^{2B} together with the carbon atom to which they are attached form a C₃₋₁₀ cycloalkyl group; and

one of Z and Y is NR³ and the other of Z and Y is CHR⁴;

wherein R³ and R⁴ are independently selected from the group consisting of hydrogen, oxo, acyl, thioacyl, and R⁵; and

wherein R⁵ is selected from the group consisting of alkyl; cycloalkyl; alkenyl; alkynyl; aryl; heterocyclyl; quaternary heterocyclyl; —OR⁹; —SR⁹; —S(O)R⁹; —S(O)₂R⁹; and —SO₃R⁹;

wherein the R⁵ alkyl; cycloalkyl; alkenyl; alkynyl; aryl; heterocyclyl; and quaternary heterocyclyl radical is substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³ OR¹⁴; —NR¹³NR¹⁴R¹⁵; —CO₂R¹³; —OM; —SO₂OM; —S₂NR¹³R¹⁴; —C(O)NR¹³R¹⁴; —C(O)OM; —COR¹³; —NR¹³C(O)R¹⁴; —NR¹³C(O)NR¹⁴ R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and

wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ radical optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷—NR⁷R⁸; —SR⁷; —S(O)R⁷; —SO₂R⁷; —SO₃R⁷; —CO₂R⁷; —CONR⁷R⁸; —N⁺R⁷R⁸R⁹A⁻; —P(O)R⁷R⁸; —PR⁷R⁸; —P⁺R⁷R⁸R⁹A⁻; and —P(O)(OR⁷)OR⁸; and

wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ radical optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and

wherein R⁷ and R⁸ are independently selected from the group consisting of hydrogen; and alkyl; and

wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and

wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or

R¹¹ and R¹² together with the carbon atom to which they are attached form a cyclic ring; and

wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or

wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or

wherein R¹⁴ and R¹⁵ together with the nitrogen atom to which they are attached form a cyclic ring; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaiminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A⁻; —S⁺R⁹R¹⁰A-; and carbohydrate residue; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and

wherein R¹⁶ and R¹⁷ are independently selected from the group consisting of R⁹ and M; and

wherein A⁻ is a pharmaceutically acceptable cation and M is a pharmaceutically acceptable cation; and

one or more R⁶ radicals are independently selected from the group consisting of R⁵, hydrogen; halogen; —CN; —NO₂; alkyl; cycloalkyl; polyalkyl; haloalkyl; hydroxyalkyl; alkenyl; alkynyl; aryl; heterocyclyl; quaternary heterocyclyl; arylalkyl; heterocyclylalkyl; polyether; acyloxy; —OR¹³; —NR¹³R¹⁴; —SR¹³; —S(O)R¹³; —S(O)₂R¹³; —SO₃R¹³; —S⁺R¹³R¹⁴A⁻; —NR¹³OR¹⁴; —NR¹³NR¹⁴R¹⁵; —CO₂R¹³; —OM; —SO₂OM; —SO₂NR¹³R¹⁴; —NR¹⁴C(O)R¹; —C(O)NR¹³R¹⁴; —C(O)OM; —COR¹³; —OR⁸; —S(O)ₙNR¹³R¹⁴; —NR³R⁸; —NR⁸OR; —N⁺R¹³R¹⁴R¹⁵A⁻; —PR¹³R¹⁴; P(O)R¹³R¹⁴; —P⁺R¹³R¹⁴R¹⁵A⁻; amino acid residue; peptide residue; polypeptide residue; and carbohydrate residue;

wherein the R⁶ alkyl; cycloalkyl; polyalkyl; haloalkyl; hydroxyalkyl; alkenyl; alkynyl; aryl; heterocyclyl; arylalkyl; heterocyclylalkyl; polyether; acyloxy radicals optionally may be further substituted with one or more radicals selected from the group consisting of halogen; —CN; oxo; —OR¹⁶; —NR⁹R¹⁰; —N⁺R⁹R¹⁰R^{w}A⁻; —SR^{16;}

—S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹R¹²A⁻; —S⁺R⁹R¹⁰A⁻; and carbohydrate residue; and

wherein the R⁶ quaternary heterocyclyl radical optionally may be substituted with one or more radicals selected from the group consisting of halogen; —CN; —NO₂; oxo; alkyl; cycloalkyl; polyalkyl; haloalkyl; hydroxyalkyl; alkenyl; alkynyl; aryl; heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴ R¹⁵; —CO₂R¹³; OM; —SO₂OM; —SO₂NR¹³R¹⁴; —C(O)NR¹³R¹⁴; —C(O)OM; —COR¹³; —P(O)R¹³R¹⁴; —P¹³R¹⁴; —P⁺R¹³R¹⁴R¹⁵A⁻; —P¹³)OR¹⁴); —S⁺R¹³R¹⁴A⁻; —N⁺R¹³R¹⁴R¹⁵A⁻; and carbohydrate residue; and

wherein the R⁶ radicals comprising carbon optionally may have one or more carbons replaced by —O—; —NR¹³—; —N⁺R¹³R¹⁴A⁻-; —S—; —SO—; —SO₂—; —S⁺R¹³A⁻-; —PR¹³—; —P(O)R¹³; —PR¹³R¹⁴; —P⁺R¹³R¹⁴A⁻; phenylene; amino acid residue; peptide residue; polypeptide residue; carbohydrate residue; polyether; or polyalkyl; wherein said phenylene; amino acid residue; peptide residue; polypeptide residue; carbohydrate residue; and polyalkyl optionally may have one or more carbons replaced by —O—; —NR⁹—; —N⁺R⁹R¹⁰A⁻-; —S—; —SO—; —SO₂—S⁺R⁹A⁻-; —PR⁹—; —P⁺R⁹R¹⁰A⁻-; or —P(O)R⁹—; and

wherein R¹⁸ is selected from the group consisting of alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl; and

wherein the R¹⁸ alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl radicals optionally may be substituted with one or more radicals selected from the group consisting of halogen; —CN; NO₂; oxo; —OR⁹; —NR⁹R¹⁰; —N⁺R⁹R¹¹R¹²A⁻; —SR⁹; —S(O)R⁹; —SO₂R⁹; —SO₃R⁹; —CO₂R⁹; —CONR⁹R¹⁰; —SO₂OM; —SO₂NR⁹R¹⁰; —PR⁹R¹⁰; —P(OR¹³)OR¹⁴; —PO(OR¹⁶)OR¹⁷; and —C(O)OM; or

a pharmaceutically acceptable salt, solvate, or prodrug thereof;

provided that at least one of R³, R⁴ and R⁶ is R⁵; and

provided that at least one of the following conditions is satisfied:

(a) the R⁵ moiety possesses an overall positive charge; and/or

(b) the R⁵ moiety comprises a quaternary ammonium group or a quaternary amine salt; and/or

(c) the R⁵ moiety comprises a phosphonic acid group or at least two carboxyl groups; and/or

(d) the R⁵ moiety comprises a polyethylene glycol group having a molecular weight of at least 1000.

In one embodiment of the compounds of Formula I, R⁵ is aryl substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SO₂N¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and

wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ aryl optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷—CONR⁷R⁸—N R⁷R⁸R⁹A⁻; —P(O)R⁷R⁸; —P⁺R⁷R⁸; —P⁺R⁷R⁸R⁹A⁻; and —P(O)(OR⁷)OR⁸; and

wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ aryl optionally may have one or more carbons replaced by —O—; —NR⁷—; —N⁺R⁷R⁸A⁻-; —S—; —SO—; —SO₂—; —S⁺R⁷A⁻; —PR⁷ —; —P(O)R⁷—; —P⁺R⁷R⁸A⁻-; or phenylene; and

wherein R⁷ and R⁸ are independently selected from the group consisting of hydrogen; and alkyl; and

wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and

wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or

R¹¹ and R¹² together with the carbon atom to which they are attached form a cyclic ring; and

wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or

wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or

wherein R¹⁴ and R¹⁵ together with the nitrogen atom to which they are attached form a cyclic ring; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰—N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —P⁺R⁹R¹⁰R¹A⁻; —S⁺R⁹R¹⁰A⁻; and carbohydrate residue; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻; —P(O)R⁹—; phenylene; carbohydrate residue; amino acid residue; peptide residue; or polypeptide residue; and

wherein R¹⁶ and R¹⁷ are independently selected from the group consisting of R⁹ and M; and

wherein A⁻ is a pharmaceutically acceptable anion and M is a pharmaceutically acceptable cation.

In another embodiment of the compounds of Formula I, R⁵ is:

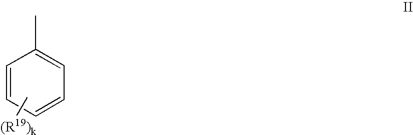

wherein

k is 0, 1, 2, 3 or 4; and

one or more R¹⁹ are independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR³NR⁴R⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and

wherein the R¹⁹ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A⁻; —P(O)R⁷R⁸; —PR⁷R⁸—P⁺R⁷R⁸R⁹A⁻; and —P(O)(OR⁷)OR⁸; and

wherein the R¹⁹ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and

wherein R⁷ and R⁸ are independently selected from the group consisting of hydrogen; and alkyl; and

wherein R⁹, R¹⁰ and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and

wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or

R¹¹ and R¹² together with the carbon atom to which they are attached form a cyclic ring; and

wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or

wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or

wherein R¹⁴ and R¹⁵ together with the nitrogen atom to which they are attached form a cyclic ring; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N+R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A-; —S⁺R⁹R¹⁰A-; and carbohydrate residue; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and

wherein R¹⁶ and R¹⁷ are independently selected from the group consisting of R⁹ and M; and

wherein A⁻ is a pharmaceutically acceptable anion and M is a pharmaceutically acceptable cation.

In another embodiment, R⁵ is:

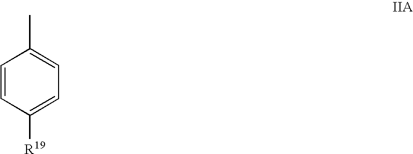

wherein R¹⁹ is as defined above.

In another embodiment, R⁵ is:

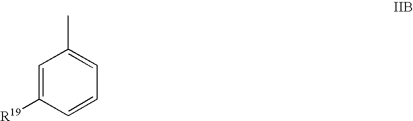

wherein R¹⁹ is as defined above.

In another embodiment:

R¹⁹ is independently selected from the group consisting of —OR¹³, —NR¹³R¹⁴, —NR¹³C(O)R¹⁴, —OC(O)NR¹³R¹⁴, and —NR¹³SO₂R¹⁴ and

wherein R¹³, R¹⁴, and R¹⁵ are independently selected from the group consisting of alkyl, polyether, aryl, quaternary heterocycle, arylalkyl, heterocyclylalkyl, quaternary heterocyclylalkyl, alkylheterocyclylalkyl, and alkylammoniumalkyl,

wherein alkyl optionally has one or more carbons replaced by O or N⁺R⁹R¹⁰A⁻, and

wherein R¹³ R¹⁴ and R¹⁵ are optionally substituted with one or more groups selected from the group consisting of hydroxy, carboxy, alkyl, quaternary heterocyclylalkyl, —SR⁹, —S(O)R⁹, —S(O)₂R⁹, —S(O)₃R⁹, —NR⁹R¹⁰, N⁺R⁹R¹¹R¹²A⁻, —CONR⁹R¹⁰, and —PO(OR¹⁶)OR¹⁷, and

wherein R⁹ and R¹⁰ are independently selected from the group consisting of hydrogen, alkyl, heterocyclylalkyl, carboxyalkyl, carboalkoxyalkyl, and carboxyalkylheterocycle; and

wherein R¹¹ and R¹² are independently alkyl; and

wherein A⁻ is a pharmaceutically acceptable anion and M is a pharmaceutically acceptable cation.

In another embodiment:

R¹⁹ is independently selected from the group consisting of −0R¹³, —NR¹³R¹⁴, —NR¹³C(O)R¹⁴—OC(O)NR¹³R¹⁴, and —NR¹³SO₂R¹⁴, and

wherein R¹³, R¹⁴, and R¹⁵ are independently selected from the group consisting of polyether, aryl, quaternary heterocycle, arylalkyl, heterocyclylalkyl, quaternary heterocyclylalkyl, and alkylheterocyclylalkyl,

wherein R¹³, R¹⁴, and R¹⁵ are optionally substituted with one or more groups selected from the group consisting of hydroxy, carboxy, alkyl, quaternary heterocyclylalkyl, —SR⁹, —S(O)R⁹, —S(O)₂R⁹, —S(O)₃R⁹, —NR⁹R¹⁰, N⁺R⁹R¹¹R¹²A⁻, —CONR⁹R¹⁰, and —PO(OR¹⁶)OR¹⁷, and

wherein R⁹ and R¹⁰ are independently selected from the group consisting of hydrogen, alkyl, heterocyclylalkyl, carboxyalkyl, carboalkoxyalkyl, and carboxyalkylheterocycle; and

wherein R¹¹ and R¹² are independently alkyl; and

wherein A⁻ is a pharmaceutically acceptable anion and M is a pharmaceutically acceptable cation.

In another embodiment, R¹⁹ is selected from the group consisting of:

TABLE 1

| | R¹⁹ |
|---|---|
| (1) | 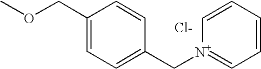 |
| (2) | 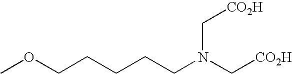 |
| (3) | 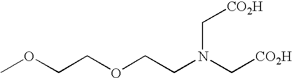 |
| (4) | 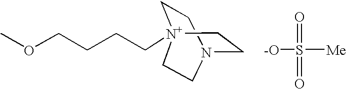 |
| (5) | 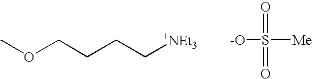 |
| (6) | 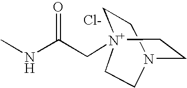 |
| (7) | 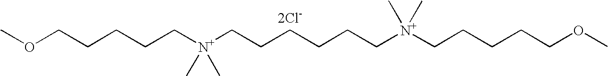 |
| (8) | 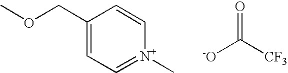 |
| (9) | 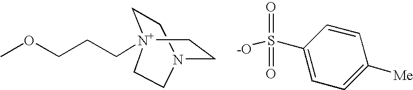 |
| (10) | 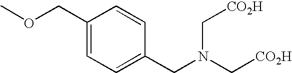 |
| (11) | 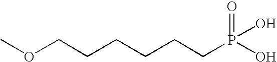 |
| (12) | 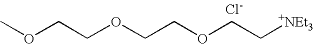 |
| (13) | 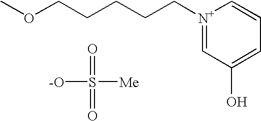 |
| (14) | 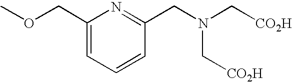 |
| (15) | 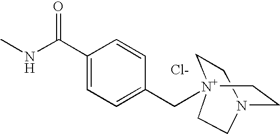 |
| (15a) | 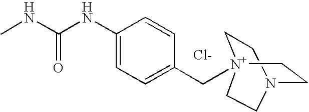 |
| (16) | 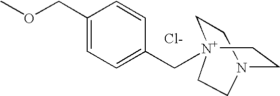 |
| (17) | 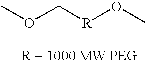 |
| (18) | 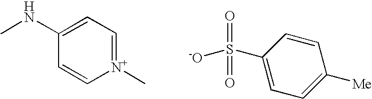 |
| (19) | 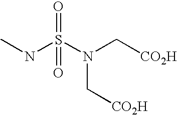 |
| (20) | 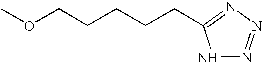 |
| (21) | 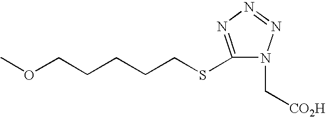 |
| (22) | 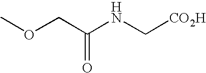 |
| (23) | 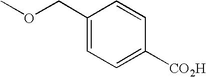 |
| | 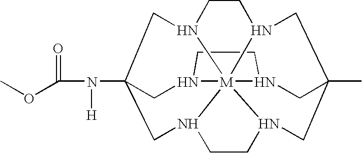 |
| (24) | M = Co^{II,III}, Mn^{II,III}, Fe^{II,III}, Ni^{II,III}, |
| | Cr^{III}, Cu^{II}, Zn^{II}, Cd^{II}, Ga^{III}, In^{III}, V^{IV}, |
| | Ru^{II}, Pr^{IV}, Rh^{III} or Ir^{III} |
| (25) | 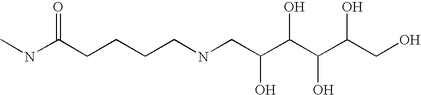 |
| (26) | 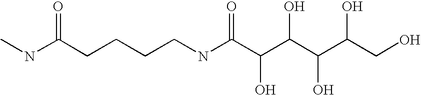 |
| (27) | 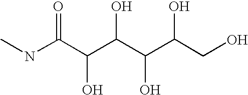 |
| (28) | 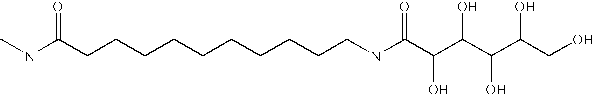 |
| (29) | 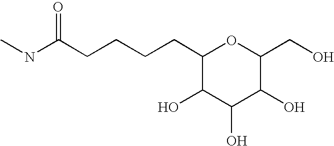 |
| (30) | 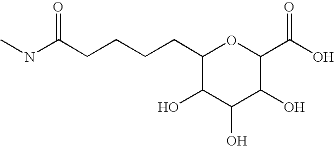 |
| (31) | 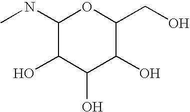 |
| (32) | 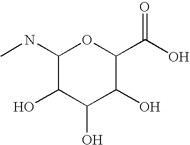 |
| (33) | 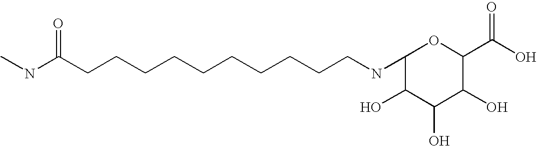 |
| (34) | 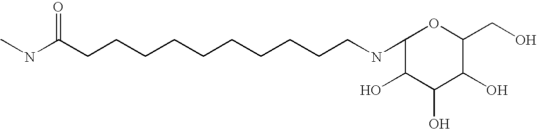 |
| (35) | 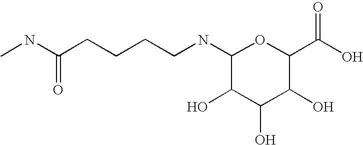 |
| (36) | 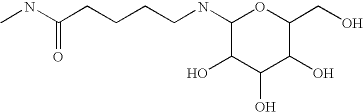 |
| (37) | 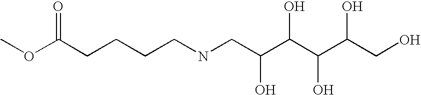 |
| (38) | 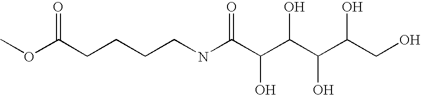 |
| (39) | 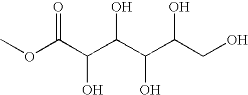 |
| (40) | 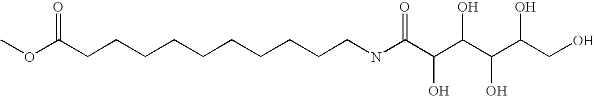 |
| (41) | 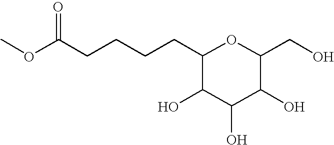 |
| (42) | 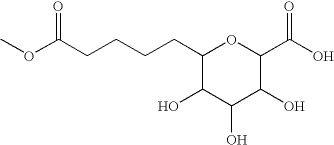 |
| (43) | 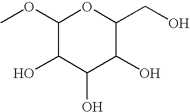 |
| (44) | 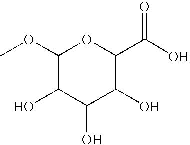 |
| (45) | 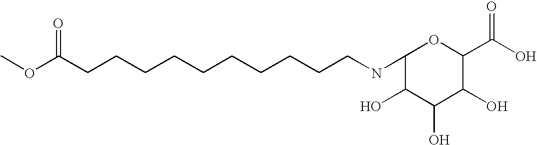 |
| (46) | 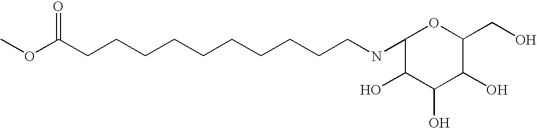 |
| (47) | 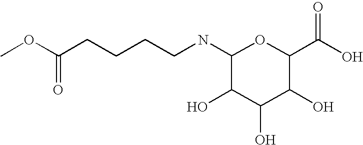 |
| (48) | 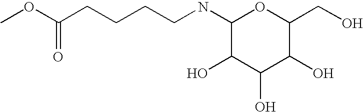 |
| (49) | 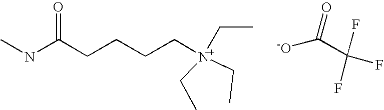 |
| (50) | 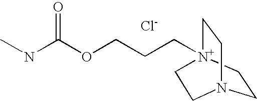 |
| (51) | 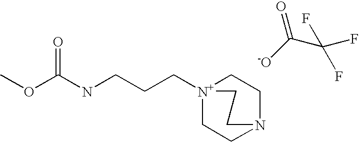 |
| (52) | 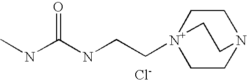 |
| (53) | 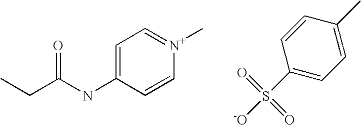 |
| (54) | 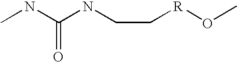 |
| (55) | 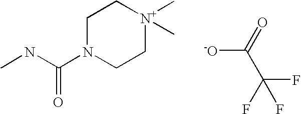 |
| (56) | 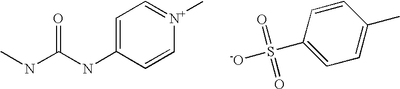 |
| (57) | 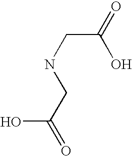 |
| (58) | 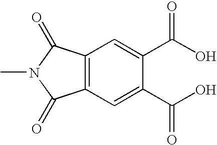 |
| (59) | 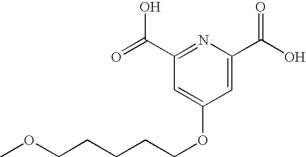 |
| (60) | 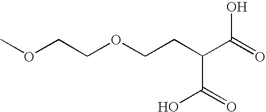 |
| (61) | 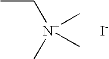 |
| (62) | 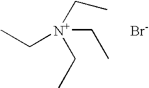 |
| (63) | 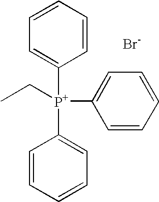 |
| (64) | 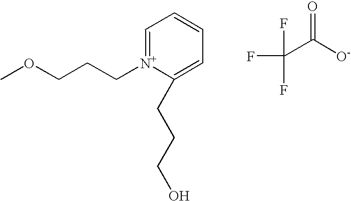 |
| (65) | 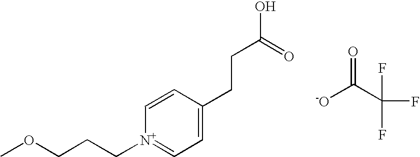 |
| (66) | 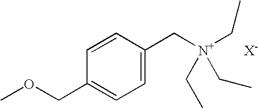 |
| (67) | 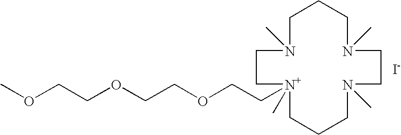 |
| (68) | 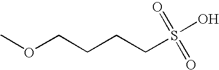 |
| (69) | 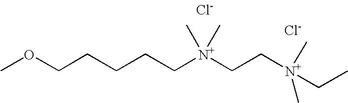 |
| (70) | 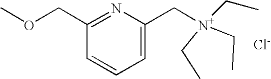 |

Optionally, R¹⁹ may be selected from the following: (1)-(24), (25)-(48) or (49)-(70) from Table 1. Further, R¹⁹ may be acidic or contain a quaternary ammonium nitrogen. Even further, R¹⁹ may be selected from the following: (1)-(5), (6)-(10), (11)-(15), (16)-(20), (21)-(25), (26)-(30), (31)-(35), (36)-(40), (41)-(45), (46)-(50), (51)-(55), (56)-(60), (61)-(65), (66)-(70), or combinations thereof.

In another embodiment of the compounds of Formula I, R³ is R⁵; and

R⁴ is selected from the group consisting of hydrogen and alkyl.

In another embodiment of the compounds of Formula I, R³ is selected from the group consisting of hydrogen and alkyl; and R⁴ is R⁵.

In another embodiment of the compounds of Formula I:

R³ is R⁵; and

R⁴ is selected from the group consisting of hydrogen; oxo; alkyl; cycloalkyl; aryl; heterocyclyl; acyl, thioacyl, and —OR⁹;

wherein the R⁴ alkyl; cycloalkyl; aryl; heterocyclyl radical is substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R 13R¹⁴A⁻; and N⁺R¹³R¹⁴R¹⁵A⁻; and

wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁴ radical optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸ R⁹A-; —P(O)R⁷R⁸; —PR⁷R⁸; —P⁺R⁷R⁸R⁹A⁻; and

—P(O)(OR⁷)OR⁸; and

wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁴ radical optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and

wherein R⁷ and R⁸ are independently selected from the group consisting of hydrogen; and alkyl; and

wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and

wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or

R¹¹ and R¹² together with the carbon atom to which they are attached form a cyclic ring; and

wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or

wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or

wherein R¹⁴ and R¹⁵ together with the nitrogen atom to which they are attached form a cyclic ring; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N+R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A⁻; —S⁺R R¹⁰A⁻; and carbohydrate residue; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻; —P(O)R⁹—; phenylene; carbohydrate residue; amino acid residue; peptide residue; or polypeptide residue; and

wherein R¹⁶ and R¹⁷ are independently selected from the group consisting of R⁹ and M; and

wherein A⁻ is a pharmaceutically acceptable cation and M is a pharmaceutically acceptable cation.

In another embodiment of the compounds of Formula I:

R³ is selected from the group consisting of hydrogen; oxo; alkyl; cycloalkyl; aryl; heterocyclyl; acyl, thioacyl, and —OR⁹;

wherein the R³ alkyl; cycloalkyl; aryl; heterocyclyl radical is substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R 14 P⁺R¹³R¹⁴R¹⁵A⁻; —P(OR¹³)OR¹⁴; —S⁺R¹³R¹⁴A; and —N⁺R¹³R¹⁴R¹⁵ A⁻; and

wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³ radical optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷—NR⁷R⁸; —SR⁷; —S(O)R⁷; —SO₂R⁷; —SO₃R⁷; CO₂R⁷; —CONR⁷R⁸; —N⁺R⁷R⁸R⁹A⁻; —P(O)R⁷R⁸; —PR⁷R⁸; —P⁺R⁷R⁸R⁹A⁻; and —P(O)(OR⁷)OR⁸; and

wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³ radical optionally may have one or more carbons replaced by —O—; —NR⁷—; —N⁺R⁷R⁸A⁻-; —S—; —SO—; —SO₂—; —S+A⁻-; —PR⁷—; —P(O)R⁷—; —P⁺R⁷R⁸A⁻-; or phenylene; and

wherein R⁷ and R⁸ are independently selected from the group consisting of hydrogen; and alkyl; and

wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and

wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or

R¹¹ and R¹² together with the carbon atom to which they are attached form a cyclic ring; and

wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or

wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or

wherein R¹⁴ and R¹⁵ together with the nitrogen atom to which they are attached form a cyclic ring; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰R¹¹A-; —S⁺R⁹R¹⁰A-; and carbohydrate residue; and

wherein the R³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and

wherein R¹⁶ and R¹⁷ are independently selected from the group consisting of R⁹ and M; and

wherein A⁻ is a pharmaceutically acceptable cation and M is a pharmaceutically acceptable cation; and

R⁴ is R⁵.

Within the compounds of Formula I is a class of compounds of specific interest corresponding to Formula IA:

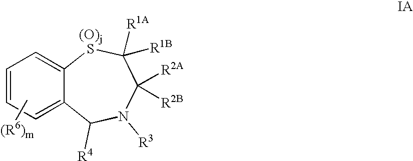

wherein: j=1 or 2;

R^{1A} and R^{1B} are independently selected from hydrogen and alkyl; and

R^{2A} and R^{2B} are independently selected from hydrogen, alkyl, alkenyl, alkynyl, cycloalkyl, cycloalkylalkyl, aryl, and aralkyl; or

R^{2A} and R^{2B} together with the carbon atom to which they are attached form a C₃₋₇ cycloalkyl group; and

independently selected from the group consisting of hydrogen, oxo, acyl, thioacyl, and R⁵; and

j, m, R³, R⁴ and R⁶ are as previously defined above for the compounds of Formula I;

provided that at least one of R³, R⁴ and R⁶ is R⁵; and

provided that the R⁵ alkyl, cycloalkyl, aryl, heterocyclyl, and —OR⁹ radicals are not substituted with —O(CH₂)₁₋₄NR′R″R′″ wherein R′, R″ and R′″ are independently selected from hydrogen and alkyl; and

provided that at least one of the following conditions is satisfied:

(a) the R⁵ moiety possesses an overall positive charge; and/or

(b) the R⁵ moiety comprises a quaternary ammonium group or a quaternary amine salt; and/or

(c) the R⁵ moiety comprises at least two carboxy groups.

Within the compounds of Formula I is another class of compounds of specific interest corresponding to Formula IB:

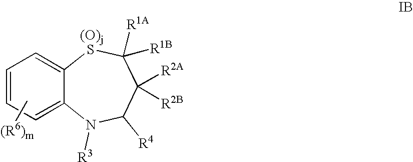

wherein: j=1 or 2;

R^{1A} and R^{1B} are independently selected from hydrogen and alkyl; and

R^{2A} and R^{2B} are independently selected from hydrogen, alkyl, alkenyl, alkynyl, cycloalkyl, cycloalkylalkyl, aryl, and aralkyl; or

R^{2A} and R^{2B} together with the carbon atom to which they are attached form a C₃₋₇ cycloalkyl group; and

independently selected from the group consisting of hydrogen, oxo, acyl, thioacyl, and R⁵; and

j, m, R³, R⁴ and R⁶ are as previously defined above for the compounds of Formula I;

provided that at least one of R³, R⁴ and R⁶ is R⁵; and

provided that the R⁵ alkyl, cycloalkyl, aryl, heterocyclyl, and —OR⁹ radicals are not substituted with —O(CH₂)₁₋₄NR′R″R′″ wherein R′, R″ and R′″ are independently selected from hydrogen and alkyl; and

provided that at least one of the following conditions is satisfied:

(a) the R⁵ moiety possesses an overall positive charge; and/or

(b) the R⁵ moiety comprises a quaternary ammonium group or a quaternary amine salt; and/or

(c) the R⁵ moiety comprises at least two carboxy groups.

Within the compounds of Formula I is a class of compounds of particular interest corresponding to Formula m:

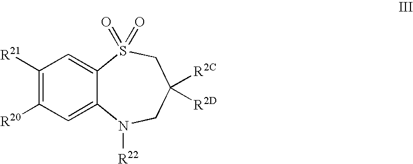

wherein:

R^{2C} and R^{2D} are independently selected from C₁₋₆ alkyl; and

R²⁰ is selected from the group consisting of halogen and R²³;

R²¹ is selected from the group consisting of hydroxy, alkoxy, and R²³; and

wherein R²³ is aryl substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³—NR¹³R¹⁴ SR¹³; —S(O)R³; —SO₂R¹³; —SO₃R¹³; —NR¹³OR¹⁴; —NR¹³NR¹⁴R¹⁵; —CO₂R³; OM; —SO₂OM; —SO₂NR¹³R¹⁴; C(O)NR¹³R¹⁴; —C(O)OM; —COR¹³ NR¹³C(O)R¹⁴; —NR¹³C(O)NR¹⁴R!⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴—P(O)R¹³R ¹⁴; —P⁺R¹³ R¹⁴R¹⁵A-; —P(OR¹³)OR¹⁴; —S⁺R¹³R¹⁴A; and —N⁺R¹³R¹⁴R¹⁵A⁻; and

wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R²³ aryl optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷ R⁸R⁹A⁻; —P(O)R⁷R⁸; —PR⁷R⁸; —P⁺R⁷R⁸R⁹A⁻; and —P(O)(OR⁷)OR⁸; and

wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R²³ aryl optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A³¹ -; or phenylene; and

wherein R⁷ and R⁸ are independently selected from the group consisting of hydrogen; and alkyl; and

wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and

wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or

R¹¹ and R¹² together with the carbon atom to which they are attached form a cyclic ring; and

wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or

wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or

wherein R¹⁴ and R¹⁵ together with the nitrogen atom to which they are attached form a cyclic ring; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R^{10; —PO(OR}¹⁶)OR¹⁷; —P⁹R¹⁰; —P⁺R⁹R¹⁰R¹¹A-; —S⁺R⁹R¹⁰A-; and carbohydrate residue; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and

wherein R¹⁶ and R¹⁷ are independently selected from the group consisting of R⁹ and M; and

wherein A⁻ is a pharmaceutically acceptable anion and M is a pharmaceutically acceptable cation; and

R²² is unsubstituted phenyl or R²³; or

a pharmaceutically acceptable salt, solvate, or prodrug thereof;

provided that at least one of R²⁰, R²¹ and R²² is R²³.

Preferably, R²³ is:

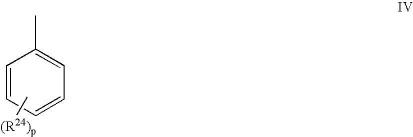

wherein

p is 0, 1, 2, 3 or 4; and

one or more R²⁴ are independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether, —OR¹³; —NR¹³R¹⁴; —SR¹³; —S(O)R¹³; —SO₂R¹³; —SO₃R¹³; —NR¹³NR¹⁴; R¹⁵; —CO₂R¹³, —OM; —SO₂OM; —SO₂NR¹³R¹⁴; —C(O)NR¹³R¹⁴; —C(O)OM; —COR¹³; —NR³ C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and

wherein the R²⁴ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A⁻; —P(O)R⁷R⁸; —PR⁷R⁸—P⁺R⁷R⁸R⁹A⁻; and —P(O)(OR⁷)OR⁸; and

wherein the R²⁴ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and

wherein R⁷ and R⁸ are independently selected from the group consisting of hydrogen; and alkyl; and

wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and

wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or

R¹¹ and R¹² together with the carbon atom to which they are attached form a cyclic ring; and

wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or

wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or

wherein R¹⁴ and R¹⁵ together with the nitrogen atom to which they are attached form a cyclic ring; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N+R⁹R¹⁰R^{w}A⁻; —SR¹⁶; —S(O)R⁹; —SO₂R⁹; —SO₃R¹⁶; —CO₂R¹⁶; —CONR⁹R¹⁰, —SO₂NR⁹R¹⁰; —PO(OR¹⁶OR¹⁷; —P⁹R¹⁰; —P⁺R⁹R¹⁰R¹¹A-; —S⁺R⁹R¹⁰A-; and carbohydrate residue; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and

wherein R¹⁶ and R¹⁷ are independently selected from the group consisting of R⁹ and M; and

wherein A⁻ is a pharmaceutically acceptable anion and M is a pharmaceutically acceptable cation.

In one embodiment, R²³ is:

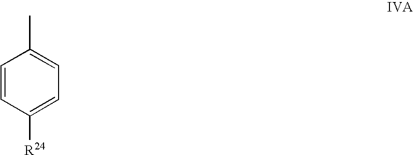

wherein R²⁴ is as defined above.

In another embodiment, R²³ is:

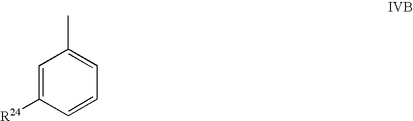

wherein R²⁴ is as defined above.

In another embodiment:

R²⁴ is independently selected from the group consisting of —OR¹³, —NR¹³R¹⁴, NR¹³C(O)R¹⁴—OC(O)NR¹³R¹⁴, and —NR¹³SO₂R¹⁴ and

wherein R¹³, R¹⁴, and R¹⁵ are independently selected from the group consisting of alkyl, polyether, aryl, quaternary heterocycle, arylalkyl, heterocyclylalkyl, quaternary heterocyclylalkyl, alkylheterocyclylalkyl, and alkylammoniumalkyl,

wherein alkyl optionally has one or more carbons replaced by O or N⁺R⁹R¹⁰A-, and

wherein R¹³, R¹⁴, and R¹⁵ are optionally substituted with one or more groups selected from the group consisting of hydroxy, carboxy, alkyl, quaternary heterocyclylalkyl, —SR⁹, —S(O)R⁹, —S(O)₂R⁹, —S(O)₃R⁹, —NR⁹R¹⁰, N⁺R⁹R¹¹R¹²A⁻, —CONR⁹R¹⁰, and —PO(OR¹⁶)OR¹⁷, and

wherein R⁹ and R¹⁰ are independently selected from the group consisting of hydrogen, alkyl, heterocyclylalkyl, carboxyalkyl, carboalkoxyalkyl, and carboxyalkylheterocycle; and

wherein R¹¹ and R¹² are independently alkyl; and

wherein A⁻ is a pharmaceutically acceptable anion and M is a pharmaceutically acceptable cation.

In another embodiment, R²⁴ is selected from the group consisting of:

TABLE 2

| | R²⁴ |
|---|---|
| (1) | 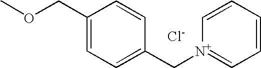 |
| (2) | 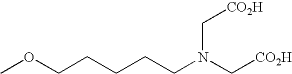 |
| (3) | 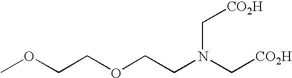 |
| (4) | 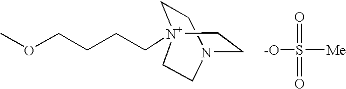 |
| (5) | 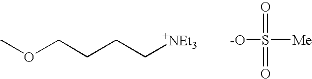 |
| (6) | 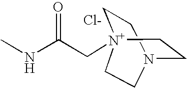 |
| (7) | 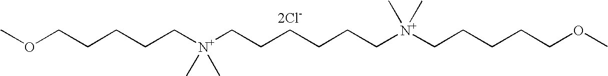 |
| (8) | 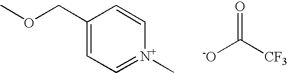 |
| (9) | 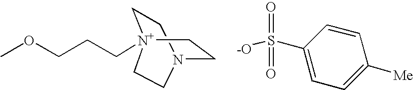 |
| (10) | 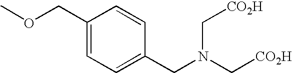 |
| (11) | 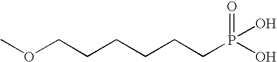 |
| (12) | 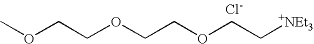 |
| (13) | 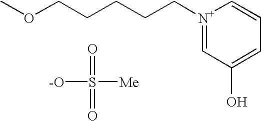 |
| (14) | 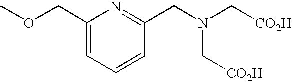 |
| (15) | 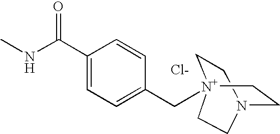 |
| (15a) | 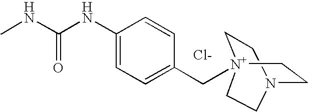 |
| (16) | 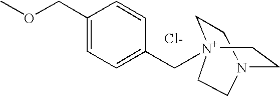 |
| (17) | 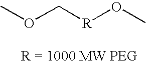 |
| (18) | 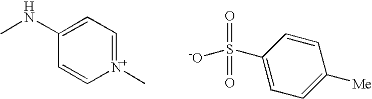 |
| (19) | 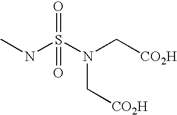 |
| (20) | 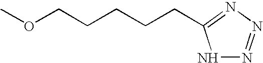 |
| (21) | 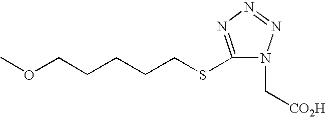 |
| (22) | 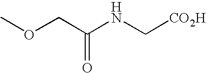 |
| (23) | 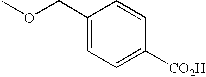 |
| | 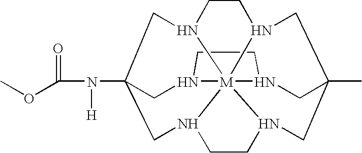 |
| (24) | M = Co^{II,III}, Mn^{II,III}, Fe^{II,III}, Ni^{II,III}, |
| | Cr^{III}, Cu^{II}, Zn^{II}, Co^{II}, Ga^{III}, In^{III}, V^{IV}, |
| | R^{II}, Pr^{IV}, Rh^{III} or Ir^{III} |
| (25) | 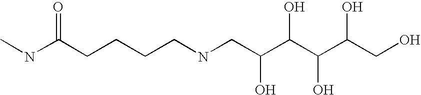 |
| (26) | 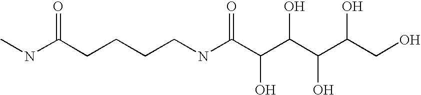 |
| (27) | 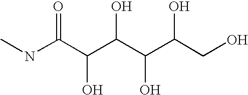 |
| (28) | 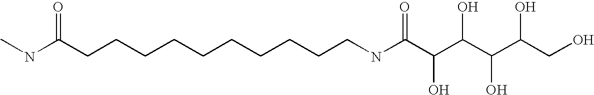 |
| (29) | 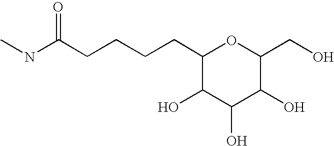 |
| (30) | 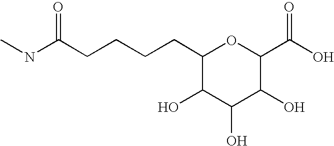 |
| (31) | 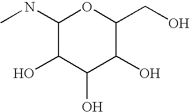 |
| (32) | 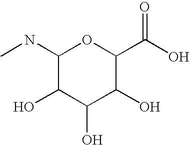 |
| (33) | 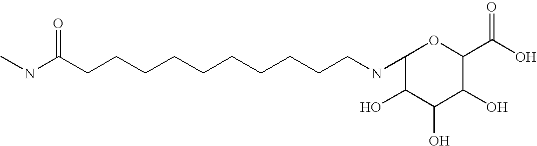 |
| (34) | 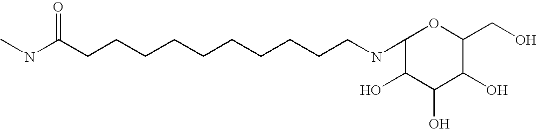 |
| (35) | 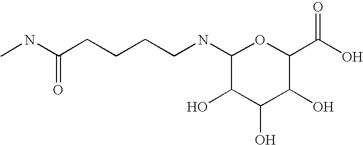 |
| (36) | 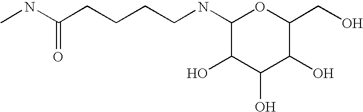 |
| (37) | 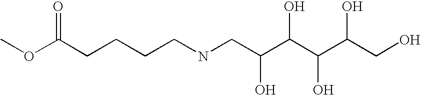 |
| (38) | 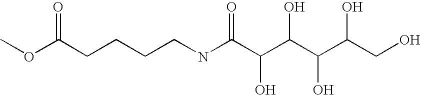 |
| (39) | 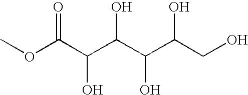 |
| (40) | 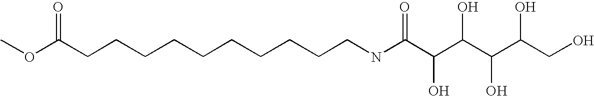 |
| (41) | 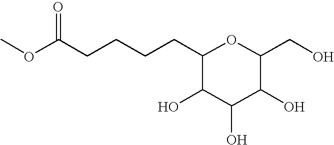 |
| (42) | 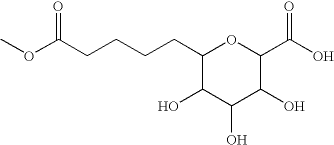 |
| (43) | 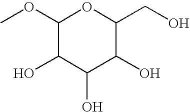 |
| (44) | 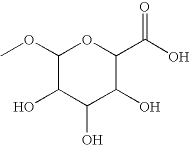 |
| (45) | 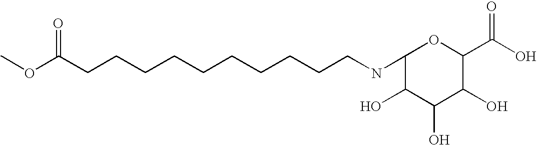 |
| (46) | 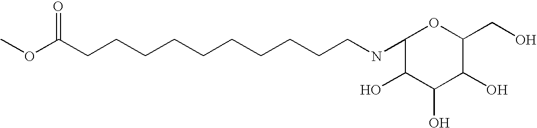 |
| (47) | 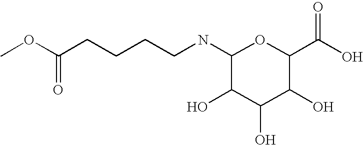 |
| (48) | 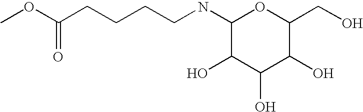 |
| (49) | 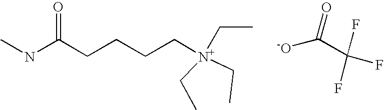 |
| (50) | 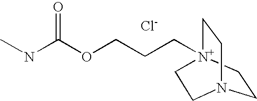 |
| (51) | 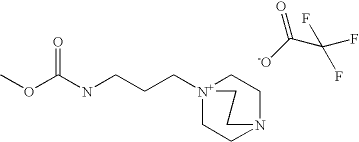 |
| (52) | 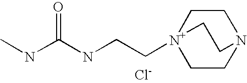 |
| (53) | 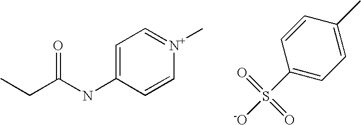 |
| (54) | 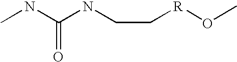 |
| (55) | 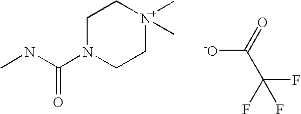 |
| (56) | 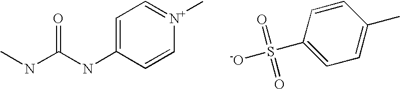 |
| (57) | 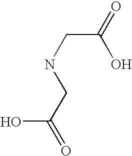 |
| (58) | 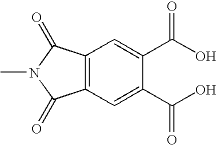 |
| (59) | 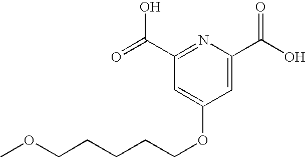 |
| (60) | 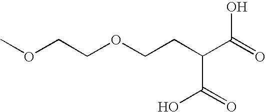 |
| (61) | 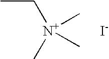 |
| (62) | 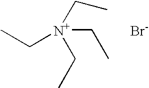 |
| (63) | 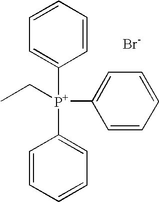 |
| (64) | 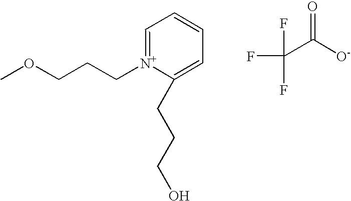 |
| (65) | 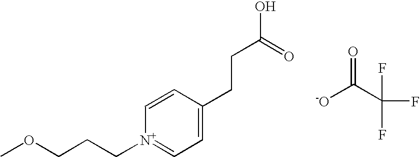 |
| (66) | 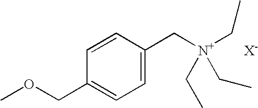 |
| (67) | 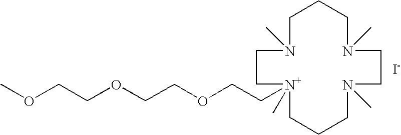 |
| (68) | 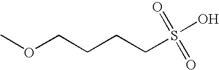 |
| (69) | 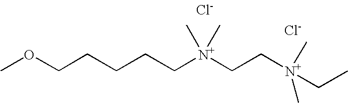 |
| (70) | 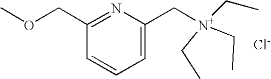 |

Optionally, R²⁴ may be selected from the following: (1)-(24), (25)-(48) or (49)-(70) from Table 2. Further, R²⁴ may be acidic or contain a quaternary ammonium nitrogen. Even further, R²⁴ may be selected from the following: (1)-(5), (6)-(10), (11)-(15), (16)-(20), (21)-(25), (26)-(30), (31)-(35), (36)-(40), (41)-(45), (46)-(50), (51)-(55), (56)-(60), (61)-(65), (66)-(70), or combinations thereof.

In another embodiment of the compounds of Formula III, R²⁰ is chloro, and R²¹ is selected from the group consisting of hydroxy and methoxy.

In another embodiment of the compounds of Formula III, one of R^{2C} and R^{2D} is ethyl and the other of R^{2C} and R^{2D} is n-butyl; R²⁰ is chloro; and R²¹ is hydroxy.

In another embodiment of the compounds of Formula III, one of R^{2C} and R^{2D} is ethyl and the other of R^{2C} and R^{2D} is n-butyl; R²⁰ is chloro; and R²¹ is methoxy.

Within the compounds of Formula I is another class of compounds of particular interest corresponding to Formula V:

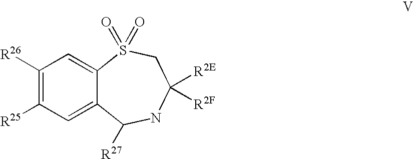

wherein:

R^{2E} and R^{2F} are independently selected from C₁ alkyl; and

R²⁵ and R²⁶ are independently selected from the group consisting of hydrogen, alkoxy, and R²⁸;

wherein R²⁸ is aryl substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³ R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and

wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R²⁸ aryl optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸ ; —SR⁷; —S(O)R⁷; —SO₂R⁷; —SO₃R⁷; —CO₂R⁷; —CONR⁷R⁸; —N+R⁷R⁸R⁹A-; —P(O)R⁷R⁸; —PR⁷R⁸; —P⁺R⁷R⁸R⁹A⁻; and —P(O)(OR⁷)OR⁸; and

wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R²⁸ aryl optionally may have one or more carbons replaced by —O—; —NR⁷—; —N⁺R⁷R⁸A⁻-; —S—; —SO—; —SO₂—; —S⁺R⁷A⁻; —PR⁷—; —P(O)R⁷—; —P⁺R⁷R⁸A⁻-; or phenylene; and

wherein R⁷ and R⁸ are independently selected from the group consisting of hydrogen; and alkyl; and

wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and

wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹ and —CONR⁹R¹⁰; or

R¹¹ and R¹² together with the carbon atom to which they are attached form a cyclic ring; and

wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or

wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or

wherein R¹⁴ and R¹⁵ together with the nitrogen atom to which they are attached form a cyclic ring; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; NR⁹R¹⁰; —N⁺R⁹R¹⁰R^{w}A⁻; —SR¹⁶; —S(O)R⁹; —SO₂R⁹—SO₃R¹⁶; —CO₂ R¹⁶; —CONR⁹R¹⁰; —SO₂NR⁹R¹⁰; —PO(OR¹⁶)OR¹⁷; —P⁹R¹⁰; —P⁺R⁹R¹⁰ R¹¹A-; —S⁺R⁹R¹⁰A⁻; and carbohydrate residue; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and

wherein R¹⁶ and R¹⁷ are independently selected from the group consisting of R⁹ and M; and

wherein A⁻ is a pharmaceutically acceptable anion and M is a pharmaceutically acceptable cation; and

R²⁷ is unsubstituted phenyl or R²⁸; or

a pharmaceutically acceptable salt, solvate, or prodrug thereof;

provided that at least one of R²⁵, R²⁶ and R²⁷ is R²⁸.

Preferably, R²⁸ is:

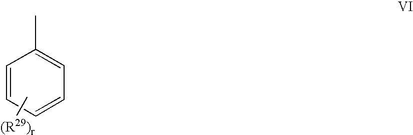

wherein

r is 0, 1, 2, 3 or 4; and

one or more R²⁹ are independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴—SR¹³; —S(O)R¹³; —SO₂R¹³; —SO₃R¹³; —NR¹³OR¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A; —P(OR¹³)OR¹⁴; —S⁺R¹³R¹⁴A⁻; and —N⁺R¹³R¹⁴R¹⁵A⁻; and

wherein the R²⁹ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A⁻; —P(O)R⁷R⁸; PR⁷PR⁸; —PR⁷R⁸; —P⁺R⁷R⁸R⁹A⁻; and —P(O)(OR⁷)OR⁸; and

wherein the R²⁹ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and

wherein R⁷ and R⁸ are independently selected from the group consisting of hydrogen; and alkyl; and

wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and

wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹ and —CONR⁹R¹⁰; or

R¹¹ and R¹² together with the carbon atom to which they are attached form a cyclic ring; and

wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or

wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or

wherein R¹⁴ and R¹⁵ together with the nitrogen atom to which they are attached form a cyclic ring; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂ R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A-; —S⁺R⁹R¹⁰A⁻; and carbohydrate residue; and.

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and

wherein R¹⁶ and R¹⁷ are independently selected from the group consisting of R⁹ and M; and

wherein A⁻ is a pharmaceutically acceptable anion and M is a pharmaceutically acceptable cation.

In one embodiment, R²⁸ is:

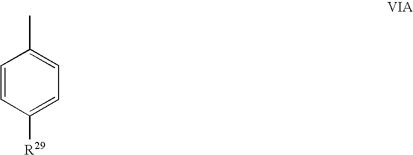

wherein R²⁹ is as defined above.

In another embodiment, R²⁸ is:

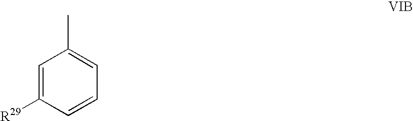

wherein R²⁹ is as defined above.

In another embodiment:

R²⁹ is independently selected from the group consisting of —OR¹³, —NR¹³R¹⁴, —NR¹³C(O)R¹⁴, —OC(O)NR¹³R¹⁴, and —NR¹³SO₂R¹⁴, and

wherein R¹³, R¹⁴, and R¹⁵ are independently selected from the group consisting of alkyl, polyether, aryl, quaternary heterocycle, arylalkyl, heterocyclylalkyl, quaternary heterocyclylalkyl, alkylheterocyclylalkyl, and alkylammoniumalkyl,

wherein alkyl optionally has one or more carbons replaced by O or N⁺R⁹R¹⁰A⁻, and

wherein R¹³, R¹⁴, and R¹⁵ are optionally substituted with one or more groups selected from the group consisting of hydroxy, carboxy, alkyl, quaternary heterocyclylalkyl, —SR⁹, —S(O)R⁹, —S(O)₂R⁹, —S(O)₃R⁹, —NR⁹R¹⁰, —N⁺R⁹R¹¹R¹²A⁻, —CONR⁹R¹⁰, and —PO(OR¹⁶)OR¹⁷, and

wherein R⁹ and R¹⁰ are independently selected from the group consisting of hydrogen, alkyl, heterocyclylalkyl, carboxyalkyl, carboalkoxyalkyl, and carboxyalkylheterocycle; and

wherein R¹¹ and R¹² are independently alkyl; and

wherein A⁻ is a pharmaceutically acceptable anion and M is a pharmaceutically acceptable cation.

In another embodiment, R²⁹ is selected from the group consisting of:

TABLE 3

| | R²⁹ |
|---|---|
| (1) | 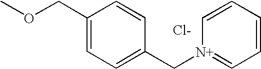 |
| (2) | 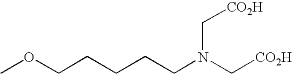 |
| (3) | 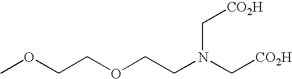 |
| (4) | 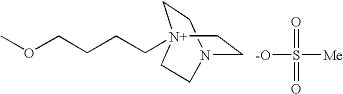 |
| (5) | 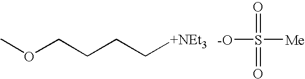 |
| (6) | 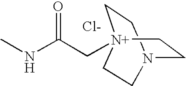 |
| (7) | 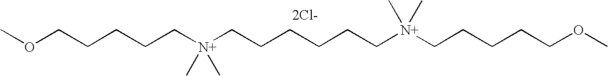 |
| (8) | 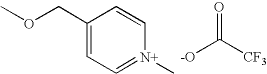 |
| (9) | 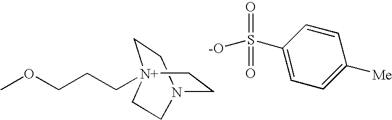 |
| (10) | 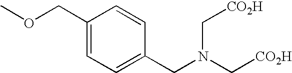 |
| (11) | 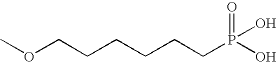 |
| (12) | 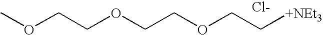 |
| (13) | 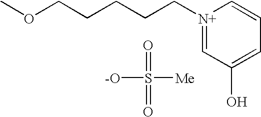 |
| (14) | 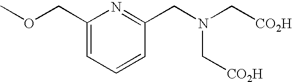 |
| (15) | 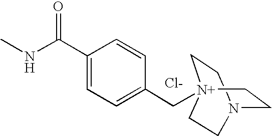 |
| (15a) | 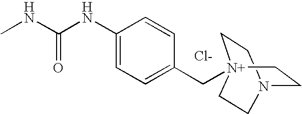 |
| (16) | 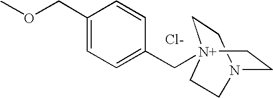 |
| (17) | 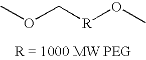 |
| (18) | 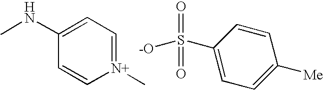 |
| (19) | 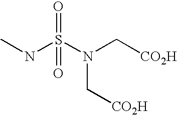 |
| (20) | 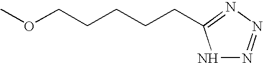 |
| (21) | 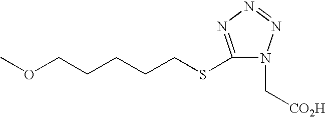 |
| (22) | 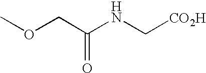 |
| (23) | 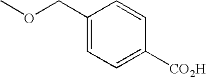 |
| (24) | 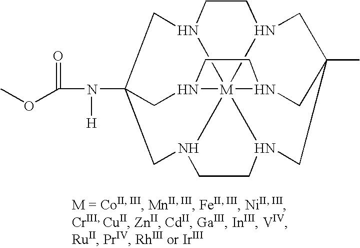 |
| (25) | 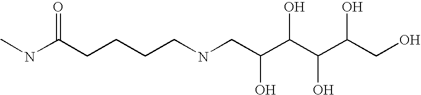 |
| (26) | 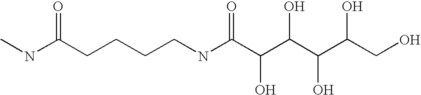 |
| (27) | 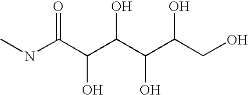 |
| (28) | 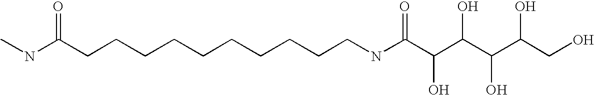 |
| (29) | 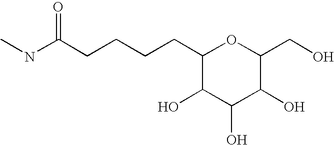 |
| (30) | 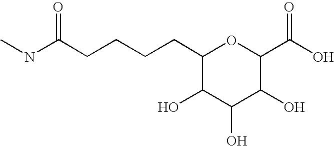 |
| (31) | 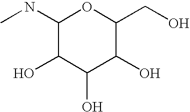 |
| (32) | 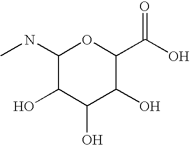 |
| (33) | 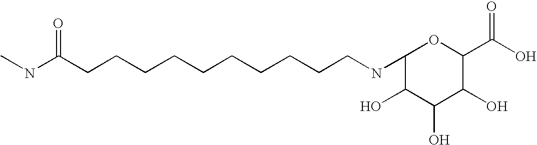 |
| (34) | 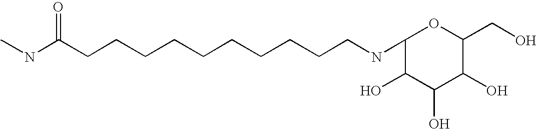 |
| (35) | 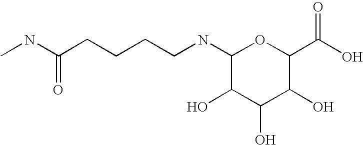 |
| (36) | 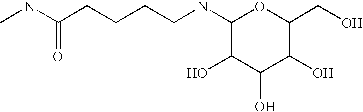 |
| (37) | 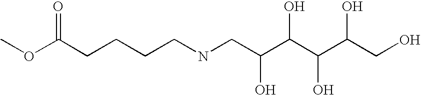 |
| (38) | 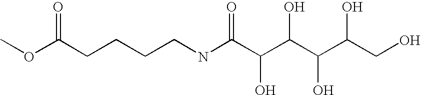 |
| (39) | 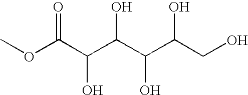 |
| (40) | 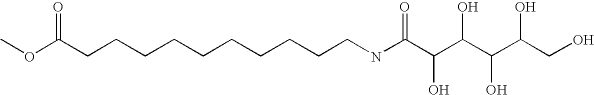 |
| (41) | 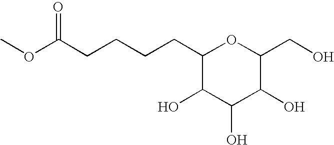 |
| (42) | 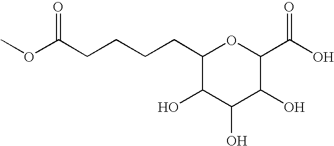 |
| (43) | 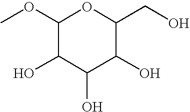 |
| (44) | 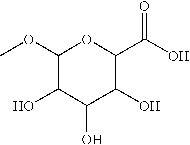 |
| (45) | 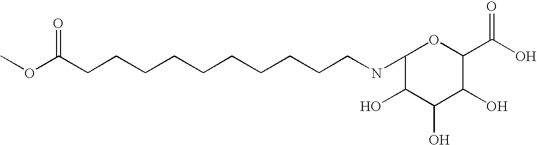 |
| (46) | 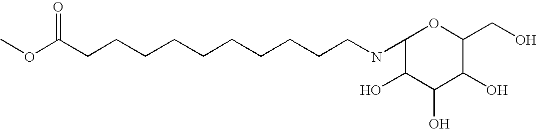 |
| (47) | 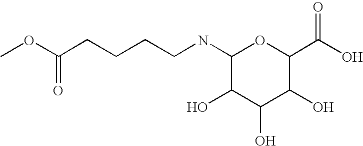 |
| (48) | 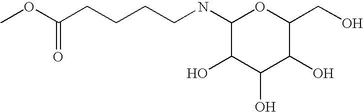 |
| (49) | 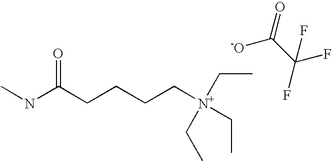 |
| (50) | 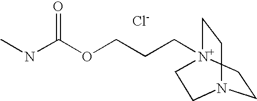 |
| (51) | 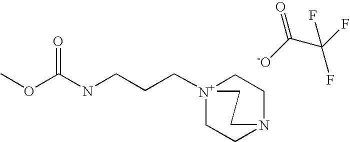 |
| (52) | 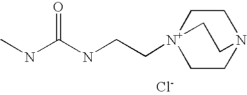 |
| (53) | 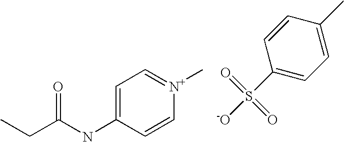 |
| (54) | 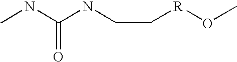 |
| (55) | 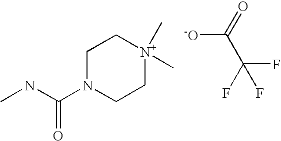 |
| (56) | 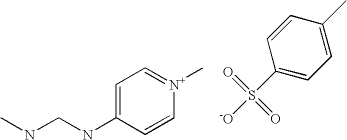 |
| (57) | 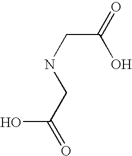 |
| (58) | 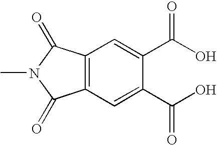 |
| (59) | 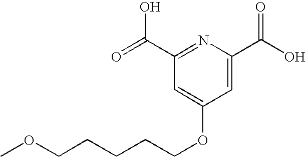 |
| (60) | 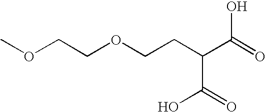 |
| (61) | 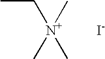 |
| (62) | 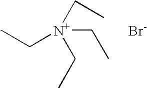 |
| (63) | 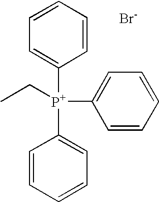 |
| (64) | 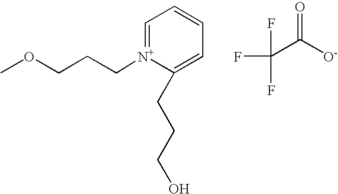 |
| (65) | 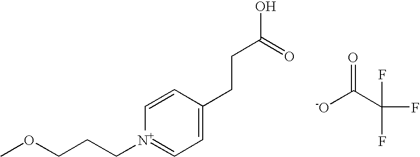 |
| (66) | 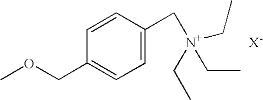 |
| (67) | 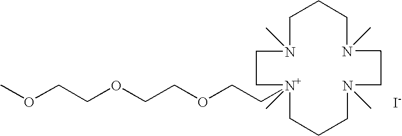 |
| (68) | 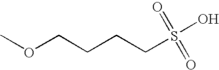 |
| (69) | 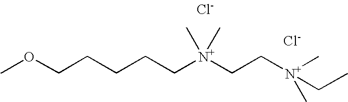 |
| (70) | 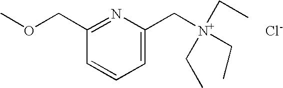 |

Optionally, R²⁹ may be selected from the following: (1)-(24), (25)-(48) or (49)-(70) from Table 3. Further, R²⁹ may be acidic or contain a quaternary ammonium nitrogen. Even further, R²⁹ may be selected from the following: (1)-(5), (6)-(10), (11)-(15), (16)-(20), (21)-(25), (26)-(30), (31)-(35), (36)-(40), (41)-(45), (46)-(50), (51)-(55), (56)-(60), (61)-(65), (66)-(70), or combinations thereof.

In another embodiment of the compounds of Formula V, R²⁵ and R²⁶ are independently selected from hydrogen and methoxy.

In another embodiment of the compounds of Formula V, one of R^{2E} and R^{2F} is ethyl and the other of R^{2E} and R^{2F} is n-butyl; and R²⁵ and R²⁶ are hydrogen.

In another embodiment of the compounds of Formula V, one of R^{2E} and R^{2F} is ethyl and the other of R^{2E} and R^{2F} is n-butyl; and R²⁵ and R²⁶ are methoxy.

Within the compounds of Formula I is another class of compounds of particular interest corresponding to Formula VII:

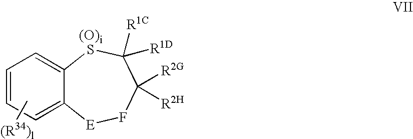

wherein:

i is 0, 1 or 2; and

1 is 0, 1, 2, 3 or 4; and R^{1C} and R^{1D} are independently selected from hydrogen and alkyl; and

R^{2G} and R^{2H} are independently selected from hydrogen, alkyl, alkenyl, alkynyl, cycloalkyl, cycloalkylalkyl, aryl and aralkyl; or

R^{2G} and R^{2H} together with the carbon atom to which they are attached form a C₃₋₁₀ cycloalkyl group; and one of E and F is NR³′ and the other of E and F is CHR³¹;

wherein R³⁰ and R³¹ are independently selected from the group consisting of hydrogen; oxo; alkyl; cycloalkyl; aryl; heterocyclyl; acyl, thioacyl, —OR⁹, and R³²;

wherein the R³⁰ and R³¹ alkyl; cycloalkyl; aryl; heterocyclyl radicals are independently substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; _NR¹³SO₂NR¹⁴R¹⁵; —PR³R¹⁴; —P(O)R¹³R¹⁴; —P⁺R¹³R¹⁴R A; —P(OR³)OR⁴; —S⁺R¹³R¹⁴A⁻; and —N⁺R¹³R¹⁴R¹⁵A⁻; and wherein the R³⁰ and R³′ alkyl; cycloalkyl; aryl; heterocyclyl radicals are independently substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³ SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and

wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³⁰ and R³¹ radicals optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A⁻; —P(O)R⁷R⁸; —PR⁷R⁸; —P⁺R⁷R⁸R⁹A⁻; and —P(O)(OR⁷)OR⁸; and

wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³⁰ and R³¹ radicals optionally may have one or more carbons replaced by —O—; —NR⁷—; —N⁺R⁷ R⁸A⁻-; —S—; —SO—; —SO₂—; —S⁺R⁷A⁻-; —PR⁷—; —P(O)R⁷—; —P⁺R⁷R⁸A⁻-; or phenylene; and

wherein R⁷ and R⁸ are independently selected from the group consisting of hydrogen; and alkyl; and

wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and

wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or

R¹¹ and R¹² together with the carbon atom to which they are attached form a cyclic ring; and

wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or

wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or

wherein R¹⁴ and R¹⁵ together with the nitrogen atom to which they are attached form a cyclic ring; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂ R¹⁶—CONR⁹R¹⁰; —SO₂NR⁹R¹⁰; —PO(OR¹⁶)OR¹⁷; —P⁹R¹⁰; —P⁺R⁹R¹⁰R¹¹ A-; —S⁺R⁹R¹⁰A⁻; and carbohydrate residue; and

wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and

wherein R¹⁶ and R¹⁷ are independently selected from the group consisting of R⁹ and M; and

wherein A is a pharmaceutically acceptable cation and M is a pharmaceutically acceptable cation; and

R³² is selected from the group consisting of cycloalkyl, aryl and heterocyclyl, wherein said cycloalkyl, aryl and heterocyclyl are substituted with —N(H)—X—R³³ or —O—X—R³³ and wherein:

X is selected from the group consisting of:

—(C═O)ₛ-alkyl-;

—(C═O)ₛ-alkyl-NH—;

—(C═O)ₛ-alkyl-O—;

—(C═O)ₛ-alkyl-(C═O)ₜ; and

a covalent bond;

R³³ is selected from selected from the group consisting of monosaccharides, disaccharides, and polysaccharides, wherein said monosaccharides, disaccharides, and polysaccharides may be protected with one or more sugar protecting groups;

s and t are independently 0 or 1; and

one or more R³⁴ radicals are independently selected from the group consisting of R³², hydrogen; halogen; —CN; —NO₂; alkyl; cycloalkyl; polyalkyl; haloalkyl; hydroxyalkyl; alkenyl; alkynyl; aryl; heterocyclyl; quaternary heterocyclyl; arylalkyl; heterocyclylalkyl; polyether; acyloxy; —OR¹³; —NR¹³R¹⁴; —SR¹³; S(O)R¹³; —S(O)₂R¹³; —SO₃R¹³; —S⁺R¹³R¹⁴A⁻; —NR¹³OR¹⁴; —NR¹³NR¹⁴R¹⁵; —CO₂R¹³; —OM; —SO₂OM; —SO₂NR¹³R¹⁴; —NR¹⁴C(O)R¹³; —C(O)NR¹³R¹⁴; —C(O)OM; —COR¹³; —OR¹⁸; S(O)ₙNR¹³R¹⁴; —NR¹³R¹⁴; —NR¹⁸OR¹⁴; —N⁺R¹³R¹⁴R¹⁵A⁻; PR¹³R¹⁴; —P(O)R¹³R¹⁴; —P⁺R¹³R¹⁴R¹⁵A⁻; amino acid residue; peptide residue; polypeptide residue; and carbohydrate residue;

wherein the R³⁴ alkyl; cycloalkyl; polyalkyl; haloalkyl; hydroxyalkyl; alkenyl; alkynyl; aryl; heterocyclyl; arylalkyl; heterocyclylalkyl; polyether; acyloxy radicals optionally may be further substituted with one or more radicals selected from the group consisting of halogen; —CN; oxo; —OR¹⁶; —NR⁹R¹⁰; N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹¹R¹²A⁻; —S⁺R⁹R¹⁰A⁻; and carbohydrate residue; and

wherein the R³⁴ quaternary heterocyclyl radical optionally may be substituted with one or more radicals selected from the group consisting of halogen; —CN; —NO₂; oxo; alkyl; cycloalkyl; polyalkyl; haloalkyl; hydroxyalkyl; alkenyl; alkynyl; aryl; heterocyclyl; arylalkyl; heterocyclylalkyl; polyether; —OR¹³; —NR¹³R¹⁴; —SR; —S(O)R¹³; —SO₂ R¹³; —SO₃R¹³; —NR¹³OR¹⁴; —NR¹³NR¹⁴R¹⁵; —CO₂R¹³; OM; —SO₂OM; —SO₂NR¹³R¹⁴; —C(O)NR¹³R¹⁴; —C(O)OM; —COR¹³; —P(O)R¹³R¹⁴; —P¹³R¹⁴; —P⁺R¹³R¹⁴A⁻; —P(OR¹³)OR¹⁴; —S⁺R¹³R¹⁴A⁻; —N⁺R¹³R¹⁴R¹⁵A⁻; and carbohydrate residue; and

wherein the R³⁴ radicals comprising carbon optionally may have one or more carbons replaced by —O—; —N⁺R¹³R¹⁴A⁻-; —S—; —SO—; —SO₂—; —S⁺R¹³A⁻-; —PR¹³—; —P(O)R¹³—; —P⁺R¹³R¹⁴A⁻; phenylene; amino acid residue; peptide residue; polypeptide residue; carbohydrate residue; polyether; or polyalkyl; wherein said phenylene; amino acid residue; peptide residue; polypeptide residue; carbohydrate residue; and polyalkyl optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; or —P(O)R⁹—; and

wherein R¹⁸ is selected from the group consisting of alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl; and

wherein the R¹⁸ alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl radicals optionally may be substituted with one or more radicals selected from the group consisting of halogen; —CN; NO₂; oxo; —OR⁹; —NR⁹R¹⁰; —N⁺R⁹R¹¹R¹² A⁻; —SR⁹; —S(O)R⁹; —SO₂R⁹; —SO₃R⁹; —CO₂R⁹; —CONR⁹R¹⁰; —SO₂OM; —SO₂NR⁹R¹⁰; —PR⁹R¹⁰; —P(OR¹³)OR¹⁴; —PO(OR¹⁶)OR¹⁷; and C(O)OM; or

a pharmaceutically acceptable salt, solvate, or prodrug thereof;

provided that at least one of R³⁰, R³¹ and R³⁴ is R³².

Preferably, R³² is phenyl substituted with —N(H)—X—R³³ or —O—X—R³³ wherein:

X is selected from the group consisting of:

—(C═O)ₛ-alkyl-;

—(C═O)ₛ-alkyl-NH—;

—(C═O)ₛ-alkyl-O—;

—(C═O)ₛ-alkyl-(C═O)ₜ; and

a covalent bond;

R³³ is selected from selected from the group consisting of monosaccharides, disaccharides, and polysaccharides; and

s and t are independently 0 or 1.

In one embodiment, R³² is phenyl substituted at the para-position with —N(H)—X—R³³ or —O—X—R³³ wherein:

X is selected from the group consisting of:

—(C═O)ₛ-alkyl-;

—(C═O)ₛ-alkyl-NH—;

—(C═O)ₛ-alkyl-O—;

—(C═O)ₛ-alkyl-(C═O)ₜ; and

a covalent bond; and

R³³ is selected from selected from the group consisting of monosaccharides, disaccharides, and polysaccharides; and

s and t are independently 0 or 1.

In another embodiment, R³² is phenyl substituted at the meta-position with —N(H)—X—R³³ or —O—X—R³³ wherein:

X is selected from the group consisting of:

—(C═O)ₛ-alkyl-;

—(C═O)ₛ-alkyl-NH—;

—(C═O)ₛ-alkyl-O—;

—(C═O)ₛ-alkyl-(C═O)ₜ; and

a covalent bond; and

R³³ is selected from selected from the group consisting of monosaccharides, disaccharides, and polysaccharides; and

s and t are independently 0 or 1;

In another embodiment, R³² is phenyl substituted with a radical selected from the group consisting of:

TABLE 4

| | R³² |
|---|---|
| (1) | 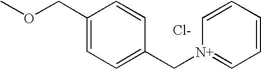 |
| (2) | 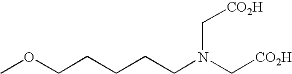 |
| (3) | 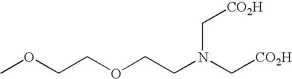 |
| (4) | 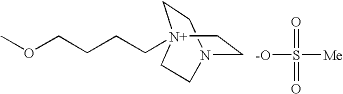 |
| (5) | 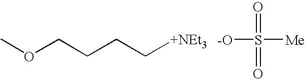 |
| (6) | 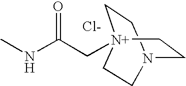 |
| (7) | 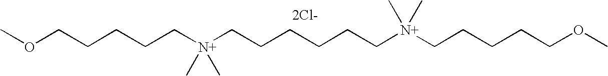 |
| (8) | 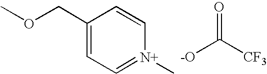 |
| (9) | 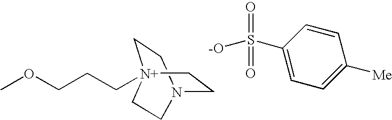 |
| (10) | 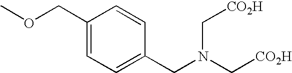 |
| (11) | 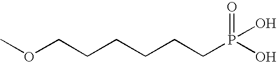 |
| (12) | 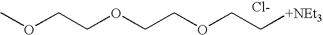 |
| (13) | 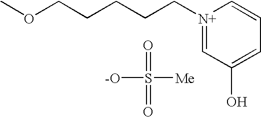 |
| (14) | 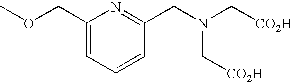 |
| (15) | 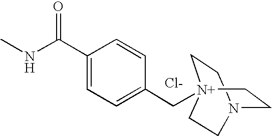 |
| (15a) | 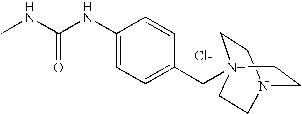 |
| (16) | 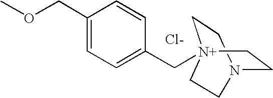 |
| (17) | 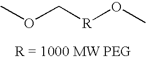 |
| (18) | 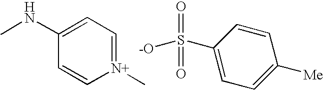 |
| (19) | 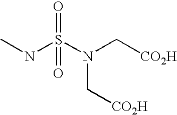 |
| (20) | 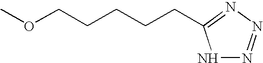 |
| (21) | 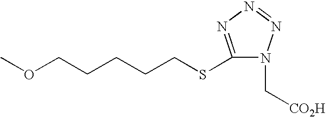 |
| (22) | 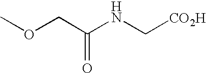 |
| (23) | 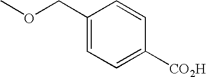 |
| (24) | 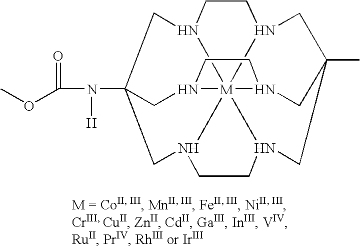 |
| (25) | 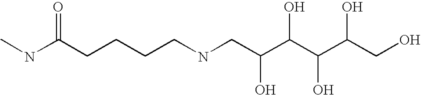 |
| (26) | 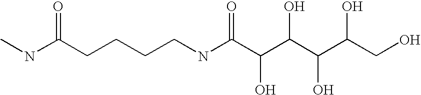 |
| (27) | 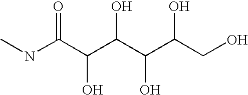 |
| (28) | 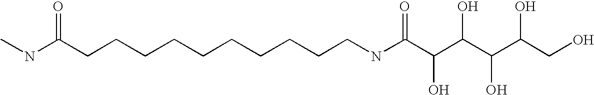 |
| (29) | 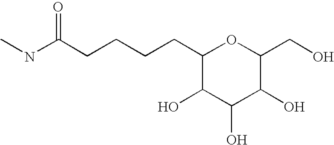 |
| (30) | 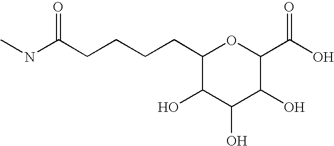 |
| (31) | 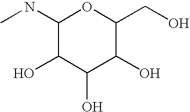 |
| (32) | 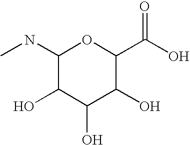 |
| (33) | 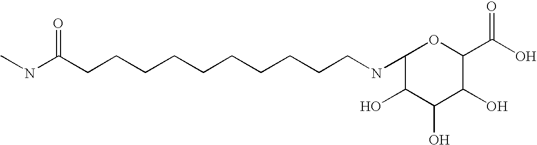 |
| (34) | 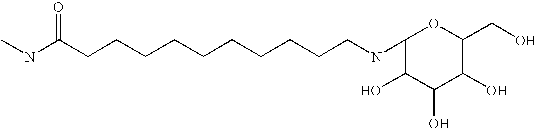 |
| (35) | 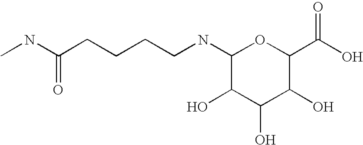 |
| (36) | 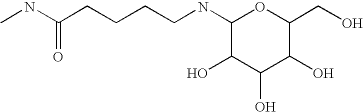 |
| (37) | 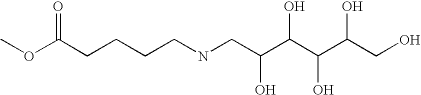 |
| (38) | 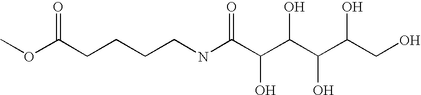 |
| (39) | 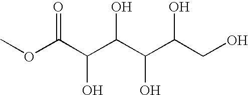 |
| (40) | 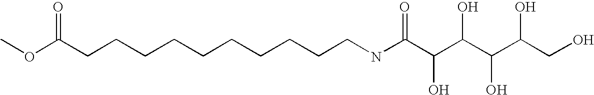 |
| (41) | 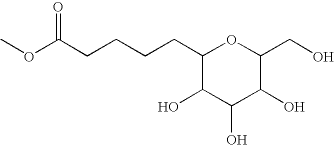 |
| (42) | 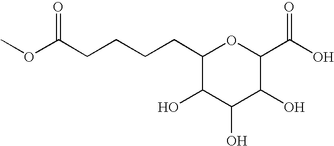 |
| (43) | 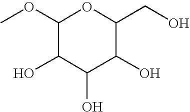 |
| (44) | 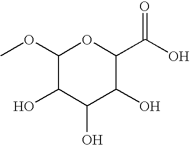 |
| (45) | 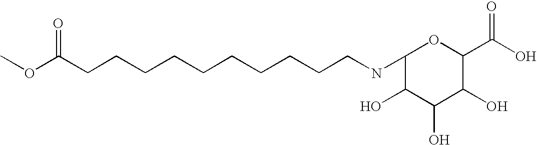 |
| (46) | 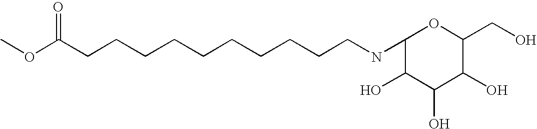 |
| (47) | 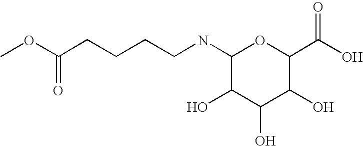 |
| (48) | 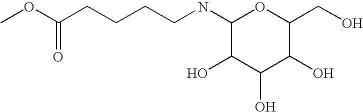 |
| (49) | 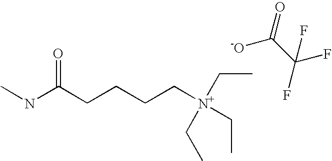 |
| (50) | 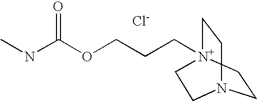 |
| (51) | 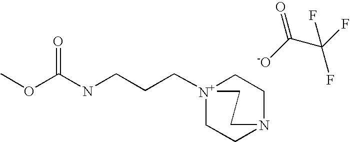 |
| (52) | 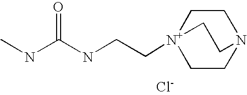 |
| (53) | 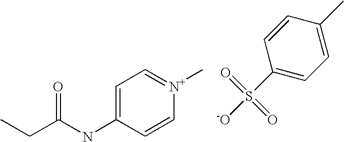 |
| (54) | 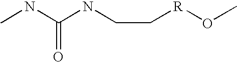 |
| (55) | 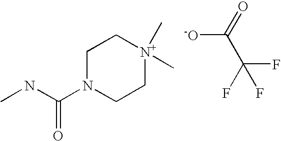 |
| (56) | 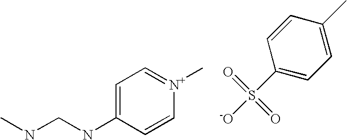 |
| (57) | 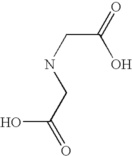 |
| (58) | 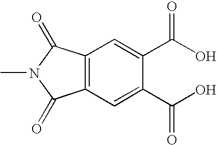 |
| (59) | 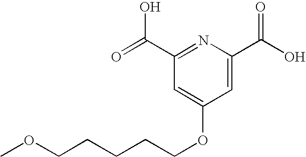 |
| (60) | 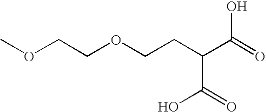 |
| (61) | 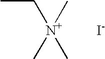 |
| (62) | 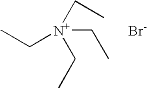 |
| (63) | 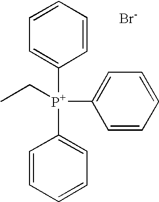 |
| (64) | 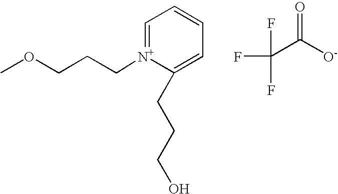 |
| (65) | 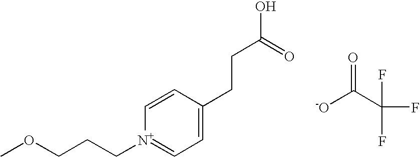 |
| (66) | 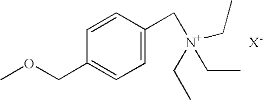 |
| (67) | 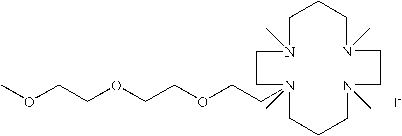 |
| (68) | 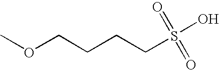 |
| (69) | 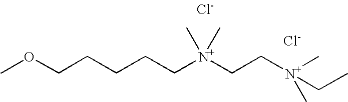 |
| (70) | 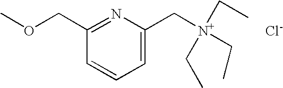 |

Optionally, R³² may be selected from the following: (1)-(24), (25)-(48) or (49)-(70) from Table 4. Further, R³² may be acidic or contain a quaternary ammonium nitrogen. Even further, R³² may be selected from the following: (1)-(5), (6)-(10), (11)-(15), (16)-(20), (21)-(25), (26)-(30), (31)-(35), (36)-(40), (41)-(45), (46)-(50), (51)-(55), (56)-(60), (61)-(65), (66)-(70), or combinations thereof.

In another embodiment of the compounds of Formula VII, R³⁰ is R³²; and R³¹ is selected from the group consisting of hydrogen and alkyl.

In another embodiment of the compounds of Formula VII, R³⁰ is selected from the group consisting of hydrogen and alkyl; and R³¹ is R³².

Within the compounds of Formula VII is a class of compounds of specific interest corresponding to Formula VIIA:

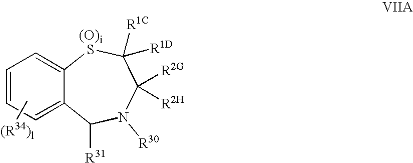

wherein:

i is 0, 1 or 2; and

1 is 0, 1, 2, 3 or 4; and

R^{1C} and R^{1D} are independently selected from hydrogen and alkyl and

R^{2G} and R^{2H} are independently selected from hydrogen, alkyl, alkenyl, alkynyl, cycloalkyl, cycloalkylalkyl, aryl and aralkyl; or

R^{2G} and R^{2H} together with the carbon atom to which they are attached form a C₃₋₇ cycloalkyl group; and

i, 1, R³⁰, R³¹ and R³⁴ are as previously defined above for compounds of Formula VII.

Within the compounds of Formula VII is a class of compounds of specific interest corresponding to Formula VIIB:

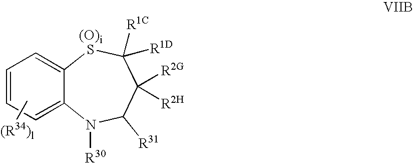

wherein:

i is 0, 1 or 2; and

1 is 0, 1, 2, 3 or 4; and

R^{1C} and R^{1D} are independently selected from hydrogen and alkyl; and

R^{2G} and R^{2H} are independently selected from hydrogen, alkyl, alkenyl, alkynyl, cycloalkyl, cycloalkylalkyl, aryl and aralkyl; or

R^{2G} and R^{2H} together with the carbon atom to which they are attached form a C₃₋₇ cycloalkyl group; and

i, 1, R³⁰, R³¹ and R³⁴ are as previously defined above for compounds of Formula VI.

Within the compounds of Formula VII is a class of compounds of particular interest corresponding to Formula VIII:

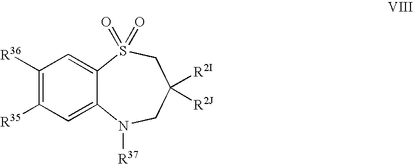

wherein:

R^{2I} and R^{2J} are independently selected from C₁₋₆ alkyl; and

R³⁵ is selected from the group consisting of halogen and R³⁸;

R³⁶ is selected from the group consisting of hydroxy, alkoxy, and R³⁸;

wherein R³⁸ is selected from the group consisting of cycloalkyl, aryl and heterocyclyl, wherein said cycloalkyl, aryl and heterocyclyl are substituted with —N(H)—X—R³⁹ or —O—X—R³⁹ and wherein:

X is selected from the group consisting of:

—(C═O)ᵤ-alkyl-;

—(C═O)ᵤ-alkyl-NH—;

—(C═O)ᵤ-alkyl-O—;

—(C═O)ᵤ-alkyl-(C═O)ᵥ; and

a covalent bond; and

R³⁹ is selected from selected from the group consisting of monosaccharides, disaccharides, and polysaccharides, wherein said monosaccharides, disaccharides, and polysaccharides may be protected with one or more sugar protecting groups; and

u and v are independently 0 or 1; and

R³⁷ is unsubstituted phenyl or R³⁸; or

a pharmaceutically acceptable salt, solvate, or prodrug thereof;

provided that at least one of R³⁵, R³⁶ and R³⁷ is R³⁸.

Preferably, R³⁸ is phenyl substituted with —N(H)—X—R³⁹ or —O—X—R³⁹ wherein:

X is selected from the group consisting of:

—(C═O)ᵤ-alkyl-;

—(C═O)ᵤ-alkyl-NH—;

—(C═O)ᵤ-alkyl-O—;

—(C═O)ₛ-alkyl-(C═O)ᵥ; and

a covalent bond; and

R³⁹ is selected from selected from the group consisting of monosaccharides, disaccharides, and polysaccharides; and

u and v are independently 0 or 1.

In one embodiment, R³⁸ is phenyl substituted at the para-position with —N(H)—X—R³⁹ or —O—X—R³⁹ wherein:

X is selected from the group consisting of:

—(C═O)ᵤ-alkyl-;

—(C═O)ᵤ-alkyl-NH—;

—(C═O)ᵤ-alkyl-O—;

—(C═O)ᵤ-alkyl-(C═O)ᵥ; and

a covalent bond; and

R³⁹ is selected from selected, from the group consisting of monosaccharides, disaccharides, and polysaccharides; and

u and v are independently 0 or 1.

In another embodiment, R³⁸ is phenyl substituted at the meta-position with —N(H)—X—R³⁹ or —O—X—R³⁹ wherein:

X is selected from the group consisting of:

—(C═O)ᵤ-alkyl-;

—(C═O)ᵤ-alkyl-NH—;

—(C═O)ᵤ-alkyl-O—;

—(C═O)ᵤ-alkyl-(C═O)ᵥ; and

a covalent bond; and

R³⁹ is selected from selected from the group consisting of monosaccharides, disaccharides, and polysaccharides; and

u and v are independently 0 or 1.

In another embodiment, R³⁸ is phenyl substituted with a radical selected from the group consisting of:

TABLE 5

| | R³⁸ |
|---|---|
| (1) | 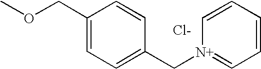 |
| (2) | 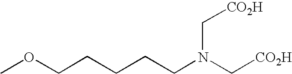 |
| (3) | 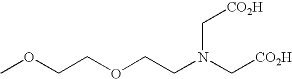 |
| (4) | 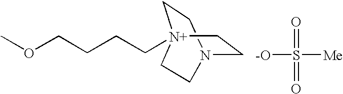 |
| (5) | 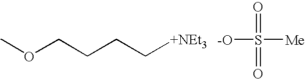 |
| (6) | 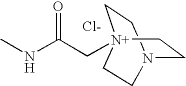 |
| (7) | 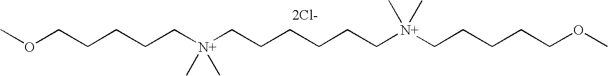 |
| (8) | 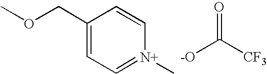 |
| (9) | 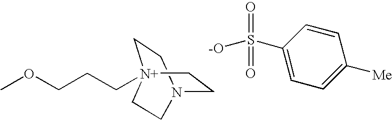 |
| (10) | 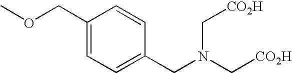 |
| (11) | 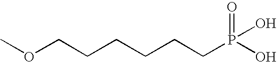 |
| (12) | 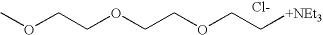 |
| (13) | 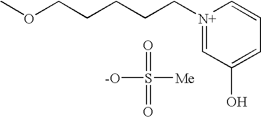 |
| (14) | 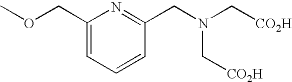 |
| (15) | 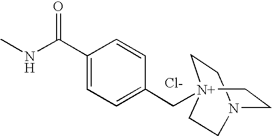 |
| (15a) | 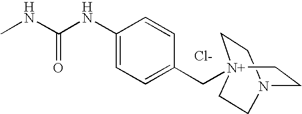 |
| (16) | 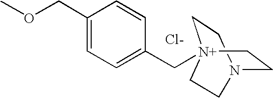 |
| (17) | 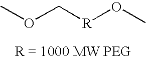 |
| (18) | 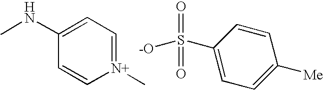 |
| (19) | 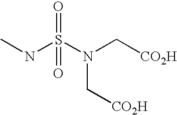 |
| (20) | 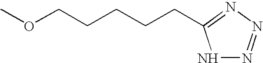 |
| (21) | 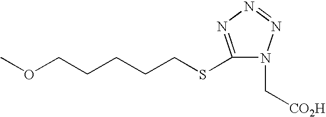 |
| (22) | 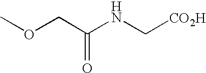 |
| (23) | 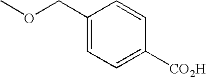 |
| (24) | 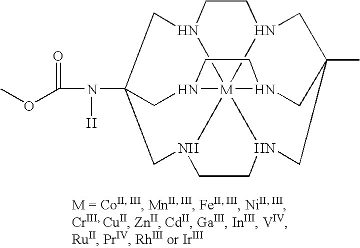 |
| (25) | 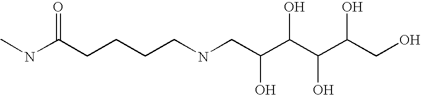 |
| (26) | 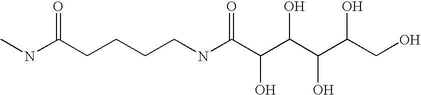 |
| (27) | 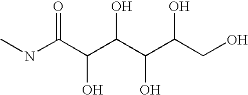 |
| (28) | 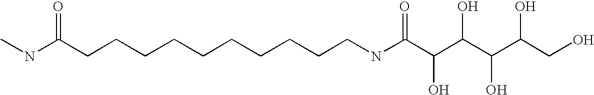 |
| (29) | 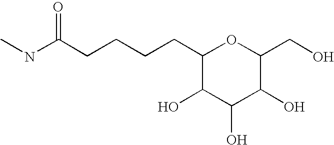 |
| (30) | 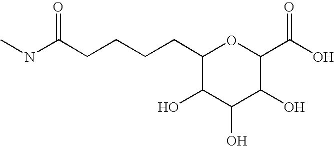 |
| (31) | 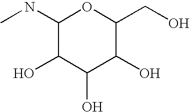 |
| (32) | 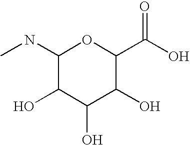 |
| (33) | 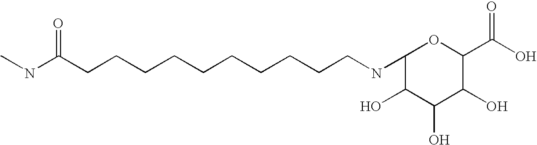 |
| (34) | 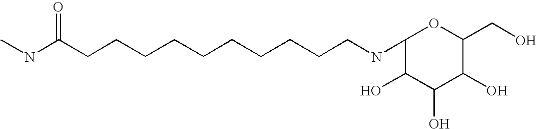 |
| (35) | 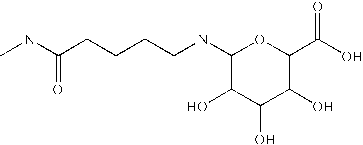 |
| (36) | 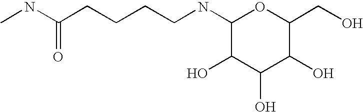 |
| (37) | 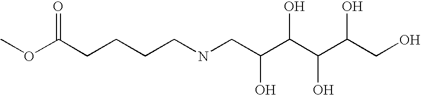 |
| (38) | 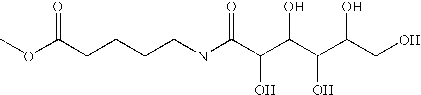 |
| (39) | 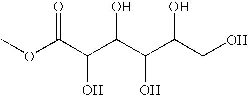 |
| (40) | 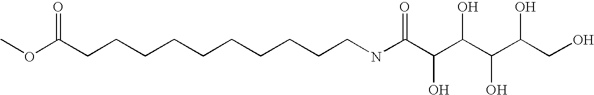 |
| (41) | 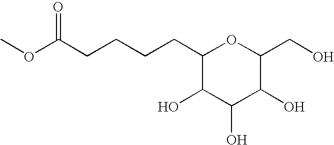 |
| (42) | 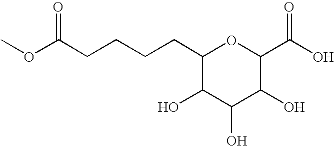 |
| (43) | 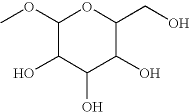 |
| (44) | 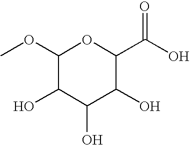 |
| (45) | 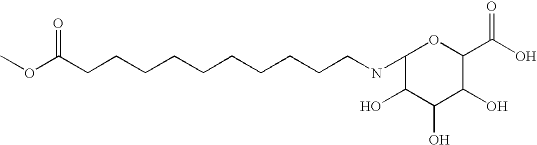 |
| (46) | 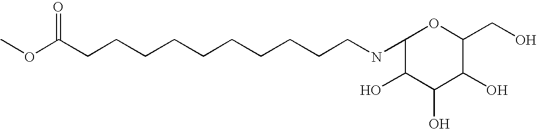 |
| (47) | 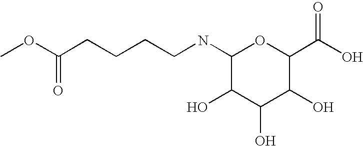 |
| (48) | 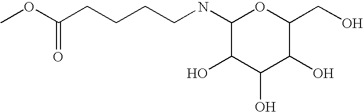 |
| (49) | 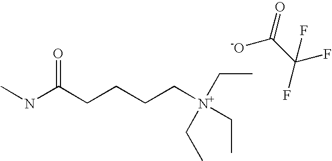 |
| (50) | 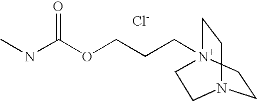 |
| (51) | 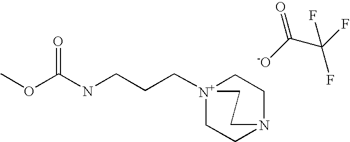 |
| (52) | 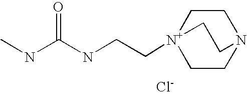 |
| (53) | 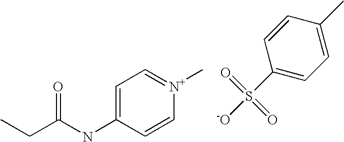 |
| (54) | 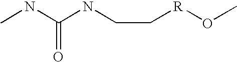 |
| (55) | 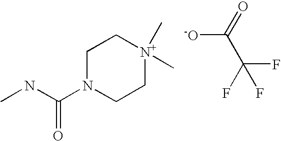 |
| (56) | 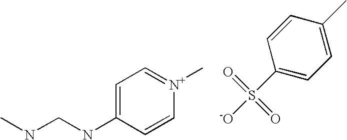 |
| (57) | 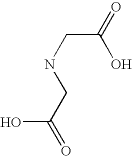 |
| (58) | 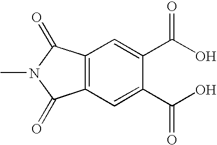 |
| (59) | 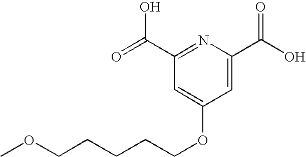 |
| (60) | 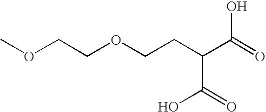 |
| (61) | 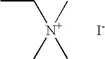 |
| (62) | 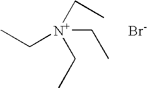 |
| (63) | 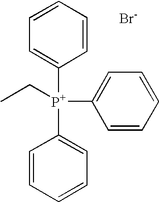 |
| (64) | 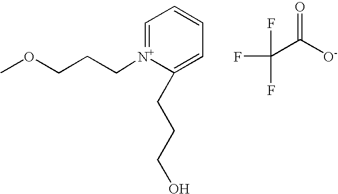 |
| (65) | 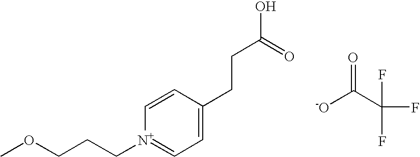 |
| (66) | 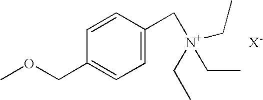 |
| (67) | 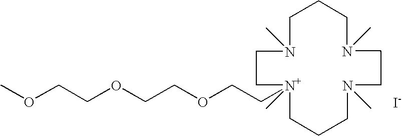 |
| (68) | 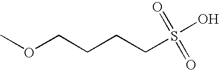 |
| (69) | 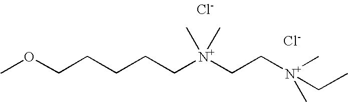 |
| (70) | 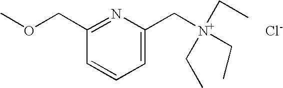 |

Optionally, R³⁸ may be selected from the following: (1)-(24), (25)-(48) or (49)-(70) from Table 5. Further, R³⁸ may be acidic or contain a quaternary ammonium nitrogen. Even further, R³⁸ may be selected from the following: (1)-(5), (6)-(10), (11)-(15), (16)-(20), (21)-(25), (26)-(30), (31)-(35), (36)-(40), (41)-(45), (46)-(50), (51)-(55), (56)-(60), (61)-(65), (66)-(70), or combinations thereof.

In another embodiment of the compounds of Formula VIII, R³⁵ is chloro, and R³⁶ is selected from the group consisting of hydroxy and methoxy.

In another embodiment of the compounds of Formula VIII, one of R^{2I} and R^{2J} is ethyl and the other of R^{2I} and R^{2J} is n-butyl; R³⁵ is chloro; and R³⁶ is hydroxy.

In another embodiment of the compounds of Formula VIII, one of R^{2I} and R^{2J} is ethyl and the other of R^{2I} and R^{2J} is n-butyl; R³⁵ is chloro; and R³⁶ is methoxy.

Within the compounds of Formula VII is a class of compounds of particular interest corresponding to Formula IX:

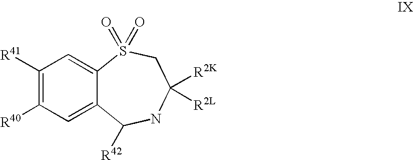

wherein:

R^{2K} and R^{2L} are independently selected from C₁₋₆ alkyl; and

R⁴⁰ and R⁴¹ are independently selected from the group consisting of hydrogen, alkoxy, and R⁴³;

wherein R⁴³ is selected from the group consisting of cycloalkyl, aryl and heterocyclyl, wherein said cycloalkyl, aryl and heterocyclyl are substituted with —N(H)—X—R⁴⁴ or —O—X—R⁴⁴ and wherein:

X is selected from the group consisting of:

—(C═O)ₐ-alkyl-;

—(C═O)ₐ-alkyl-NH—;

—(C═O)ₐ-alkyl-O—;

—(C═O)ₐ-alkyl-(C═O)_{b}; and

a covalent bond; and

R⁴⁴ is selected from selected from the group consisting of monosaccharides, disaccharides, and polysaccharides, wherein said monosaccharides, disaccharides, and polysaccharides may be protected with one or more sugar protecting groups; and

a and b are independently 0 or 1; and

R⁴² is unsubstituted phenyl or R⁴³; or

a pharmaceutically acceptable salt, solvate, or prodrug thereof; provided that at least one of R⁴⁰, R⁴¹ and R⁴² is R⁴³.

Preferably, R⁴³ is phenyl substituted with —N(H)—X—R⁴⁴ or —O—X—R⁴⁴ wherein:

X is selected from the group consisting of:

—(C═O)ₐ-alkyl-;

—(C═O)ₐ-alkyl-NH—;

—(C═O)ₐ-alkyl-O—;

—(C═O)ₐ-alkyl-(C═O)_{b}; and

a covalent bond; and

R⁴⁴ is selected from selected from the group consisting of monosaccharides, disaccharides, and polysaccharides; and

a and b are independently 0 or 1.

In one embodiment, R⁴³ is phenyl substituted at the para-position with —N(H)—X—R⁴⁴ or —O—X—R⁴⁴ wherein:

X is selected from the group consisting of:

—(C═O)ₐ-alkyl-;

—(C═O)ₐ-alkyl-NH—;

—(C═O)ₐ-alkyl-O—;

—(C═O)ₐ-alkyl-(C═O)_{b}; and

a covalent bond; and

R⁴⁴ is selected from selected from the group consisting of monosaccharides, disaccharides, and polysaccharides; and

a and b are independently 0 or 1.

In another embodiment, R⁴³ is phenyl substituted at the meta-position with —N(H)—X—R⁴⁴ or —O—X—R⁴⁴ wherein:

X is selected from the group consisting of:

—(C═O)ₐ-alkyl-;

—(C═O)ₐ-alkyl-NH—;

—(C═O)ₐ-alkyl-O—;

—(C═O)ₐ-alkyl-(C═O)_{b}; and

a covalent bond; and

R⁴⁴ is selected from selected from the group consisting of monosaccharides, disaccharides, and polysaccharides; and

a and b are independently 0 or 1.

In another embodiment, R⁴³ is phenyl substituted with a radical selected from the group consisting of:

TABLE 6

| | R⁴³ |
|---|---|
| (1) | 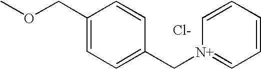 |
| (2) | 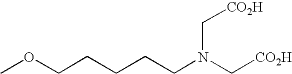 |
| (3) | 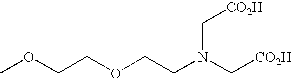 |
| (4) | 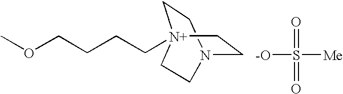 |
| (5) | 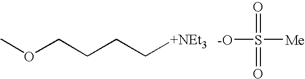 |
| (6) | 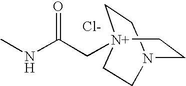 |
| (7) | 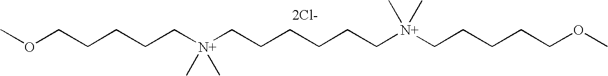 |
| (8) | 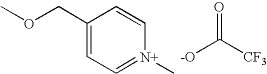 |
| (9) | 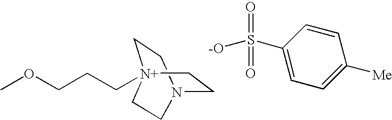 |
| (10) | 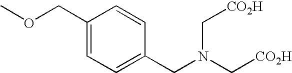 |
| (11) | 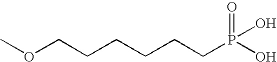 |
| (12) | 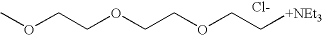 |
| (13) | 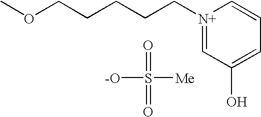 |
| (14) | 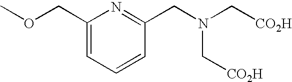 |
| (15) | 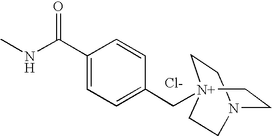 |
| (15a) | 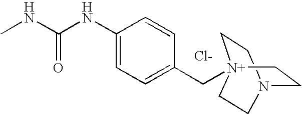 |
| (16) | 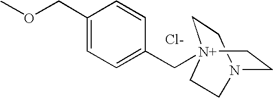 |
| (17) | 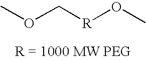 |
| (18) | 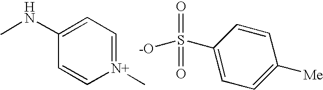 |
| (19) | 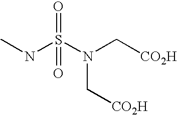 |
| (20) | 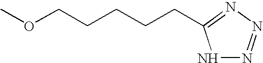 |
| (21) | 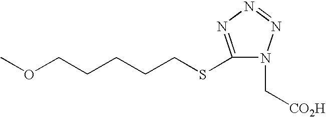 |
| (22) | 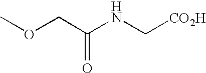 |
| (23) | 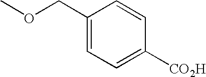 |
| (24) | 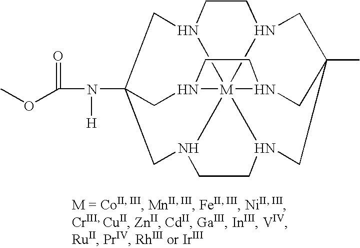 |
| (25) | 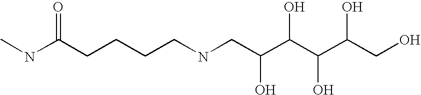 |
| (26) | 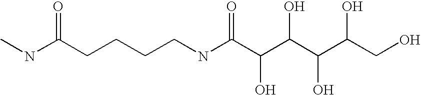 |
| (27) | 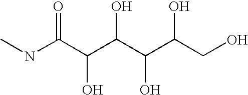 |
| (28) | 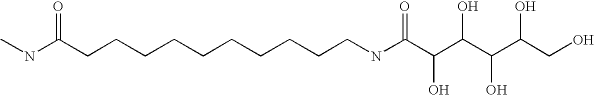 |
| (29) | 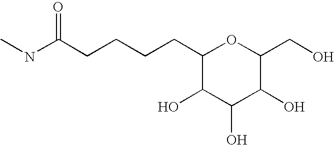 |
| (30) | 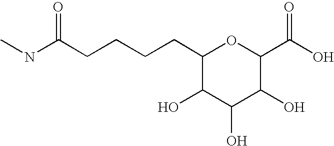 |
| (31) | 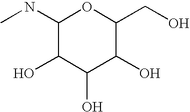 |
| (32) | 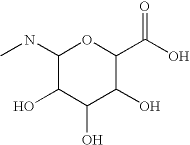 |
| (33) | 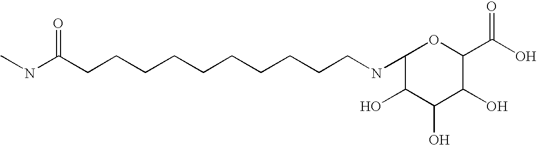 |
| (34) | 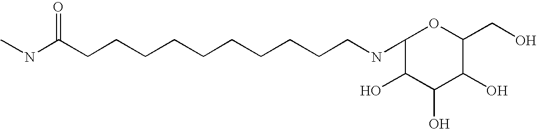 |
| (35) | 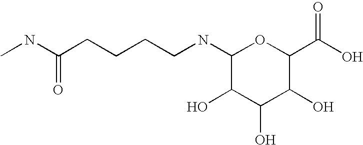 |
| (36) | 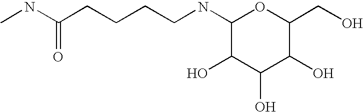 |
| (37) | 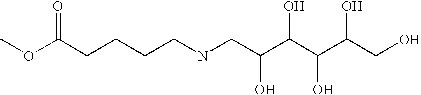 |
| (38) | 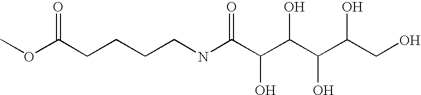 |
| (39) | 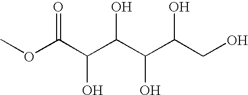 |
| (40) | 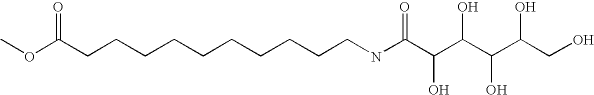 |
| (41) | 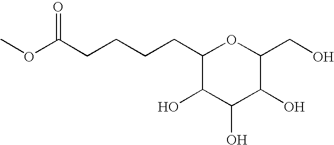 |
| (42) | 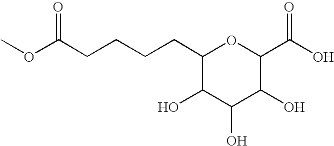 |
| (43) | 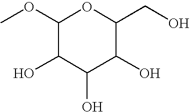 |
| (44) | 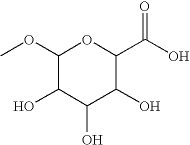 |
| (45) | 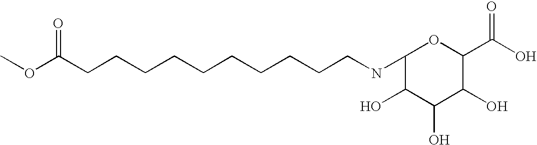 |
| (46) | 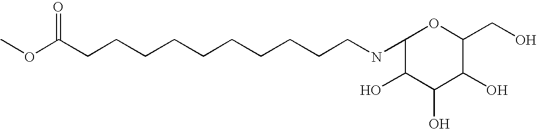 |
| (47) | 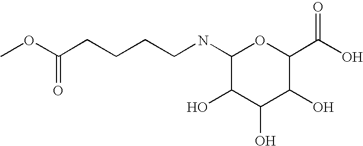 |
| (48) | 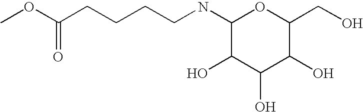 |
| (49) | 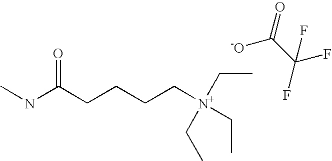 |
| (50) | 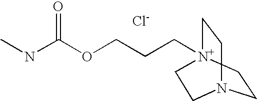 |
| (51) | 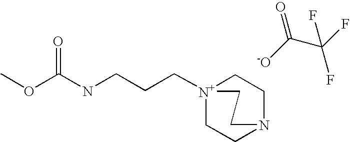 |
| (52) | 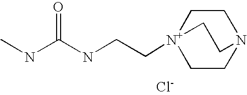 |
| (53) | 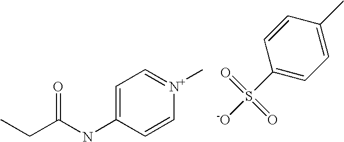 |
| (54) | 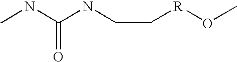 |
| (55) | 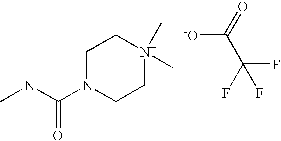 |
| (56) | 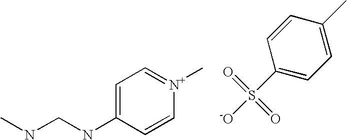 |
| (57) | 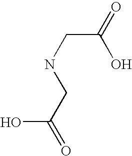 |
| (58) | 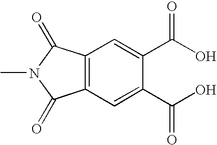 |
| (59) | 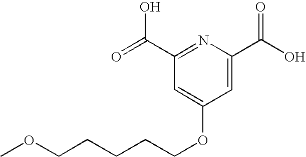 |
| (60) | 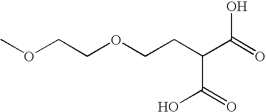 |
| (61) | 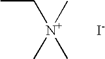 |
| (62) | 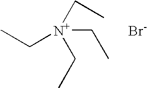 |
| (63) | 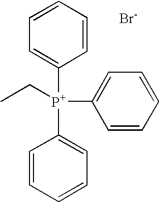 |
| (64) | 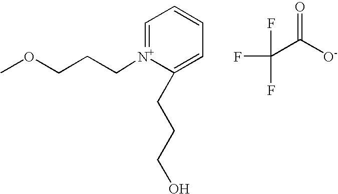 |
| (65) | 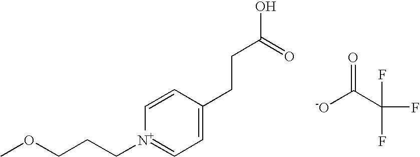 |
| (66) | 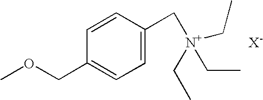 |
| (67) | 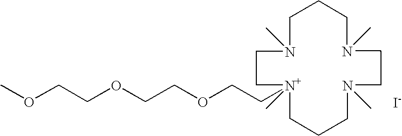 |
| (68) | 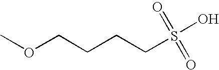 |
| (69) | 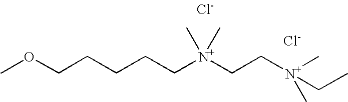 |
| (70) | 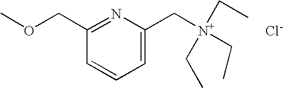 |

Optionally, R⁴³ may be selected from the following: (1)-(24), (25)-(48) or (49)-(70) from Table 6. Further, R⁴³ may be acidic or contain a quaternary ammonium nitrogen. Even further, R⁴³ may be selected from the following: (1)-(5), (6)-(10), (11)-(15), (16)-(20), (21)-(25), (26)-(30), (31)-(35), (36)-(40), (41)-(45), (46)-(50), (51)-(55), (56)-(60), (61)-(65), (66)-(70), or combinations thereof.

In another embodiment of the compounds of Formula IX, R⁴⁰ and R⁴¹ are independently selected from hydrogen and methoxy.

In another embodiment of the compounds of Formula IX, one of R^{2K} and R^{2L} is ethyl and the other of R^{2K} and R^{2L} is n-butyl; and R⁴⁰ and R⁴¹ are hydrogen.

In another embodiment of the compounds of Formula IX, one of R^{2K} and R^{2L} is ethyl and the other of R^{2K} and R^{2L} is n-butyl; and R⁴⁰ and R⁴¹ are methoxy.

In each of the various embodiments of the invention described above, at least one or more of the following conditions preferably are satisfied:

(1) j is 1 or 2. Preferably, j is 2; and/or

(2) The substituents at the 2-position of the benzothiazepine are independently selected from the group consisting of hydrogen and alkyl. Preferably, these substituents are hydrogen; and/or

(3) The substitutents at the 3-position of the benzothiazepine are independently selected from the group consisting of hydrogen and alkyl. Preferably, these substituents are independently selected from the group consisting of C₁₋₆ alkyl. More preferably, these substituents are selected from the group consisting of ethyl, propyl and butyl. Still more preferably, either (a) one of these substituents is ethyl and the other of these substituents is n-butyl, or (b) both of these substituents are n-butyl; and/or

(4) The 4-position nitrogen substituent (e.g., R³, when the compound is a 1,4-benzothiazepine) or one or both the 4-position carbon substituents (e.g., one or two group(s) at the 4-position carbon, when the compound is a 1,5-benzothiazepine) of the benzothiazepine are independently selected from the group consisting of hydrogen and hydroxy; and/or

(5) The 5-position nitrogen substituent (e.g., R³, when the compound is a 1,5-benzothiazepine) or one of the 5-position carbon substituents (e.g., R⁴, when the compound is a 1,4-benzothiazepine) of the benzothiazepine is substituted aryl wherein said aryl is substituted with (a) a moiety possessing an overall positive charge; and/or (b) a moiety comprising a quaternary ammonium group or a quaternary amine salt; and/or (c) a moiety comprising a phosphonic acid group or at least, two carboxyl groups. Preferably, this substituent is substituted phenyl. More preferably, this substituent is phenyl that is glucuronidated or monosubstituted with a radical selected from the group consisting of —OR¹³, —NR¹³C(O)R¹⁴, —NR¹³C(O)NR¹⁴R¹⁵, —NR¹³CO₂R¹⁴, —OC(O)R¹³, —OC(O)NR¹³R¹⁴, —NR¹³SOR¹⁴, —NR¹³SO₂R¹⁴, —NR¹³SONR¹⁴R¹⁵, and —NR¹³SO₂NR¹⁴R¹⁵ wherein R¹³, R¹⁴ and R¹⁵ are as previously defined for compounds of Formula I. Still more preferably, this substituent is phenyl that is monosubstituted with a radical selected from the group consisting of —OR¹³ and —NR³SO₂NR¹⁴R¹⁵. Still more preferably, this substituent is phenyl substituted at the para or meta position with —OR¹³ or —NR¹³SO₂NR¹⁴R¹⁵ wherein R¹³ comprises a quaternary heterocycle, quaternary heteroaryl, carboxy or substituted amino; and/or

(6) When the compound is a 1,4-benzothiazepine, the other substituent at the 5-position carbon of the benzothiazepine is hydrogen; and/or

(7) One or more substitutents of the benzo ring of the benzothiazepine are independently selected from the group consisting of halogen, —OR¹³ and —NR¹³R¹⁴, wherein R¹³R¹⁴ are as previously defined for compounds of Formula I. Preferably, the substitutents of the benzo ring are independently selected from the group consisting of halogen, hydroxy, alkoxy, amino, alkylamino and dialkylamino. Still more preferably, the substituents are independently selected from the group consisting of chloro, methoxy and dimethylamino.

Alternative Forms of Novel Compounds

Also included in the family of compounds of Formulae I, IA, IB, III, V, VII, VIII and IX are (1) the stereoisomers thereof, (b) the pharmaceutically-acceptable salts thereof, (c) the tautomers thereof, (d) the protected acids and the conjugate acids thereof, and (e) the prodrugs thereof.

The stereoisomers of these compounds may include, but are not limited to, enantiomers, diastereomers, racemic mixtures and other mixtures thereof. Such stereoisomers can be prepared and separated using conventional techniques, either by reacting enantiomeric starting materials, or by separating isomers of compounds of the present invention. Isomers may include geometric isomers, for example cis isomers or trans isomers across a double bond. All such isomers are contemplated among the compounds of the present invention. Such isomers may be used in either pure form or in admixture with those inhibitors described above.

The protected acids of these compounds include, but are not limited to, protected acids such as esters, hydroxyamino derivatives, amides and sulfonamides. Thus, for example, primary and secondary amines can be reacted with carboxylic acid substituted forms of the compounds of Formulae I, IA, IB, III, V, VII, VIII and IX to form amides which can be useful as prodrugs. Preferred amines are heterocyclicamines, including optionally substituted aminothiazoles, optionally substituted amino-isoxazoles, optionally substituted aminopyridines, optionally substituted aniline derivatives, optionally substituted sulfonamides, optionally substituted aminocarboxylic acids, and the like. The esters, hydroxyamino derivatives and sulfonamides can be prepared from the acids by methods known to one skilled in the art.

Pharmaceutically-acceptable salts include salts commonly used to form alkali metal salts and to form addition salts of free acids or free bases. The nature of the salt is not critical, provided that it is pharmaceutically-acceptable. Suitable pharmaceutically-acceptable acid addition salts of compounds of Formulae I, IA, IB, III, V, VII, VIII and IX may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, salicyclic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, N-hydroxybutyric, salicyclic, galactaric and galacturonic acid.

Suitable pharmaceutically-acceptable base addition salts of compounds of Formulae I, IA, IB, III, V, VII, VIII and IX include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, histidine, glucamine, isopropylamine, lysine, morpholine, N-ethyl morpholine, piperazine, piperidine, triethylamine, trimethylamine. The above salts may be prepared by conventional means from the corresponding compounds of the invention by reacting, for example, the appropriate acid or base with the compounds of Formulae I, IA, IB, III, V, VII, VIII and IX.

Dosages and Treatment Regimen

Dosage levels of the compounds of Formulae I, IA, IB, III, V, VII, VIII and IX typically are on the order of about 0.001 mg to about 10,000 mg daily, with preferred levels of about 0.005 mg to about 1,000 mg daily, more preferred levels of about 0.008 to about 100 mg daily, and still more preferred levels of about 0.05 mg to about 50 mg daily.

The dosage regimen to prevent, treat, give relief from, or ameliorate a hyperlipidemic condition or disorder, or to otherwise protect against or treat further high cholesterol plasma or blood levels with the combinations and compositions of the present invention is selected in accordance with a variety of factors. These factors include the type, age, weight, sex, diet, and medical condition of the patient, the severity of the disease, the route of administration, pharmacological considerations such as the activity, efficacy, pharmacokinetics and toxicology profiles of the particular inhibitors employed, whether a drug delivery system is utilized, and whether the inhibitors are administered with other active ingredients. Thus, the dosage regimen actually employed may vary widely and therefore deviate from the preferred dosage regimen set forth above.

Initial treatment of a patient suffering from a hyperlipidemic condition or disorder can begin with the dosages indicated above. Treatment generally should be continued as necessary over a period of several weeks to several months or years until the hyperlipidemic condition or disorder has been controlled or eliminated. Patients undergoing treatment with the combinations or compositions disclosed herein can be routinely monitored, for example, by measuring serum LDL and total cholesterol levels by any of the methods well-known in the art, to determine the effectiveness of the combination therapy. Continuous analysis of such data permits modification of the treatment regimen during therapy so that optimal effective amounts of each type of inhibitor are administered at any time, and so that the duration of treatment can be determined as well. In this way, the treatment regimen/dosing schedule can be rationally modified over the course of therapy so that the lowest amount of inhibitor that exhibits satisfactory effectiveness is administered, and so that administration is continued only so long as is necessary to successfully treat the hyperlipidemic condition.

The total daily dose of each drug can be administered to the patient in a single dose, or in proportionate multiple subdoses. Subdoses can be administered two to six times per day. Doses can be in immediate release form or sustained release form effective to obtain desired results.

Pharmaceutical Compositions

For the prophylaxis or treatment of the conditions and disorders referred to above, the compound can be administered as the compound per se. Alternatively, pharmaceutically-acceptable salts are particularly suitable for medical applications because of their greater aqueous solubility relative to the parent compound.

The compounds of the present invention also can be presented with an acceptable carrier in the form of a pharmaceutical composition. The carrier must be acceptable in the sense of being compatible with the other ingredients of the composition and must not be deleterious to the recipient. The carrier can be a solid or a liquid, or both, and preferably is formulated with the compound as a unit-dose composition, for example, a tablet, which can contain from 0.05% to 95% by weight of the active compounds. Other pharmacologically active substances can also be present, including other compounds useful in the treatment of a hyperlipidemic condition.

The active compounds of the present invention may be administered by any suitable route, preferably in the form of a pharmaceutical composition adapted to such a route, and in a dose effective for the treatment intended. The active compounds and compositions, for example, may be administered orally, pulmonarily, mucosally, intravascularly, intraperitoneally, subcutaneously, intramuscularly or topically. Unit dose formulations, particularly orally administrable unit dose formulations such as tablets or capsules, generally contain, for example, from about 0.001 to about 500 mg, preferably about 0.005 mg to about 100 mg, and more preferably from about 0.01 to about 50 mg, of the active ingredient. In the case of pharmaceutically acceptable salts, the weights indicated above for the active ingredient refer to the weight of the pharmaceutically active ion derived from the salt.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. If administered per os, the compounds may be admixed with, for example, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration.

Oral delivery of the compounds of the present invention can include formulations, as are well known in the art, to provide immediate delivery or prolonged or sustained delivery of the drug to the gastrointestinal tract by any number of mechanisms. Immediate delivery formulations include, but are not limited to, oral solutions, oral suspensions, fast-dissolving tablets or capsules, disintegrating tablets and the like. Prolonged or sustained delivery formulations include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. The intended effect is to extend the time period over which the active drug molecule is delivered to the site of action (for example, the ileum for the ASBT inhibitor) by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethyl-cellulose phthalate and anionic polymers of methacrylic acid and methacrylic acid methyl ester. Such prolonged or sustained delivery formulations preferably are in dispersed form at the time they reach the ileum.

Pharmaceutical compositions suitable for oral administration can be presented in discrete units, such as capsules, cachets, lozenges, or tablets, each containing a predetermined amount of at least one compound of the present invention; as a powder or granules; as a solution or a suspension in an aqueous or non-aqueous liquid; or as an oil-in-water or water-in-oil emulsion. As indicated, such compositions can be prepared by any suitable method of pharmacy which includes the step of bringing into association the inhibitor(s) and the carrier (which can constitute one or more accessory ingredients). In general, the compositions are prepared by uniformly and intimately admixing the inhibitor(s) with a liquid or finely divided solid carrier, or both, and then, if necessary, shaping the product. For example, a tablet can be prepared by compressing or molding a powder or granules of the inhibitors, optionally with one or more assessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made, for example, by molding the powdered compound in a suitable machine.

Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Pharmaceutical compositions suitable for buccal (sub-lingual) administration include lozenges comprising a compound of the present invention in a flavored base, usually sucrose, and acacia or tragacanth, and pastilles comprising the inhibitors in an inert base such as gelatin and glycerin or sucrose and acacia.

Formulations for parenteral administration, for example, may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Pharmaceutically acceptable carriers encompass all the foregoing and the like. The pharmaceutical compositions of the invention can be prepared by any of the well-known techniques of pharmacy, such as admixing the components. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

Methods of Use

The present invention also includes methods for the treatment of a hyperlipidemic condition or conditions in a subject, including the prophylactic or preventative treatment of a hyperlipidemic condition or conditions in a subject, comprising administering to a subject, particularly a subject in need thereof, a therapeutically effective amount of a compound of Formulae I, IA, IB, III, V, VII, VII or IX.

The present invention further includes methods for the treatment of gallstones in a subject, including the prophylactic or preventative treatment of a hyperlipidemic condition or conditions in a subject, comprising administering to a subject, particularly a subject in need thereof, a therapeutically effective amount of a compound of Formulae I, IA, IB, III, V, VII, VIII or IX.

The methods and compounds of the present invention may be used alone or in conjunction with additional therapies and/or compounds known to those skilled in the art in the prevention or treatment of hyperlipidemia. Alternatively, the methods and compounds described herein may be used, partially or completely, in conjunctive therapy. By way of example, the compounds may be administered alone or in conjunction with other anti-hyperlipidemic agents, such as together with HMG-Co—A reductase inhibitors, bile acid sequestering agents, fibric acid derivatives, nicotinic acid, and/or probucol.

Definitions

The term “subject” as used herein includes an animal, preferably a mammal, and particularly a human, who has been the object of treatment, observation or experiment.

The term “treatment” includes any process, action, application, therapy, or the like, wherein a subject is subject to medical aid with the object of improving the subject's condition, directly or indirectly.

The terms “prophylaxis” and “prevention” include either preventing the onset of a clinically evident hyperlipidemic condition or disorder altogether or preventing the onset of a preclinically evident stage of a hyperlipidemic condition or disorder in individuals. These terms encompass the prophylactic treatment of a subject at risk of developing a hyperlipidemic condition or disorder such as, but not limited to, atherosclerosis and hypercholesterolemia.

The term “combination therapy” or “co-therapy” means the administration of two or more therapeutic agents to treat a hyperlipidemic condition and/or disorder, for example atherosclerosis and hypercholesterolemia. Such administration encompasses co-administration of these therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner. In either case, the treatment regimen will provide beneficial effects of the drug combination in treating the hyperlipidemic condition.

The phrase “therapeutically-effective” qualifies the amount of each agent that will achieve the goal of improvement in hyperlipidemic condition or disorder severity and the frequency of incidence over treatment of each agent by itself, while avoiding adverse side effects typically associated with alternative therapies.

The term “pharmaceutically acceptable” is used adjectivally herein to mean that the modified noun is appropriate for use in a pharmaceutical product. Pharmaceutically acceptable cations, for example, include metallic ions and organic ions. More preferred metallic ions include, but are not limited to appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

The term “prodrug” includes a compound that is a drug precursor that, following administration to a subject and subsequent absorption, is converted to an active species in vivo via some process, such as metabolic conversion. Other products from the conversion process are easily disposed of by the body. More preferred prodrugs produce products from the conversion process that are generally accepted as safe. For example, the prodrug may be an acylated form of the active compound.

The term “ASBT inhibitor” includes a compound capable of inhibiting absorption of bile acids from the intestine into the circulatory system of a mammal, such as a human. This includes increasing the fecal excretion of bile acids, as well as reducing the blood plasma or serum concentrations of cholesterol and cholesterol ester, and more specifically, reducing LDL and VLDL cholesterol. Conditions or diseases which benefit from the prophylaxis or treatment by bile acid transport inhibition include, for example, a hyperlipidemic condition such as atherosclerosis.

Where the term “alkyl” is used, either alone or within other terms such as “haloalkyl”, and “hydroxyalkyl”, it includes linear or branched radicals having one to about twenty carbon atoms or, preferably, one to about twelve carbon atoms. More preferred alkyl radicals are “lower alkyl” radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like. Even more preferred are lower alkyl radicals having one to three carbon atoms.

Where the term “alkenyl” is used, either alone or within other terms such as “arylalkenyl”, it includes linear or branched radicals having at least one carbon—carbon double bond of two to about twenty carbon atoms or, preferably, two to about twelve carbon atoms. More preferred alkenyl radicals are “lower alkenyl” radicals having two to about six carbon atoms. Examples of alkenyl radicals include ethenyl, propenyl, allyl, propenyl, butenyl and 4-methylbutenyl.

The terms “alkenyl” and “lower alkenyl”, include radicals having “cis” and “trans” orientations, or alternatively, “E” and “Z” orientations.

The term “alkynyl” includes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The term “cycloalkyl” includes saturated carbocyclic radicals having three to about twelve carbon atoms. More preferred cycloalkyl radicals are “lower cycloalkyl” radicals having three to about ten carbon atoms. Examples of such radicals include cyclopropyl, cyclobutyl, cyclopentyl and cyclohexyl. The term “cycloalkyl” additionally encompasses spiro systems wherein the cycloalkyl ring has a carbon ring atom in common with the seven-membered heterocyclic ring of the benzothiazepine.

The term “cycloalkenyl” includes partially unsaturated carbocyclic radicals having three to twelve carbon atoms. Cycloalkenyl radicals that are partially unsaturated carbocyclic radicals that contain two double bonds (that may or may not be conjugated) can be called “cycloalkyldienyl”. More preferred cycloalkenyl radicals are “lower cycloalkenyl” radicals having four to about ten carbon atoms. Examples of such radicals include cyclobutenyl, cyclopentenyl and cyclohexenyl.

The term “halo” and “halogen” includes halogens such as fluorine, chlorine, bromine or iodine atoms. The term “haloalkyl” includes radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” includes radicals having one to six carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perfluoroalkyl” includes alkyl radicals having all hydrogen atoms replaced with fluoro atoms. Examples include trifluoromethyl and pentafluoroethyl.

The term “hydroxyalkyl” includes linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl. Even more preferred are lower hydroxyalkyl radicals having one to three carbon atoms.

The term “aryl”, alone or in combination, includes a carbocyclic aromatic system containing one or more rings wherein such rings may be attached together in a pendent manner or may be fused. The term “aryl” includes aromatic radicals such as phenyl, naphthyl, tetrahydronaphthyl, indane, biphenyl, and anthracenyl. More preferred aryl is phenyl. Said “aryl” group may have one to three substituents such as lower alkyl, hydroxy, halo, haloalkyl, nitro, cyano, alkoxy and lower alkylamino.

The term “heterocyclyl” includes saturated, partially saturated and unsaturated heteroatom-containing ring-shaped radicals, where the heteroatoms may be selected from nitrogen, sulfur and oxygen. Preferred heterocyclyl are 3-10 membered ring heterocyclyl, particularly 5-8 membered ring heterocyclyl. Examples of saturated heterocyclic radicals include saturated 3 to 6-membered heteromonocyclic groups containing 1 to 4 nitrogen atoms [e.g. pyrrolidinyl, imidazolidinyl, piperidino, piperazinyl]; saturated 3 to 6-membered heteromonocyclic groups containing 1 to 2 oxygen atoms and 1 to 3 nitrogen atoms [e.g. morpholinyl]; saturated 3 to 6-membered heteromonocyclic groups containing 1 to 2 sulfur atoms and 1 to 3 nitrogen atoms [e.g., thiazolidinyl]. Examples of partially saturated heterocyclyl radicals include dihydrothiophene, dihydropyran, dihydrofuran and dihydrothiazole. Examples of unsaturated heterocyclic radicals, also termed “heteroaryl” radicals, include unsaturated 5 to 6 membered heteromonocyclyl groups containing 1 to 4 nitrogen atoms, for example, pyrrolinyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated condensed heterocyclic groups containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo [1,5-b]pyridazinyl]; unsaturated 3 to 6-membered heteromonocyclic groups containing an oxygen atom, for example, pyranyl, 2-furyl, 3-furyl, etc.; unsaturated 5 to 6-membered heteromonocyclic groups containing a sulfur atom, for example, 2-thienyl, 3-thienyl, etc.; unsaturated 5- to 6-membered heteromonocyclic groups containing 1 to 2 oxygen atoms and 1 to 3 nitrogen atoms, for example, isoxazolyl, oxadiazolyl [e.g., 1,2,4-oxadiazolyl, 1,3,4-oxadiazolyl, 1,2,5-oxadiazolyl]; unsaturated condensed heterocyclic groups containing 1 to 2 oxygen atoms and 1 to 3 nitrogen atoms [e.g. benzoxazolyl, benzoxadiazolyl]; unsaturated 5 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl]; unsaturated condensed heterocyclic groups containing 1 to 2 sulfur atoms and 1 to 3 nitrogen atoms [e.g., benzothiazolyl, benzothiadiazolyl] and the like. The term also includes radicals where heterocyclic radicals are fused with aryl radicals. Examples of such fused bicyclic radicals include benzofuran, benzothiophene, and the like. Said “heterocyclyl” group may have 1 to 3 substituents such as lower alkyl, hydroxy, oxo, amino and lower alkylamino.

Heterocyclic radicals can include fused or unfused radicals, particularly 3-10 membered fused or unfused radicals. Preferred examples of heteroaryl radicals include benzofuryl, 2,3-dihydrobenzofuryl, benzothienyl, indolyl, dihydroindolyl, chromanyl, benzopyran, thiochromanyl, benzothiopyran, benzodioxolyl, benzodioxanyl, pyridyl, thienyl, thiazolyl, furyl, and pyrazinyl. More preferred heteroaryl radicals are 5- or 6-membered heteroaryl, containing one or two heteroatoms selected from sulfur nitrogen and oxygen, selected from thienyl, furanyl, thiazolyl, imidazolyl, pyrazolyl, isoxazolyl, isothiazolyl, pyridyl, piperidinyl and pyrazinyl.

The term “heteroaryl” includes a fully unsaturated heterocyclyl.

In either “heterocyclyl” or “heteroaryl,” the point of attachment to the molecule of interest can be at the heteroatom or elsewhere within the ring.

The term “triazolyl” includes all positional isomers. In all other heterocyclyl and heteroaryl which contain more than one ring heteroatom and for which isomers are possible, such isomers are included in the definition of said heterocyclyl and heteroaryl.

The term “quaternary heterocyclyl” includes a heterocyclyl in which one or more of the heteroatoms, for example, nitrogen, sulfur, phosphorus or oxygen, has such a number of bonds that it is positively charged (and therefore the term is intended to encompass both ternary and quaternary positively charged structures). The point of attachment of the quaternary heterocyclyl to the molecule of interest can be at a heteroatom or elsewhere.

The term “quaternary heteroaryl” includes a heteroaryl in which one or more of the heteroatoms, for example, nitrogen, sulfur, phosphorus or oxygen, has such a number of bonds that it is positively charged (and therefore the term is intended to encompass both ternary and quaternary positively charged structures). The point of attachment of the quaternary heteroaryl to the molecule of interest can be at a heteroatom or elsewhere.

The term “oxo” includes a doubly bonded oxygen.

The term “polyalkyl” includes a branched or straight hydrocarbon chain having a molecular weight up to about 20,000, more preferably up to about 10,000, and most preferably up to about 5,000.

The term “polyether” includes a polyalkyl wherein one or more carbons are replaced by oxygen, wherein the polyether has a molecular weight up to about 20,000, more preferably up to about 10,000, and most preferably up to about 5,000.

The term “polyalkoxy” includes a polymer of alkylene oxides, wherein the polyalkoxy has a molecular weight up to about 20,000, more preferably up to about 10,000, and most preferably up to about 5,000.

The term “carbohydrate residue” encompasses residues derived from carbohydrates such as, but is not limited to, mono-, di-, tri-, tetra- and polysaccharides wherein the polysaccharides can have a molecular weight of up to about 20,000, for example, hydroxypropyl-methylcellulose or chitosan residue; compounds derived from aldoses and ketoses with 3 to 7 carbon atoms and which belong to the D- or L-series; aminosugars; sugar alcohols; and saccharic acids. Nonlimiting specific examples of such carbohydrates include glucose, mannose, fructose, galactose, ribose, erythrose, glycerinaldehyde, sedoheptulose, glucosamine, galactosamine, glucoronic acid, galacturonic acid, gluconic acid, galactonic acid, mannoic acid, glucamine, 3-amino-1,2-propanediol, glucaric acid and galactaric acid.

The term “peptide residue” includes polyamino acid residue containing up to about 100 amino acid units.

The term “polypeptide residue” includes a polyamino acid residue containing from about 100 amino acid units to about 1000 amino acid units, more preferably from about 100 amino acid units to about 750 amino acid units, and most preferably from about 100 amino acid units to about 500 amino acid units.

The term “alkylammoniumalkyl” includes an an —NH₂ group or a mono-, di- or tri-substituted amino group, any of which is bonded to an alkyl wherein said alkyl is bonded to the molecule of interest.

The term “sulfo” includes a sulfo group, —SO₃H, and its salts.

The term “sulfoalkyl” includes an alkyl group to which a sulfonate group is bonded, wherein said alkyl is bonded to the molecule of interest.

The term “aralkyl” includes aryl-substituted alkyl radicals. Preferable aralkyl radicals are “lower aralkyl” radicals having aryl radicals attached to alkyl radicals having one to six carbon atoms. Even more preferred are lower aralkyl radicals having phenyl attached to alkyl portions having one to three carbon atoms. Examples of such radicals include benzyl, diphenylmethyl and phenylethyl. The aryl in said aralkyl may be additionally substituted with halo, alkyl, alkoxy, halkoalkyl and haloalkoxy.

The term “arylalkenyl” includes aryl-substituted alkenyl radicals. Preferable arylalkenyl radicals are “lower arylalkenyl” radicals having aryl radicals attached to alkenyl radicals having

The term “heterocyclylalkyl” includes an alkyl radical that is substituted with one or more heterocyclyl groups. Preferable heterocyclylalkyl radicals are “lower heterocyclylalkyl” radicals having one or more heterocyclyl groups attached to an alkyl radical having one to ten carbon atoms.

The term “heteroarylalkyl” includes an alkyl radical that is substituted with one or more heteroaryl groups. Preferable heteroarylalkyl radicals are “lower heteroarylalkyl” radicals having one or more heteroaryl groups attached to an alkyl radical having one to ten carbon atoms.

The term “quaternary heterocyclylalkyl” includes an alkyl radical that is substituted with one or more quaternary heterocyclyl groups. Preferable quaternary heterocyclylalkyl radicals are “lower quaternary heterocyclylalkyl” radicals having one or more quaternary heterocyclyl groups attached to an alkyl radical having one to ten carbon atoms.

The term “quaternary heteroarylalkyl” includes an alkyl radical that is substituted with one or more quaternary heteroaryl groups. Preferable quaternary heteroarylalkyl radicals are “lower quaternary heteroarylalkyl” radicals having one or more quaternary heteroaryl groups attached to an alkyl radical having one to ten carbon atoms.

The term “alkylheteroarylalkyl” includes a heteroarylalkyl radical that is substituted with one or more alkyl groups. Preferable alkylheteroarylalkyl radicals are “lower alkylheteroarylalkyl” radicals with alkyl portions having one to ten carbon atoms.

The term “alkoxy” includes an alkyl radical which is attached to the molecule of interest by oxygen, such as a methoxy radical. More preferred alkoxy radicals are “lower alkoxy” radicals having one to six carbon atoms. Examples of such radicals include methoxy, ethoxy, propoxy, iso-propoxy, butoxy and tert-butoxy.

The term “carboxy” includes the carboxy group, —CO₂H, and its salts.

The term “carboxyalkyl” includes an alkyl radical that is substituted with one or more carboxy groups. Preferable carboxyalkyl radicals are “lower carboxyalkyl” radicals having one or more carboxy groups attached to an alkyl radical having one to six carbon atoms.

The term “carboxyheterocyclyl” includes a heterocyclyl radical that is substituted with one or more carboxy groups.

The term “carboxyheteroaryl” includes a heteroaryl radical that is substituted with one or more carboxy groups.

The term “carboalkoxyalkyl” includes an alkyl radical that is substituted with one or more alkoxycarbonyl groups. Preferable carboalkoxyalkyl radicals are “lower carboalkoxyalkyl” radicals having one or more alkoxycarbonyl groups attached to an alkyl radical having one to six carbon atoms.

The term “carboxyalkylamino” includes an amino radical that is mono- or di-substituted When used in combination, for example “alkylaryl” or “arylalkyl,” the individual terms listed above have the meaning indicated above.

The term “acyl” includes an organic acid group in which the hydroxy of the carboxy group has been removed. Examples of acyl groups include, but are not limited to, acetyl and benzoyl.

The term “hydrocarbyl” refers to radicals consisting exclusively of the elements carbon and hydrogen. These radicals include, for example, alkyl, cycloalkyl, alkenyl, cycloalkenyl, alkynyl, and aryl moieties. These radicals also include alkyl, cycloalkyl, alkenyl, cycloalkenyl, alkynyl, and aryl moieties substituted with other aliphatic or cyclic hydrocarbon groups, such as alkaryl, alkenaryl and alkynaryl. Preferably, these moieties comprise 1 to 20 carbon atoms, 1-10 carbons or 1-6 carbons.

The term “a substituted hydrocarbyl” refers to a hydrocarbyl radical that is substituted with a group comprising at least one atom other than carbon, such as but not limited to, halogen, oxygen, nitrogen, sulfur and phosphorus. Examples of such substituted hydrocarbyl include hydrocarbyl radicals substituted with groups such as, but not limited to, lower alkoxy such as methoxy, ethoxy, and butoxy; halogen such as chloro and fluoro; ethers; acetals; ketals; esters; heterocyclyl such as furyl and thienyl; alkanoxy; hydroxy; protected hydroxy; acyl; acyloxy; nitro; cyano; amino; and amido. Substituted hydrocarbyl also includes hydrocarbyl radicals in which a carbon chain atom is replaced with a heteroatom such as nitrogen, oxygen, sulfur, or a halogen.

The additional terms used to describe the substituents of the compounds of the present invention and not specifically defined herein are defined in a similar manner to that illustrated in the above definitions.

General Synthetic Procedures

The compounds of the present invention can be synthesized according to the general synthetic procedures set forth below. The substituents of the compounds shown in the following procedures generally have the same definition as the substituents at the corresponding position in the compounds of Formulae I, IA, IB, III, V, VII, VIII and/or IX, except where further noted.

The synthetic methods for the preparation of the 1,4-benzothiazepines disclosed, for example, in WO93/16055, WO94/18183, WO94/18184, WO96/05188, WO98/05657, U.S. Pat. No. 5,910,494, and U.S. Pat. No. 6,020,330 can be modified as illustrated below and in the working examples to prepare the 1,4-benzothiazapine compounds of the present invention.

In particular, the 1,4-benzothiazapine compounds of the present invention can be prepared as set forth in Scheme I below.

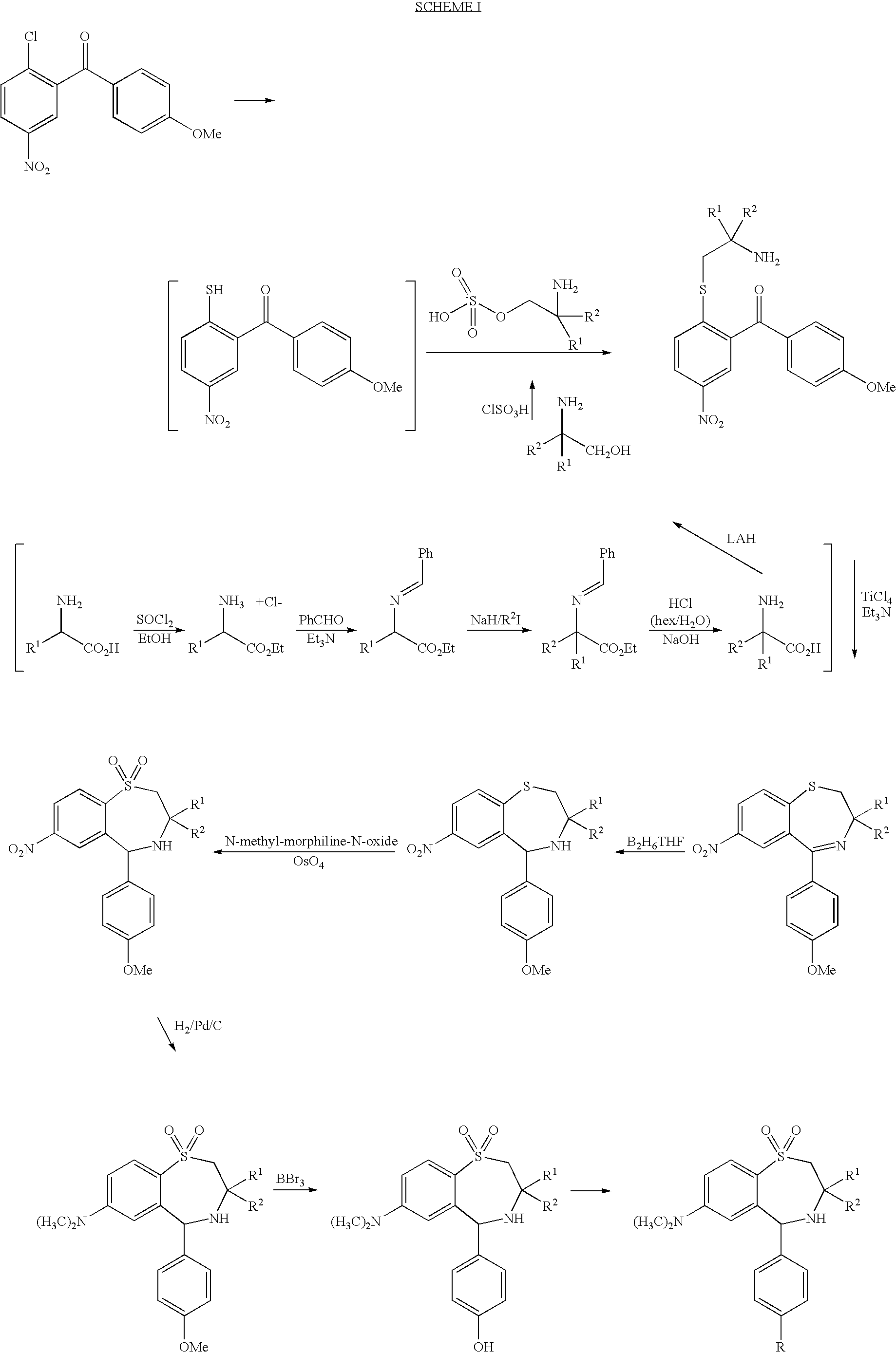

The 5-position phenyl group of the 1,4-benzothiazepine intermediates of Scheme I can be further substituted (with R) as specifically disclosed in this application or, for example, by substituting the 5-position phenyl group through suitable modification of the methods disclosed in U.S. Pat. No. 5,994,391 and WO99/64409.

The synthetic methods for the preparation of 1,5-benzothiazepines disclosed, for example, in WO96/16051 and WO99/35135 can be modified as illustrated below and in the working examples to prepare the 1,5-benzothiazapine compounds of the present invention.

In particular, the 1,5-benzothiazapine compounds of the present invention can be prepared as set forth in Scheme II below.

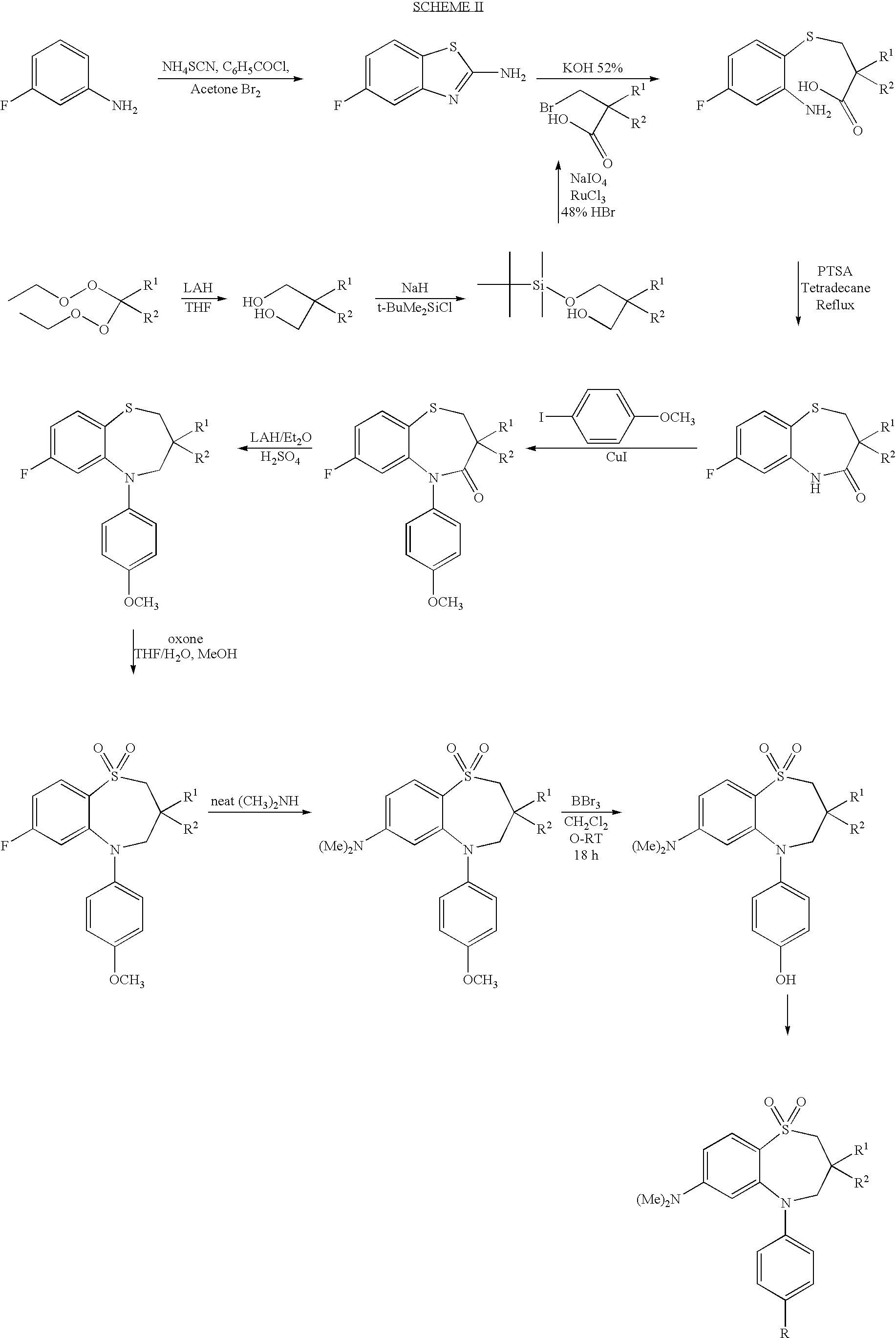

The 5-position phenyl group of the 1,5-benzothiazepine intermediates can be can be further substituted (with R) as specifically disclosed in this application or, for example, by substituting the 5-position phenyl group through suitable modification of the methods disclosed in U.S. Pat. No. 5,994,391 and WO99/64409.

Working Examples

The following examples contain detailed descriptions of the methods of preparation of the compounds of the present invention. These detailed descriptions fall within the scope, and serve to exemplify, the above-described general synthetic procedures which form part of the invention. These detailed descriptions are presented for illustrative purposes only and are not intended as a restriction on the scope of the invention. All parts are by weight and temperatures are in degrees centigrade unless otherwise indicated. The preparation of the reagents used in these Examples is either specifically disclosed herein or such reagents are commercially available.

The following abbreviations are used in the examples below:

Me—methyl

Et—ethyl

EtOH—ethanol

Et₃N—triethylamine

HCl—hydrochloric acid

LAH—lithium aluminum hydride

LiOH—lithium hydroxide

MeOH—methanol

NaOH—sodium hydroxide

Ph—phenyl

PTSA—para-toluene sulfonic acid

RT—room temperature

THF—tetrahydrofuran

### EXAMPLE 1

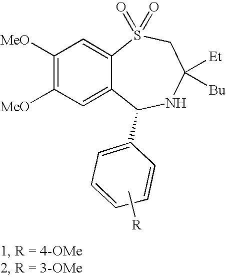

Compounds 1 and 2 are prepared in accordance with the procedure set forth in Synthetic Example 1 of patent application WO96/05188, except that 4-methoxybenzoyl chloride and 3-methoxybenzoyl chloride, respectively, are substituted for benzoyl chloride in step (h).

### EXAMPLE 2

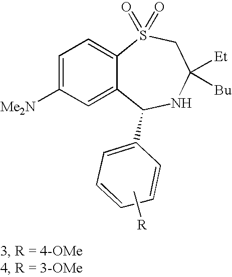

Step 1: Preparation of 4-fluorophenyl Substituted Intermediate

A 4-fluorophenyl substituted intermediate is prepared in accordance with the procedure set forth in Synthetic Example 1 of patent application WO96/05188, except that 4-fluorophenol is substituted for 3,4-dimethoxyphenol and 4-methoxybenzoyl chloride (or 3-methoxybenzoyl chloride) is substituted for benzoyl chloride in step (h) of Synthetic Example 1.

Step 2: Preparation of Dimethylamino-Compound

A Fisher porter bottle is fitted with a nitrogen line and magnetic stirrer. The system is purged with nitrogen. The 4-fluorophenyl substituted intermediate (62.6 mmol) obtained from Step 1 is added and the vessel is sealed and cooled to −78° C. Dimethylamine (17.1 g, 379 mmol) is condensed using a CO₂/acetone bath and added to the reaction vessel. The mixture is allowed to warm to room temperature and heated to 60° C. After 20 hours, the reaction mixture is allowed to cool and dissolved in ethyl ether. The ether solution is washed with water and then with saturated aqueous sodium chloride. It is then dried with magnesium sulfate, filtered, and concentrated in vacuo to give compound 3 (or compound 4 when 3-methoxybenzoyl chloride is used in Step 1).

### EXAMPLE 3

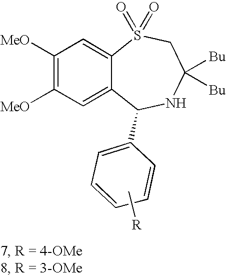

Step 1: Preparation of 2-amino-2-butylhexyl Hydrogen Sulfate

A 2-amino-2-butylhexyl hydrogen sulfate is prepared in accordance with the procedure set forth in steps (a) through (g) of Synthetic Example 1 of patent application WO96/05188, except that 2-aminohexanoic acid is substituted for 2-aminobutyric acid in step (a) of Synthetic Example 1.

Step 2: Preparation of the Dimethoxybenzothiazepine

Compounds 7 and 8 are prepared in accordance with the procedure set forth in steps (h) through (O) of Synthetic Example 1 of patent application WO96/05188, except that 2-amino-2-butylhexyl hydrogen sulfate (obtained from Step 1) is substituted for 2-amino-2-ethylhexyl hydrogen sulfate, and either 4-methoxybenzoyl chloride (for Compound 7) or 3-methoxybenzoyl chloride (for Compound 8) is substituted for benzoyl chloride in Step (h) of Synthetic Example 1.

### EXAMPLE 4

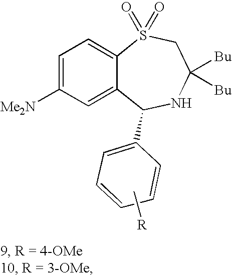

Step 1: Preparation of 4-fluorophenyl Substituted Intermediate

A 4-fluorophenyl substituted intermediate is prepared in accordance with the procedure set forth in steps (h) through (O) of Synthetic Example 1 of patent application WO96/05188, except that (a) 4-fluorophenol is substituted for 3,4-dimethoxyphenol in step (h) of Synthetic Example 1, (b) either 4-methoxybenzoyl chloride (for Compound 9) or 3-methoxybenzoyl chloride (for Compound 10) is substituted for benzoyl chloride in step (h) of Synthetic Example 1, and (c) 2-amino-2-butylhexyl hydrogen sulfate (obtained from Example 3, Step 1) is substituted for 2-amino-2-ethylhexyl hydrogen sulfate in step (h) of Synthetic Example 1.

Step 2: Preparation of Dimethylamino-Compound

A Fisher porter bottle is fitted with nitrogen line and magnetic stirrer. The system is purged with nitrogen. The 4-fluorophenyl substituted intermediate (62.6 mmol, obtained from Step 1) is added, and the vessel is sealed and cooled to −78° C. Dimethylamine (17.1 g, 379 mmol) is condensed using a CO₂/acetone bath and added to the reaction vessel. The mixture is allowed to warm to room temperature and heated to 60° C. After 20 hours, the reaction mixture is allowed to cool and dissolved in ethyl ether. The ether solution is washed with water and then saturated aqueous sodium chloride. It is then dried (magnesium sulfate), filtered, and concentrated in vacuo to give either Compound 9 (when 4-methoxybenzoyl chloride is used in Step 1) or 10 (when 3-methoxybenzoyl chloride is used in Step 1).

### EXAMPLE 5

### Alternate Route to Compounds 3 and 9

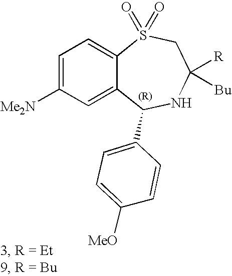

Step 1: Preparation of Chlorobenzophenone

In an inert atmosphere, 68.3 g of phosphorus pentachloride (0.328 mole Aldrich 15,777-5) is placed into a 2-necked 500 mL round bottom flask. The flask is fitted with a nitrogen inlet adapter and suba seal. The flask is removed from the inert atmosphere and a nitrogen purge is begun. 50 mL of anhydrous chlorobenzene (Aldrich 28,451-3) is added to the phosphorus pentachloride via syringe and the solution is stirred with a magnetic stir bar. 60 g of 2-chloro-5-nitrobenzoic acid (0.298 mole Aldrich 12,511-3) is slowly added to the chlorobenzene solution under nitrogen purge. After stirring at room temperature for about 20 hours, the solution is placed in an oil bath and heated at 50° C. for 1 hour. The chlorobenzene is removed from the solution by high vacuum. The residue is washed with anhydrous hexane to yield a dry acid chloride having a weight of 61.95 g. The acid chloride is stored in an inert and dry atmosphere.

In an inert atmosphere, the acid chloride is mixed with 105 mL of anhydrous anisole (0.97 mole Aldrich 29,629-5) in a 2-necked 500 mL round bottom flask. The flask is fitted with an addition funnel and a nitrogen inlet adapter and removed from inert atmosphere. The reaction solution is chilled with an ice bath and a nitrogen purge is begun. 45.1 g of aluminum chloride (0.34 moles Aldrich 29,471-3) is placed in a solid addition funnel and the aluminum chloride is slowly added to the chilled solution. After the addition is complete, the solution is allowed to warm to room temperature and stirred overnight. The reaction is quenched by pouring the solution into a mixture of 300 mL 1N HCl and ice. The resulting solution is stirred for 15 minutes and extracted twice with ether. The organic layers are combined and extracted twice with 2% sodium hydroxide, and then twice with deionized water. The extracted organic layer is then dried with magnesium sulfate, filtered and further dried using a rotovap. Remaining anisole is then removed by high vacuum. The resulting product is crystallized from 90% ethanol 10% ethyl acetate and dried on a vacuum line.

Step 2: Preparation of Thiobenzophenone Intermediate

10.12 g (0.036 moles) of the chlorobenzophenone intermediate obtained from Step 1 is combined with 200 mL of anhydrous dimethylsulfoxide and placed in a 500 mL round bottom flask equipped with a magnetic stir bar. The flask is fitted with a water condenser, nitrogen inlet, and stopper. 1.84 g of Li₂S (0.040 moles Aldrich 21,324-1) is added. The flask is placed in an oil bath and heated overnight at 75° C. under nitrogen. The solution is cooled to room temperature. 500 mL of 5% acetic acid is prepared in a 2 liter beaker and then added slowly to the solution in the flask while stirring is maintained in the flask. After 30 minutes, the solution is extracted with ether three times, dried (magnesium sulfate), filtered and concentrated in vacuo to give a thiobenzophenone intermediate.

Step 3: Preparation of Thioamine Intermediate

A solution of the thiobenzophenone intermediate (198 mmol, obtained from Step 2) in butyl acetate (300 mL) is added to a solution of the ethyl-butyl amine (for compound 3) or butyl-butyl amine (for compound 9) (obtained as set forth in Synthetic Example 1 of WO96/05188 or in Example 3, Step 1 of the present application) in water (250 mL). The reaction mixture is stirred and heated to 93° C. Sodium hydroxide (18.9 g) in water (250 mL) is added dropwise. After complete addition, the reaction is stirred an additional 25 minutes at 93° C. and then cooled to room temperature. The organic layer is separated, dried and concentrated to give a thioamine intermediate.

Step 4: Preparation of the Cyclic Imine Intermediate

The thioamine intermediate (194 mmol, obtained from Step 3) is dissolved in 2,6-lutidine. p-Toluenesulfonic acid (0.70 g) is added and the reaction mixture is refluxed using a Dean Stark trap. After 22 hours of refluxing, the reaction mixture is concentrated in vacuo. Chromatography on silica gel gives the purified cyclic imine intermediate.

Step 5: Preparation of the Cylic Amine Intermediate

A 1M solution of diborane in tetrahydrofuran (200 mL) is added to a solution of the cyclic imine intermediate (167 mmol, obtained from Step 4) in tetrahydrofuran (350 mL). The reaction mixture is stirred at room temperature for 17 hours and then 6N HCl (150 mL) is added. The tetrahydrofuran is removed under reduced pressure and the aqueous residue basified with 50% sodium hydroxide. The resulting solution is extracted with ethyl acetate and the ethyl acetate layer is separated, dried and concentrated in vacuo. Purification by chromatography on silica gel gives the cyclic amine intermediate.

Step 6: Preparation of the Sulfone Intermediate

A solution of the cyclic amine intermediate (66.2 mmol, obtained from Step 5) in trifluoroacetic acid (125 mL) is added to 30% water (18.8 g) in trifluoroacetic acid (100 mL). The reaction mixture is stirred at room temperature for 17 hours and then poured into water (800 mL). 50% sodium hydroxide is then added until the mixture reaches a pH of 10. The reaction mixture is layered with ethyl acetate and stirred for 1 hour. The organic layer is separated, dried and concentrated in vacuo. The residue is purified by recrystallization or chromatography on silica gel to give the desired sulfone intermediate.

Step 7: Preparation of the Dimethylamino-Benzothiazepine

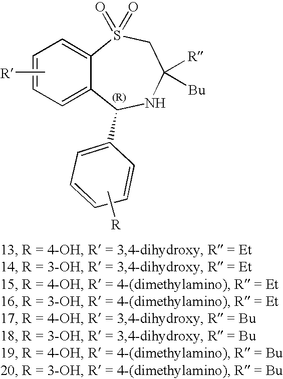

The sulfone intermediate (20.4 mmol, obtained from Step 6) and ethanol (160 mL) are placed in a 300 mL Parr reactor. Formaldehyde (15.3 mL, 189 mmol, 37 weight percent in water) and 10% Pd/Carbon (1.45 g) are added. The reactor is heated to 55° C. under hydrogen overnight. The reactor is cooled and purged with nitrogen. The solution is filtered through celite while washing with ether. The mixture is concentrated in vacuo, redissolved with ether, and washed with water. The organic layer is dried (magnesium sulfate), filtered and concentrated in vacuo to give the desired dimethylamino-benzothiazepine 3 or 9.

### EXAMPLE 6

A 250-mL, 3-neck, round-bottom flask is equipped with a nitrogen gas adaptor and magnetic stirrer. The system is purged with nitrogen. The corresponding methoxy-compound 1, 2, 3, 4, 7, 8, 9 or 10 (14.0 mmol) and trichloromethate (150 mL) are added to the flask. The reaction mixture is cooled to −78° C. and boron tribromide (10.50 g/41.9 mmol per methoxy group) is added. The mixture is allowed to warm to room temperature. After 4 hours, the reaction mixture is cooled to 0° C. and quenched with 10% K₂CO₃ (100 mL). After 10 minutes, the layers are separated and the aqueous layer is extracted two times with ethyl ether. The trichloromethane and ether extracts are combined, washed with saturated aqueous sodium chloride, dried (magnesium sulfate), filtered, and concentrated in vacuo to give the desired product 13, 14, 15, 16, 17, 18, 19 or 20. See, M. Kitamura et al., J. Am. Chem. Soc., 106, 3252-57 (1984).

### EXAMPLE 7

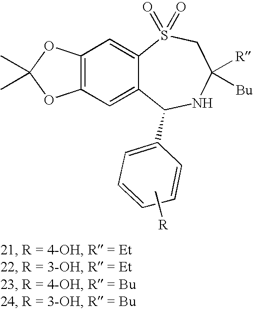

A 250-mL, 3-neck, round-bottom flask is equipped with a nitrogen gas adaptor and magnetic stirrer. The system is purged with nitrogen. The corresponding hydroxy-compound 13, 14, 17 or 18 (14.0 mmol) and dimethylformamide (150 mL) are added to the flask. The reaction mixture is cooled to 5° C. Acetone (48 mL), 2,2-dimethoxypropane (9.6 mL), and pyridinium p-toluenesulfonate (0.54 g) are added to the reaction mixture. After 24 hours, Amberlite IRA 402 (a strongly basic anion exchanger, quaternary ammonium type resin, chloride form, available from Sigma Chemical) is added to neutralize the catalyst. After 24 hours, the resin is removed by filtration, and the filtrate concentrated in vacuo to give the desired product 21, 22, 23 or 24. See, M. Kitamura et al., J. Am. Chem. Soc., 106, 3252-57 (1984).

### EXAMPLE 8

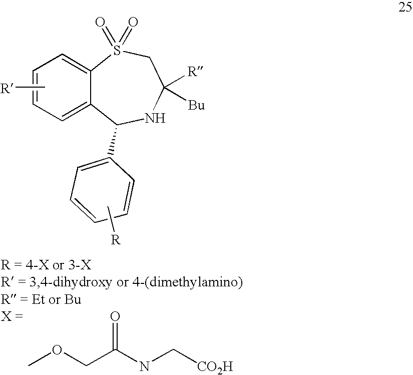

Step 1: Preparation of Glycine Ester Intermediate

To a solution of (13.9 mmol) of 15, 16, 19, 20, 21, 22, 23 or 24 (obtained from Example 6 or 7) and 2.9 g (21.0 mmol) of potassium carbonate in 100 mL of acetone is added 3.8 g (21.0 mmol) of N-(chloroacetyl)glycine ethyl ester and 50 mg (0.14 mmol) of tetrabutylammonium iodide. The reaction mixture is heated to reflux for 2 days, cooled to ambient temperature and stirred for 20 hours. It is then partitioned between ethyl acetate and water. The organic layer is washed with brine, dried over magnesium sulfate, and concentrated in vacuo to afford a glycine ester intermediate.

Step 2: Preparation of Acid

A solution of the glycine ester intermediate (12.1 mmol, obtained from Step 1) and 1.5 g LiOH.H₂O (36.3 mmol) in 60 mL of tetrahydrofuran and 60 mL of water is heated to 45° C. for 2 hours. The solution is then cooled to ambient temperature, acidified with 1 N HCl and partitioned between ethyl acetate and water. The organic layer is washed with brine, dried over magnesium sulfate, and concentrated in vacuo to give the desired compound 25. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate, and concentrated in vacuo to give the desired product 25. See, e.g., Y. Leblanc et al., J. Org. Chem., 51, 189-93 (1986).

### EXAMPLE 9

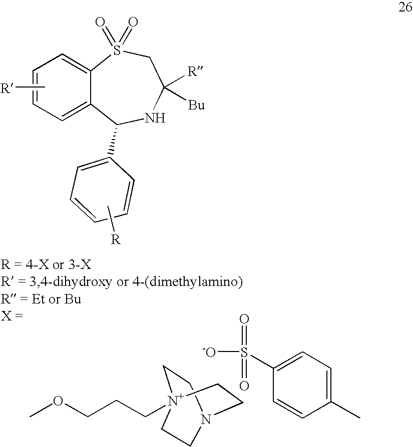

Step 1: Preparation of Propyl Tosylate Intermediate

A solution of 15, 16, 19, 20, 21, 22, 23 or 24 (10.9 mmol, obtained from Example 6 or 7) in acetone (100 mL) at 25° C. under nitrogen is treated with powdered K₂CO₃ (3.8 g, 27.2 mmol, 2.5 equivalents) and 1,3-propanediol di-p-tosylate (13.0 g, 32.6 mmol, 3.0 equivalents), and the resulting mixture is stirred at 65° C. for 21 hours. The cream-colored slurry is cooled to 25° C. and is filtered through a sintered glass funnel. The filtrate is concentrated and the residue dissolved in ethyl acetate (150 mL). The organic layer is washed with saturated aqueous sodium bicarbonate (2×150 mL) and saturated aqueous sodium chloride (2×150 mL), and is dried (magnesium sulfate) and concentrated in vacuo to afford a propyl tosylate intermediate. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate, and concentrated in vacuo to give a propyl tosylate intermediate. See, e.g., Y. Leblanc et al., J. Org. Chem., 51, 189-93 (1986).

Step 2: Preparation of Quaternary Salt

A solution of the propyl tosylate intermediate (1.56 mmol, obtained from Step 1) in acetonitrile (15 mL) at 25° C. under nitrogen is treated with diazabicyclo[2,2,2]octane (“DABCO”, 0.26 g, 2.34 mmol, 1.5 equivalents) and stirred at 50° C. for 6 hours and then at 25° C. for 14 hours. The pale amber solution is cooled to 25° C. and concentrated in vacuo to give the desired compound 26.

### EXAMPLE 10

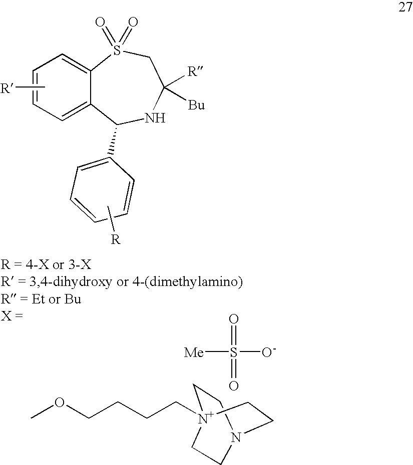

Step 1: Preparation of Butyl Mesylate Intermediate

A mixture of 2.18 mmol of 15, 16, 19, 20, 21, 22, 23 or 24 (obtained from Example 6 or 7), 2.68 g (10.88 mmol) of busulfan, and 1.50 g (10.88 mmol) of potassium carbonate in 20 mL of acetone is stirred at reflux overnight. The mixture is concentrated in vacuo and the crude is dissolved in 30 mL of ethyl acetate. The insoluble solid is filtered off and the filtrate is concentrated in vacuo. The resulting white foam is chromatographed through a silica gel column and eluted with 30% ethyl acetate/hexane to give the butyl mesylate intermediate. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate and concentrated in vacuo to give a butyl mesylate intermediate. See, e.g., Y. Leblanc et al., J. Org. Chem., 51, 189-93 (1986).

Step 2: Preparation of Quaternary Salt

A solution of 0.85 mmol of the butyl mesylate intermediate (obtained from Step 1) and 191 mg (1.71 mmol) of diazabicyclo[2,2,2]octane in 10 mL of acetonitrile is stirred at 80° C. for 4 hours. The reaction mixture is concentrated in vacuo to yield a white foam. The foam is crushed and washed with ether. The solid is filtered off and dried in vacuo to give the desired compound 27.

### EXAMPLE 11

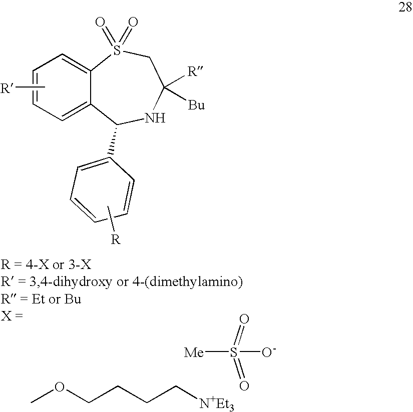

A solution of 1.64 mmol of the butyl mesylate intermediate (obtained from Example 10, Step 1) and 15 mL of triethylamine in 10 mL of acetonitrile is heated at 50° C. for 2 days. The solvent is evaporated and the residue triturated with ether and ethyl acetate to afford the desired product 28.

### EXAMPLE 12

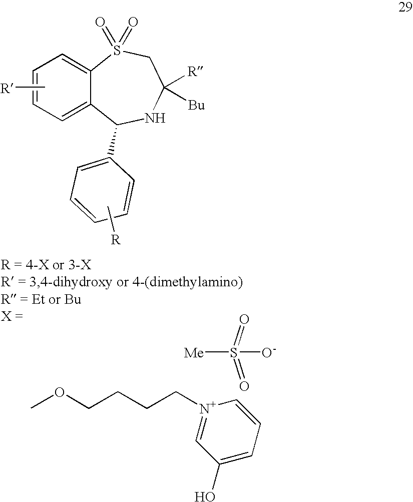

A solution of 1.64 mmol of the butyl mesylate intermediate (obtained from Example 10, Step 1) and 234 mg (2.46 mmol) of 3-hydroxy-pyridine in 1 mL of dimethylformamide is heated at 70° C. for 20 hours. The solvent is evaporated and the residue triturated with ether and ethyl acetate to afford the desired product 29.

### EXAMPLE 13

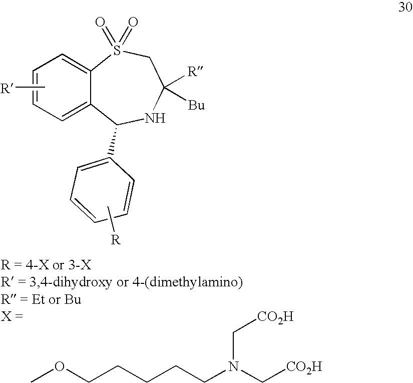

Step 1: Preparation of Pentyl Bromide Intermediate

13.1 mmol of 15, 16, 19, 20, 21, 22, 23 or 24 (obtained from Example 6 or 7) is added to a stirred solution of 0.63 g (15.72 mmol, 60% dispersion) of sodium hydride in 85 mL of dimethylformamide. The resulting solution is stirred at ambient temperature for 1 hour. 37.7 g (163.75 mmol) of 1,5-dibromopentane is added to the solution and the solution stirred overnight at ambient temperature. The dimethylformamide is removed in vacuo and the residue is extracted with ethyl acetate and washed with brine. The extract is dried over magnesium sulfate, and the concentrated residue purified by column chromatography to give a pentyl bromide intermediate.

Step 2: Preparation of Diester Intermediate

A mixture of 14.1 mmol of the pentyl bromide intermediate (obtained from Step 1), −65 g (0.35 mol) of diethylaminodiacetate and 7.5 g (71 mmol) of anhydrous Na₂CO₃ is stirred at 160° C. for 3 hours. The reaction mixture is diluted with water and extracted with methylene chloride. The volatiles are removed in vacuo to give the diester intermediate. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate and concentrated in vacuo to give a diester intermediate. See, e.g., Y. Leblanc et al., J. Org. Chem., 51, 189-93 (1986).

Step 3: Preparation of Diacid

The mixture of the diester intermediate (obtained from Step 2) and 2.7 g (64.3 mmol) of lithium hydroxide in tetrahydrofuran (75 mL) and water (50 mL) is stirred at 40° C. for 18 hours. The reaction mixture is acidified with 1% HCl and extracted with methylene chloride. The residue is triturated with hexane and filtered to give the desired compound 30.

### EXAMPLE 14

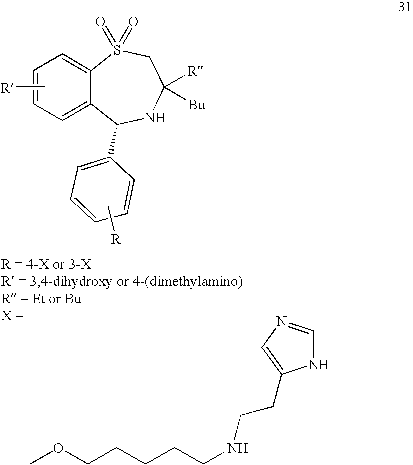

Step 1: Preparation of Pentyl Iodide Intermediate

To a solution of 15, 16, 19, 20, 21, 22, 23 or 24 (6.53 mmol, obtained from Example 6 or 7) in 100 mL of dimethylformamide is added 198 mg (7.83 mmol) of 95% sodium hydride. The mixture is stirred 15 minutes at room temperature and diiodopentane is added. After 1 hour at room temperature the mixture is diluted in ethyl acetate and water. The aqueous layer is extracted with ethyl acetate and the combined organic layer washed with brine, dried over magnesium sulfate and concentrated in vacuo. The residue is chromatographed over silica gel, eluting with hexane/ethyl acetate (1/5) to afford a pentyl iodide intermediate. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate, and concentrated in vacuo to give the pentyl iodide intermediate. See, e.g., Y. Leblanc et al., J. Org. Chem., 51, 189-93 (1986).

Step 2: Preparation of Amino-Histamine

A solution of the pentyl iodide intermediate (1.53 mmol, obtained from Step 1) and 3.4 g (30.6 mmol) of histamine is heated to 50° C. for 17 hours. The mixture is dissolved in ethyl acetate and saturated sodium bicarbonate. The organic layer is washed with brine, dried over magnesium sulfate, and concentrated in vacuo. The residue is triturated with ether to afford the desired compound 31.

### EXAMPLE 15

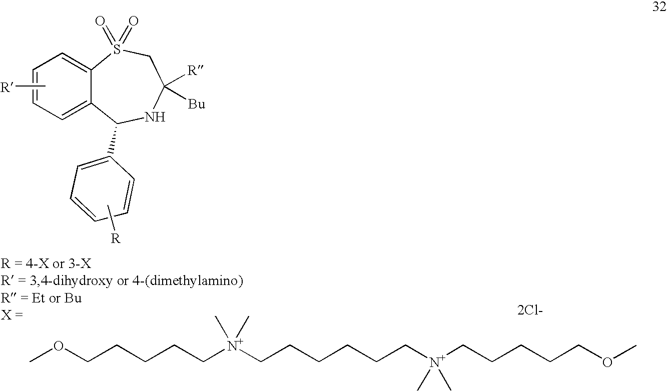

The pentyl bromide intermediate (1.64 mmol, obtained from Example 13, Step 1) and N,N,N′,N′-tetramethyl-1,6-hexanediamine (0.100 g, 0.580 mmol) in 5 mL of acetonitrile are placed in a 4 oz. Fischer Porter bottle. The reaction vessel is purged with nitrogen, sealed, equipped with magnetic stirrer and heated to 50° C. After 15 hours, the reaction mixture is cooled to ambient temperature and concentrated in vacuo to give a foamy solid. The solid is dissolved in acetonitrile and precipitated with ethyl ether to give the desired dibromide salt. The dibromide salt is converted to its corresponding dichloride salt using Biorad AG 2-X8 resin (a quaternary ammonium styrene type resin, chloride form, available from Biorad Laboratories) and eluting with 70% H₂O/CH₃CN to give the desired compound 32.

### EXAMPLE 16

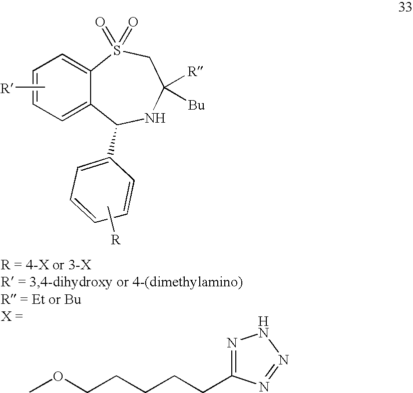

Step 1: Preparation of Pentyl Bromide Intermediate

To a stirred suspension of 1.01 g (25.4 mmol, 60% oil dispersion) of sodium hydride in 150 mL of dimethylformamide is added 19.5 mmol of 15, 16, 19, 20, 21, 22, 23 or 24 (obtained from Example 6 or 7) in portions. After 30 minutes, the reaction mixture is cooled in a water bath (15° C.) and 4.48 g (195 mmol) of 1,5-dibromopentane is added. The reaction mixture is stirred at ambient temperature for 1.5 hours and quenched with 50 mL of saturated ammonium chloride. The reaction mixture is then diluted with ethyl acetate, washed with water, washed with brine, dried over magnesium sulfate, filtered and concentrated in vacuo to a pentyl bromide intermediate.

Step 2: Preparation of Pentyl Nitrile Intermediate

To a stirred solution of 0.621 mmol of the pentyl bromide intermediate (obtained from Step 1) in 1 mL of dimethylsulfoxide is added 37 mg (0.745 mmol) of sodium cyanide. The reaction mixture is stirred at ambient temperature for 16 hours. The reaction mixture is concentrated under a nitrogen stream and the residue partitioned between ethyl acetate and water. The organic layer is washed with brine, dried over magnesium sulfate, filtered, and concentrated in vacuo to afford a pentyl nitrile intermediate.

Step 3: Preparation of Tetrazole

A solution of 0.5 mmol of the pentyl nitrile intermediate (obtained from Step 2) and 666 mg (3.23 mmol) of azidotrimethyltin in 5 mL of toluene is stirred with heating at 80° C. for 60 hours. The reaction mixture is concentrated under a nitrogen stream. Purification by reversed phase chromatography (Waters-Delta preparative scale HPLC) using 60% water/acetonitrile yields the desired compound 33. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate and concentrated in vacuo to give the desired compound 33. See, e.g., Y. Leblanc et al., J. Org. Chem., 51, 189-93 (1986).

### EXAMPLE 17

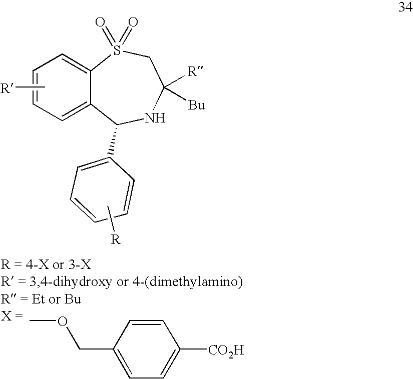

Step 1: Preparation of Benzoate Intermediate

To a solution of 1.15 mmol of 15, 16, 19, 20, 21, 22, 23 or 24 (obtained from Example 6 or 7) in 10 mL dimethylformamide is added 35 mg (1.39 mmol) of 95% sodium hydride. The reaction mixture is stirred for 10 minutes. To the reaction mixture is added 525 mg (2.29 mmol) of methyl 4-(bromomethyl)benzoate and the reaction mixture is stirred for an additional 16 hours. Water (100 mL) is added to the reaction mixture. The reaction mixture is extracted with ethyl acetate, washed with brine, dried over magnesium sulfate, filtered and the solvent evaporated to afford a benzoate intermediate. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate, and concentrated in vacuo to give a benzoate intermediate. See, e.g., Y. Leblanc et al., J. Org. Chem., 51, 189-93 (1986).

Step 2: Preparation of Acid

A solution of 0.84 mmol of the benzoate intermediate (obtained from Step 1) and 325 mg (2.53 mmol) of KOSi(CH₃)₃ (Aldrich) in 16 mL tetrahydrofuran is stirred for 3.5 hours. The tetrahydrofuran is evaporated and water is added. The solution is extracted with ethyl acetate, dried over magnesium sulfate, filtered and the solvent evaporated to afford the desired compound 34.

### EXAMPLE 18

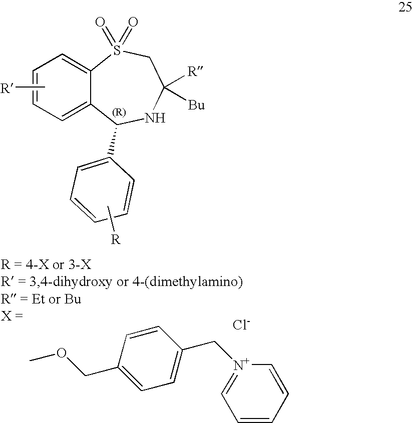

Step 1: Preparation of Chlorobenzyl Intermediate

A solution of 15, 16, 19, 20, 21, 22, 23 or 24 (10.9 mmol, obtained from Example 6 or 7) in acetone (100 mL) at 25° C. under nitrogen is treated with powdered K₂CO₃ (2.3 g, 16.3 mmol, 1.5 equivalents) and a,a′-dichloro-p-xylene (6.7 g, 38.1 mmol, 3.5 equivalents) and the resulting solution is stirred at 65° C. for 48 hours. The reaction mixture is cooled to 25° C. and concentrated to 1/5 of its original volume. The residue is dissolved in ethyl acetate (150 mL) and washed with water (2×150 mL). The aqueous layer is extracted with ethyl acetate (2×150 mL) and the combined organic extracts are washed with saturated aqueous sodium chloride (2×150 mL). The combined extracts are dried (magnesium sulfate) and concentrated in vacuo to provide a chlorobenzyl intermediate. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate and concentrated in vacuo to give a chlorobenzyl intermediate. See, e.g., Y. Leblanc et al., J. Org. Chem., 51, 189-93 (1986).

Step 2: Preparation of Quaternary Salt

A solution of the chlorobenzyl intermediate (1.7 mmol, obtained from Step 1) in acetonitrile (5 mL) at 25° C. under nitrogen is treated with pyridine (5 mL) and stirred at 35° C. for 36 hours. The pale amber solution is cooled to 25° C. and concentrated in vacuo to give the desired compound 35.

### EXAMPLE 19

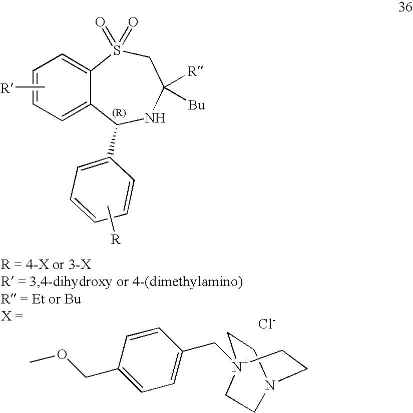

Under nitrogen, a solution of 14.5 mmol of the chlorobenzyl intermediate (obtained from a procedure similar to the one outlined in Example 18, Step 1) in 60 mL of acetonitrile is added dropwise over a 30 minute period to a solution of 2.9 g (26.2 mmol) of diazabicyclo[2,2,2]octane in 40 mL of acetonitrile at 35° C. During the addition, a colorless precipitate is formed. The summary is stirred at 35° C. for an additional 2 hours. The product is collected and washed with 1 L of acetonitrile to give the desired compound 36.

### EXAMPLE 19A

Compound 36 also can be prepared in accordance with an alternative synthetic scheme illustrated below in Scheme III:

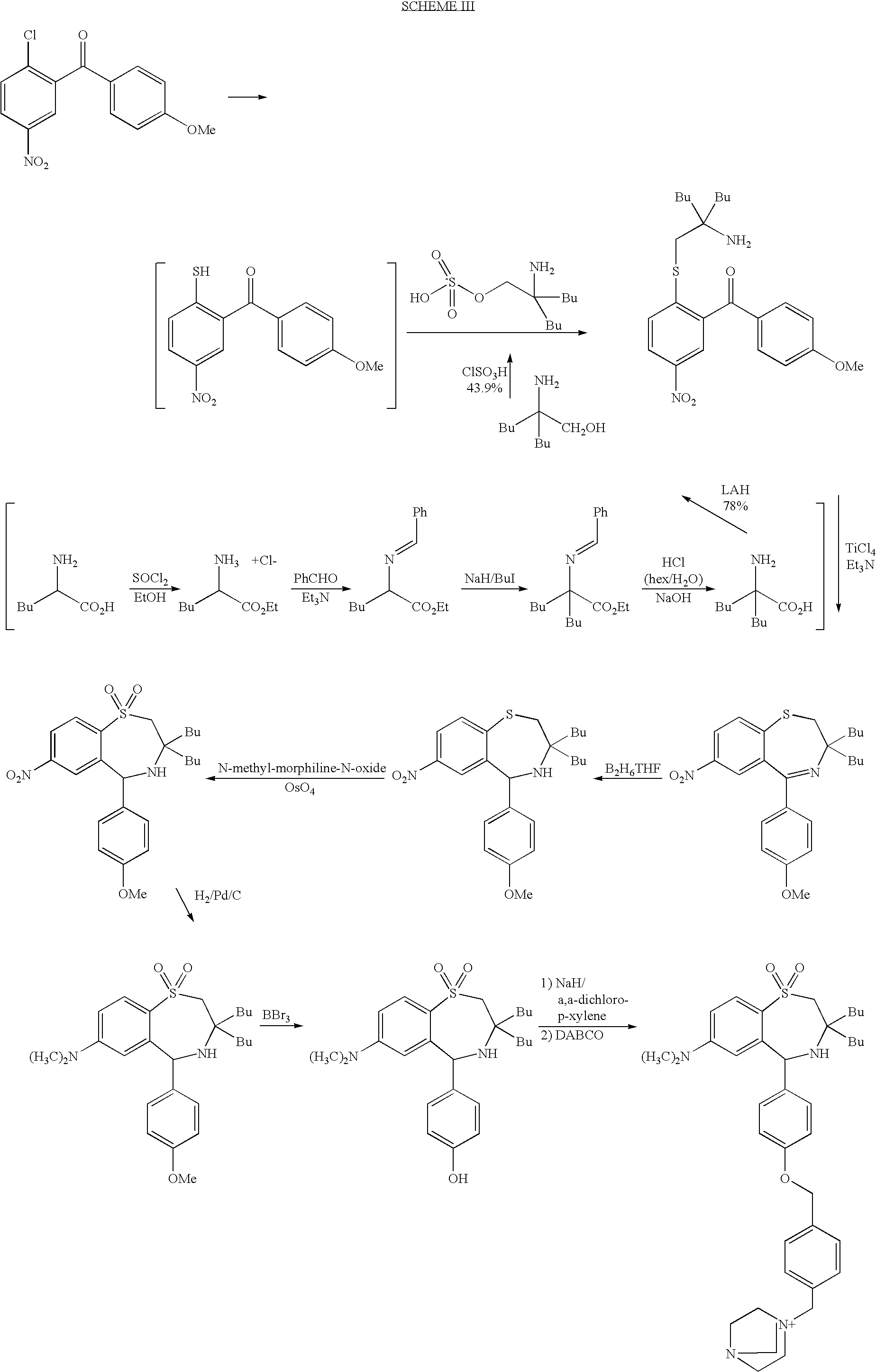

### EXAMPLE 20

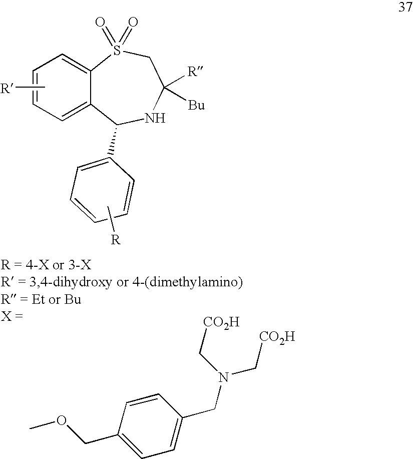

Step 1: Preparation of Chlorobenzyl Intermediate

To a stirred solution of 144 mg (3.59 mmol, 60% dispersion) of sodium hydride in 29 mL of dimethylformamide is added 3.26 mmol of 15, 16, 19, 20, 21, 22, 23 or 24 (obtained from Example 6 or 7), and the resulting solution is stirred at ambient temperature for 45 minutes. To the solution is added 7.13 g (40.75 mmol) of dichloro-p-xylene, and the mixture is stirred overnight. The dimethylformamide is removed in vacuo and the residue is extracted with ethyl acetate and washed with brine. The extract is dried over magnesium sulfate and concentrated in vacuo to give a chlorobenzyl intermediate.

Step 2: Preparation of Amino Diester

A mixture of 1.72 mmol of the chlorobenzyl intermediate (obtained from Step 1), 1.63 g (8.6 mmol) of diethylaminodiacetate, and 0.72 g (8.6 mmol) of sodium bicarbonate in 30 mL of dimethylformamide is stirred at 100° C. for 6 hours. The dimethylformamide is removed in vacuo and the residue is extracted with ether and washed with brine. The extract is dried over magnesium sulfate and the concentrated residue is purified by column chromatography to give an amino diester intermediate. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate and concentrated in vacuo to give a diester intermediate. See, e.g., Y. Leblanc et al., J. Org. Chem., 51, 189-93 (1986).

Step 3: Preparation of Amino Diacid

A solution of 1.15 mmol of the dibenzyl ester (obtained from Step 2) and 0.232 g (5.52 mmol) of lithium hydroxide in 30 mL of tetrahydrofuran and 30 mL of water is stirred at 40° C. under nitrogen for 4 hours. The reaction mixture is diluted with ether and washed with 1% HCl. The aqueous layer is extracted twice with ether, and the combined extracts are washed with brine, dried over magnesium sulfate, and concentrated in vacuo to give the desired compound 37.

### EXAMPLE 21

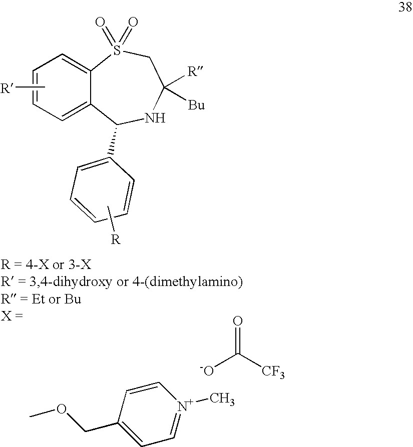

Step 1: Preparation of Picolyl Intermediate

1.4 g (60% oil dispersion, 35 mmol) of sodium hydride is added to a stirred solution of 26.1 mmol of 15, 16, 19, 20, 21, 22, 23 or 24 (obtained from Example 6 or 7) in 200 mL dimethylformamide. The reaction mixture is stirred at ambient temperature for one hour. A solution of 4-picolyl-chloride hydrochloride is prepared by treating 5.99 g (36.5 mmol) of 4-picolyl chloride hydrochloride with cold saturated sodium bicarbonate solution and extracting the solution with diethyl ether. The ethereal extract is washed with brine, dried over magnesium sulfate, and filtered. The reaction mixture is then cooled in an ice bath and the solution of 4-picolyl chloride hydrochloride in diethyl ether added. The reaction mixture is stirred at ambient temperature for 17 hours. The reaction mixture is quenched with 25 mL of saturated ammonium chloride and diluted with 600 mL ethyl acetate. It is then washed with 4×250 mL water, washed with brine, dried over magnesium sulfate, filtered and concentrated in vacuo. Purification by silica gel chromatography (Waters-Delta 500 preparative scale HPLC) using 60% ethyl acetate/hexanes yields a picolyl intermediate.

Step 2: Preparation of Quaternary Salt

To a stirred solution of 0.74 mmol of the picolyl intermediate (obtained from Step 4) in 10 mL of acetonitrile and 3 mL of methylene chloride, 137 mg (0.97 mmol) of iodomethane is added. The reaction is stirred at ambient temperature for 16 hours and then concentrated under a nitrogen stream. Purification by reversed phase chromatography provides the desired compound 38. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. Ion exchange with trifluoroacetate anion yields the desired compound 38. See, e.g., Y. Leblanc et al., J. Org. Chem., 51, 189-93 (1986).

### EXAMPLE 22

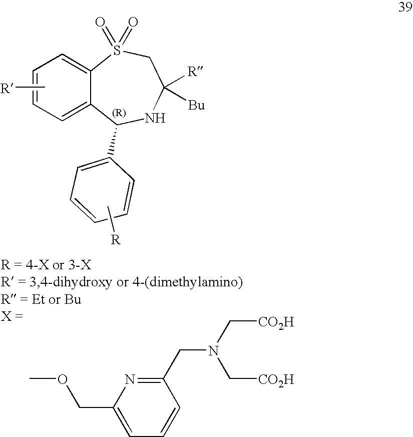

Step 1: Preparation of Picolinyl Chloride Intermediate

Anhydrous K₂CO₃ (0.45 g, 3.2 mmol), tetrabutylammonium iodide (0.1 g, 0.2 mmol) and 2,6-bischloromethylpyridine (1.2 g, 10.8 mmol) are added to a flask containing a solution of 15, 16, 19, 20, 21, 22, 23 or 24 (2.1 mmol, obtained from Example 6 or 7) in acetone (50 mL). The flask is equipped with nitrogen gas adapter and magnetic stirrer. The reaction mixture is heated to reflux overnight. After 18 hours, the reaction is diluted with ether and washed with water and brine (30 mL). The organic layers are dried over magnesium sulfate, filtered and concentrated in vacuo. Chromatographic purification through silica gel, eluting with 25% ethyl acetate/hexane, yields a picolyl chloride intermediate.

Step 2: Preparation of Pyridinyl Diester Intermediate

A mixture of diethylaminodiacetate (8 g, 68 mmol) and sodium carbonate (0.63 g, 5.9 mmol) is treated with the picolyl chloride intermediate (1.2 mmol, obtained from Step 1) and the reaction mixture is stirred at 160° C. for three hours. The reaction mixture is cooled and diluted with ether and washed with 1% HCl, water (25 mL), and brine (50 mL). The combined extracts are dried over magnesium sulfate, filtered and concentrated in vacuo. The residue is purified by distillation in a Kugelrohr to provide a pyridinyl diester intermediate. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate and concentrated in vacuo to give the pyridinyl diester intermediate. See, e.g., Y. Leblanc et al., J. Org. Chem., 51, 189-93 (1986).

Step 3: Preparation of Pyridinyl Diacid

A mixture of pyridine-aminodiacetate intermediate (0.93 mmol, obtained from Step 2), and lithium hydroxide monohydrate (0.18 g, 4.5 mmol) in tetrahydrofuran/water (25.0 mL, 1:1) is stirred at 40° C. overnight (18 hours). The reaction mixture is then diluted with ether and washed with 1% HCl, water (20 mL), and brine (30 mL). The organic layers are dried over magnesium sulfate, filtered and concentrated in vacuo to give the desired compound 39.

### EXAMPLE 23

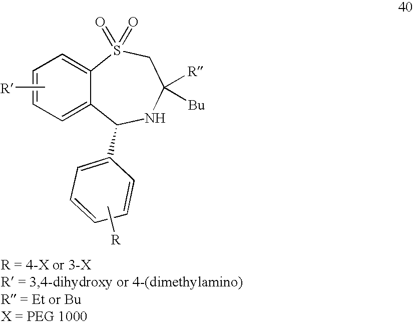

Step 1: Preparation of Monomethyl PEG Mesylate Intermediate

To a solution of 20 g of monomethyl ether PEG in 100 mL of methylene chloride, 2.2 g (22 mmol) of triethyl amine is added. To this solution, 2.5 g (22 mmol) of methanesulfonyl chloride is added dropwise at 0° C. The resulting solution is stirred overnight at ambient temperature. The triethyl amine hydrochloride is filtered off to give a monomethyl PEG mesylate intermediate which is used in the next step without further purification and characterization.

Step 2: Preparation of Polyethylene-Linked Benzothiepene

A mixture of 38 mg (1.52 mmol 95%) of sodium hydride and 1.52 mmol of 15, 16, 19, 20, 21, 22, 23 or 24 (obtained from Example 6 or 7) in 5.5 mL of dimethylformamide is stirred at ambient temperature under nitrogen for 30 minutes. To the solution, 0.55 g (0.51 mmol) of the mesylate PEG intermediate (obtained from Step 1) in 5.5 mL of dimethylformamide is added. The resulting solution is stirred overnight under nitrogen at 50° C. The dimethylformamide is removed in vacuo and the residue extracted with methylene chloride and washed with brine. The extract is then dried over magnesium sulfate and the concentrated residue purified by column chromatography to give the desired compound 40. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate and concentrated in vacuo to give the desired compound 40. See, e.g., Y. Leblanc et al., J. Org. Chem., 51, 189-93 (1986).

### EXAMPLE 24

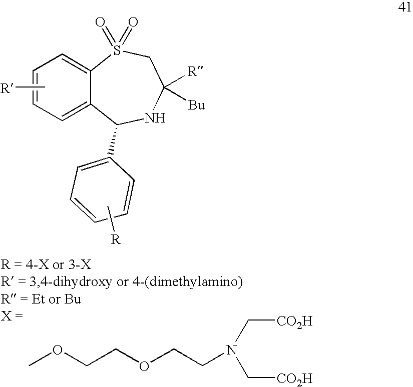

A mixture of 10.7 mmol of 15, 16, 19, 20, 21, 22, 23 or 24 (obtained from Example 6 or 7), 11.45 g of diethyliminodiacetate, and 1.14 g of sodium carbonate is held at 160° C. for 3.5 hours, diluted with brine and extracted with methylene chloride. The methylene chloride layer is washed with brine, dried (magnesium sulfate) and concentrated in vacuum. The residue is kugelrohr distilled at 0.5 torr at 120° C. to remove excess diethyliminodiacetate and to give a residue. A mixture of this residue, 0.8 g of lithium hydroxide, 25 mL of tetrahydrofuran, and 25 mL of water is held at 45° C. for 3 days and then concentrated in vacuum to remove the tetrahydrofuran. The residual aqueous solution is diluted with 25 mL of water, acidified to pH 2 and extracted with methylene chloride (2×50 ml). The methylene chloride layer is dried (magnesium sulfate) and concentrated in vacuo to give the desired compound 41. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate, and concentrated in vacuo to give the desired compound 41. See, e.g., Y. Leblanc et al., J. Org. Chem., 51, 189-93 (1986).

### EXAMPLE 25

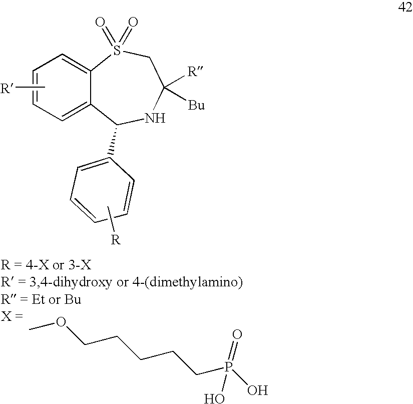

Step 1: Preparation of Pentyl Bromide Intermediate

A solution of 15, 16, 19, 20, 21, 22, 23 or 24 (1.09 mmol, obtained from Example 6 or 7) in 5 mL of dimethylformamide is added via a syringe to a stirred solution of 36 mg of 95% sodium hydride (1.41 mmol) in 5 mL of dimethylformamide at −10° C. in an acetone-dry ice bath. The resulting solution is stirred at −10° C. for 30 minutes. A solution of 1.25 g of 1,5-dibromopentane (5.45 mmol) in 5 mL of dimethylformamide is then added. The mixture is stirred at −10° C. for another 30 minutes and allowed to warm up to room temperature and stirred for 1 hour. The reaction mixture is then quenched with water at 0° C. and extracted with ethyl acetate. The ethyl acetate layer is dried over magnesium sulfate and concentrated in vacuo. The crude product is chromatographed on silica gel column to give a pentyl bromide intermediate.

Step 2: Preparation of Phosphonic Acid

A stirred solution of 400 mg of the pentyl bromide intermediate (0.66 mmol, obtained from Step 1) in 2 mL of tris(trimethylsilyl) phosphite is refluxed at 100° C. overnight. The reaction mixture is cooled to room temperature and 30 mL of 50% methanol/water solution is added. The reaction mixture is stirred at room temperature for 5 hours. The reaction mixture is concentrated in vacuo and the resulting aqueous solution is extracted with methylene chloride. The methylene chloride solution is dried over magnesium sulfate and concentrated in vacuo to yield the desired product 42. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate and concentrated in vacuo to give the desired product 42. See, e.g., Y. Leblanc et al., J. Org. Chem., 51, 189-93 (1986).

### EXAMPLE 26

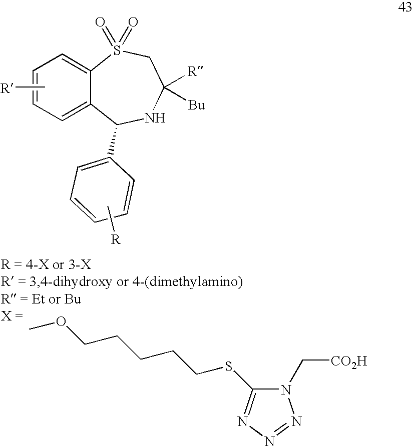

A mixture of 0.325 g (1.78 mmol) of 5-mercaptotetrazoleacetic acid sodium salt, 1.0 g of potassium carbonate, and 30 mL of dimethylformamide is stirred for 2 hours and then charged with 1.74 mmol of the pentyl bromide intermediate (Example 13, Step 1). The reaction mixture is stirred for 20 hours at room temperature and concentrated in vacuum. The residue is then stirred in ether and water (100 mL each). The resulting precipitate is combined with the aqueous layer, acidified with concentrated HCl and extracted with methylene chloride. The methylene chloride layer is dried (magnesium sulfate) and concentrated in vacuo to yield the desired product 43. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate, and concentrated in vacuo to give the desired product 43. See, e.g., Y. Leblanc et al., J. Org. Chem., 51, 189-93(1986).

### EXAMPLE 27

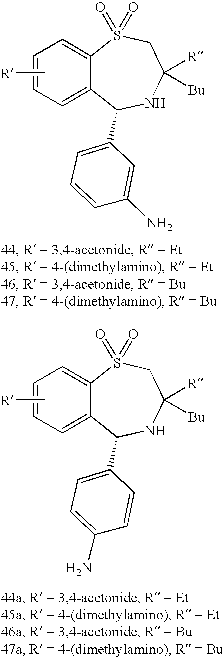

Step 1: Preparation of Triflic Intermediate

Triflic anhydride (4.1 mL, 24.4 mmol, 1.1 equivalents) is added dropwise to a solution of 22.13 mmol of compound 15, 16, 19, 20, 21, 22, 23 or 24 (obtained from Example 6 or 7) in pyridine (42 mL) at 0° C. under nitrogen gas. Upon completion of the triflic anhydride addition, the bath is removed and the reaction stirred at room temperature for 21 hours. The pyridine is removed in vacuo and the resulting oil is taken up in water (100 mL) and extracted three times with ethyl acetate (45 mL each). The combined organics are washed with 10% CuSO₄ (100 mL) and brine (100 mL), and then dried over magnesium sulfate, filtered and the solvent evaporated. The residue is purified by chromatography on silica gel to give a triflic intermediate.

Step 2: Preparation of Imine Intermediate

To a solution of 19.28 mmol of the triflate intermediate (prepared in Step 1), palladium (II) acetate (433 mg, 1.93 mmol, 10 mol %), racemic 2,2′-bis-(biphenylphosphenyl)-1,1′-binaphthyl (1.41 g, 2.26 mmol, 12 mol %) and cesium carbonate (8.86 g, 27.2 mmol, 2.0 equivalents) in 114 mL of tetrahydrofuran is added 6.6 mL of benzophenone imine (39.4 mmol, 2.0 equivalents). The mixture is stirred at reflux for 4 hours, filtered through celite and the solvent removed in vacuo providing an imine intermediate.

Step 3: Preparation of Aniline

To a solution of 19.3 mmol of the crude imine intermediate (prepared in Step 2) in methanol (200 mL) is added sodium acetate (6.33 g, 77.2 mmol, 4 equivalents) and hydroxylamine hydrochloride (4.02 g, 57.9 mmol, 3 equivalents). The mixture is stirred for 1 hour and 1N sodium bicarbonate (100 mL) is added. The mixture is then extracted with methylene chloride (2×100 mL, 1×50 mL). The combined organics are washed with brine (100 mL), dried over magnesium sulfate, filtered and the solvent evaporated. The residue is purified by chromatography on silica gel to afford the desired aniline 44, 44a, 45, 45a, 46, 46a, 47 or 47a.

### EXAMPLE 28

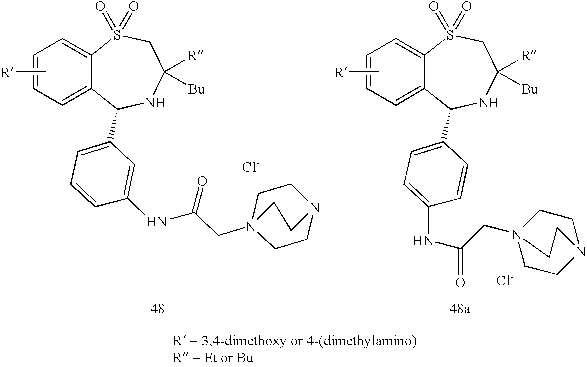

Step 1: Preparation of Chloroacetyl Intermediate

A solution of 44, 44a, 45, 45a, 46, 46a, 47 or 47a (2.2 mmol, obtained from Example 27) in methylene chloride (10 mL) at 0° C. under nitrogen is treated with N,N-di-isopropyl-ethylamine (0.53 mL, 3.1 mmol, 1.4 equivalents), followed by the dropwise addition of chloroacetyl chloride (0.21 mL, 2.6 mmol, 1.2 equivalents) over a 10 minute period. The reaction mixture is stirred and allowed to warm to 25° C. over a 2 hour period. The mixture is quenched by the addition of 1N HCl (25 mL) and the aqueous layer is extracted with ethyl acetate (2×25 mL). The combined organic extracts are washed with saturated aqueous sodium bicarbonate (2×25 mL) and brine (30 mL), and then dried (magnesium sulfate) and concentrated to give a chloroacetyl intermediate.

Step 2: Preparation of Quaternary Salt

A solution of the chloroacetyl intermediate (0.05 mmol, obtained from step 1) in acetonitrile (1 mL) at 50° C. under nitrogen is treated with diazabicyclo[2,2,2]octane (10 mg, 0.09 mmol, 1.8 equivalents) and stirred at 50° C. for 2 hours. The reaction mixture is allowed to cool to 25° C. and then concentrated to form a residue. The residue is dissolved in warm acetonitrile and tert-butyl methyl ether is added. The mixture is allowed to stand overnight to precipitate the desired compound 48 or 48a. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. Ion exchange with chloride ion will give the desired product 48 or 48a. See, e.g., Y. Leblanc et al., J. Org. Chem., 51, 189-93 (1986).

### EXAMPLE 29

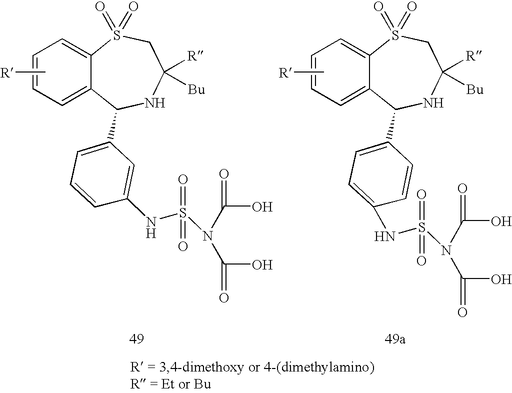

Step 1: Preparation of the Sulfonamoyl Chloride Intermediate

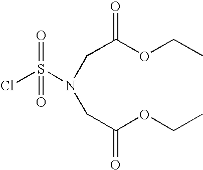

Sulfuryl chloride (27.552 g/204.1 mmol) and chloroform (50.0 mL) are combined in a 250 mL round-bottom flask. The reaction flask is purged with nitrogen, equipped with a magnetic stirrer, and cooled to 0° C. A solution of diethyl iminodiacetate (18.902 g/99.9 mmol) and triethylamine (10.112 g/99.9 mmol) is added dropwise while maintaining the temperature of the solution below 20° C. After the addition is complete, the reaction mixture is allowed to warm to room temperature. After 2 hours, the reaction mixture is poured into ice water (100 mL) and mixed well. The organic layer is separated, washed with 10% aqueous HCl (50 mL) and chilled water (2×50 mL), dried (CaCl₂), filtered, and concentrated in vacuo to give a sulfonamoyl chloride intermediate as an amber liquid (5.706 g/20%).

Step 2: Preparation of the Diester Intermediate

The 3-aminobenzothiepine 44, 44a, 45, 45a, 46, 46a, 47 or 47a (1.097 mmol, obtained from Example 27), toluene (5.00 mL), diisopropylethylamine (0.148 g/1.148 mmol), and the sulfonamoyl chloride intermediate (0.650 g/2.260 mmol, obtained from step 1) are combined in a 25 mL round-bottom flask. The reaction flask is purged with nitrogen and equipped with magnetic stirrer. After 24 hours, methylene chloride (75.0 mL) is added. The mixture is washed with aqueous sodium bicarbonate (25.0 mL) and then aqueous sodium chloride (25.0 mL), dried (magnesium sulfate), and concentrated in vacuo. Purifying by flash chromatography on silica gel eluting with ethyl acetate/hexane and concentrating in vacuo gives a diester intermediate.

Step 3: Preparation of Diacid

The diester intermediate (0.316 mmol, obtained from step 2) and tetrahydrofuran (1.00 mL) are combined in a 10 mL round-bottom flask. The reaction flask is purged with nitrogen and equipped with magnetic stirrer. A solution of LiOH.H₂O (0.030 g/0.715 mmol) in water (0.50 mL) is added. After 4 hours, additional LiOHH₂O (0.015 g/0.357 mmol) is added. After 30 minutes, water (6.0 mL) is added. The aqueous mixture is washed with diethyl ether (4×4.0 mL), and acidified with aqueous 3.0 N HCl (0.40 mL). The product precipitates and is filtered, washed with water (2.0 mL), and concentrated in vacuo. Precipitation from acetonitrile/diethyl ether/hexanes gives the desired product 49 or 49a. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. Precipitation from acetonitrile/diethyl ether/hexanes gives the desired product 49 or 49a. See, e.g., Y. Leblanc et al., J. Org. Chem., 51, 189-93 (1986).

### EXAMPLE 30

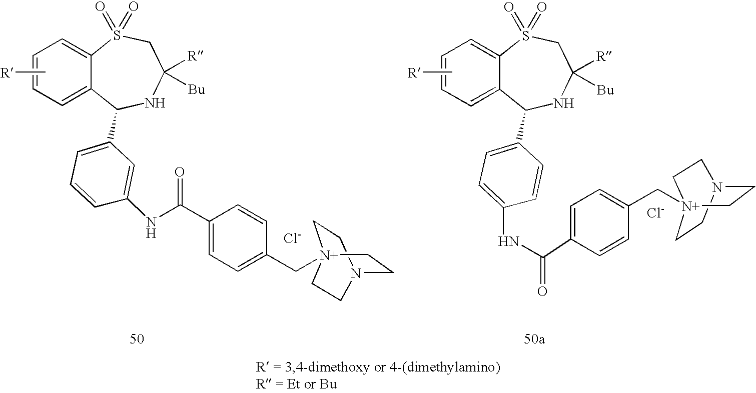

Step 1: Preparation of Chlorobenzoyl Intermediate

A solution of the 3-aminobenzothiepine 44, 44a, 45, 45a, 46, 46a, 47 or 47a (2.2 mmol, obtained from Example 27) in methylene chloride (10 mL) at 0° C. under nitrogen is treated with N,N-di-isopropyl-ethylamine (0.53 mL, 3.1 mmol, 1.4 equivalents), followed by the dropwise addition of 4-chlorobenzoyl chloride (0.455 g, 2.6 mmol, 1.2 equivalents) over a 10 minute period. The reaction mixture is stirred and allowed to warm to 25° C. over a 2 hour period. The mixture is quenched by the addition of aqueous ammonium chloride and the aqueous layer is extracted with ethyl acetate (2×25 mL). The combined organic extracts are washed with saturated aqueous sodium bicarbonate (2×25 mL) and brine (30 mL), and then dried (magnesium sulfate) and concentrated to give a chlorobenzoyl intermediate.

Step 2: Preparation of Quaternary Salt

A solution of the chlorobenzoyl intermediate (0.05 mmol, obtained from step 1) in acetonitrile (1 mL) at 50° C. under nitrogen is treated with diazabicyclo[2,2,2]octane (10 mg, 0.09 mmol, 1.8 equivalents) and stirred at 50° C. for 2 hours. The reaction mixture is allowed to cool to 25° C. and then concentrated in vacuo to give the desired compound 50 or 50a. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. Ion exchange with chloride ion will give the desired product 50 or 50a. See, e.g., Y. Leblanc et al., J. Org. Chem., 51, 189-93 (1986).

### EXAMPLE 31

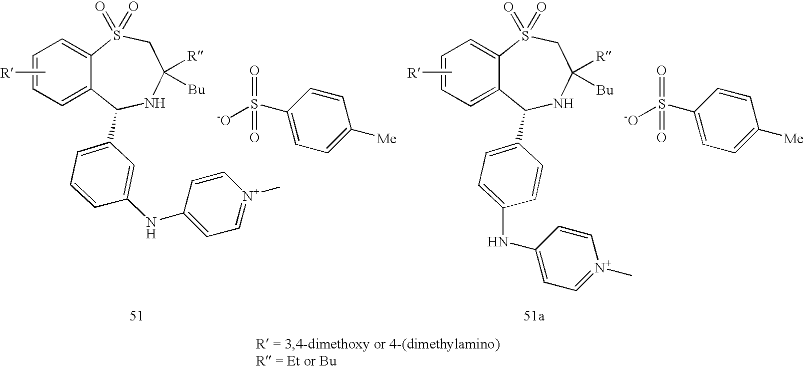

Step 1: Preparation of Pyridyl Intermediate

A solution of the 3-aminobenzothiepine 44, 44a, 45, 45a, 46, 46a, 47 or 47a (4.37 mmol, obtained from Example 26) in ethanol (14.0 mL) under nitrogen is treated with 4-bromopyridine hydrochloride (1.041 g, 5.35 mmol) and heated to reflux. After 48 hours, the reaction mixture is cooled to room temperature and concentrated in vacuo. The residue is dissolved in ethyl acetate (150 mL) and washed with aqueous sodium bicarbonate (2×70 mL) and brine (50 mL). The mixture is dried (magnesium sulfate), filtered and concentrated in vacuo. The residue is purified by flash chromatrography on silica gel to give a pyridyl intermediate.

Step 2: Preparation of Quaternary Salt

A solution of the pyridyl intermediate (3.39 mmol, obtained from step 1) in acetonitrile (18.0 mL) at room temperature under nitrogen is treated with methyl p-toluenesulfonate (0.550 mL, 3.77 mmol) and stirred for 17 hours. The reaction mixture is filtered and concentrated to give the desired compound 51 or 51a. For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. Ion exchange with tosylate anion will give the desired product 51 or 51a. See, e.g., Y. Leblanc et al., J. Org. Chem., 51, 189-93 (1986).

### EXAMPLE 32

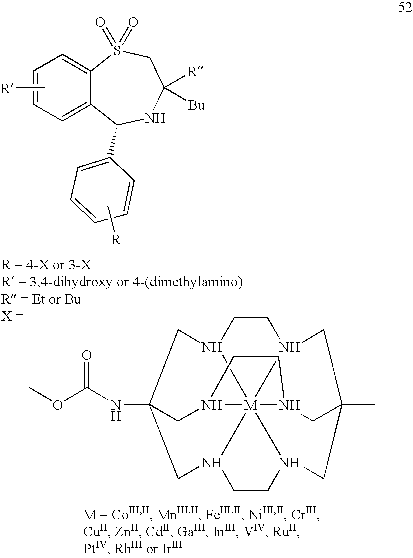

Step 1: Preparation of the Metal Cage Complex

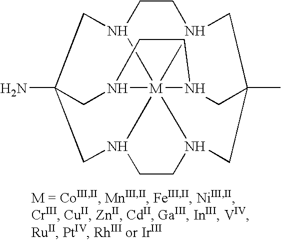

A metal cage complex is prepared as described in A. M. Sargeson et al, J. Chem. Soc., Chem Commun., 1844-1846 (1993).

Step 2: Preparation of the Carbamoyl Chloride Intermediate

The metal cage complex (2.00 mmol, obtained from Step 1) is combined with methylene chloride (15.0 mL), triethylamine (0.223 g/2.20 mmol) and phosgene (0.218 g/2.20 mmol) in a dry 25 mL round-bottom flask. After stirring overnight at room temperature, the reaction mixture is concentrated in vacuo. The residue is triturated with tetrahydrofuran (5 mL), filtered and concentrated in vacuo to give a carbamoyl chloride intermediate. See, e.g., Tetrahedron Lett., 39, 757-760 (1998).

Step 3: Preparation of the Carbamate

The hydroxybenzothiepene 15, 16, 19, 20, 21, 22, 23 or 24 (0.828 mmol, obtained from Example 6 or 7), triethylamine (0.100 g/0.994 mmol) and toluene (1.0 mL) are combined in a 10 mL round-bottom flask. The reaction flask is purged with nitrogen, equipped with a magnetic stirrer, and cooled to 0° C. A solution of the carbamoyl chloride (12% in tetrahydrofuran/1.10 mmol, obtained from Step 2) is added. After 3.5 hours, the mixture is filtered and concentrated in vacuo to give the desired carbamate 52. See, e.g., Tetrahedron Lett., 39, 757-760 (1998). For reactions with reagents 21, 22, 23 or 24, removal of the acetonide protecting group is accomplished by dissolving the substrate in 4:1 tetrahydrofuran/water at 0° C., adding excess trifluoroacetic acid, and stirring at room temperature overnight. The reaction is neutralized with ammonium hydroxide, and the tetrahydrofuran removed under reduced pressure. The product is then extracted into ethyl acetate, and concentrated in vacuo to give the diester intermediate. See, e.g., Y. Leblanc et al., J. Org. Chem., 51, 189-93 (1986).

### EXAMPLE 33

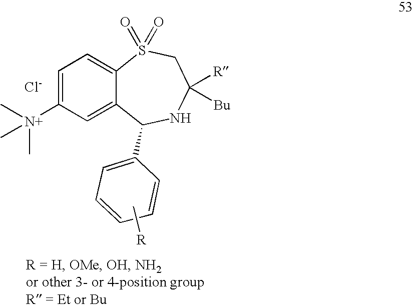

A solution of any corresponding dimethylamino analog of the desired product 53 (0.34 mmol, obtained by suitable modification of the Synthetic Examples of patent application WO96/01844 and the Examples of the present application) in acetonitrile (7.0 mL) at 50° C. under nitrogen is treated with methyl iodide (20 equivalents) and stirred for 72 hours. The reaction mixture is concentrated to form a residue. The product is then dissolved in acetonitrile and precipitated with ethyl ether. Ion exchange with chloride ion gives the desired compound 53.

### EXAMPLE 34

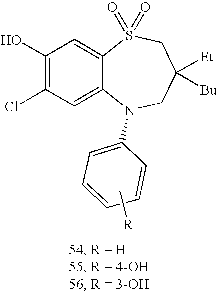

Benzothiazapine 54 is prepared as described in steps 1 through 9 of Synthetic Example 1 of patent application WO 99/35135, except that 4-iodoanisole (or 3-iodoanisole) is substituted for iodobenzene in step 7 to give Compound 55 (or Compound 56).

### EXAMPLE 35

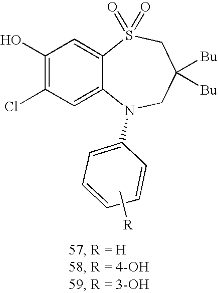

Step 1: Preparation of 2-(bromomethyl)-2-butyl-hexanoic Acid

2-(Bromomethyl)-2-butyl-hexanoic acid is prepared in accordance with the procedure set forth in steps (1) and (2) of Synthetic Example 1 of patent application WO 99/35135, except that 2,2-dibutyl-1,3-propanediol is substituted for 2-butyl-2-ethyl-1,3-propanediol in step 1 of Synthetic Example 1, step 1,2,2-Dibutyl-1,3-propanediol is prepared, for example, as set forth at column 264 of U.S. Pat. No. 5,994,391.

Step 2: Preparation of Butyl—Butyl Benzothiazepine

Compounds 57, 58 and 59 are prepared in accordance with the procedure set forth in steps (1) through (9) of Synthetic Example 1 of patent application WO99/35135, except that (a) 2-(bromomethyl)-2-butyl-hexanoic acid (obtained from Step 1) is substituted for 2-(bromomethyl)-2-ethyl-hexanoic acid in step 3 of Synthetic Example 1, and either 4-iodoanisole (for Compound 58) or 3-iodoanisole (for Compound 59) is optionally substituted for iodobenzene (for Compound 57) in step 7 of Synthetic Example 1.

### EXAMPLE 36

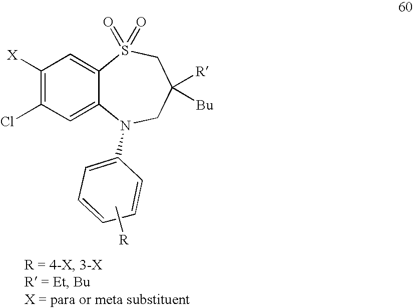

Compound 60 comprising a wide variety of R substituents can be prepared by appropriate modification of the procedures described in the above Examples starting with one of compounds 53, 54, 55 and 56 (obtained from Examples 33 and 34).

Similarly, additional 1,5-benzothiazapines can be prepared by appropriate modification of the procedures described in the above Examples. For example, the preparation of the 1,5-benzothiazepine counterpart to 1,4-benzothiazepine 36 described in Examples 19 and 19A can be prepared as illustrated in Scheme IV below:

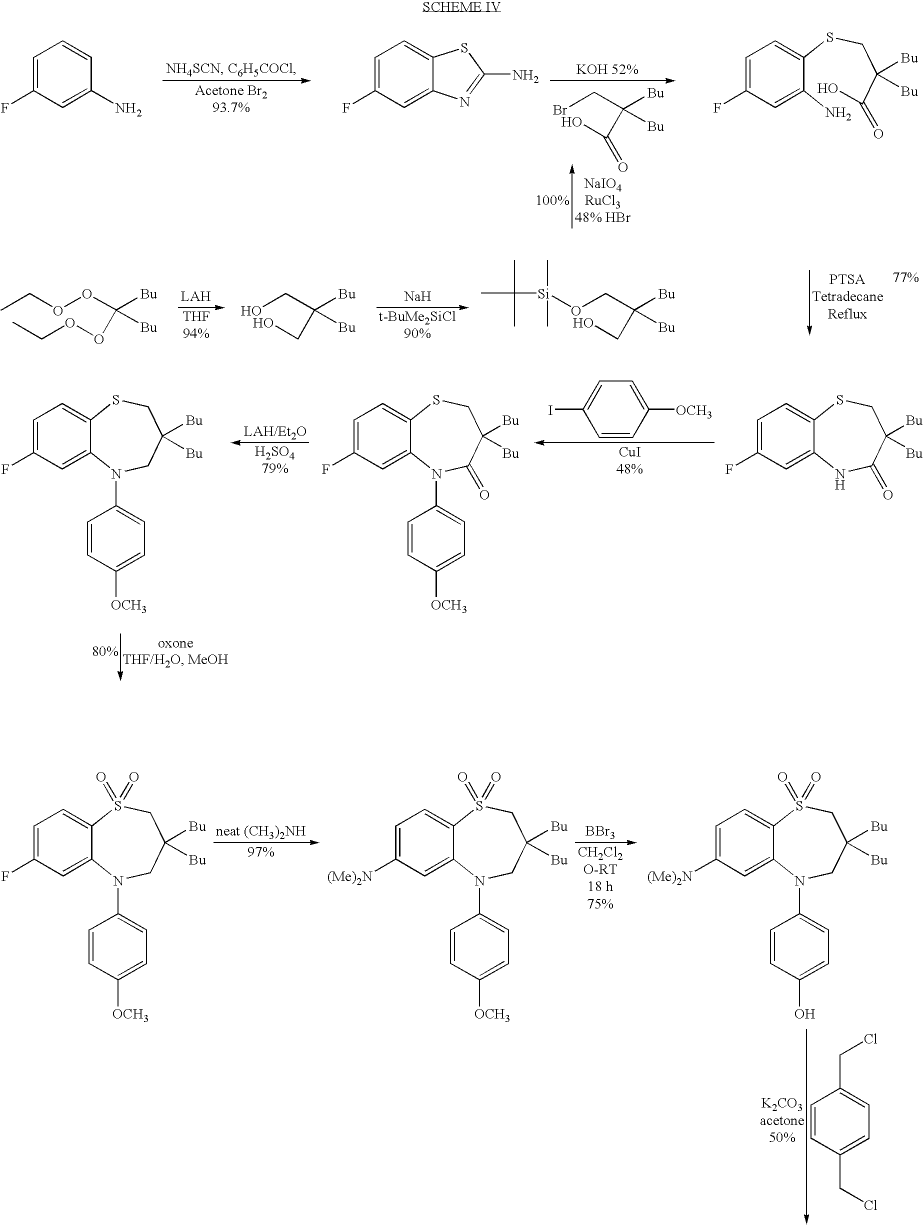

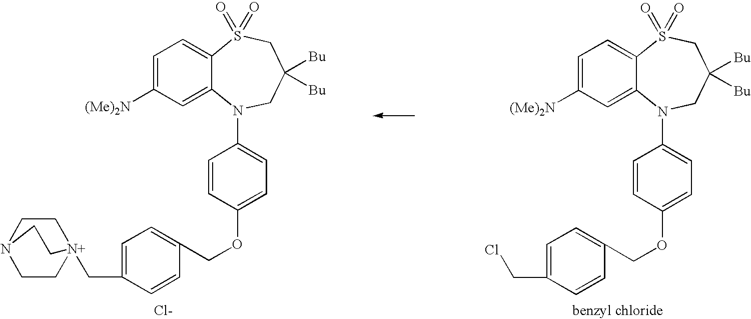

### ADDITIONAL EXAMPLES

General Comments

Chemicals were obtained from Aldrich Chemical Company and were used without further purification. ¹H and ¹³C NMR spectra were recorded on a Varian 300 spectrometer at 300 and 75 MHz respectively. The ¹H chemical shifts are reported in ppm downfield from Me₄Si. The ³C chemical shifts are reported in ppm relative to the center line of CDCl₃ (77.0 ppm). High Resolution Mass spectra were determined by Monsanto Analytical Sciences Center and microanalyses were performed by Atlantic. Microlab Inc. HPLC data was obtained on a Spectra Physics 8800 Chromatograph using a Beckman Ultrasphere C18 250×4.6 mm column. HPLC conditions: detector wavelength=254 nm, sample size=10 μL, flowrate=1.0 mL/min, mobile phase=(A) 0.1% aqueous trifluoroacetic acid: (B) acetonitrile.

HPLC Gradient:

| Time | % A | % B |
|---|---|---|
| 0 min | 85 | 15 |
| 20 min | 0 | 100 |

Synthesis of the 1,4 benzothiazepines Scheme III

Ethyl 2-aminohexanoate Hydrochloride

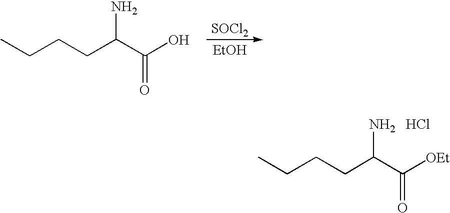

A slurry of DL-norleucine (75.0 g, 572 mmol) in absolute ethanol (400 ml) was stirred under nitrogen in an ice-water bath and thionyl chloride (71.4 g, 43.8 ml) was added dropwise. The reaction was stirred overnight at 0° C. and then gradually warmed to room temperature. The resulting slurry was heated under reflux for 3 hours. After cool down, the reaction mixture was concentrated to yield desired product (108.9 g/97%) as a light yellow solid. ¹H NMR (D₂O) δ 0.80 (t, J=7.2 Hz, 3H), 1.20 (t, J=6.9 Hz, 3H), 1.20-1.36 (m, 4H), 1.84 (m, 2H), 4.02 (t, J=6.3 Hz, 1H), 4.21 (q, J=7.4 Hz, 2H). LC/MS (ES/M+H): 160.0.

Ethyl 2-benzylideneaminohexanoate

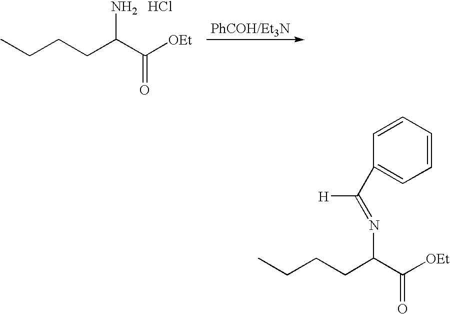

A solution of the product ethyl 2-aminohexanoate hydrochloride (from the step 1) (108 g, 552 mmol) in CH₂Cl₂ (1200 ml) and MgSO₄ (66.45 g) was stirred at room temperature under nitrogen for 20 minutes. Then, Et₃N (152 ml) was added. After stirring for another 30 minutes, xs. benzaldehyde was added dropwise. The reaction mixture was stirred at room temperature for 2 hours. MgSO₄ (66.5 g) was added and the reaction mixture was heated to reflux for 3 hours. After cool to room temperature, the reaction mixture was stirred overnight, filtered and concentrated. The resulting mixture was triturated in diethyl ether, filtered and concentrated to yield product as a yellow oil (125 g/92%). ¹H NMR (CDCl₃) δ 0.90 (t, J=6.3 Hz, 3H), 1.27 (t, J=7.4 Hz, 3H), 1.25-1.38 (m, 4H), 1.83-2.05 (m, 2H), 3.95 (t, J=6.9 Hz, 1H), 4.21 (q, J=7.2 Hz, 2H), 7.40 (m, 3H), 7.78 (d, J=8.1 Hz, 2H), 8.26 (s, 1H). LC/MS (ES/M+H): 248.1.

Ethyl 2-benzylideneamino-2-butylhexanoate

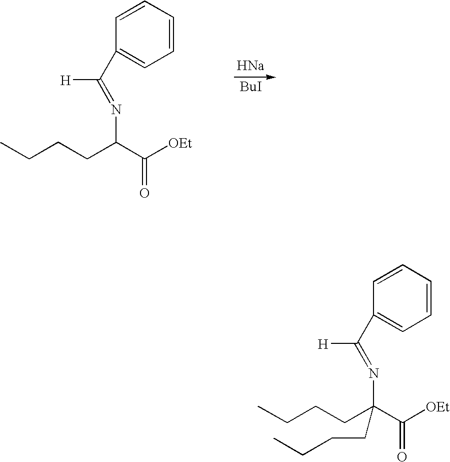

Sodium hydride (20.23 g, 60% dispersion in oil) and DMF (500 ml) were stirred under nitrogen at room temperature for 10 minutes. A solution of the product of ethyl 2-benzylideneaminohexanoate in 100 ml DMF was added dropwise. After 2 hours stirring at room temperature, a solution of BuI (102 g, 1.1 eq) in 50 ml of DMF was added dropwise and the reaction left string for overnight. The reaction mixture was poured into an ice cold mixture of water (350 ml), ether (300 ml) and ammonium chloride (74 g). The resulting organic layer was dried over potassium carbonate then concentrated to give the desired product as a yellow oil (138.75 g, yield=90.5%). ¹H NMR (CDCl₃) δ 0.90 (t, J=6.9 Hz, 6H), 1.20-1.40 (m, 8H), 1.28 (t, J=7.5 Hz, 3H), 1.80-2.00 (m, 4H), 4.22 (q, J=6.9 Hz, 2H), 7.40 (m, 3H), 7.78 (m, 2H), 8.32 (s, 1H).

Ethyl 2-amino-2-butylhexanoate

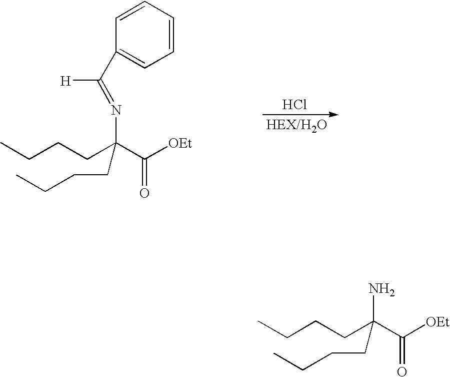

The product of ethyl 2-benzylideneamino-2-butylhexanoate was partitioned between hexane and 10% aq. HCl and stirred at room temperature for 2 hours. The aqueous layer was extracted twice with hexane (2×100 ml). The aqueous layer was added 200 ml of ethyl acetate and was chilled with an ice-water bath. Sodium hydroxide pellets were added to the mixture until the aqueous layer was at pH<10. After separation, the aqueous layer was extracted twice with EtOAc (2×100 ml). The combined EtOAc layers were dried over potassium carbonate, filtered and concentrated to give product as a colorless oil (47.1 g, yield 48.08%). ¹H NMR (CDCl₃) δ 0.89 (t, J=6.9 Hz, 6H), 1.23-1.38 (m, 6H), 1.28 (t, J=7.5 Hz, 3H), 1.45-1.77 (m, 6H), 4.15 (q, J=7.2 Hz, 2H). ¹³C NMR (CDCl₃) δ 13.9, 14.3, 23.0, 26.0, 39.9, 60.7, 60.9, 177.4.

2-Amino-2-butylhexan-1-ol

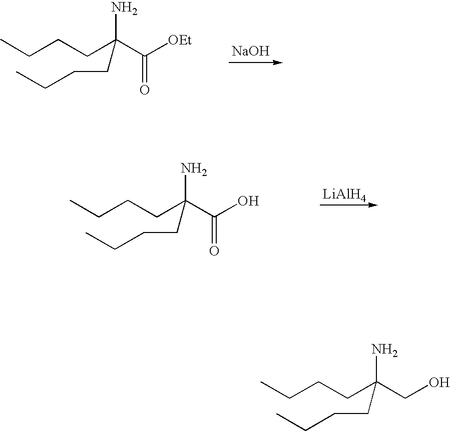

To a 1000 ml flask was added ethyl 2-amino-2-butylhexanoate (46 g), MeOH (500 ml) and water (200 ml). After adding NaOH (9.0 g), the reaction mixture was heated to reflux for 4 hours and then evaporated off solvent to give mixture of sodium 2-amino-2-butylhexanoate and sodium hydroxide. ¹H NMR (D₂O) δ 0.78 (t, J=6.9 Hz, 6H), 1.05-1.36 (m, 8H), 1.70-1.88 (m, 4H). ¹³C NMR. LC/MS (ES/M+H): 188.2.

The solid mixture was added to a 1M solution of LiAlH₄ (223 ml, 1.05 eq.) in THF. After complete addition, the reaction mixture was refluxed for 3 hours, then stirred overnight at room temperature. The mixture was cooled to about 0° C., then quenched with water (100 ml) and 1 N aq. NaOH (100 ml). The resulting solid was broke up with additional water (100 ml) and the suspension was heated at 65° C. for 10 minutes. After cooling to room temperature, diethyl ether (500 ml) was added, the mixture was stirred and filtered. The diethyl ether layer was separated, dried and concentrated in vacuo to give desired product as a solid. (31.0 g, yield 83.75%). ¹H NMR (CDCl₃) δ 0.84 (t, J=5.1H, 6H), 1.10-1.38 (m, 12H), 3.24 (s, 2H). ¹³C NMR (CDCl₃) δ 14.0, 23.3, 25.5, 36.6, 54.7, 68.2.

2-Amino-2-butylhexyl Hydrogen Sulfate

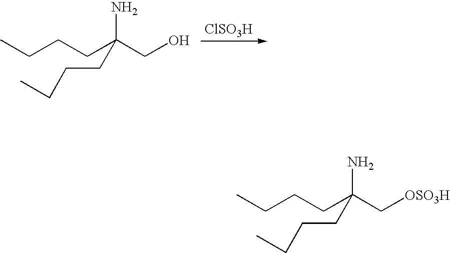

The product of 2-amino-2-butylhexanol (12 g) was dissolved in CH₂Cl₂ (120 ml) and treated with chlorosulfonic acid (13.39 g). The reaction mixture was stirred at room temperature overnight. After removing the solvent, the resulting slurry was diluted with acetone, filtered and washed with another 5 ml acetone. The white solid was dried to give 8.1 g product. (yield 46.16%). ¹H NMR (D₂O) δ 0.80 (t, J=6.6 Hz, 6H), 1.23 (m, 8H), 1.60 (m, 4H), 3.99 (s, 2H). LC/MS (ES/M+H): 154.1.

2-(2-Amino-2-butylhexylthio)-4-nitro-(4′-methoxy)-benzophenone

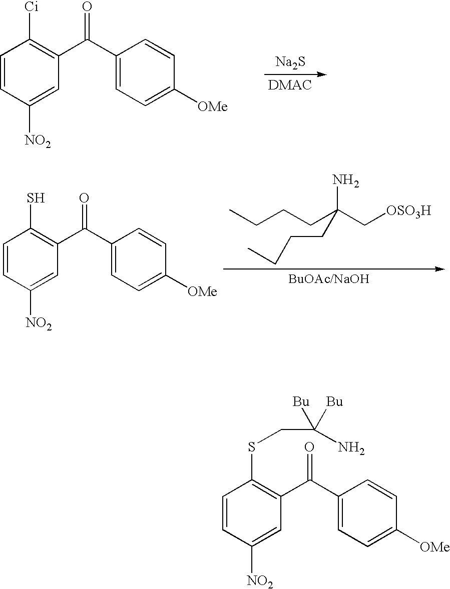

To a three necked flask was added 2-chloro-4-nitro-(4′-methoxy)-benzophenone (4 g) and 40 ml of dimethylacetamide. The reaction mixture was heated to 40° C. until the mixture become homogeneous. Sodium sulfide hydrate (Na₂S.3H₂O)(1.88 g, 1.05 eq.) and water (2 ml) were combined in a separated flask and heated to 55° C. until homogenous. The Na₂S solution was then added portionwise to the reaction mixture over 20 minutes. After stirred 4 hours at 40° C., the reaction mixture was cooled to 30° C. 2-Amino-2-butylhexyl hydrogen sulfate (3.81 g, 1.1 eq.), BuOAc (40 ml) and water (20 ml) was added. The reaction mixture was stirred and heated to an internal temperature of 93° C. and NaOH (1.42 g in 20 ml water) was added dropwise. After complete addition, the reaction was stirred an additional 1 hour at 93° C., then cooled to room temperature. After separation, the aqueous layer was extracted with EtOAc (2×50 ml). Combined organic layers were dried and concentrated in vacuo to give a yellow oil. Flash chromatography on silica gel, eluting with hexane: EtoAc (4:1-1:4), afforded the desired product (near 1.99 g) as a yellow oil. (˜31%). ¹H NMR (CDCl₃) δ 0.86 (t, J=6.44 Hz, 6H), 1.12-1.60 (m, 8H), 1.31-1.46 (m, 4H), 3.02 (s, 2H), 3.87 (s, 3H), 6.96 (d, J=8.86 Hz, 2H), 7.62 (d, J=8.86 Hz, 1H), 7.77 (d, J=8.86 Hz, 2H), 8.14 (d, J=2.42 Hz, 1H), 8.23 (dd, J=8.86, 2.42 Hz, 1H). ¹³C NMR (CDCl₃) δ 14.2, 23.4, 26.0, 39.6, 45.8, 54.6, 55.8, 114.4, 123.6, 124.8, 128.4, 129.2, 132.8, 139.7, 144.7, 147.6, 164.6, 193.2. LC/MS (ES/M+H): 445.2.

3,3-Dibutyl-2,3,4,5-tetrahydro-7-nitro-5-(4′-methoxyphenyl)-1,4-benothiazepine

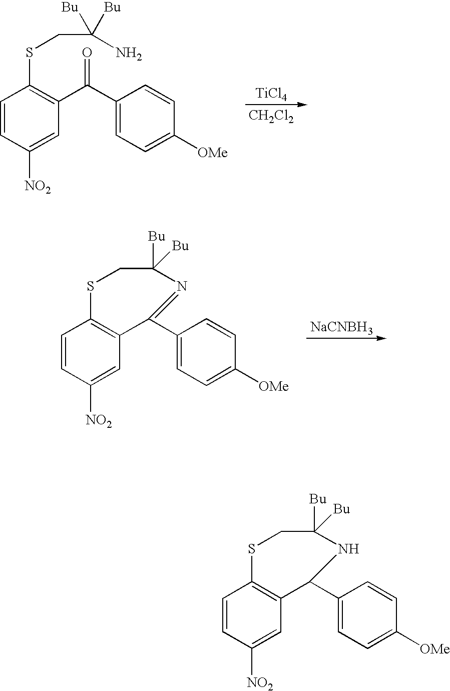

To a three necked flask was added 2-(2-amino-2-butylhexylthio)-4-nitro-(4′-methoxy)-benzophenone (1 g), triethyl amine (0.75 ml) and CH₂Cl₂ (40 ml). After the reaction mixture was stirred 20 minutes in an ice-water bath, TiCl₄ (2.25 ml of 1 M solution in CH₂Cl₂) was added via syringe. After stirring overnight, the reaction mixture was carefully quenched with a methanolic solution of NaCNBH₃ (0.84 g) and stirred for 1 hour. The reaction was basified to pH 13 with 5N aq. NaOH, extracted with EtOAc (2×100 ml) dried and evaporated to a yellow oil. Flash chromatography on silica gel, eluting with EtOAc/hexanes, provide the desired product as a yellow oil. (yield 79.67%). ¹H NMR (CDCl₃) δ 0.82-0.94 (m, 6H), 1.03-1.62 (m, 11H), 1.86-1.99 (m, 1H), 2.77 (d, J=14.6 Hz, 1H), 3.05 (d, J=14.6 Hz, 1H), 3.86 (s, 3H), 5.50 (s, 1H), 6.95 (d, J=8.66 Hz, 2H), 7.25 (d, J=8.66 Hz, 2H), 7.45 (d, J=1.81 Hz, 1H), 7.62 (d, J=8.46 Hz, 1H), 7.88 (1H, dd, J=8.46, 2.42 Hz). LC/MS (ES/M+H): 429.2.

3.3-Dibutyl-2,3,4,5-tetrahydro-7-nitro-5-(4′-methoxyphenyl)-14-benzothiazepine 1,1-dioxide

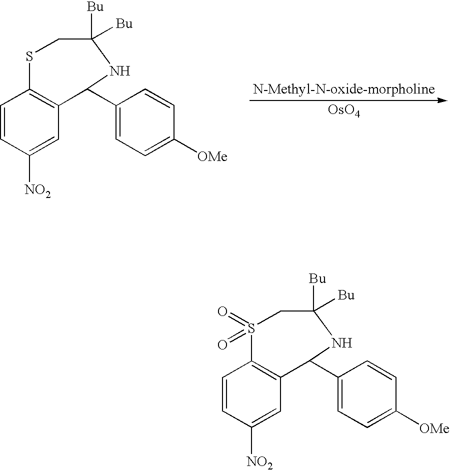

To a solution of 3,3-dibutyl-2,3,4,5-tetrahydro-7-nitro-5-(4′-methoxyphenyl)-1,4-benzothiazepine (0.91 g) in 20 ml THF and 9 ml tBuOH was added N-methyl-morpholine-N-oxide (0.74 g, 3 eq.) and OsO₄ (0.5 ml, 2.5% wt in t-butanol). The reaction mixture was stirred at room temperature overnight. The reaction mixture was transferred to a separatory funnel and partitioned between 100 ml of brine and 100 ml of EtOAc. The aqueous layer was extracted three times with EtOAc (3×25 ml). The organic layer was dried, concentrated in vacuo. The residue was purified via flash chromatography on silica gel, eluting with 20% EtOAc/hexane to give the desired product (0.82 g). (yield 83.84%). Reverse-phase HPLC: rt=18.8 min, 99.7% pure. ¹H NMR (CDCl₃) δ 0.90-0.97 (m, 6H), 1.03-1.38 (m, 8H), 1.42-1.50 (m, 2H), 1.57 (br s, 1H), 1.74-1.88 (m, 1H), 2.14-2.26 (m, 1H), 3.32 (q_{AB}, J_{AB}=15.3 Hz, Δν=91.7 Hz, 2H), 3.87 (s, 3H), 5.97 (s, 1H), 6.98 (d, J=8.66 Hz, 2H), 7.30 (d, J=8.66 Hz, 2H), 7.62 (d, J=2.02 Hz, 1H), 8.18 (dd, J=8.46, 2.21 Hz, 1H), 8.30 (d, J=8.46 Hz, 1H). ¹³C NMR (CDCl₃) δ 13.9, 14.0, 22.8, 23.0, 25.2, 31.7, 40.7, 55.3, 57.2, 63.7, 114.5, 121.8, 123.2, 128.7, 129.2, 132.6, 145.5, 148.3, 150.2, 159.3. HRMS (ES/M+H) calcd for C₂₄H₃₂N₂O₅S: 461.2102, found: 461.2105. Anal Calcd for (C₂₄H₃₂NO₅S): C, 62.58; H, 7.00; N, 6.08; S, 6.96. Found: C, 62.60; H, 7.10; N, 6.01; S, 6.83.

### EXAMPLE 2

### 3.3-Dibutyl-2.3,4,5-tetrahydro-7-dimethylamino-5-(4′-methoxyphenyl)-1,4-benzothiazepine 1,1-dioxide

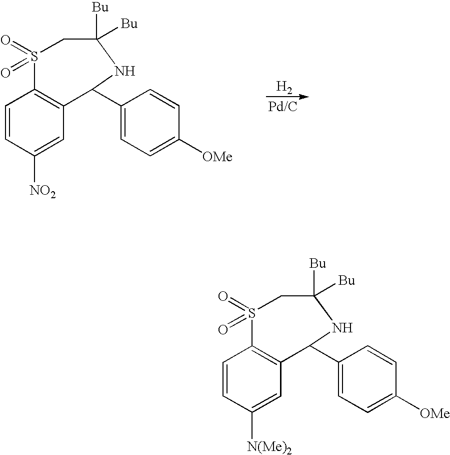

3,3-Dibutyl-2,3,4,5-tetrahydro-7-nitro-5-(4′-methoxyphenyl)-1,4-benzothiazepine 1,1-dioxide (0.768 g), MeOH (60 ml), 10% Pd/C (0.100 g) and formaldehyde (3.14 g/37% in water) were combined in a Fischer Porter bottle. Sulfuric acid (0.020 g) was added to the reaction mixture. The reactor was purged with H₂ and pressurized to 45 psig H₂. After stirring at 50° C. overnight, sodium carbonate (0.2 g) was added and the mixture stirred for 1 hour more. The reaction mixture was filtered through celite and washed with additional MeOH (20 ml). After concentrated, the residue was dissolved in EtOAc (200 ml) and washed with water (100 ml) and brine (100 ml). The organic layer was dried and concentrated in vacuo, and the resulting yellow oil was purified by flash chromatography on silica gel, eluting with EtOAc: hexane: triethylamine 20:80:1, to give 0.43 g of product. (Yield 56.23%). Reverse-phase HPLC: rt=5.9 min, 100% pure. ¹H NMR (CDCl₃) δ 0.81 (t, J=7.4 Hz, 3H), 0.86 (t, J=6.9 Hz, 3H), 1.08-1.53 (m, 10H), 1.81 (m, 1H), 2.20 (m, 1H), 2.79 (s, 6H), 3.18 (q_{AB}, J_{AB}=6.9 Hz, Δν=116.8 Hz, 2H), 3.83 (s, 3H), 5.92 (s, 1H), 5.96 (s, 1H), 6.46 (d, J=9.6 Hz, 1H), 6.90 (d, J=9.6 Hz, 2H), 7.33 (d, J=8.1 Hz, 2H), 7.87 (d, J=9.6 Hz, 1H). Anal Calcd for (C₂₆H₃₈N₂O₃S): C, 68.09; H, 8.35; N, 6.11; S, 6.99; 0, 10.46. Found: C, 66.92; H, 8.17; N, 5.99; S, 6.75; O, 10.29.

3.3-Dibutyl-2,3,4,5-tetrahydro-7-dimethylamino-5-(4′-hydroxyphenyl)-1,4-benzothiazepine 1,1-dioxide

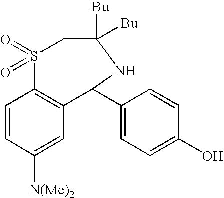

3,3-Dibutyl-2,3,4,5-tetrahydro-7-dimethylamino-5-(4′-methoxyphenyl)-1,4-benzothiazepine 1,1-dioxide (0.300 g, 0.655 mmol) was dissolved in CH₂Cl₂ (10 ml). The mixture was cooled to 0° C., and a solution of 1 M BBr₃ in CH₂Cl₂ (1.96 ml, 1.96 mmol) was added. After 15 min, the cooling bath was removed. After 3 hrs more, the reaction mixture was again cooled to 0° C. and quenched with 10% aq. HCl (9 ml). NaHCO₃ (about 1.0 g) was added until pH=7, and the mixture was extracted with CH₂Cl₂ (3×5 ml). The combined organic extracts were dried (Na₂SO₄) and concentrated in vacuo. Purification by flash chromatography on silica gel, eluting with 30% EtOAc/hexane, gave the desired product (0.250 g/86%). Reverse-phase HPLC: rt=13.6 min, 98.8% pure. ¹H NMR (CDCl₃) δ 0.81 (t, J=6.9 Hz, 3H), 0.86 (t, J=6.9 Hz, 3H), 1.05-1.46 (m, 10H), 1.80 (m, 1H), 2.23 (m, 1H), 2.80 (s, 6H), 3.18 (q_{AB}, J_{AB}=14.1 Hz, Δν=119.2 Hz, 2H), 4.79 (s, 1H), 5.92 (s, 1H), 5.96 (s, 1H), 6.47 (d, J=9.9 Hz, 1H), 6.84 (d, J=8.1 Hz, 2H), 7.49 (d, J=9.0 Hz, 2H), 7.88 (d, J=9.0 Hz, 1H). LC/MS (ES/M+H): 445. Anal Calcd for (C₂₅H₃₆N₂O₃S): C, 67.53; H, 8.17; N, 6.30; S, 7.20; O, 10.80. Found: C, 67.52; H, 8.20; N, 6.23; S, 7.18; O, 11.00.

2-[2-[2-[4-[3,3-Dibutyl-7-(dimethylamino)-2,3,4,5-tetrahydro-1,1-dioxido-1,4-benzothiazepin-5-yl]phenoxy]ethoxy]ethoxy]ethoxy iodide

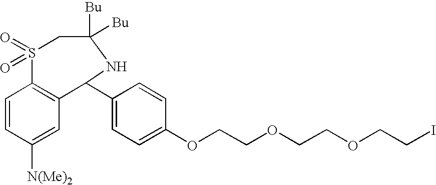

60% Sodium hydride in mineral oil (0.0100 g, 0.247 mmol) and DMF (1.0 ml) were combined in a 100 ml round-bottom flask. The mixture was cooled to 0° C., and a solution of 3,3-dibutyl-2,3,4,5-tetrahydro-7-dimethylamino-5-(4′-hydroxyphenyl)-1,4-benzothiazepine 1,1-dioxide (0.100 g, 0.225 mmol) in DMF (1.5 ml) was added. After 1 hr, 1,2-bis-(2-iodoethoxy)ethane (0.832 g, 2.249 mmol) in DMF (1.0 ml) was added. The reaction mixture was allowed to warm to room temperature and was then heated to 40° C. After 5 hrs, the reaction mixture was diluted with ethyl ether and water. The organic layer was washed with water and brine, dried (Na₂SO₄) and concentrated in vacuo. Purification by circular chromatotron on silica gel, eluting with EtOAc/hexanes gave the desired product (0.075 g/50%). Reverse-phase HPLC: rt=15.3 min, 96% pure. ¹H NMR (CDCl₃) δ 0.81 (t, J=7.2 Hz, 3H), 0.86 (t, J=6.9 Hz, 3H), 1.08-1.55 (m, 10H), 1.80 (m, 1H), 2.20 (m, 1H), 2.79 (s, 6H), 3.17 (q_{AB}, J_{AB}=14.1 Hz, Δν=113.1 Hz, 2H), 3.25 (t, J=6.9 Hz, 2H), 3.72 (m, 6H), 3.88 (t, J=4.8 Hz, 2H), 4.14 (t, J=4.8 Hz, 2H), 5.91 (s, 1H), 5.97 (s, 1H), 6.45 (d, J=9.0 Hz, 1H), 6.90 (d, J=7.8 Hz, 2H), 7.31 (d, J=7.8 Hz, 2H), 7.86 (d, J=8.7 Hz, 1H). LC/MS (ES/M+H): 687.

### EXAMPLE 4

### N-[2-[2-[2-[4-[3,3-Dibutyl-7-(dimethylamino)-2,3,4,5-tetrahydro-1,1-dioxido-1,4-benzothiazepin-5-yl]phenoxy]ethoxy]ethoxy]ethoxy]-N,N,N-triethylaminium Iodide

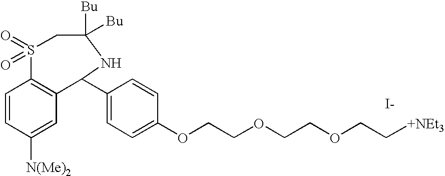

2-[2-[2-[4-[3,3-Dibutyl-7-(dimethylamino)-2,3,4,5-tetrahydro-1,1-dioxido-1,4-benzothiazepin-5-yl]phenoxy]ethoxy]ethoxy]ethoxy iodide (0.0500 g, 0.0729 mmol), Et₃N (0.50 ml, 3.59 mmol) and CH₃CN (0.80 ml) were combined in a 25 ml round-bottom flask. The mixture was heated 40° C. After 3 days, the mixture was concentrated in vacuo, and the resulting residue was washed repeatedly with ethyl ether to yield the desired product (0.040 g/70%). Reverse-phase HPLC: rt=10.9 min, 97% pure. ¹H NMR (CDCl₃) δ 0.80 (t, J=6.9 Hz, 3H), 0.86 (t, J=6.3 Hz, 3H), 1.11-1.43 (m, 10H), 1.34 (t, J=6.9 Hz, 9H), 1.78 (m, 1H), 2.15 (m, 1H), 2.80 (s, 6H), 3.24 (q_{AB}, J_{AB}=13.8 Hz, Δν=155.4 Hz, 2H), 3.50 (q, J=7.2 Hz, 6H), 3.71 (br s, 6H), 3.83 (m, 2H), 4.00 (br s, 2H), 4.11 (m, 2H), 5.92 (s, 1H), 5.96 (s, 1H), 6.47 (d, J=9.6 Hz, 1H), 6.89 (d, J=8.7 Hz, 2H), 7.33 (d, J=8.7 Hz, 2H), 7.86 (d, J=8.4 Hz, 1H). LC/MS (ES/M−HI): 659.5.

4-[[4-[3,3-Dibutyl-7-(dimethylamino)-2,3,4,5-tetrahydro-1,1-dioxido-1,4-benzothiazepin-5-yl]phenoxy]methyl]phenylmethyl Chloride

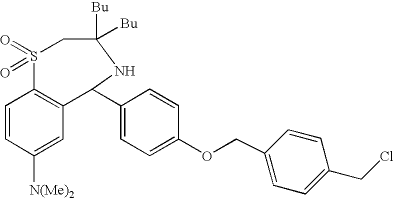

3,3-Dibutyl-2,3,4,5-tetrahydro-7-dimethylamino-5-(4′-hydroxyphenyl)-1,4-benzothiazepine 1,1-dioxide (0.070 g, 0.157 mmol), K₂CO₃ (0.033 g, 0.236 mmol) and α,α′-dichloro-p-xylene (0.2756 g, 1.574 mmol) were combined with acetone (2.50 ml). The mixture was heated to 70° C. After 48 hrs, the mixture was cooled to room temperature and concentrated in vacuo. The residue was dissolved in EtOAc and washed with water. The water washes were extracted with EtOAc. Combined organic layers were washed with brine, dried (Na₂SO₄) and concentrated in vacuo. Purification by circular chromatotron on silica gel, eluting with 20% EtOAc/hexanes, gave the desired product (0.067 g/73%). Reverse-phase HPLC: rt=19.6 min, 97% pure. ¹H NMR (CDCl₃) δ 0.81 (t, J=6.9 Hz, 3H), 0.87 (t, J=6.9 Hz, 3H), 1.12-1.50 (m, 10H), 1.83 (m, 1H), 2.20 (m, 1H), 2.79 (s, 6H), 3.18 (q_{AB}, J_{AB}=12.6 Hz, Δν=116.3 Hz, 2H), 4.59 (s, 2H), 5.08 (s, 2H), 5.91 (s, 1H), 6.00 (s, 1H), 6.48 (d, J=8.1 Hz, 1H), 6.96 (d, J=8.1 Hz, 2H), 7.32-7.44 (m, 6H), 7.88 (d, J=9.3 Hz, 1H). LC/MS (ES/M+H): 583. Anal Calcd for (C₃₃H₄₃N₂O₃SCl): C, 68.01; H, 7.44; N, 4.81; S, 5.49; O, 8.24. Found: C, 67.70; H, 7.21; N, 4.77; S, 5.35; O, 7.98.

### EXAMPLE 5

### 1-[[4-[[4-[3,3-Dibutyl-7-(dimethylamino-2,3,4,5-tetrahydro-1,1-dioxido-1,4-benzothiazepin-5-yl]phenoxy]methyl]phenyl]methyl]-4-aza-1-azoniabicyclo[2,2,2]octane Chloride

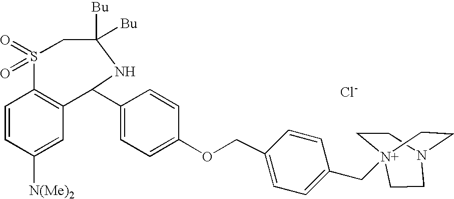

A solution of 4-[[4-[3,3-dibutyl-7-(dimethylamino)-2,3,4,5-tetrahydro-1,1-dioxido-1,4-benzothiazepin-5-yl)phenoxy]methyl]phenylmethyl chloride (0.045 g, 0.0773 mmol), in CH₃CN (1.50 ml) was added dropwise to a solution of 1,4-diazabicyclo[2,2,2]octane (0.086 g, 0.773 mmol) in CH₃CN (1.00 ml) over a period of 30 min. at 40° C. After 2 hrs, the reaction mixture was cooled to room temperature and concentrated in vacuo. The residue was washed repeatedly with ethyl ether and concentrated in vacuo to give the desired product (0.045 g/83%). Reverse-phase HPLC: rt=13.0 min, 97% pure. ¹H NMR (CDCl₃) δ 0.80 (t, J=6.9 Hz, 3H), 0.87 (t, J=6.9 Hz, 3H), 1.10-1.48 (m, 10), 1.76 (m, 1H), 2.17 (m, 1H), 2.81 (s, 6H), 3.18 (br s, 6H), 3.19 (q_{AB}, J_{AB}=14.7 Hz, Δν=113.0 Hz, 2H), 3.75 (br s, 6H), 5.08 (s, 2H), 5.12 (s, 2H), 5.92 (s, 1H), 5.99 (s, 1H), 6.47 (d, J=8.7 Hz, 1H), 6.95 (d, J=8.7 Hz, 2H), 7.35 (d, J=9.6 Hz, 2H), 7.51 (d, J=7.5 Hz, 2H), 7.65 (d, J=7.8 Hz, 2H), 7.86 (d, J=8.1 Hz, 1H). LC/MS (ES/M−HCl): 659. Anal Calcd for (C₃₉H₅₅N₄O₃SCl.3H₂O): C, 62.50; H, 8.20; N, 7.48; S, 4.28; O, 12.81. Found: C, 62.30; H, 7.82; N, 7.43; S, 4.20; O, 12.86.

N-[2-[4-[3,3-Dibutyl-7-(dimethylamino)-2,3,4,5-tetrahydro-1,1-dioxido-1,4--benzothiazepin-5-yl]phenoxy]ethyl]-N,N-diethylamine

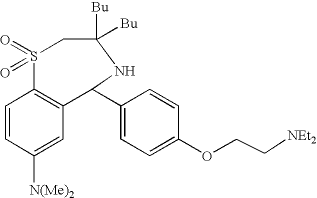

In a 250 ml round-bottom flask, NaOH (3.5 ml, 3.5 mmol, 1N in water) was added to a mixture of 2-diethylamino ethyl chloride (0.4257 g, 2.474 mmol) in ethyl ether (3.5 ml) at 0° C. The mixture was allowed to warm to room temperature and was extracted with ethyl ether. The organic layer was dried (K₂CO₃) for 2 hrs.

In a separate 100 ml round-bottom flask, NaH (0.020 g, 0.50 mmol, 60% in mineral oil) was suspended in DMF (1.0 ml). The mixture was cooled to 0° C. and a solution of PHA-404434 3,3-dibutyl-2,3,4,5-tetrahydro-7-dimethylamino-5-(4′-hydroxyphenyl)-1,4-benzothiazepine 1,1-dioxide (0.110 g, 0.2474 mmol) in DMF (1.5 ml) was added. After 0.5 hrs, the diethylamino ethyl chloride solution was added. The resulting mixture was heated to 40° C. overnight. The reaction mixture was then cooled to room temperature and diluted with ethyl ether. The mixture was washed with 5% aq. NaOH (3 ml), water (10 ml) and brine (10 ml). The organic layer was dried (Na₂SO₄) and concentrated in vacuo. Purification by reverse-phase HPLC, eluting with 5-100% CH₃CN/H₂O, gave the desired product (0.064 g/48%). Reverse-phase HPLC: rt=11.6 min, 99% pure. ¹H NMR (CDCl₃) δ 0.80 (t, J=6.9 Hz, 3H), 0.86 (t, J=6.6 Hz, 3H), 1.05 (t, J=6.9 Hz, 6H), 1.12-1.41 (m, 10H), 1.79 (m, 1H), 2.21 (m, 1H), 2.63 (q, J=7.2 Hz, 4H), 2.78 (s, 6H), 2.88 (t, J=6.0 Hz, 2H), 3.16 (q_{AB}, J_{AB}=14.4 Hz, Δν=116.1 Hz, 2H), 4.06 (t, J=6.3 Hz, 2H), 5.92 (s, 1H), 5.96 (s, 1H), 6.46 (d, J=8.7 Hz, 1H), 6.90 (d, J=9.0 Hz, 2H), 7.30 (d, J=9.0 Hz, 2H), 7.87 (d, J=8.7 Hz, 1H). LC/MS (ES/M+H): 544.

### EXAMPLE 3

### N-[2-[4-[3,3-Dibutyl-7-(dimethylamino)-2,3,4,5-tetrahydro-1,1-dioxido-1,4-benzothiazepin-5-yl]phenoxy]ethyl-N,N,N-triethylaminium Iodide

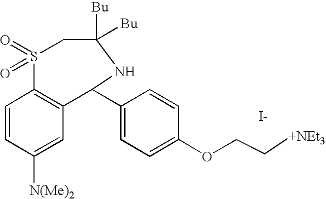

N-[2-[4-[3,3-Dibutyl-7-(dimethylamino)-2,3,4,5-tetrahydro-1,1-dioxido-1,4-benzothiazepin-5-yl]phenoxy]ethyl]-N,N-diethylamine (0.030 g, 0.0552 mmol), ethyl iodide (0.005 ml, 0.0619 mmol) and CH₃CN (0.50 ml) were combined in a 10 ml vial and heated to 40° C. After 18 hrs, additional ethyl iodide (0.010 ml, 0.124 mmol) was added. After 24 hrs more, additional ethyl iodide (0.010 ml, 0.124 mmol) was added. After 18 hrs more, the mixture was concentrated in vacuo, and the residue was washed with ethyl ether to give the desired product (0.032 g/100%). Reverse-phase HPLC: rt=12.3 min, 97% pure. ¹H NMR (CDCl₃) δ 0.80 (t, J=7.2 Hz, 3H), 0.86 (t, J=7.2 Hz, 3H), 1.08-1.40 (m, 10H), 1.49 (t, J=7.2 Hz, 9H), 1.78 (m, 1H), 2.15 (m, 1H), 2.82 (s, 6H), 3.17 (q_{AB}, J_{AB}=13.8 Hz, Δν=125.2 Hz, 2H), 3.60 (q, J=7.2 Hz, 6H), 4.10 (d, J=4.8 Hz, 2H), 4.53 (s, 2H), 5.88 (s, 1H), 6.10 (s, 1H), 6.47 (d, J=8.1 Hz, 1H), 6.92 (d, J=7.8 Hz, 2H), 7.38 (d, J=7.8 Hz, 2H), 7.87 (d, J=7.8 Hz, 1H). LC/MS(ES/M+H): 583.

Synthesis of 1,5 benzothiazepines Scheme IV

1-dimethyl,t-butlysilyl, 2.2-dibutyl-1,3-proandiol

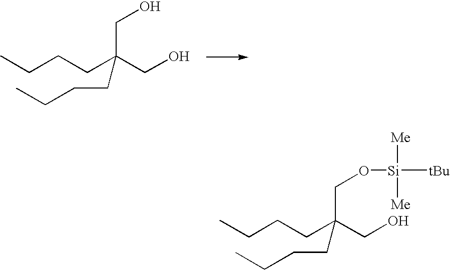

A solution of 2,2-dibutyl-1,3-propanediol (18.8 g, 100 mmol) in THF (60 ml) was added to a slurry of NaH (4.00 g, 100 mmol, 40% in mineral oil) in THF (100 ml). After 1 hr, the mixture was cooled to 0° C., and tBuMe₂SiCl (100 ml, 100 mmol, IM in THF) was added. The mixture was allowed to warm to room temp. overnight. The mixture was concentrated in vacuo, and the residue was treated with water (750 ml) and ethyl ether (60 ml). The ether layer was washed with aq. NaHCO₃ and brine, dried (Na₂SO₄) and concentrated in vacuo. Purification by flash chromatography on silica gel, eluting with 5% EtOAc/hexane, gave the desired product (27.3 g/90%). ¹H NMR (CDCl₃) δ 0.05 (s, 6H), 0.86 (s, 6H), 0.88 (s, 9H), 1.20 (m, 12H), 3.50 (s, 4H).

¹³C NMR (CDCl₃) 8-5.7, 14.0, 18.1, 23.6, 25.1, 25.8, 30.6, 40.8, 69.6, 70.2. GC/MS (ES/M−tBu): 245. Anal Calcd for (C₁₇H₃₈O₂Si): C, 67.48; H, 12.66. Found: C, 67.98; H, 12.81.

2-bromomethyl-2-butyl Hexanoic Acid

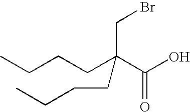

Sodium periodate (17.67 g, 82.62 mmol) and RuCl₃ (0.125 g, 0.603 mmol) were added to a solution of 2-[[(t-butyldimethylsilyl)oxy]methyl]-2-butylhexanol (10.0 g, 33.05 mmol) in CCl₄ (20.0 ml), CH₃CN (20.0 ml) and water (30.0 ml) at 0° C. After stirring for 20 hrs at room temp, the mixture was filtered through celite, extracted with CH₂Cl₂ and concentrated in vacuo. The residue was purified by flash chromatography on silica gel, eluting with 20% EtOAc/hexane. The resulting residue was taken up in 48% HBr (35 ml) and heated to reflux. After 24 hrs, the mixture was cooled to room temp, and extracted with ethyl ether (3×). The combined extracts were washed with brine, dried (Na₂SO₄) and concentrated in vacuo. Purification by sublimation at 50° C. under vacuum gave a white solid (5.26 g/60%). ¹H NMR (CDCl₃) δ 0.89 (t, J=7.0 Hz, 6H), 1.11-1.24 (m, 4H), 1.25-1.34 (m, 4H), 1.65-1.69 (m, 4H), 3.56 (s, 2H). ¹³C NMR (CDCl₃) δ 13.9, 22.9, 26.0, 34.1, 36.2, 50.2, 180.9. MS (ES/M−HBr—H): 182.6. Anal Calcd for (C¹¹H₂₁O₂Br): C, 49.82; H, 7.98; Br, 30.13. Found: C, 49.94; H, 7.96; Br, 30.30.

2-Amino-5-fluorobenzothiazole

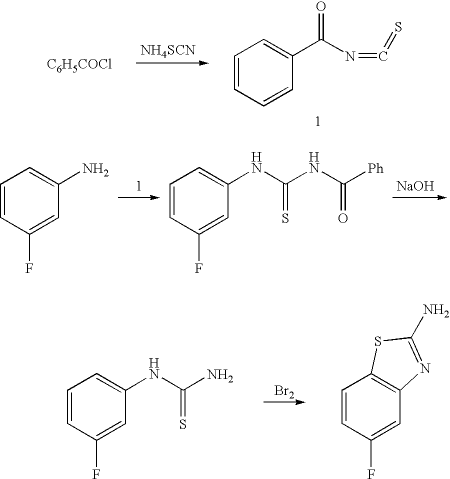

Benzoyl chloride (34.72 g, 247 mmol) was added to a mixture of ammonium thiocyanate (18.8 g, 247 mmol) and acetone (100.0 ml) at 30° C. The mixture was heated to reflux for 10 min, and then 3-fluoroaniline (25.0 g, 225 mmol) was added at 50° C. over 10 min. Additional acetone (20 ml) was added, and the mixture, was heated to reflux for 1 hr. A solution of NaOH (28.76 g, 719 mmol) in water (166 ml) was added, and the resulting solution was heated to reflux. After 1.5 hrs, the mixture was cooled to room temperature and concentrated to remove the acetone. Concentrated aq. HCl was added until pH=5.0. Then, concentrated aq. NH₄OH was added until pH=11.0. The precipitate was filtered, washed with water and dried via vacuum oven to give 3-fluorophenylthiourea (32 g/84%).

A solution of bromine (29.08 g, 182 mmol) in CH₂Cl₂ (80 ml) was added to a solution of 3-fluorophenylthiourea (31.0 g, 182 mmol) in CH₂Cl₂ (550 ml). The mixture was heated to reflux. After 3 hrs, the reaction mixture was cooled to room temp and filtered. The solid was suspended in water (1 L) and conc. NH₄OH was added until basic. The mixture was extracted with EtOAc (3×200 ml). Combined organic extracts were washed with water (150 ml) and brine (150 ml), dried (Na₂SO₄) and concentrated in vacuo to give a colored solid. Recrystallization from benzene gave the desired product as a white solid (18.9 g/50%). ¹H NMR (DMSO-d₆) δ 6.83 (t, J=7.7 Hz, 1H), 7.10 (d, J=10.5 Hz, 1H), 7.59-7.64 (m, 3H). LC/MS (ES/M+H): 168.9. Anal Calcd for (C₇H₅N₂SF): C, 49.99; H, 3.00; N, 16.66; S, 19.06. Found: C, 50.04; H, 2.95; N, 16.57; S, 18.96.

2-(((2-amino-4-fluorophenyl)thio)methyl-2-butylhexanoic Acid

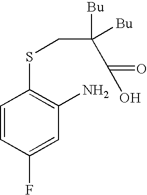

Potassium hydroxide (1.76 g, 31.52 mmol) was added to a suspension of 2-amino-5-fluorobenzothiazole (0.278 g, 1.65 mmol) in water (3.5 ml). The mixture was heated to reflux for 7 hrs and then allowed to cool to room temp. 2-(Bromomethyl)-2-butylhexanoic acid (0.44 g, 1.65 mmol) was added. After 18 hrs more, conc. aq. HCl was added until pH=4. The mixture was extracted with EtOAc, dried (Na₂SO₄), and concentrated in vacuo. Purification by flash chromatography on silica gel, eluting with 10-40% EtOAc/hexane, gave the desired product (0.459 g/85%). ¹H NMR (CD₃OD) δ 0.81 (t, J=7.0 Hz, 6H), 1.00-1.13 (m, 4H), 1.15-1.22 (m, 4H), 1.50-1.68 (m, 4H), 2.92 (s, 2H), 6.24 (dt, J=8.5, 2.6 Hz, 1H), 6.41 (dd, J=11.1, 2.8 Hz, 1H), 7.26 (dd, J=8.5, 6.4 Hz, 1H). LC/MS (ES/M−C₁₁H₂₆O₂): 141.9. Anal Calcd for (C₁₇H₂₆NO₂SF): C, 62.35; H, 8.01; N, 4.28; S, 9.77. Found: C, 62.23; H, 8.28; N, 4.09; S, 9.06.

3.3-Dibutyl-2.3-dihydro-5H-7-fluoro-1,5-benzothiazepine-4-one

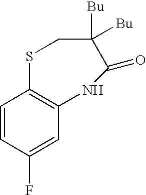

p-Toluenesulfonic acid (0.426 g, 2.24 mmol) was added to a suspension of 2-(((2-amino-4-fluorophenyl)thio)methyl)-2-butylhexanoic acid (9.1 g, 27.8 mmol) in tetradecane (130.0 ml). The mixture was heated to reflux, collecting water in a Dean-Stark trap. After 15 min, the mixture was cooled to room temp and purified by flash chromatography on silica gel, eluting with 0-10% EtOAc/hexane to give the desired product (4.60 g/50%). ¹H NMR (CDCl₃) δ 0.85 (t, J=6.8 Hz, 6H), 1.24 (m, 8H), 1.60 (m, 2H), 1.80 (m, 2H), 2.90 (s, 2H), 6.68-6.74 (m, 2H), 7.38 (dd, J=9.1, 6.4 Hz, 1H), 7.94 (s, 1H). Anal Calcd for (C₁₇H₂₄NOSF): C, 65.99; H, 7.82; N, 4.53; S, 10.34. Found: C, 66.05; H, 7.91; N, 4.56; S, 10.25.

3.3-Dibutyl-2,3-dihydro-7-fluoro-5-(4′-methoxyphenyl)-1,5-benzothiazenine-4-one

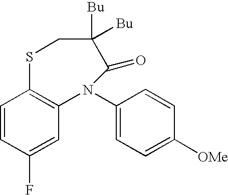

A mixture of 2,3-dihydro-3,3-dibutyl-5H-7-fluoro-1,5-benzothiazepine-4-one (4.7 g, 15.2 mmol), 4-iodoanisole (3.91 g, 16.7 mmol), K₂CO₃ (4.19 g, 30.4 mmol), CuI (0.284 g, 1.52 mmol), tris(3,6-dioxaheptyl)amine (0.182 g, 0.56 mmol) and xylenes (40 ml) were heated to reflux, collecting any water present with a Dean-Stark trap. After 48 hrs, the mixture was cooled to room temp, diluted with CHCl₃ and purified by flash chromatography on silica gel, eluting with 0-15% EtOAc/hexane, to give the desired product (6.20 g/90%). ¹H NMR (CDCl₃) δ 0.83 (t, J=6.9 Hz, 6H), 1.16-1.25 (m, 8H), 1.51 (m, 4H), 3.07 (s, 2H), 3.76 (s, 3H), 6.58 (dd, J=9.9, 2.7 Hz, 1H), 6.77-6.88 (m, 3H), 7.04 (d, J=8.7 Hz, 2H), 7.57 (dd, J=8.7, 6.6 Hz, 1H). LC/MS (ES/M+H): 416.1. Anal Calcd for (C₂₄H₃₀NO₂SF): C, 69.36; H, 7.28; N, 3.37; S, 7.70. Found: C, 69.76; H, 7.51; N, 3.39; S, 7.60.

3,3-Dibutyl-2,3,4,5-tetrahydro-7-fluoro-5-(4′-methoxyphenyl)-1,5-benzothiazepine

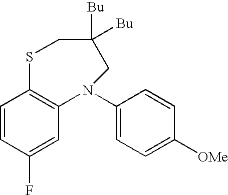

Sulfuric acid (3.56 ml, 25.7 mmol, 7.2M in THF) was added to a 1M solution of LiAlH₄ (51.0 ml, 51.0 mmol) in ethyl ether at 0° C. After 1 hr, a solution of 3,3-dibutyl-2,3-dihydro-7-fluoro-5-(4′-methoxyphenyl)-1,5-benzothiazepine-4-one (6.20 g, 14.9 mmol) in THF (45.0 ml) was added. The mixture was allowed to warm to room temp. After 3.5 hrs, the mixture was cooled to 0° C. and 30% water/THF (10 ml, v/v) was added. A solution of 1 N aq. NaOH (10.0 ml) was added, and the reaction mixture was filtered through a flitted funnel. The filtrate was extracted with ethyl ether, dried (Na₂SO₄) and concentrated in vacuo to give the desired product (4.68 g/78%). ¹H NMR (CDCl₃) δ 0.76 (t, J=6.6 Hz, 6H), 0.94-1.28 (m, 12H), 2.90 (s, 3H), 3.77 (s, 2H), 3.78 (s, 2H), 6.13 (dd, J=11.9, 2.6 Hz, 1H), 6.34-6.40 (m, 1H), 6.84 (d, J=9.1 Hz, 2H), 7.03-7.10 (m, 3H). ³C NMR (CDCl₃) δ 13.9, 23.2, 25.3, 33.1, 40.3, 41.8, 55.5, 58.3, 107.1 (d, J=22.3 Hz), 107.5 (d, J # 25.2 Hz), 114.9, 120.0, 126.3, 131.4 (d, J=9.2 Hz), 142.0, 151.3 (d, J=10.0 Hz), 155.9, 161.6 (d, J=243.0 Hz). Anal Calcd for (C₂₄H₃₂NOSF): C, 71.78; H, 8.04; N, 3.49; S, 7.97. Found: C, 71.70; H, 8.08; N, 3.59; S, 7.89.

3.3-Dibutyl-2,3,4,5-tetrahydro-7-fluoro-5-(4′-methoxyphenyl)-1,5-benzothiazepine 1,1-dioxide

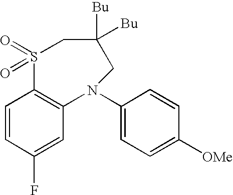

A solution of oxone (1.35 g, 2.20 mmol) in water (4.0 ml) was added to a cold (0° C.) solution of 3,3-dibutyl-2,3,4,5-tetrahydro-7-fluoro-5-(4′-methoxyphenyl)-1,5-benzothiazepine (0.40 g, 1.0 mmol) in MeOH (4.0 ml) and THF (15.0 ml). After 16 hrs, the mixture was diluted with water and extracted with CHCl₃ (3×). The combined extracts were washed with water, dried (Na₂SO₄) and concentrated in vacuo. Purification by flash chromatography on silica gel, eluting with 25% EtOAc/hexane, gave the desired

product (0.35 g/80%). ¹H NMR (CDCl₃) δ 0.77 (t, J 6.4, 6H), 0.86-1.24 (m, 8H), 1.33-1.57 (m, 4H), 3.25 (s, 2H), 3.81 (s, 3H), 3.85 (s, 2H), 6.18 (dd, J=11.7, 2.4 Hz, 1H), 6.60-6.66 (m, 1H), 6.91 (d, J=8.9 Hz, 2H), 7.12 (d, J=9.1 Hz, 2H), 7.94 (dd, J=9.1, 6.5 Hz, 1H). LC/MS (ES/M+H): 434. Anal Calcd for (C₂₄H₃₂NO₃SF): C, 66.48; H, 7.44; N, 3.23; S, 7.38. Found: C, 66.97; H, 7.63; N, 3.12; S, 7.27.

### EXAMPLE 6

### 3,3-Dibutyl-2,3,4,5-tetrahydro-7-dimethylamino-5-(4′-methoxyphenyl)-1,5-benzothiazepine 1,1-dioxide

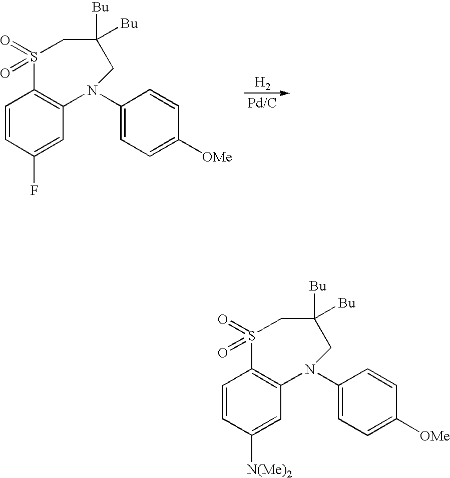

In a Fischer Porter bottle, a solution of 3,3-dibutyl-2,3,4,5-tetrahydro-7-fluoro-5-(4′-methoxyphenyl)-1,5-benzothiazepine 1,1-dioxide (1.00 g, 2.30 mmol) in THF (7.0 ml) was cooled to 0° C. Dimethylamine (23.0 ml, 46 mmol, 2M in THF) was added, and the vessel was closed and heated to 110° C. After 16 hrs, the reaction mixture was cooled to room temp and concentrated in vacuo. Purification by flash chromatography on silica gel, eluting with 25% EtOAc/hexane, gave the desired product (0.30 g/28%). ¹H NMR (CDCl₃) δ 0.76 (t, J=6.8 Hz, 6H), 1.00-1.15 (m, 8H), 1.42-1.51 (m, 4H), 2.79 (s, 6H), 3.15 (s, 2H), 3.71 (s, 2H), 3.79 (s, 3H), 5.81 (d, J=2.4 Hz, 1H), 6.34 (dd, J=9.1, 2.4 Hz, 1H), 6.84 (d, J=8.9 Hz, 2H), 7.11 (d, J=8.9 Hz, 2H).7.79 (d, J=9.1 Hz, 1H). LC/MS (ES/M+H): 459.1. Anal Calcd for (C₂₆H₃₈N₂O₃S): C, 68.08; H, 8.36; N, 6.11; S, 6.89. Found: C, 68.19; H, 8.28; N, 6.04; S, 6.90.

3,3-Dibutyl-2,3,4,5-tetrahydro-7-dimethylamino-5-(4′-hydroxyphenyl)-1,5-benzothiazepine 1,1-dioxide

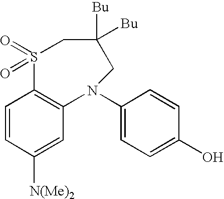

A solution of 3,3-dibutyl-2,3,4,5-tetrahydro-7-dimethylamino-5-(4′-methoxyphenyl)-1,5-benzothiazepine 1,1-dioxide (0.460 g, 1.003 mmol) in CH₂Cl₂ (6.0 ml) was cooled to 0° C. Boron tribromide (1.40 ml, 1.49 mmol, IM in CH₂Cl₂) was added, and the mixture was allowed to warm to room temperature. After 18 hrs, the mixture was cooled to 0° C., and water (4.0 ml) was added. The mixture was extracted with CH₂Cl₂ (3×). Combined extracts were dried (Na₂SO₄) and concentrated in vacuo. Purification by flash chromatography on silica gel, eluting with 30% EtOAc/hexane, gave the desired product (0.30 g/75%). ¹H NMR (CDCl₃) δ 0.78 (t, J=6.8 Hz, 6H), 1.07-1.16 (m, 8H), 1.43 (m, 4H), 2.80 (s, 6H), 3.17 (s, 2H), 3.70 (s, 2H), 5.12 (s, 1H), 5.81 (d, J=2.4 Hz, 1H), 6.33 (dd, J=9.1, 2.6 Hz, 1H), 6.79 (d, J=8.9 Hz, 2H), 7.06 (d, J=8.7 Hz, 2H), 7.80 (d, J=9.1 Hz, 1H). LC/MS (ES/M+H): 445 Anal Calcd for (C₂₅H₃₆N₂O₃S): C, 67.53; H, 8.17; N, 6.30; S, 7.20. Found: C, 67.37; H, 8.04; N, 6.23; S, 7.15.

2-[2-[4-[3,3-Dibutyl-7-(dimethylamino)-2,3,4,5-tetrahydro-1,1-dioxido-1,5-benzothiazepin-5-yl]phenoxy]ethoxy]ethoxy]ethoxy Iodide

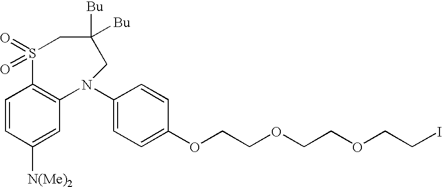

3,3-Dibutyl-2,3,4,5-tetrahydro-7-dimethylamino-5-(4′-hydroxyphenyl)-1,5-benzothiazepine 1,1-dioxide (0.50 g, 1.12 mmol) was reacted with 1,2-bis(2-iodoethoxy)ethane (4.16 g, 11.24 mmol), according to the procedure described for PHA-426998 above, to give the desired product (0.500 g/71%). ¹H NMR (CDCl₃) δ 0.76 (t, J=6.6 Hz, 6H), 1.03-1.14 (m, 8H), 1.43 (1m, 4H), 2.79 (s, 6H), 3.14 (s, 2H), 3.25 (t, J=7.0 Hz, 2H), 3.69-3.78 (m, 8H), 3.86 (t, J=4.8 Hz, 2H), 4.10 (t, J=4.4 Hz, 2H), 5.79 (d, 2.2 Hz, 1H), 6.33 (dd, J=8.9, 2.4 Hz, 1H), 6.85 (d, J=8.9 Hz, 2H), 7.09 (d, J=8.9 Hz, 2H), 7.78 (d, J=9.1 Hz, 1H). Reverse-phase HPLC: rt=28.3 min, 99% pure. LC/MS (ES/M+H): 687.23. Anal Calcd for (C₃₃H₄₇N₃O₅SI): C, 54.21; H, 6.90; N, 4.08; S, 4.66. Found: C, 54.20; H, 6.76; N, 4.03; S, 4.54.

### EXAMPLE 8

### N-[2-[2-[2-[4-[3,3-Dibutyl-7-(dimethylamino)-2.3,4,5-tetrahydro-1,1-dioxido-1,5-benzothiazepin-5-yl]phenoxy]ethoxy]ethoxy]ethoxy]-N,N,N-triethylaminium Iodide

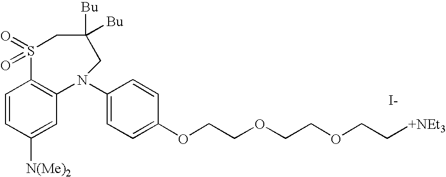

2-[2-[2-[4-[3,3-Dibutyl-7-(dimethylamino)-2,3,4,5-tetrahydro-1,1-dioxido-1,5-benzothiazepin-5-yl]phenoxy]ethoxy]ethoxy]ethoxy iodide (0.34 g, 0.496 mmol) was reacted with Et₃N (1.00 g, 9.88 mmol), according to the procedure described for PHA-426999E above, to give the desired product (0.270 g/69%). ¹H NMR (CDCl₃) δ 0.77 (t, J=6.6 Hz, 6H), 1.03-1.21 (m, 8H), 1.34 (t, J=7.2 Hz, 9H), 1.35-1.55 (m, 4H), 2.82 (s, 6H), 3.11 (s, 2H), 3.50 (q, J=7.2 Hz, 6H), 3.64-3.72 (m, 8H), 3.81 (m, 2H), 4.00 (br s, 2H), 4.08 (m, 2H), 5.89 (d, J=2.4 Hz, 1H), 6.37 (dd, J=9.1, 2.4 Hz, 1H), 6.82 (d, J=8.9 Hz, 2H), 7.06 (d, J=8.9 Hz, 2H), 7.78 (d, J=9.1 Hz, 1H). Reverse-phase HPLC: rt=6.8 min, 98% pure. LC/MS (ES/M−HI): 659.5. Anal Calcd for (C₃₇H₆₂N₃O₅SI): C, 56.39; H, 7.94; N, 5.34; S, 4.06; O, 10.16; I, 16.12. Found: C, 55.01; H, 7.95; N, 5.32; S, 3.96; O, 11.06; I, 16.80.

4-[[4-[3,3-Dibutyl-7-(dimethylamino)-2,3,4,5-tetrahydro-1,1-dioxido-1,5-benzothiazepin-5-yl]phenoxy]methyl]phenylmethyl Chloride

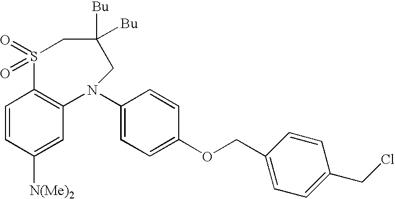

3,3-Dibutyl-2,3,4,5-tetrahydro-7-dimethylamino-5-(4′-hydroxyphenyl)-1,5-benzothiazepine 1,1-dioxide (0.50 g, 1.12 mmol) was reacted with α,α′-dichloro-p-xylene (1.968 g, 11.24 mmol), according to the procedure described for PHA-404691 above, to give the desired product (0.300 g/50%). ¹H NMR (CDCl₃) δ 0.76 (t, J=6.6 Hz, 6H), 0.98-1.14 (m, 8H), 1.43 (m, 4H), 2.79 (s, 6H), 3.14 (s, 2H), 3.69 (s, 2H), 4.58 (s, 2H), 5.04 (s, 2H), 5.81 (d, J=2.4 Hz, 1H), 6.33 (dd, J=9.1, 2.4 Hz, 1H), 6.89 (d, J=8.9 Hz, 2H), 7.10 (d, J=8.9 Hz, 2H), 7.39 (m, 4H), 7.79 (d, J=9.1 Hz, 1H). LC/MS (ES/M+1): 583. HRMS (ES/M+H) calcd for C₃₃H₄₃N₂O₃SCI: 583.2761, found: 583.2773. Anal Calcd for (C₃₃H₄₃N₂O₃SCl): C, 68.01; H, 7.44; N, 4.81; S, 5.49. Found: C, 67.97; H, 7.41; N, 4.83; S, 5.41.

### EXAMPLE 9

### 1-[[-4-[[4-[3,3-Dibutyl-7-(dimethylamino)-2,3,4,5-tetrahydro-1,1-dioxido-1,5-benzothiazepin-5-yl]phenoxy]methyl]phenyl]methyl]-4-aza-1-azoniabicyclo[2,2,2]octane Chloride

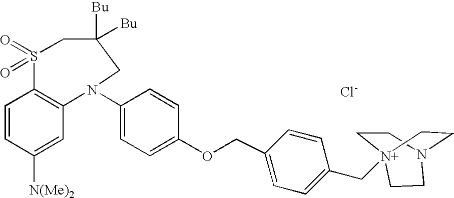

4-[[4-[3,3-Dibutyl-7-(dimethylamino)-2,3,4,5-tetrahydro-1,1-dioxido-1,5-benzothiazepin-5-yl]phenoxy]methyl]phenylmethyl chloride (0.10 g, 0.17 mmol) was reacted with 1,4-diazabicyclo[2,2,2]octane, according to the procedure described for PHA-409705E, to give desired product (0.100 g/84%). ¹H NMR (CDCl₃) δ 0.76 (t, J-6.6 Hz, 6H), 1.06-1.21 (m, 8H), 1.42 (m, 4H), 2.82 (s, 6H), 3.12 (s, 2H), 3.14 (t, J=7.0 Hz, 6H), 3.66 (s, 2H), 3.73 (t, J=7.0 Hz, 6H), 5.04 (s, 2H), 5.07 (s, 2H), 5.88 (d, J=2.4 Hz, 1H), 6.36 (dd, J=11.4, 2.6 Hz, 1H), 6.88 (d, J=8.9 Hz, 2H), 7.09 (d, J=9.1 Hz, 2H), 7.51 (d, J=8.0 Hz, 2H), 7.63 (d, J=8.0 Hz, 2H), 7.79 (d, J=9.1 Hz, 1H). Reverse-phase HPLC: rt=18.1 min, 98.1% pure. LC/MS (ES/M−Cl): 659. HRMS (ES/M−Cl) calcd for C₃₉H₅₅N₄O₃SCl: 659.3995, found: 659.4021. Anal Calcd for (C₃₉H₅₅N₄O₃SCl.3H₂O): C, 62.50; H, 8.20; N, 7.48; S, 4.28; O, 12.81. Found: C, 62.87; H, 7.93; N, 7.40; S, 4.29; O, 11.37.

N-[2-[4-[3,3-Dibutyl-7-(dimethylamino)-2,3,4,5-tetrahydro-1,1-dioxido-1,5-benzothiazepin-5-yl]phenoxy]ethyl]-N,N-diethylamine

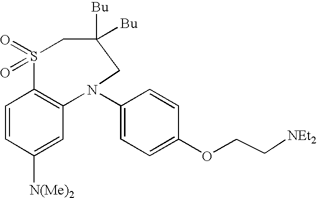

3,3-Dibutyl-2,3,4,5-tetrahydro-7-dimethylamino-5-(4′-hydroxyphenyl)-1,5-benzothiazepine 1,1-dioxide (0.50 g, 1.12 mmol) was reacted with 2-diethylamino ethyl chloride (1.935 g, 11.24 mmol), according to the procedure described for the corresponding 1,4-benzothiazepine above, to give the desired product (0.500 g/81%). ¹H NMR (CDCl₃) δ 0.76 (t, J=6.6 Hz, 6H), 0.97-1.17 (m, 8H), 1.06 (t, J=7.2 Hz, 6H), 1.43 (m, 4H), 2.63 (dd, J=14.3, 7.0 Hz, 4H), 2.78 (s, 6H), 2.85 (t, J=6.0 Hz, 2H), 3.15 (s, 2H), 3.70 (s, 2H), 4.02 (t, J=6.2 Hz, 2H), 5.78 (d, J=2.4 Hz, 1H), 6.31 (dd, J=9.1, 2.4 Hz, 1H), 6.84 (d, J=8.9 Hz, 2H), 7.09 (d, J=8.9 Hz, 2H), 7.78 (d, J=9.1 Hz, 1H). Reverse-phase HPLC: rt=5.4 min, 99% pure. LC/MS (ES/M+H): 544. Anal Calcd for (C₃₁H₄₉N₃O₃S): C, 68.47; H, 9.08; N, 7.73; S, 5.90; O, 8.83. Found: C, 67.99; H, 8.92; N, 7.79; S, 5.86; O, 9.19.

### EXAMPLE 7

### N-[2-[4-[3,3-Dibutyl-7-(dimethylamino)-2,3,4,5-tetrahydro-1,1-dioxido-1,5-benzothiazepin-5-yl]phenoxy]ethyl]-N,N,N-triethylaminium iodide

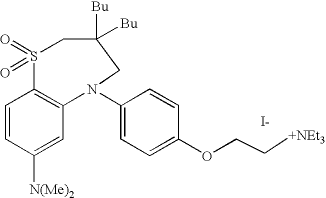

N-[2-[4-[3,3-Dibutyl-7-(dimethylamino)-2,3,4,5-tetrahydro-1,1-dioxido-1,5-benzothiazepin-5-yl]phenoxy]ethyl]-N,N-diethylamine (0.29 g, 0.533 mmol) was reacted with 20 eq. EtI, according to the procedure described for PHA-427823E above, to give the desired quat salt (0.200 g/54%). ¹H NMR (CDCl₃) d 0.77 (t, J=6.6 Hz, 6H), 1.04-1.21 (m, 8H), 1.36-1.45 (m, 4H), 1.46 (t, J=7.0 Hz, 9H), 2.84 (s, 6H), 3.10 (s, 2H), 3.57 (q, J=7.4 Hz, 6H), 3.61 (s, 2H), 4.04 (m, 2H), 4.48 (m, 2H), 5.95 (s, 1H), 6.40 (dd, J=9.1, 2.2 Hz, 1H), 6.86 (d, J=8.9 Hz, 2H), 7.07 (d, J=8.9 Hz, 2H), 7.80 (d, J=9.1 Hz, 1H). Reverse-phase HPLC: rt=16.9 min, 95% pure. LC/MS (ES/M−HI): 571.9. Anal Calcd for (C₃₃H₅₄N₃O₃SI): C, 56.64; H, 7.78; N, 6.00; S, 4.58; O, 6.86; I, 18.13. Found: C, 54.94; H, 7.71; N, 5.76; S, 4.58; O, 8.21; I, 18.44.

Various Compounds Made

In accordance with the above-described procedures, the following compounds can be/were made:

1,4 benzathiazapines

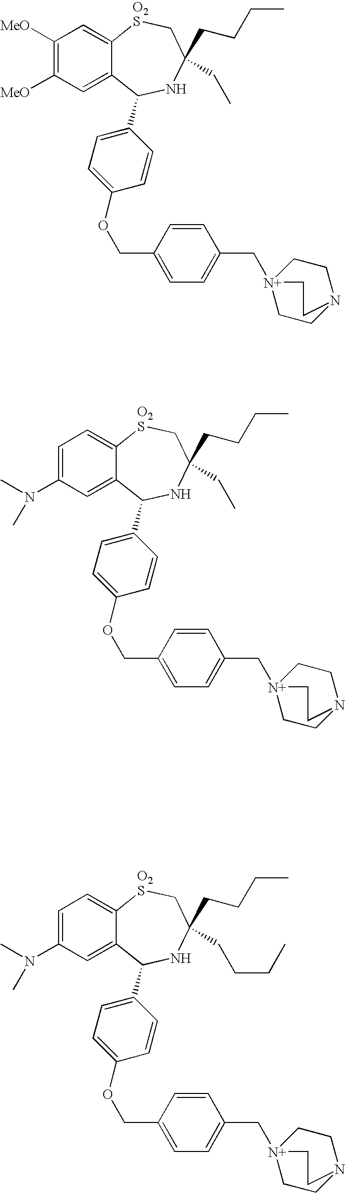

Further, utilizing the biological evaluation procedures outlined below, the IC₅₀ (50% inhibitory concentrations) values indicated in Table 7 below were determined.

TABLE 7
1,5-benzothiazepines

| Name | | IC₅₀ (uM) |
|---|---|---|
| SC-86288 | 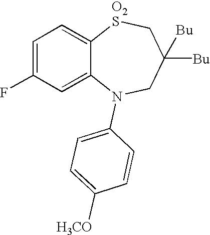 | 0.320 |
| SC-86287 | 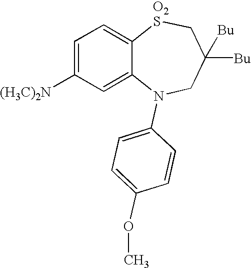 | 0.289 SC-86436 free phenol 0.21 |
| PHA00400670 | 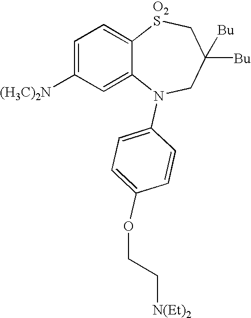 | 26.4 |
| PHA00400884E | 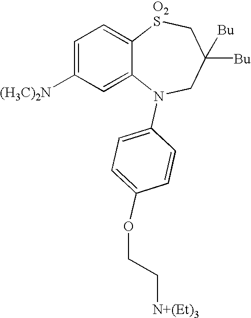 | >1 |
| PHA00400885E | 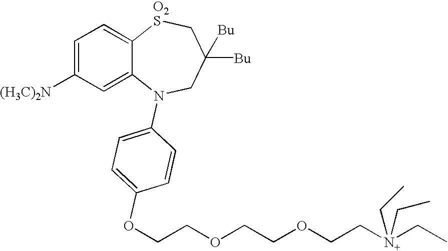 | 0.30 |
| PHA00384640E | 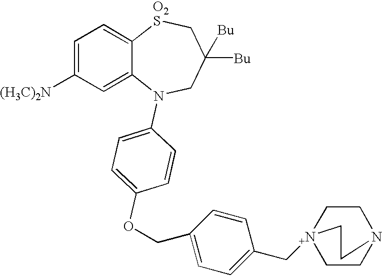 | 0.031 |

Utilizing the above-noted procedures, the following compounds were prepared:

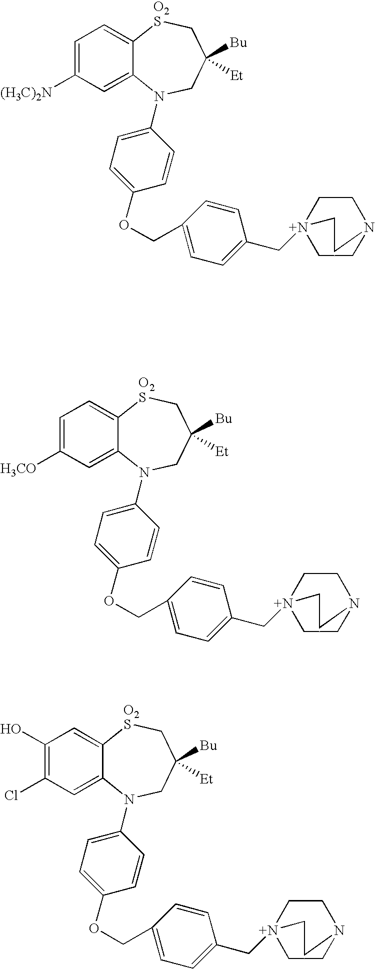

Furthermore, the additional IC₅₀ values were determined as noted in Table 8 below.

TABLE 8
1, 4 and 1, 5 benzothiazipines

| | | IC₅₀ | |
|---|---|---|---|
| compound # | Structure | (nanomolar) | |
| 1 chiral R, R | 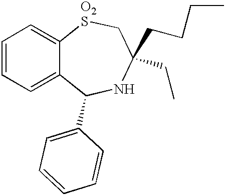 | 600 | nM |
| 2 racemic | 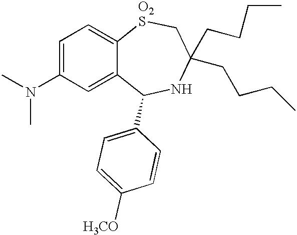 | 497 | nM |
| 3 racemic | 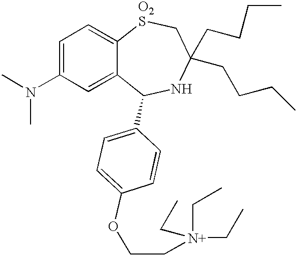 | 2092 | nM |
| 4 racemic | 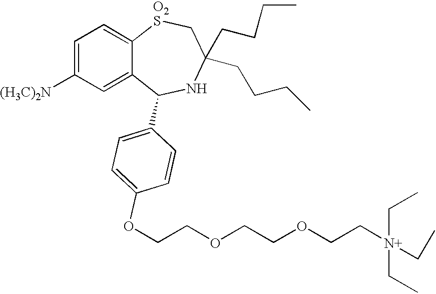 | 74 | nM |
| 5 racemic | 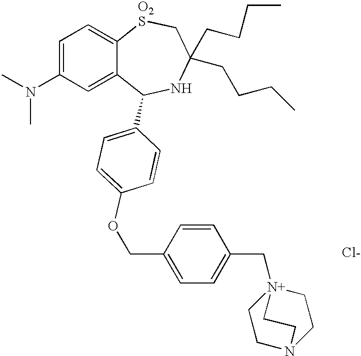 | 22 | nM |
| 6 | 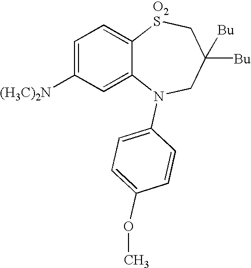 | 289 | nM |
| 7 | 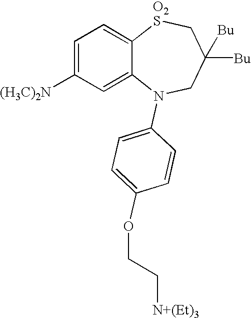 | 15084 | nM |
| 8 | 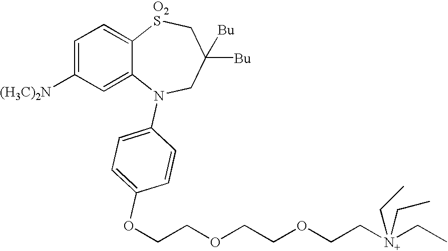 | 300 | nM |
| 9 | 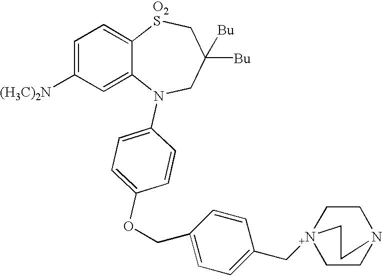 | 30 | nM |

BIOLOGICAL EVALUATION

The utility of the compounds of the present invention is shown by the following assays. These assays are performed in vitro and in animal models essentially using a procedure recognized to show the utility of the present invention.

In Vitro Assay of Compounds that Inhibit IBAT-Mediated Uptake of [¹⁴C]-Taurocholate (TC) in H₁₄Cells

Baby hamster kidney cells (BHK) transfected with the cDNA of human IBAT (H14 cells) are seeded in 96 well Top-Count tissue culture plates at 60,000 cells/well for assays run within 24 hours of seeding, 30,000 cells/well for assays run within 48 hours, and 10,000 cells/well for assays run within 72 hours.

On the day of assay, the cell monolayer is gently washed once with 100 mL assay buffer (Dulbecco's Modified Eagle's medium with 4.5 g/L glucose plus 0.2% (w/v) fatty acid free bovine serum albumin ((FAF)BSA). To each well 50 mL of a two-fold concentrate of test compound in assay buffer is added along with 50 mL of 6 mM [¹⁴C]-taurocholate in assay buffer (final concentration of 3 mM [¹⁴C]-taurocholate). The cell culture plates are incubated 2 hours at 37° C. prior to gently washing each well twice with 100 mL 4° C. Dulbecco's phosphate-buffered saline (PBS) containing 0.2% (w/v) (FAF)BSA. The wells are then gently washed once with 100 mL 4° C. PBS without (FAF)BSA. To each 200 mL of liquid, scintillation counting fluid is added. The plates are heat sealed and shaken for 30 minutes at room temperature prior to measuring the amount of radioactivity in each well on a Packard Top-Count instrument.

In Vitro Assay of Compounds that Inhibit Uptake of [¹⁴C]-Alanine

The alanine uptake assay is performed in an identical fashion to the taurocholate assay, with the exception that labeled alanine is substituted for the labeled taurocholate.

In Vivo Assay of Compounds that Inhibit Rat Ileal Uptake of [¹⁴C]-Taurocholate into Bile

(See Une et al. “Metabolism of 3a,7β-dihydroxy-7β-methyl-5β-cholanoic acid and 3a, 7β-dihydroxy-7a-methyl-5β-cholanoic acid in hamsters”, Biochimica et Biophysica Acta, Vol. 833, pp. 196-202 (1985)).

Male wistar rats (200-300 g) are anesthetized with inactin @100 mg/kg. Bile ducts are cannulated with a 10″ length of PE10 tubing. The small intestine is exposed and laid out on a gauze pad. A canulae (⅛″ luer lock, tapered female adapter) is inserted at 12 cm from the junction of the small intestine and the cecum. A slit is cut at 4 cm from this same junction (utilizing a 8 cm length of ileum). 20 mL of warm Dulbecco's phosphate buffered saline, pH 6.5 (“PBS”) is used to flush out the intestine segment. The distal opening is cannulated with a 20 cm length of silicone tubing (0.02″ I.D.×0.037″ O.D.). The proximal cannulae is hooked up to a peristaltic pump and the intestine is washed for 20 minutes with warm PBS at 0.25 ml/minute. Temperature of the gut segment is monitored continuously.

At the start of the experiment, 2.0 mL of control sample ([¹⁴C]-taurocholate @ 0.05 ml/mL with 5 mM cold taurocholate) is loaded into the gut segment with a 3 mL syringe and bile sample collection is begun. Control sample is infused at a rate of 0.25 ml/minute for 21 minutes. Bile samples fractions are collected every 3 minutes for the first 27 minutes of the procedure. After the 21 minutes of sample infusion, the ileal loop is washed out with 20 mL of warm PBS (using a 30 mL syringe), and then the loop is washed out for 21 minutes with warm PBS at 0.25 ml/minutes. A second perfusion is initiated as described above but with test compound being administered as well (21 minutes administration followed by 21 minutes of wash out) and bile sampled every 3 minutes for the first 27 minutes. If necessary, a third perfusion is performed as above that typically contains the control sample.

Measurement of Hepatic Cholesterol Concentration (HEPATIC CHOL)

Liver tissue is weighed and homogenized in chloroform:methanol (2:1). After homogenization and centrifugation the supernatant is separated and dried under nitrogen. The residue is dissolved in isopropanol and the cholesterol content is measured enzymatically, using a combination of cholesterol oxidase and peroxidase, as described by Allain, C. A., et al. (1974) Clin. Chem. 20, 470.

Measurement of Hepatic HMG CoA-Reductase Activity (HMG COA)

Hepatic microsomes are prepared by homogenizing liver samples in a phosphate/sucrose buffer, followed by centrifugal separation. The final pelleted material is resuspended in buffer and an aliquot is assayed for HMG CoA reductase activity by incubating for 60 minutes at 37° C. in the presence of ¹⁴C-HMG-CoA (Dupont-NEN). The reaction is stopped by adding 6N HCl followed by centrifugation. An aliquot of the supernatant is separated, by thin-layer chromatography, and the spot corresponding to the enzyme product is scraped off the plate, extracted and radioactivity is determined by scintillation counting. (Reference: Akerlund, J. and Bjorkhem, I. (1990) J. Lipid Res. 31, 2159).

Determination of Serum Cholesterol (SER.CHOL. HDL-CHOL. TGI and VLDL+LDL)

Total serum cholesterol (SER.CHOL) is measured enzymatically using a commercial kit from Wako Fine Chemicals (Richmond, Va.); Cholesterol C11, Catalog No. 276-64909. HDL cholesterol (HDL-CHOL) is assayed using this same kit after precipitation of VLDL and LDL with Sigma Chemical Co. HDL Cholesterol reagent, Catalog No. 352-3 (dextran sulfate method). Total serum triglycerides (blanked) (TGI) are assayed enzymatically with Sigma Chemical Co. GPO-Trinder, Catalog No. 337-B. VLDL and LDL (VLDL+LDL) cholesterol concentrations are calculated as the difference between total and HDL cholesterol.

Measurement of Hepatic Cholesterol 7a-Hydroxylase Activity (7a-OHase)

Hepatic microsomes are prepared by homogenizing liver samples in a phosphate/sucrose buffer, followed by centrifugal separation. The final pelleted material is resuspended in buffer and an aliquot is assayed for cholesterol 7a-hydroxylase activity by incubating for 5 minutes at 37° C. in the presence of NADPH. Following extraction into petroleum ether, the organic solvent is evaporated and the residue is dissolved in acetonitrile/methanol. The enzymatic product is separated by injecting an aliquot of the extract onto a C₁₈ reversed phase HPLC column and quantitating the eluted material using UV detection at 240 nm. (Reference: Horton, J. D., et al. (1994) J. Clin. Invest. 93, 2084).

Rat Gavage Assay

Male Wister rats (275-300 g) are administered IBAT inhibitors using an oral gavage procedure. Drug or vehicle (0.2% Tween 80 in water) is administered once a day (9:00-10:00 a.m.) for 4 days at varying dosages in a final volume of 2 mL per kilogram of body weight. Total fecal samples are collected during the final 48 hours of the treatment period and analyzed for bile acid content using an enzymatic assay as described below. Compound efficacy is determined by comparison of the increase in fecal bile acid (FBA) concentration in treated rats to the mean FBA concentration of rats in the vehicle group.

Measurement of Fecal Bile Acid Concentration (FBA)

Total fecal output from individually housed hamsters is collected for 24 or 48 hours, dried under a stream of nitrogen, pulverized and weighed. Approximately 0.1 gram is weighed out and extracted into an organic solvent (butanol/water). Following separation and drying, the residue is dissolved in methanol and the amount of bile acid present is measured enzymatically using the 3a-hydroxysteroid steroid dehydrogenase reaction with bile acids to reduce NAD. (Reference: Mashige, F., et al. (1981) Clin. Chem. 27, 1352).

[³H]taurocholate Uptake in Rabbit Brush Border Membrane Vesicles (BBMV)

Rabbit Ileal brush border membranes are prepared from frozen ileal mucosa by the calcium precipitation method describe by Malathi et al. (Reference: (1979) Biochimica Biophysica Acta, 554, 259). The method for measuring taurocholate is essentially as described by Kramer et al. (Reference: (1992) Biochimica Biophysica Acta, 1111, 93) except the assay volume is 200 μL instead of 100 μL. Briefly, at room temperature a 190 μL solution containing 2 μM [³H]-taurocholate (0.75 μCi), 20 mM tris, 100 mM sodium chloride, 100 mM mannitol pH 7.4 is incubated for 5 seconds with 10 μL of brush border membrane vesicles (60-120 μg protein). The incubation is initiated by the addition of the BBMV while vortexing and the reaction is stopped by the addition of 5 mL of ice cold buffer (20 mM Hepes-tris, 150 mM KCl) followed immediately by filtration through a nylon filter (0.2 μm pore) and an additional 5 mL wash with stop buffer.

Acyl-CoA: Cholesterol Acyl Transferase (ACAT)

Hamster liver and rat intestinal microsomes are prepared from tissue as described previously (Reference: (1980) J. Biol. Chem. 255, 9098) and used as a source of ACAT enzyme. The assay consists of a 2.0 mL incubation containing 24 μM Oleoyl-CoA (0.05 μCi) in a 50 mM sodium phosphate, 2 mM DTT pH 7.4 buffer containing 0.25% BSA and 200 μg of microsomal protein. The assay is initiated by the addition of oleoyl-CoA. The reaction is allowed to proceed for 5 minutes at 37° C. and is terminated by the addition of 8.0 mL of chloroform/methanol (2:1). To the extraction is added 125 μg of cholesterol oleate in chloroform methanol to act as a carrier and the organic and aqueous phases of the extraction are separated by centrifugation after thorough vortexing. The chloroform phase is taken to dryness and then spotted on a silica gel 60 thin layer chromatography plate and developed in hexane/ethyl ether (9:1). The amount of cholesterol ester formed is determined by measuring the amount of radioactivity incorporated into the cholesterol oleate spot on the thin layer chromatography plate with a Packard instaimager.

Various additional compounds noted in Tables 9-10 below can be/were made according to the procedures outlined above.

TABLE 9

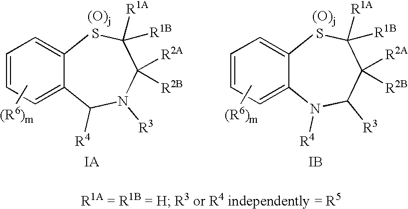

| Compound | R^{2A} | R^{2B} | R⁵ |
|---|---|---|---|
| 101 | ethyl | n-butyl | phenyl |
| 102 | ethyl | n-butyl | phenyl |
| 103 | n-butyl | Ethyl | phenyl |
| 104 | ethyl | n-butyl | phenyl |
| 105 | ethyl | n-butyl | phenyl |
| 106 | ethyl | n-butyl | phenyl |
| 107 | n-butyl | Ethyl | 4-(decyloxy)phenyl |
| 108 | ethyl | n-butyl | phenyl |
| 109 | ethyl | n-butyl | 4-(decyloxy)phenyl |
| 110 | ethyl | n-butyl | phenyl |
| 111 | n-butyl | Ethyl | 4-hydroxyphenyl |
| 112 | ethyl | n-butyl | 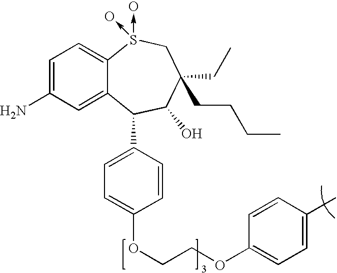 |
| 113 | ethyl | n-butyl | 4-hydroxyphenyl |
| 114 | ethyl | n-butyl | 4-methoxyphenyl |
| 115 | n-butyl | ethyl | 4-methoxyphenyl |
| 116 | ethyl | n-butyl | 4-methoxyphenyl |
| 117 | n-butyl | ethyl | phenyl |
| 118 | ethyl | n-butyl | phenyl |
| 119 | ethyl | n-butyl | phenyl |
| 120 | n-butyl | ethyl | phenyl |
| 121 | ethyl | n-butyl | phenyl |
| 122 | n-butyl | ethyl | phenyl |
| 123 | ethyl | n-butyl | phenyl |
| 124 | n-butyl | ethyl | phenyl |
| 125 | ethyl | n-butyl | phenyl |
| 126 | n-butyl | ethyl | 4-fluorophenyl |
| 127 | n-butyl | ethyl | 4-fluorophenyl |
| 128 | Ethyl | n-butyl | 4-fluorophenyl |
| 129 | Ethyl | n-butyl | 4-fluorophenyl |
| 131 | Ethyl | n-butyl | 4-fluorophenyl |
| 132 | Ethyl | n-butyl | phenyl |
| 133 | Ethyl | n-butyl | phenyl |
| 134 | Ethyl | n-butyl | phenyl |
| 135 | Ethyl | n-butyl | phenyl |
| 136 | Ethyl | n-butyl | phenyl |
| 137 | n-butyl | ethyl | phenyl |
| 138 | n-butyl | ethyl | phenyl |
| 139 | n-butyl | ethyl | phenyl |
| 140 | | | |
| 141 | | | |
| 142 | Ethyl | n-butyl | H |
| 143 | Ethyl | n-butyl | 3-methoxyphenyl |
| 144 | Ethyl | n-butyl | 4-fluorophenyl |
| 262 | Ethyl | n-butyl | 3-methoxyphenyl |
| 263 | Ethyl | n-butyl | H |
| 264 | Ethyl | n-butyl | 3-trifluoromethylphenyl |
| 265 | Ethyl | n-butyl | H |
| 266 | Ethyl | n-butyl | 3-hydroxyphenyl |
| 267 | Ethyl | n-butyl | 3-hydroxyphenyl |
| 268 | Ethyl | n-butyl | 4-fluorophenyl |
| 269 | Ethyl | n-butyl | H |
| 270 | Ethyl | n-butyl | 4-fluorophenyl |
| 271 | Ethyl | n-butyl | 3-methoxyphenyl |
| 272 | Ethyl | n-butyl | H |
| 273 | Ethyl | n-butyl | H |
| 274 | Ethyl | n-butyl | 4-fluorophenyl |
| 275 | Ethyl | n-butyl | H |
| 276 | Ethyl | n-butyl | 3-methoxyphenyl |
| 277 | Ethyl | n-butyl | 3-fluorophenyl |
| 278 | Ethyl | n-butyl | 2-fluorophenyl |
| 279 | Ethyl | n-butyl | 3-fluorophenyl |
| 280 | Ethyl | n-butyl | 2-fluorophenyl |
| 281 | Ethyl | n-butyl | 4-fluorophenyl |
| 282 | Ethyl | n-butyl | 4-fluorophenyl |
| 283 | Ethyl | n-butyl | H |
| 284 | Ethyl | n-butyl | 4-fluorophenyl |
| 286 | Ethyl | ethyl | phenyl |
| 287 | Ethyl | ethyl | phenyl |
| 288 | methyl | methyl | phenyl |
| 289 | n-butyl | n-butyl | phenyl |
| 290 | n-butyl | n-butyl | phenyl |
| 291 | n-butyl | n-butyl | phenyl |
| 292 | n-butyl | n-butyl | 4-fluorophenyl |
| 293 | n-butyl | n-butyl | phenyl |
| 294 | n-butyl | n-butyl | phenyl |
| 295 | Ethyl | n-butyl | 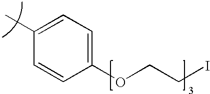 |
| 296 | Ethyl | n-butyl | 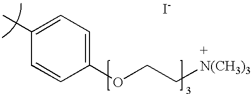 |
| 1000 | Ethyl | n-butyl | 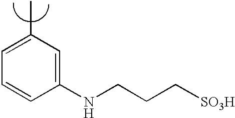 |
| 1001 | Ethyl | n-butyl | 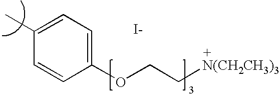 |
| 1002 | Ethyl | n-butyl | 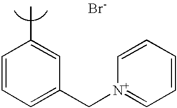 |
| 1003 | Ethyl | n-butyl | 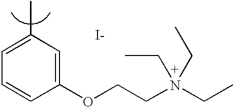 |
| 1004 | Ethyl | n-butyl | 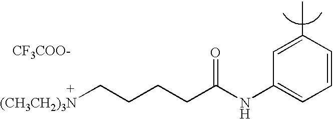 |
| 1005 | n-butyl | n-butyl | 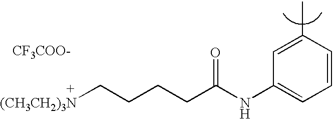 |
| 1006 | n-butyl | n-butyl | 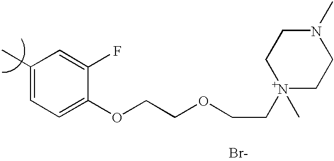 |
| 1007 | n-butyl | n-butyl | 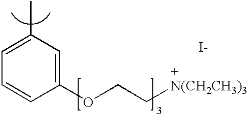 |
| 1008 | n-butyl | n-butyl | 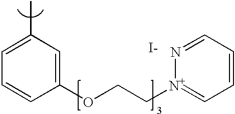 |
| 1009 | n-butyl | n-butyl | 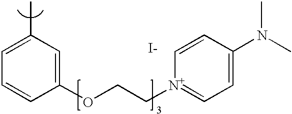 |
| 1010 | n-butyl | n-butyl | 3-fluoro-4-methoxyphenyl |
| 1011 | n-butyl | n-butyl | 3-fluoro-4-(5-triethylammoniumpenyloxy)phenyl, trifluoroacetate salt |
| 1012 | n-butyl | n-butyl | 4-hydroxyphenyl |
| 1013 | n-butyl | n-butyl | 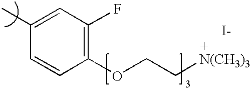 |
| 1014 | n-butyl | n-butyl | 4-methoxyphenyl |
| 1015 | n-butyl | n-butyl | 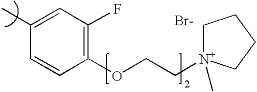 |
| 1016 | n-butyl | n-butyl | 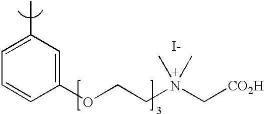 |
| 1017 | n-butyl | n-butyl | 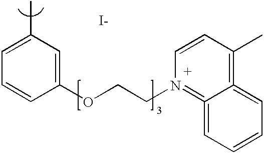 |
| 1018 | n-butyl | n-butyl | 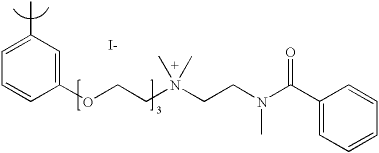 |
| 1019 | n-butyl | n-butyl | 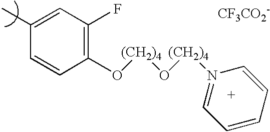 |
| 1020 | n-butyl | n-butyl | 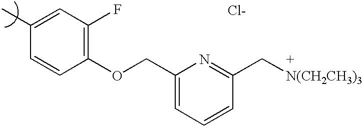 |
| 1021 | n-butyl | n-butyl | 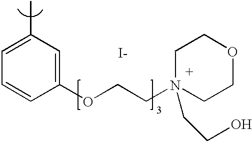 |
| 1022 | n-butyl | n-butyl | 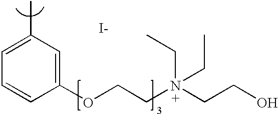 |
| 1023 | n-butyl | n-butyl | 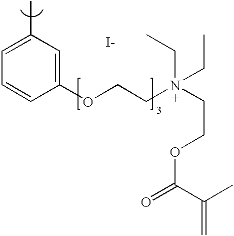 |
| 1024 | n-butyl | n-butyl | 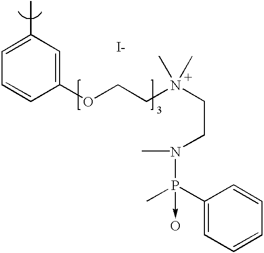 |
| 1025 | n-butyl | n-butyl | 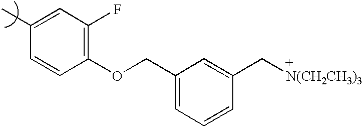 |
| 1026 | n-butyl | n-butyl | 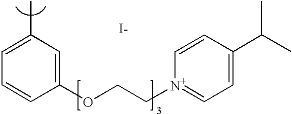 |
| 1027 | n-butyl | n-butyl | 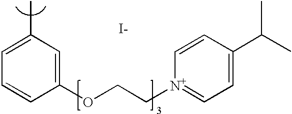 |
| 1028 | n-butyl | n-butyl | 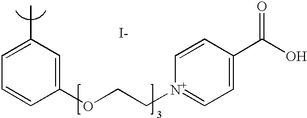 |
| 1029 | n-butyl | n-butyl | 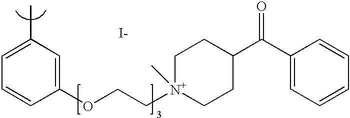 |
| 1030 | n-butyl | n-butyl | 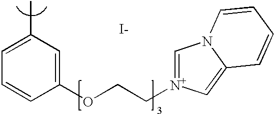 |
| 1031 | n-butyl | n-butyl | 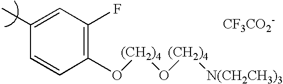 |
| 1032 | n-butyl | n-butyl | 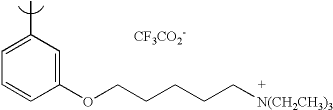 |
| 1033 | n-butyl | n-butyl | 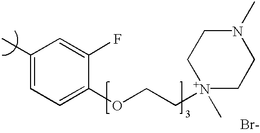 |
| 1034 | n-butyl | n-butyl | 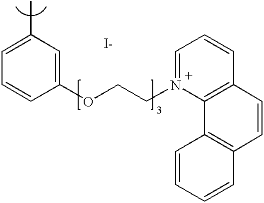 |
| 1035 | n-butyl | n-butyl | 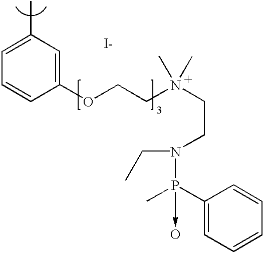 |
| 1036 | n-butyl | n-butyl | 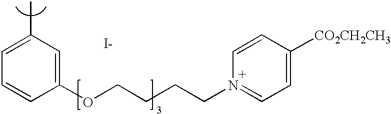 |
| 1037 | n-butyl | n-butyl | 4-hydroxyphenyl |
| 1038 | n-butyl | n-butyl | 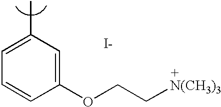 |
| 1039 | n-butyl | n-butyl | phenyl |
| 1040 | n-butyl | n-butyl | 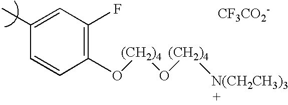 |
| 1041 | n-butyl | n-butyl | 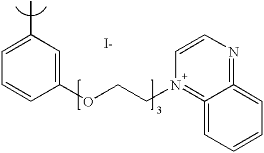 |
| 1042 | n-butyl | n-butyl | 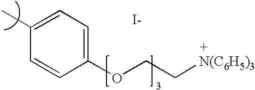 |
| 1043 | n-butyl | n-butyl | 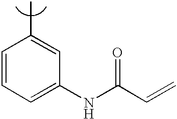 |
| 1044 | n-butyl | n-butyl | 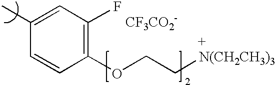 |
| 1045 | n-butyl | n-butyl | 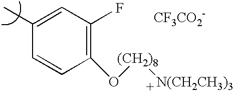 |
| 1046 | n-butyl | n-butyl | 3-aminophenyl |
| 1047 | n-butyl | n-butyl | 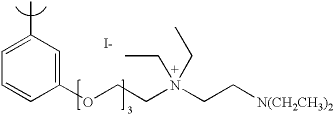 |
| 1048 | n-butyl | n-butyl | 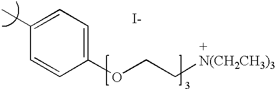 |
| 1049 | n-butyl | n-butyl | 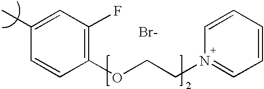 |
| 1050 | n-butyl | n-butyl | 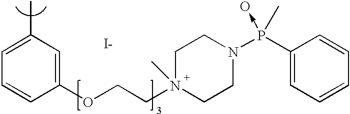 |
| 1051 | n-butyl | n-butyl | 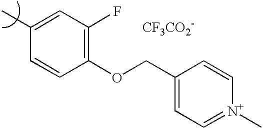 |
| 1052 | n-butyl | n-butyl | 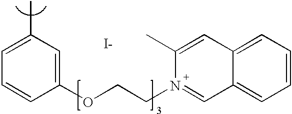 |
| 1053 | n-butyl | n-butyl | 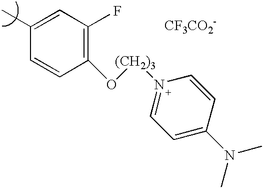 |
| 1054 | n-butyl | n-butyl | 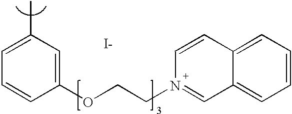 |
| 1055 | n-butyl | n-butyl | 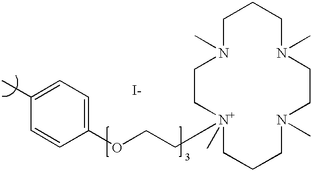 |
| 1056 | n-butyl | n-butyl | 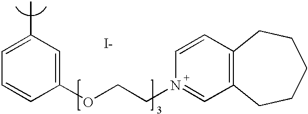 |
| 1057 | n-butyl | n-butyl | 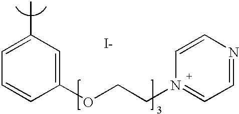 |
| 1058 | n-butyl | n-butyl | 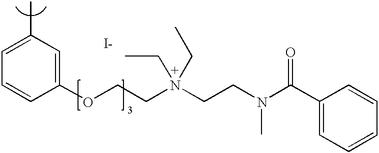 |
| 1059 | n-butyl | n-butyl | 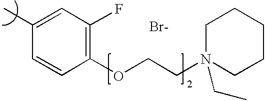 |
| 1060 | Ethyl | n-butyl | 3-fluoro-4-methoxyphenyl |
| 1061 | n-butyl | n-butyl | 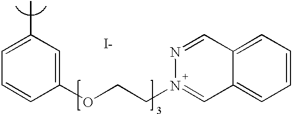 |
| 1062 | n-butyl | n-butyl | 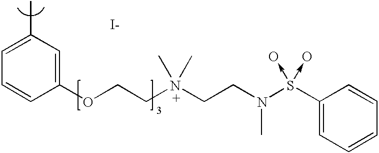 |
| 1063 | n-butyl | n-butyl | 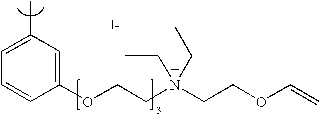 |
| 1064 | n-butyl | n-butyl | 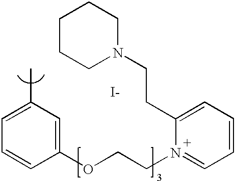 |
| 1065 | n-butyl | n-butyl | 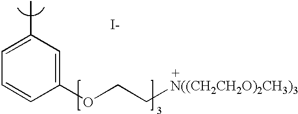 |
| 1066 | n-butyl | n-butyl | 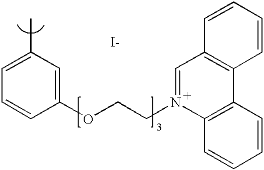 |
| 1067 | n-butyl | n-butyl | thiophen-3-yl |
| 1068 | n-butyl | n-butyl | 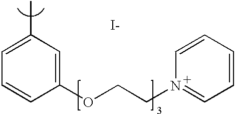 |
| 1069 | n-butyl | n-butyl | phenyl |
| 1070 | n-butyl | n-butyl | 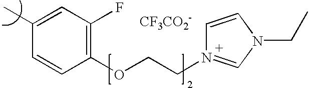 |
| 1071 | n-butyl | n-butyl | 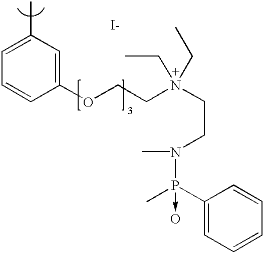 |
| 1072 | n-butyl | n-butyl | 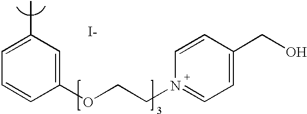 |
| 1073 | n-butyl | n-butyl | 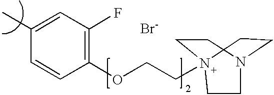 |
| 1074 | Ethyl | n-butyl | 3-fluoro-4-methoxyphenyl |
| 1075 | n-butyl | n-butyl | 4-fluorophenyl |
| 1076 | n-butyl | n-butyl | 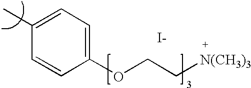 |
| 1077 | n-butyl | n-butyl | 3-hydroxymethylphenyl |
| 1078 | Ethyl | n-butyl | 4-hydroxyphenyl |
| 1079 | Ethyl | n-butyl | 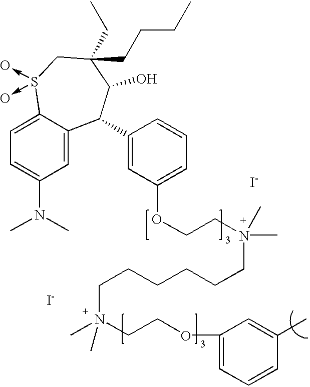 |
| 1080 | n-butyl | n-butyl | 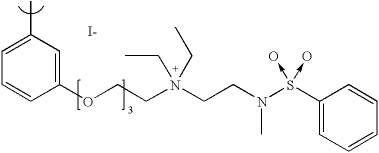 |
| 1081 | n-butyl | n-butyl | 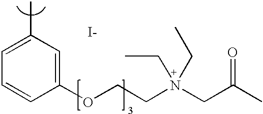 |
| 1082 | n-butyl | n-butyl | 2-pyridyl |
| 1083 | n-butyl | n-butyl | 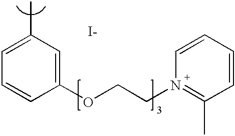 |
| 1084 | n-butyl | n-butyl | 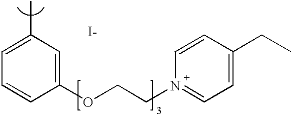 |
| 1085 | n-butyl | n-butyl | thiophen-3-yl |
| 1086 | n-butyl | n-butyl | 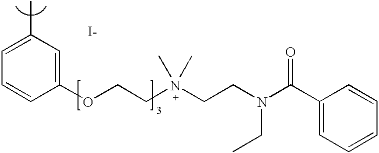 |
| 1087 | n-butyl | n-butyl | 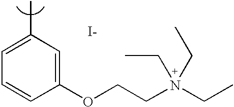 |
| 1088 | Ethyl | n-butyl | 3,4-methylenedioxyphenyl |
| 1089 | Ethyl | n-butyl | 4-methoxyphenyl |
| 1090 | n-butyl | n-butyl | 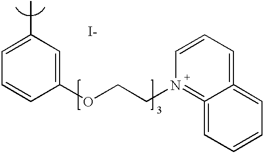 |
| 1091 | n-butyl | n-butyl | 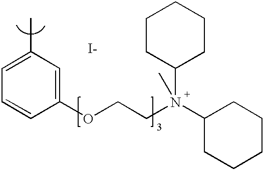 |
| 1092 | n-butyl | n-butyl | 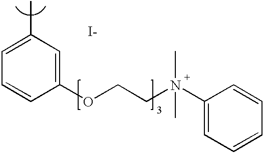 |
| 1093 | n-butyl | n-butyl | 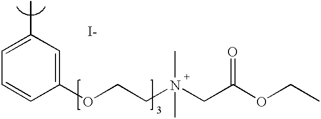 |
| 1094 | n-butyl | n-butyl | 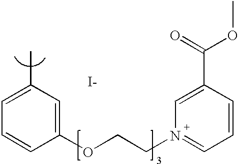 |
| 1095 | n-butyl | n-butyl | 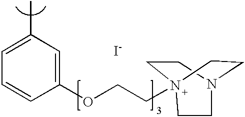 |
| 1096 | n-butyl | n-butyl | 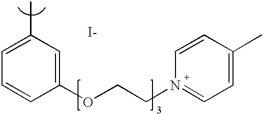 |
| 1097 | n-butyl | n-butyl | 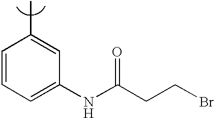 |
| 1098 | n-butyl | n-butyl | 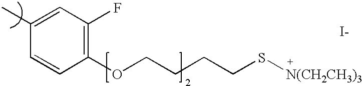 |
| 1099 | Ethyl | n-butyl | 4-methoxyphenyl |
| 1100 | n-butyl | n-butyl | 4-methoxyphenyl |
| 1101 | n-butyl | n-butyl | 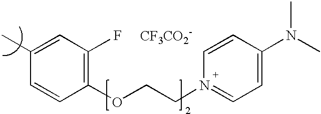 |
| 1102 | n-butyl | n-butyl | 3-carboxymethylphenyl |
| 1103 | n-butyl | n-butyl | 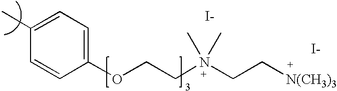 |
| 1104 | n-butyl | n-butyl | 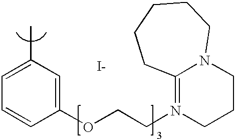 |
| 1105 | n-butyl | n-butyl | 5-piperonyl |
| 1106 | n-butyl | n-butyl | 3-hydroxyphenyl |
| 1107 | n-butyl | n-butyl | 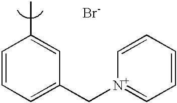 |
| 1108 | n-butyl | n-butyl | 3-pyridyl |
| 1109 | n-butyl | n-butyl | 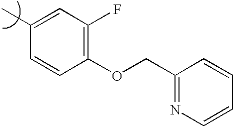 |
| 1110 | n-butyl | n-butyl | 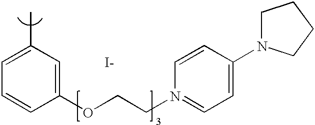 |
| 1111 | n-butyl | n-butyl | 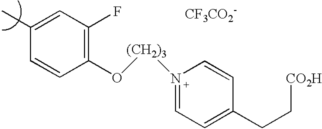 |
| 1112 | n-butyl | n-butyl | 4-pyridyl |
| 1113 | n-butyl | n-butyl | 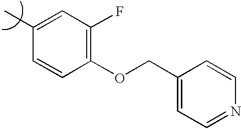 |
| 1114 | n-butyl | n-butyl | 3-methoxyphenyl |
| 1115 | n-butyl | n-butyl | 4-fluorophenyl |
| 1116 | Ethyl | n-butyl | 3-tolyl |
| 1117 | Ethyl | n-butyl | 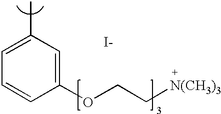 |
| 1118 | Ethyl | n-butyl | 3-fluoro-4-hydroxyphenyl |
| 1119 | n-butyl | n-butyl | 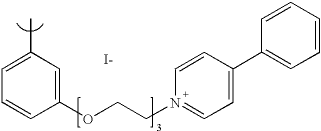 |
| 1120 | n-butyl | n-butyl | 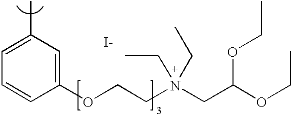 |
| 1121 | n-butyl | n-butyl | 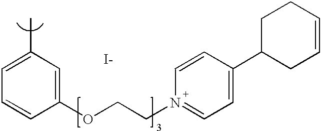 |
| 1122 | n-butyl | n-butyl | 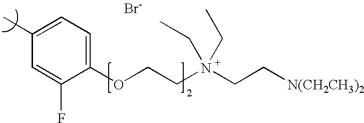 |
| 1123 | n-butyl | n-butyl | phenyl |
| 1124 | n-butyl | n-butyl | 3-methoxyphenyl |
| 1125 | n-butyl | n-butyl | 3-chloro-4-methoxyphenyl |
| 1126 | Ethyl | n-butyl | 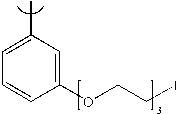 |
| 1127 | n-butyl | n-butyl | 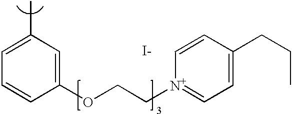 |
| 1128 | n-butyl | n-butyl | 3-fluoro-4-hydroxyphenyl |
| 1129 | n-butyl | n-butyl | 4-fluorophenyl |
| 1130 | n-butyl | n-butyl | 3-chloro-4-fluorophenyl |
| 1131 | Ethyl | n-butyl | 4-methoxyphenyl |
| 1132 | n-butyl | n-butyl | 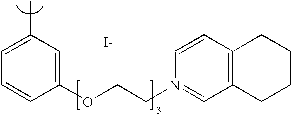 |
| 1133 | n-butyl | n-butyl | 4-cyanomethylphenyl |
| 1134 | Ethyl | n-butyl | 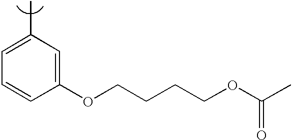 |
| 1135 | n-butyl | n-butyl | 3,4-dimethoxyphenyl |
| 1136 | n-butyl | n-butyl | 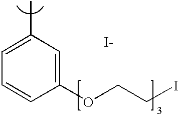 |
| 1137 | n-butyl | n-butyl | 4-fluorophenyl |
| 1138 | n-butyl | n-butyl | 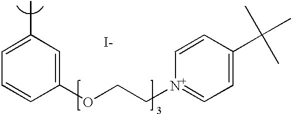 |
| 1139 | n-butyl | n-butyl | 3,4-difluorophenyl |
| 1140 | n-butyl | n-butyl | 3-methoxyphenyl |
| 1141 | n-butyl | n-butyl | 4-fluorophenyl |
| 1142 | n-butyl | n-butyl | 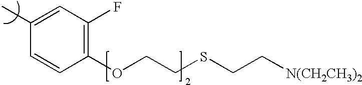 |
| 1143 | n-butyl | n-butyl | H |
| 1144 | n-butyl | n-butyl | 5-piperonyl |
| 1145 | n-butyl | n-butyl | 4-methoxyphenyl |
| 1146 | n-butyl | n-butyl | 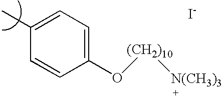 |
| 1147 | n-butyl | n-butyl | 3-methoxyphenyl |
| 1148 | n-butyl | n-butyl | 4-fluorophenyl |
| 1149 | n-butyl | n-butyl | 4-fluorophenyl |
| 1150 | n-butyl | n-butyl | 3-methoxyphenyl |
| 1151 | n-butyl | ethyl | 3-fluoro-4-methoxyphenyl |
| 1152 | n-butyl | n-butyl | phenyl |
| 1153 | n-butyl | n-butyl | 4-fluorophenyl |
| 1154 | n-butyl | n-butyl | 3-methoxyphenyl |
| 1155 | n-butyl | n-butyl | 4-fluorophenyl |
| 1156 | n-butyl | n-butyl | 4-fluorophenyl |
| 1157 | n-butyl | n-butyl | 4-fluorophenyl |
| 1158 | n-butyl | n-butyl | 4-pyridinyl, hydrochloride salt |
| 1159 | n-butyl | ethyl | phenyl |
| 1160 | n-butyl | n-butyl | 4-fluorophenyl |
| 1161 | n-butyl | n-butyl | 3,5-dichloro-4-methoxyphenyl |
| 1162 | n-butyl | n-butyl | phenyl |
| 1163 | n-butyl | n-butyl | 3-(dimethylamino)phenyl |
| 1164 | n-butyl | n-butyl | 4-pyridinyl |
| 1165 | n-butyl | n-butyl | 3-fluoro-4-methoxyphenyl |
| 1166 | n-butyl | n-butyl | 3-hydroxyphenyl |
| 1167 | n-butyl | n-butyl | 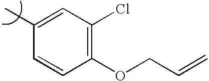 |
| 1168 | n-butyl | n-butyl | 4-hydroxyphenyl |
| 1169 | n-butyl | n-butyl | phenyl |
| 1170 | n-butyl | n-butyl | 3-methoxyphenyl |
| 1171 | n-butyl | n-butyl | 4-(trifluoromethylsulfonyloxy)phenyl |
| 1172 | n-butyl | n-butyl | 4-pyridinyl |
| 1173 | n-butyl | n-butyl | 4-fluorophenyl |
| 1174 | Ethyl | n-butyl | 3-methoxyphenyl |
| 1175 | Ethyl | n-butyl | 3-methoxyphenyl |
| 1176 | n-butyl | n-butyl | 4-fluorophenyl |
| 1177 | n-butyl | n-butyl | 3-methoxyphenyl |
| 1178 | n-butyl | n-butyl | 3-(trifluoromethylsulfonyloxy)phenyl |
| 1179 | n-butyl | n-butyl | phenyl |
| 1180 | n-butyl | n-butyl | phenyl |
| 1181 | n-butyl | n-butyl | 4-fluorophenyl |
| 1182 | n-butyl | n-butyl | 4-(dimethylamino)phenyl |
| 1183 | n-butyl | n-butyl | 3-methoxyphenyl |
| 1184 | n-butyl | n-butyl | 4-fluorophenyl |
| 1185 | n-butyl | n-butyl | 4-fluorophenyl |
| 1186 | n-butyl | n-butyl | phenyl |
| 1187 | n-butyl | n-butyl | 4-fluorophenyl |
| 1188 | n-butyl | n-butyl | 4-methoxyphenyl |
| 1189 | n-butyl | n-butyl | 3,4-difluoropbenyl |
| 1190 | n-butyl | n-butyl | 2-bromophenyl |
| 1191 | n-butyl | n-butyl | 4-(dimethylamino)phenyl |
| 1192 | n-butyl | n-butyl | 3-(dimethylamino)phenyl |
| 1193 | n-butyl | n-butyl | 4-(2-(2-methylpropyl))phenyl |
| 1194 | n-butyl | n-butyl | 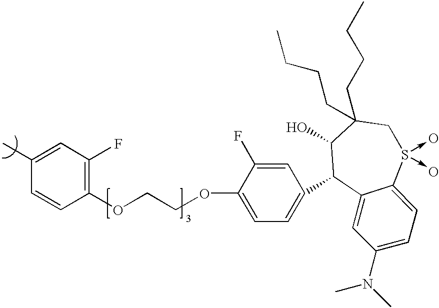 |
| 1195 | n-butyl | n-butyl | 4-methoxyphenyl |
| 1196 | n-butyl | n-butyl | 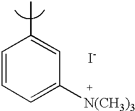 |
| 1197 | n-butyl | ethyl | phenyl |
| 1198 | n-butyl | n-butyl | 4-(pyridinyl-N-oxide) |
| 1199 | n-butyl | n-butyl | 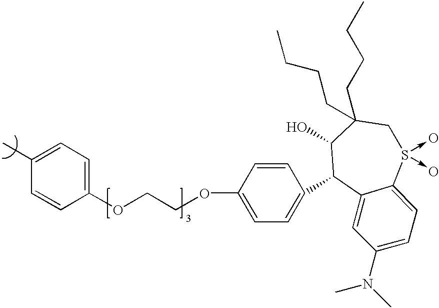 |
| 1200 | n-butyl | n-butyl | H |
| 1201 | n-butyl | n-butyl | H |
| 1202 | n-butyl | n-butyl | 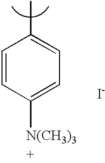 |
| 1203 | n-butyl | n-butyl | 5-piperazinyl |
| 1204 | n-butyl | n-butyl | 4-fluorophenyl |
| 1205 | n-butyl | n-butyl | 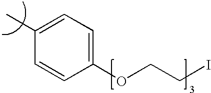 |
| 1206 | n-butyl | n-butyl | 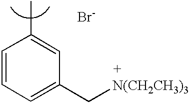 |
| 1207 | n-butyl | n-butyl | 3,5-dichlorophenyl |
| 1208 | n-butyl | n-butyl | 4-methoxyphenyl |
| 1209 | n-butyl | n-butyl | phenyl |
| 1210 | n-butyl | n-butyl | 2-(dimethylamino)phenyl |
| 1211 | Ethyl | n-butyl | 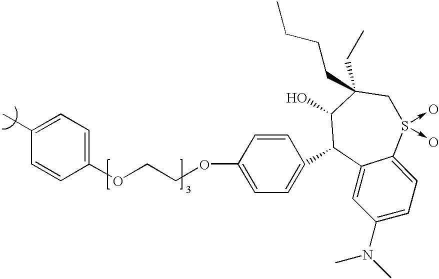 |
| 1212 | n-butyl | n-butyl | 4-methoxyphenyl |
| 1213 | n-butyl | ethyl | H |
| 1214 | n-butyl | ethyl | phenyl |
| 1215 | n-butyl | n-butyl | 4-methoxyphenyl |
| 1216 | Ethyl | n-butyl | 5-piperonyl |
| 1217 | n-butyl | n-butyl | 4-carboxyphenyl |
| 1218 | n-butyl | n-butyl | 4-methoxyphenyl |
| 1219 | n-butyl | n-butyl | 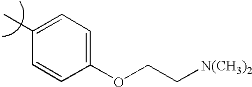 |
| 1220 | n-butyl | n-butyl | 3-methoxyphenyl |
| 1221 | n-butyl | n-butyl | 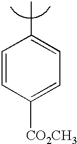 |
| 1222 | n-butyl | n-butyl | 3-methoxyphenyl |
| 1223 | n-butyl | n-butyl | phenyl |
| 1224 | n-butyl | n-butyl | 3-nitrophenyl |
| 1225 | n-butyl | ethyl | 3-methylphenyl |
| 1226 | Ethyl | n-butyl | 5-piperonyl |
| 1227 | n-butyl | n-butyl | 4-fluorophenyl |
| 1228 | n-butyl | n-butyl | 2-pyrrolyl |
| 1229 | n-butyl | n-butyl | 3-chloro-4-hydroxyphenyl |
| 1230 | n-butyl | n-butyl | phenyl |
| 1231 | n-butyl | n-butyl | 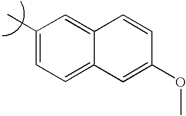 |
| 1232 | n-butyl | n-butyl | 3-thiophenyl |
| 1233 | n-butyl | n-butyl | 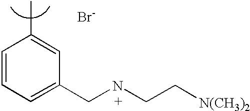 |
| 1234 | n-butyl | n-butyl | 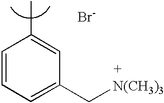 |
| 1235 | n-butyl | n-butyl | 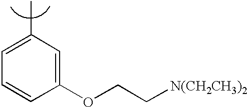 |
| 1236 | n-butyl | n-butyl | 4-(bromomethyl)phenyl |
| 1237 | n-butyl | n-butyl | 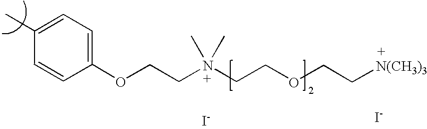 |
| 1238 | n-butyl | n-butyl | 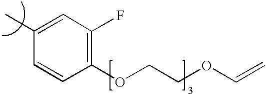 |
| 1239 | n-butyl | n-butyl | 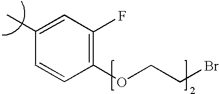 |
| 1240 | n-butyl | n-butyl | 4-methoxy-3-methylphenyl |
| 1241 | n-butyl | n-butyl | 3-(dimethylaminomethyl)phenyl |
| 1242 | n-butyl | n-butyl | 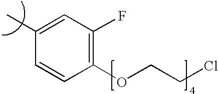 |
| 1243 | n-butyl | n-butyl | 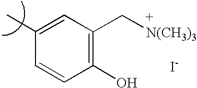 |
| 1244 | n-butyl | n-butyl | 3-methoxyphenyl |
| 1245 | n-butyl | n-butyl | 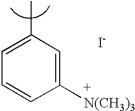 |
| 1246 | n-butyl | n-butyl | 3-(bromomethyl)phenyl |
| 1247 | n-butyl | n-butyl | 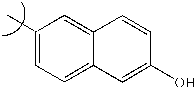 |
| 1248 | n-butyl | n-butyl | 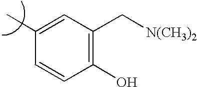 |
| 1249 | n-butyl | n-butyl | 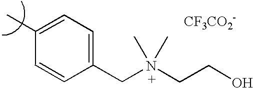 |
| 1250 | n-butyl | n-butyl | 3-(dimethylamino)phenyl |
| 1251 | n-butyl | n-butyl | 1-naphthyl |
| 1252 | n-butyl | n-butyl | 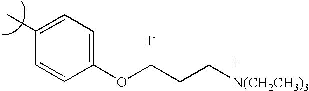 |
| 1253 | n-butyl | n-butyl | 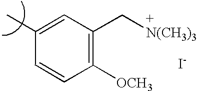 |
| 1254 | n-butyl | n-butyl | 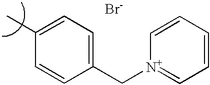 |
| 1255 | n-butyl | n-butyl | 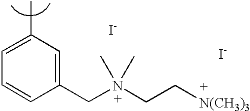 |
| 1256 | n-butyl | n-butyl | 3-nitrophenyl |
| 1257 | n-butyl | n-butyl | phenyl |
| 1258 | n-butyl | n-butyl | 4-fluorophenyl |
| 1259 | Ethyl | n-butyl | H |
| 1260 | Ethyl | n-butyl | 3-hydroxyphenyl |
| 1261 | n-butyl | n-butyl | 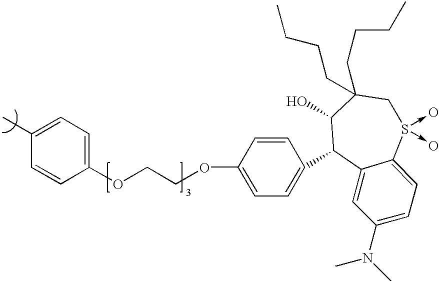 |
| 1262 | n-butyl | n-butyl | 2-thiophenyl |
| 1263 | n-butyl | n-butyl | 5-piperonyl |
| 1264 | n-butyl | n-butyl | 4-fluorophenyl |
| 1265 | n-butyl | n-butyl | 4-fluorophenyl |
| 1266 | n-butyl | n-butyl | 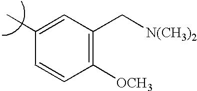 |
| 1267 | n-butyl | ethyl | 5-piperonyl |
| 1268 | n-butyl | n-butyl | 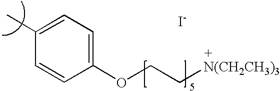 |
| 1269 | n-butyl | n-butyl | 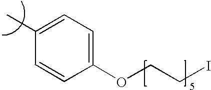 |
| 1270 | n-butyl | n-butyl | 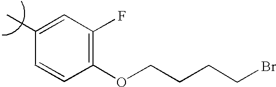 |
| 1271 | n-butyl | n-butyl | 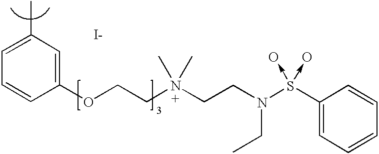 |
| 1272 | n-butyl | n-butyl | 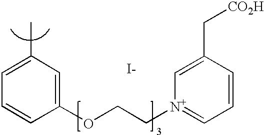 |
| 1273 | n-butyl | n-butyl | 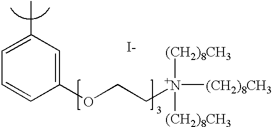 |
| 1274 | n-butyl | n-butyl | 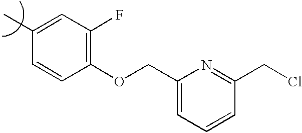 |
| 1275 | n-butyl | n-butyl | 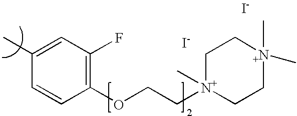 |
| 1276 | n-butyl | n-butyl | 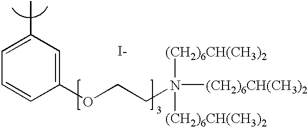 |
| 1277 | n-butyl | n-butyl | 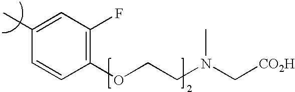 |
| 1278 | n-butyl | n-butyl | 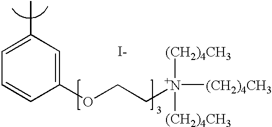 |
| 1279 | n-butyl | n-butyl | 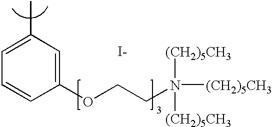 |
| 1280 | n-butyl | n-butyl | 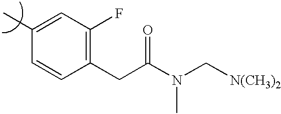 |
| 1281 | n-butyl | n-butyl | 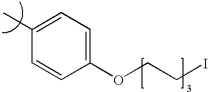 |
| 1282 | Ethyl | n-butyl | 3-fluoro-4-methoxyphenyl |
| 1283 | n-butyl | n-butyl | 4-hydroxymethylphenyl |
| 1284 | n-butyl | n-butyl | 4-fluorophenyl |
| 1285 | n-butyl | ethyl | phenyl |
| 1286 | n-butyl | n-butyl | 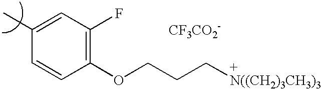 |
| 1287 | n-butyl | ethyl | 4-hydroxyphenyl |
| 1288 | n-butyl | n-butyl | 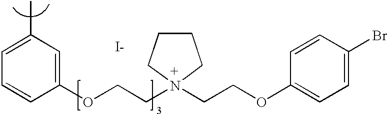 |
| 1289 | n-butyl | n-butyl | 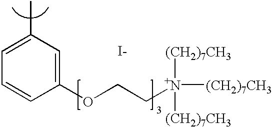 |
| 1290 | n-butyl | n-butyl | 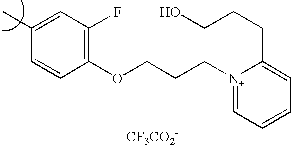 |
| 1291 | n-butyl | n-butyl | 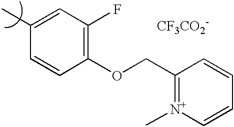 |
| 1292 | n-butyl | n-butyl | 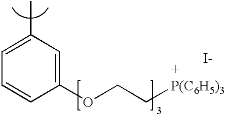 |
| 1293 | n-butyl | n-butyl | 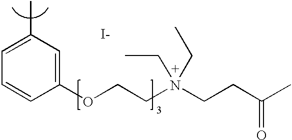 |
| 1294 | n-butyl | n-butyl | 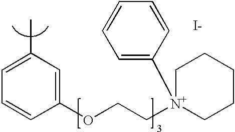 |
| 1295 | n-butyl | n-butyl | 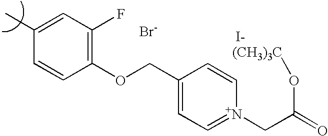 |
| 1296 | n-butyl | n-butyl | 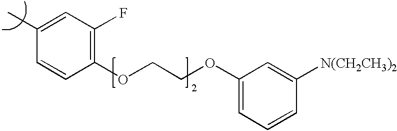 |
| 1297 | n-butyl | n-butyl | 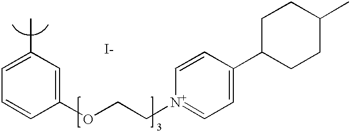 |
| 1298 | n-butyl | n-butyl | 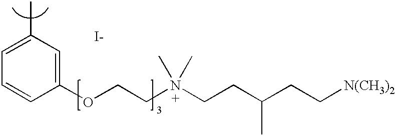 |
| 1299 | n-butyl | n-butyl | 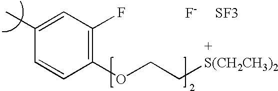 |
| 1300 | n-butyl | ethyl | H |
| 1301 | n-butyl | n-butyl | 3-methoxyphenyl |
| 1302 | n-butyl | n-butyl | 3-hydroxyphenyl |
| 1303 | n-butyl | n-butyl | 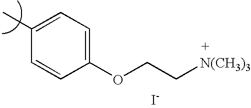 |
| 1304 | n-butyl | n-butyl | 3-methoxyphenyl |
| 1305 | n-butyl | n-butyl | 4-fluorophenyl |
| 1306 | n-butyl | n-butyl | 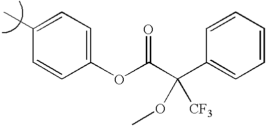 |
| 1307 | n-butyl | n-butyl | H |
| 1308 | Ethyl | n-butyl | 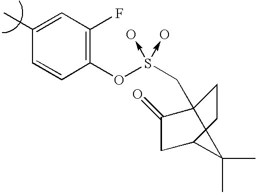 |
| 1309 | n-butyl | n-butyl | 4-methoxyphenyl |
| 1310 | Ethyl | n-butyl | phenyl |
| 1311 | n-butyl | ethyl | phenyl |
| 1312 | n-butyl | ethyl | phenyl |
| 1313 | n-butyl | ethyl | phenyl |
| 1314 | Ethyl | n-butyl | phenyl |
| 1315 | Ethyl | n-butyl | phenyl |
| 1316 | n-butyl | ethyl | phenyl |
| 1317 | n-butyl | ethyl | phenyl |
| 1318 | Ethyl | n-butyl | phenyl |
| 1319 | Ethyl | n-butyl | 3-methoxyphenyl |
| 1320 | Ethyl | n-butyl | phenyl |
| 1321 | n-butyl | ethyl | phenyl |
| 1322 | n-butyl | n-butyl | 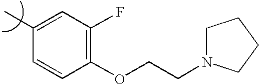 |
| 1323 | n-butyl | n-butyl | 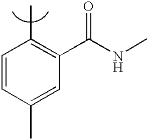 |
| 1324 | n-butyl | n-butyl | 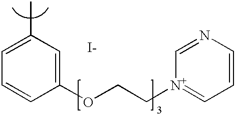 |
| 1325 | n-butyl | n-butyl | 4-((diethylamino)methyl)phenyl |
| 1326 | n-butyl | n-butyl | 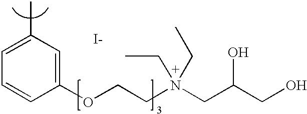 |
| 1327 | n-butyl | n-butyl | 3-fluoro-4-hydroxy-5-iodophenyl |
| 1328 | n-butyl | n-butyl | 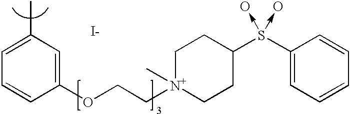 |
| 1329 | n-butyl | n-butyl | 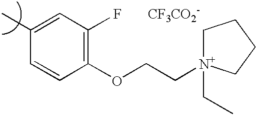 |
| 1330 | n-butyl | n-butyl | 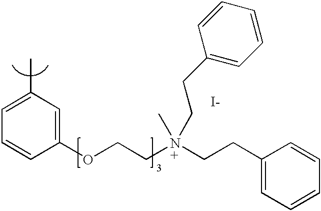 |
| 1331 | n-butyl | n-butyl | 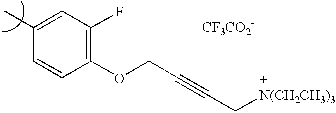 |
| 1332 | n-butyl | n-butyl | 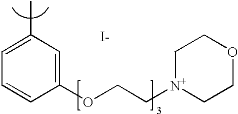 |
| 1333 | n-butyl | n-butyl | 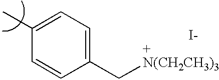 |
| 1334 | n-butyl | n-butyl | 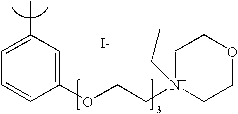 |
| 1335 | n-butyl | n-butyl | 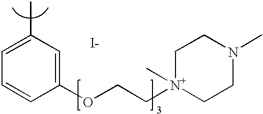 |
| 1336 | n-butyl | n-butyl | 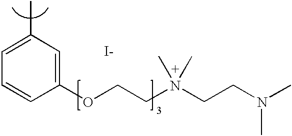 |
| 1337 | n-butyl | n-butyl | 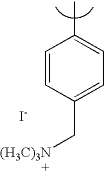 |
| 1338 | n-butyl | n-butyl | 4-methoxyphenyl |
| 1339 | n-butyl | n-butyl | 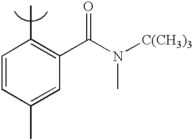 |
| 1340 | n-butyl | ethyl | 5-piperonyl |
| 1341 | n-butyl | n-butyl | 3-methoxyphenyl |
| 1342 | n-butyl | n-butyl | 5-piperonyl |
| 1343 | Ethyl | n-butyl | phenyl |
| 1344 | n-butyl | n-butyl | 3-fluoro-4-methoxyphenyl |
| 1345 | Ethyl | n-butyl | phenyl |
| 1346 | Ethyl | n-butyl | phenyl |
| 1347 | n-butyl | n-butyl | 3-fluoro-4-methoxyphenyl |
| 1348 | isobutyl | isobutyl | phenyl |
| 1349 | Ethyl | n-butyl | phenyl |
| 1350 | n-butyl | n-butyl | 3-fluoro-4-methoxyphenyl |
| 1351 | n-butyl | n-butyl | 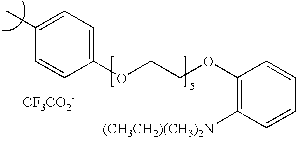 |
| 1352 | n-butyl | n-butyl | 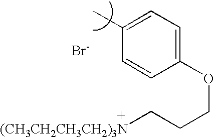 |
| 1353 | n-butyl | n-butyl | 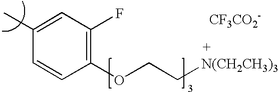 |
| 1354 | n-butyl | n-butyl | 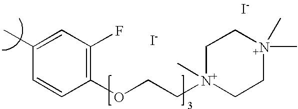 |
| 1355 | n-butyl | n-butyl | 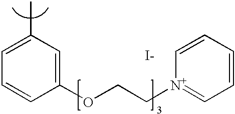 |
| 1356 | n-butyl | n-butyl | 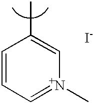 |
| 1357 | n-butyl | n-butyl | 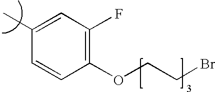 |
| 1358 | n-butyl | n-butyl | 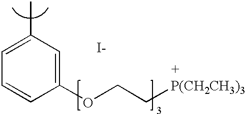 |
| 1359 | n-butyl | n-butyl | 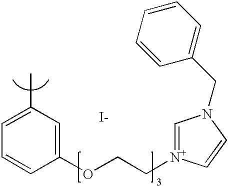 |
| 1360 | n-butyl | n-butyl | 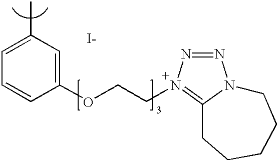 |
| 1361 | n-butyl | n-butyl | 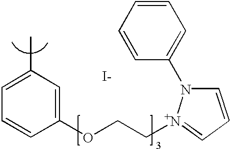 |
| 1362 | n-butyl | n-butyl | 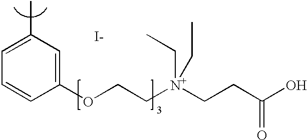 |
| 1363 | n-butyl | n-butyl | 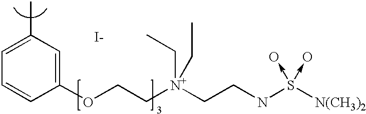 |
| 1364 | n-butyl | n-butyl | 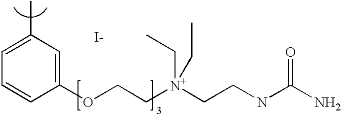 |
| 1365 | n-butyl | n-butyl | 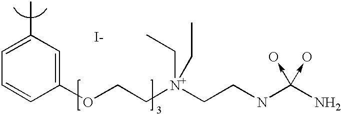 |
| 1366 | n-butyl | n-butyl | 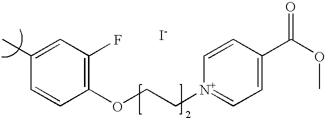 |
| 1367 | n-butyl | n-butyl | 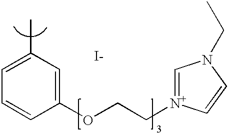 |
| 1368 | n-butyl | n-butyl | 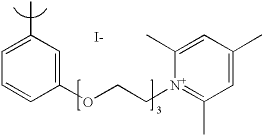 |
| 1369 | n-butyl | n-butyl | 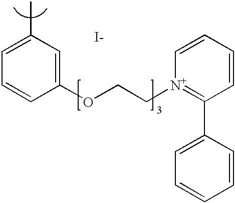 |
| 1370 | n-butyl | n-butyl | 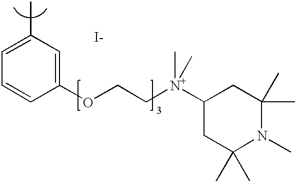 |
| 1371 | n-butyl | n-butyl | 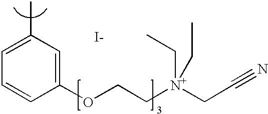 |
| 1372 | n-butyl | n-butyl | 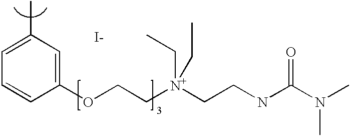 |
| 1373 | n-butyl | n-butyl | 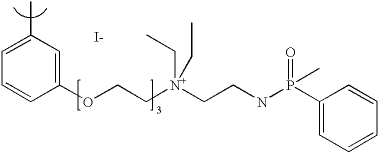 |
| 1374 | n-butyl | n-butyl | 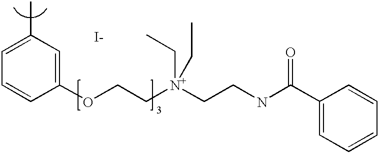 |
| 1375 | n-butyl | n-butyl | 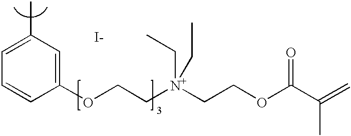 |
| 1376 | n-butyl | n-butyl | 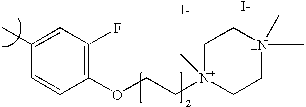 |
| 1377 | n-butyl | n-butyl | 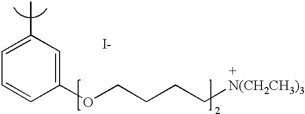 |
| 1378 | n-butyl | n-butyl | 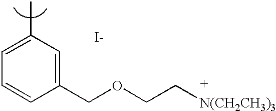 |
| 1379 | n-butyl | n-butyl | 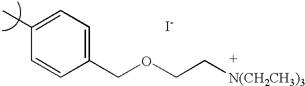 |
| 1380 | n-butyl | n-butyl | 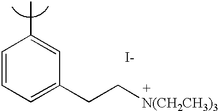 |
| 1381 | n-butyl | n-butyl | 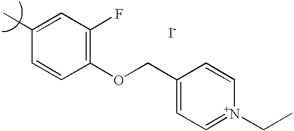 |
| 1382 | n-butyl | n-butyl | 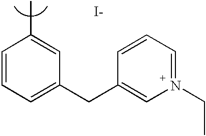 |
| 1383 | n-butyl | n-butyl | 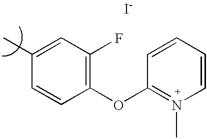 |
| 1384 | n-butyl | n-butyl | 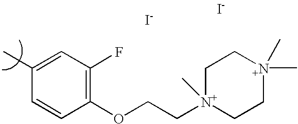 |
| 1385 | n-butyl | n-butyl | 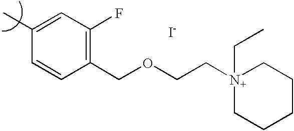 |
| 1386 | n-butyl | n-butyl | 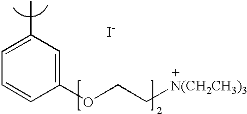 |
| 1387 | n-butyl | n-butyl | 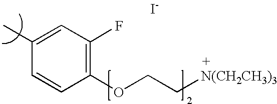 |
| 1388 | n-butyl | n-butyl | 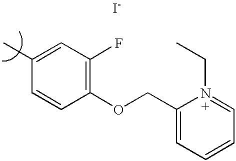 |
| 1389 | n-butyl | n-butyl | 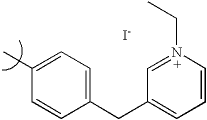 |
| 1390 | n-butyl | n-butyl | 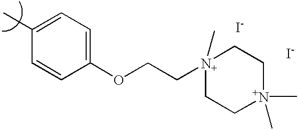 |
| 1391 | n-butyl | n-butyl | 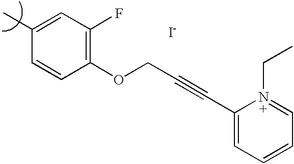 |
| 1392 | n-butyl | n-butyl | 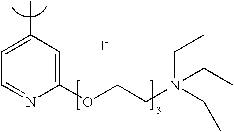 |
| 1393 | n-butyl | n-butyl | 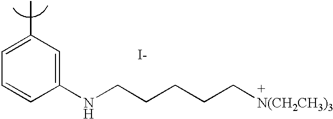 |
| 1394 | n-butyl | n-butyl | 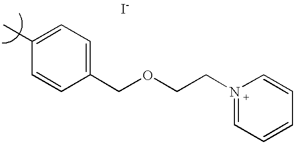 |
| 1395 | n-butyl | n-butyl | 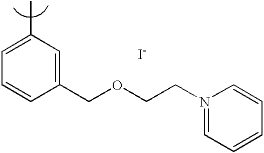 |
| 1396 | n-butyl | n-butyl | 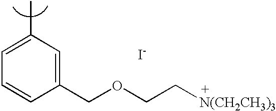 |
| 1397 | n-butyl | n-butyl | 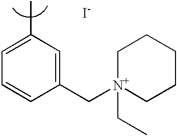 |
| 1398 | n-butyl | n-butyl | 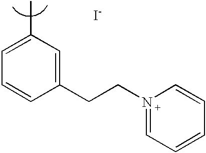 |
| 1399 | n-butyl | n-butyl | 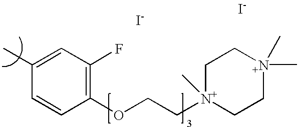 |
| 1400 | n-butyl | n-butyl | 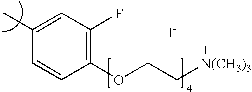 |
| 1401 | n-butyl | n-butyl | 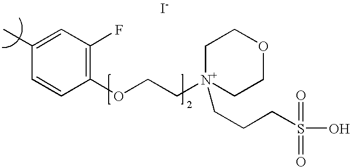 |
| 1402 | n-butyl | n-butyl | 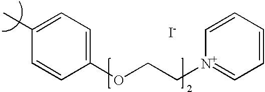 |
| 1403 | n-butyl | n-butyl | 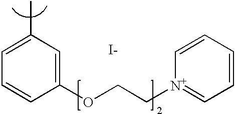 |
| 1404 | n-butyl | n-butyl | 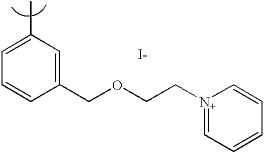 |
| 1405 | n-butyl | n-butyl | 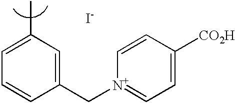 |
| 1406 | n-butyl | n-butyl | 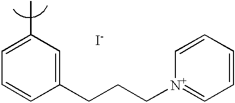 |
| 1407 | n-butyl | n-butyl | 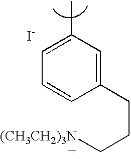 |
| 1408 | n-butyl | n-butyl | 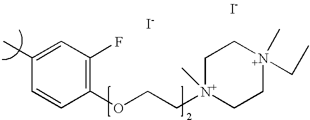 |
| 1409 | n-butyl | n-butyl | 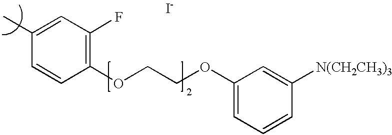 |
| 1410 | n-butyl | n-butyl | 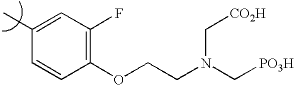 |
| 1411 | n-butyl | n-butyl | 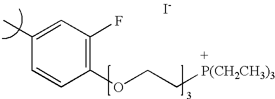 |
| 1412 | n-butyl | n-butyl | 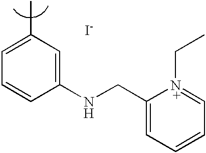 |
| 1413 | n-butyl | n-butyl | 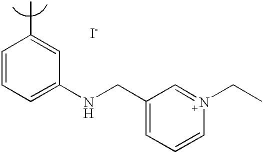 |
| 1414 | n-butyl | n-butyl | 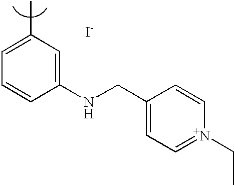 |
| 1415 | n-butyl | n-butyl | 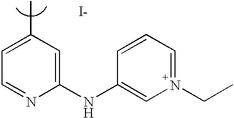 |
| 1416 | n-butyl | n-butyl | 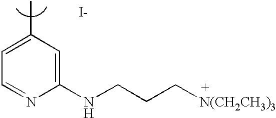 |
| 1417 | n-butyl | n-butyl | 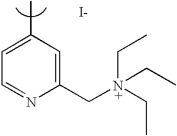 |
| 1418 | n-butyl | n-butyl | 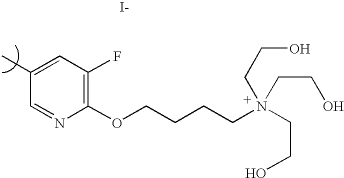 |
| 1419 | n-butyl | n-butyl | 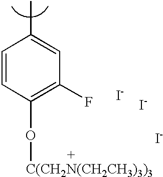 |
| 1420 | n-butyl | n-butyl | 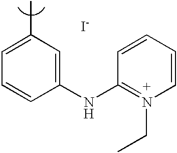 |
| 1421 | n-butyl | n-butyl | 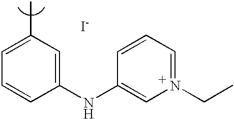 |
| 1422 | n-butyl | n-butyl | 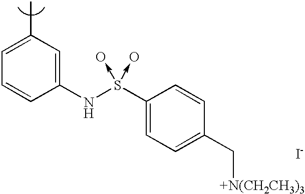 |
| 1423 | n-butyl | n-butyl | 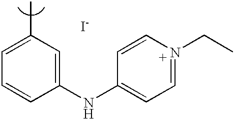 |
| 1424 | n-butyl | n-butyl | 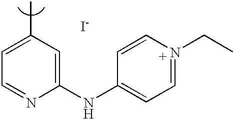 |
| 1425 | n-butyl | n-butyl | 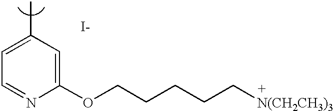 |
| 1426 | n-butyl | n-butyl | 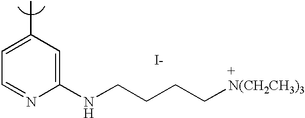 |
| 1427 | n-butyl | n-butyl | 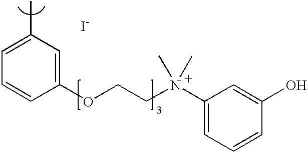 |
| 1428 | n-butyl | n-butyl | 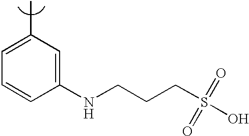 |
| 1429 | n-butyl | n-butyl | 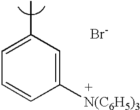 |
| 1430 | n-butyl | n-butyl | 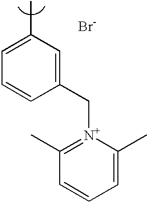 |
| 1431 | n-butyl | n-butyl | 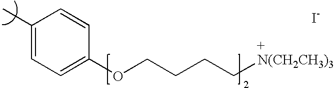 |
| 1432 | n-butyl | n-butyl | 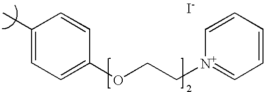 |
| 1433 | n-butyl | n-butyl | 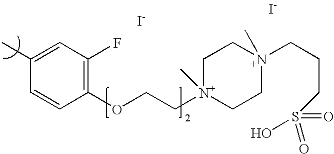 |
| 1434 | n-butyl | n-butyl | 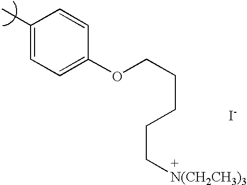 |
| 1435 | n-butyl | n-butyl | 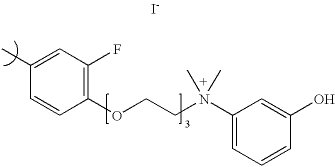 |
| 1436 | n-butyl | n-butyl | 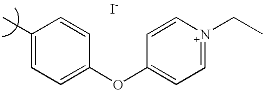 |
| 1437 | n-butyl | n-butyl | 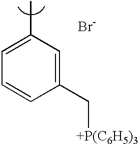 |
| 1438 | n-butyl | n-butyl | 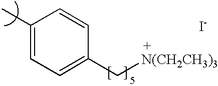 |
| 1439 | n-butyl | n-butyl | 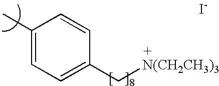 |
| 1440 | n-butyl | n-butyl | 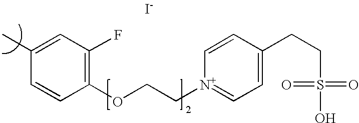 |
| 1441 | n-butyl | n-butyl | 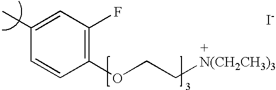 |
| 1442 | n-butyl | n-butyl | 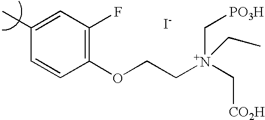 |
| 1443 | n-butyl | n-butyl | 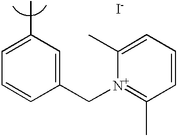 |
| 1444 | n-butyl | n-butyl | 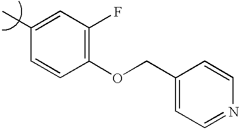 |
| 1445 | n-butyl | n-butyl | 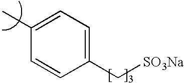 |
| 1446 | n-butyl | n-butyl | 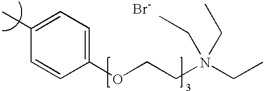 |
| 1447 | n-butyl | n-butyl | 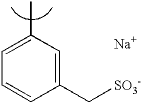 |
| 1448 | n-butyl | n-butyl | 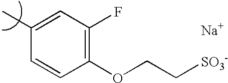 |
| 1449 | n-butyl | n-butyl | 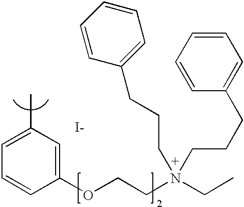 |
| 1450 | n-butyl | n-butyl | phenyl |
| 1451 | n-butyl | n-butyl | 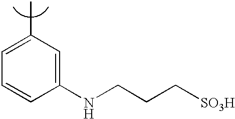 |

TABLE 10
(continuation of Table 9 substituents)

| | |
|---|---|
| 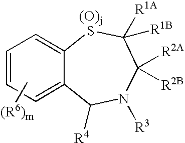 | IA |
| 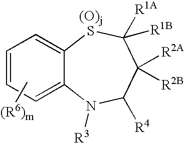 | IB |

| Com- | |
|---|---|
| pound | |
| Number | (R⁶)ₘ |
| 101 | 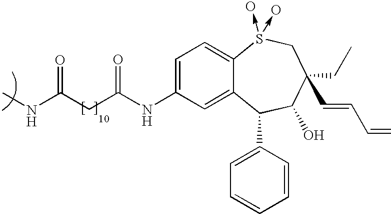 |
| 102 | 7-trimethylammonium iodide |
| 103 | 7-trimethylammonium iodide |
| 104 | 7-dimethylamino |
| 105 | 7-methanesulfonamido |
| 106 | 7-(2′-bromoacetamido) |
| 107 | 7-amino |
| 108 | 7-(hexylamido) |
| 109 | 7-amino |
| 110 | 7-acetamido |
| 111 | 7-amino |
| 112 | 7-amino |
| 113 | 7-amino |
| 114 | 7-amino |
| 115 | 7-(O-benzylcarbamato) |
| 116 | 7-(O-benzylcarbamato) |
| 117 | 7-(O-benzylcarbamato) |
| 118 | 7-(O-benzylcarbamato) |
| 119 | 7-(O-tert-butylcarbamato) |
| 120 | 7-(O-benzylcarbamato) |
| 121 | 7-amino |
| 122 | 7-amino |
| 123 | 7-hexylamino |
| 124 | 7-(hexylamino) |
| 125 | 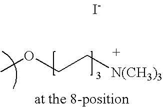 |
| 126 | 7-(O-benzylcarbamato) |
| 127 | 7-amino |
| 128 | 7-(O-benzylcarbamato) |
| 129 | 7-amino |
| 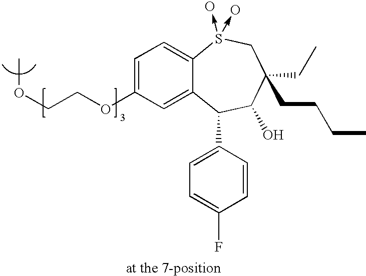 | |
| 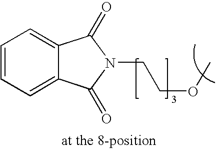 | |
| 133 | 8-(hexyloxy) |
| 134 | 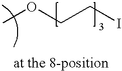 |
| 135 | 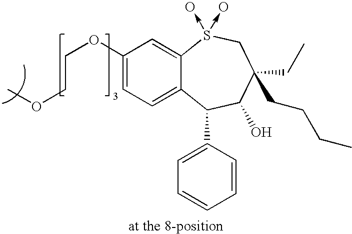 |
| 136 | 8-hydroxy |
| 137 | 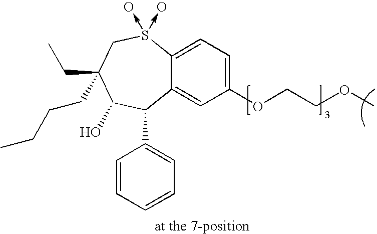 |
| 138 | 8-acetoxy |
| 139 | 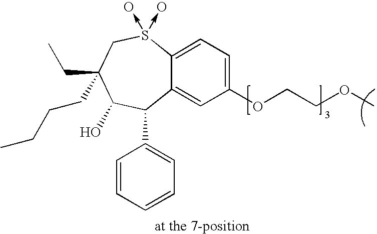 |
| 140 | |
| 141 | |
| 142 | 7-methylmercapto |
| 143 | 7-methylmercapto |
| 144 | 7-(N-azetidinl) |
| 262 | 7-methoxy |
| 263 | 7-methoxy |
| 264 | 7-methoxy |
| 265 | 7-methoxy |
| 266 | 7-hydroxy |
| 267 | 7-methoxy |
| 268 | 7-methoxy |
| 269 | 7-methoxy |
| 270 | 7-hydroxy |
| 271 | 7-bromo |
| 272 | 7-bromo |
| 273 | 7-fluoro |
| 274 | 7-fluoro |
| 275 | 7-fluoro |
| 276 | 7-fluoro |
| 277 | 7-methoxy |
| 278 | 7-methoxy |
| 279 | 7-methoxy |
| 280 | 7-methoxy |
| 281 | 7-methylmercapto |
| 282 | 7-methyl |
| 283 | 7-methyl |
| 284 | 7-(4′-morpholino) |
| 286 | 7-(O-benzylcarbamato) |
| 287 | 7-amino |
| 288 | 7-amino |
| 289 | 7-amino |
| 290 | 7-amino |
| 291 | 7-(O-benzylcarbamato) |
| 292 | 7-amino |
| 293 | 7-benzylamino |
| 294 | 7-dimethylamino |
| 295 | 7-amino |
| 296 | 7-amino |
| 1000 | 7-dimethylamino |
| 1001 | 7-dimethylamino |
| 1002 | 7-dimethylamino |
| 1003 | 7-dimethylamino |
| 1004 | 7-dimethylamino |
| 1005 | 7-dimethylamino |
| 1006 | 7-dimethylamino |
| 1007 | 7-dimethylamino |
| 1008 | 7-dimethylamino |
| 1009 | 7-dimethylamino |
| 1010 | 7-dimethylamino |
| 1011 | 7-dimethylamino |
| 1012 | 7-dimethylamino; 9-methoxy |
| 1013 | 7-dimethylamino |
| 1014 | 7-dimethylamino; 9-methoxy |
| 1015 | 7-dimethylamino |
| 1016 | 7-dimethylamino |
| 1017 | 7-dimethylamino |
| 1018 | 7-dimethylamino |
| 1019 | 7-dimethylamino |
| 1020 | 7-dimethylamino |
| 1021 | 7-dimethylamino |
| 1022 | 7-dimethylamino |
| 1023 | 7-dimethylamino |
| 1024 | 7-dimethylamino |
| 1025 | 7-dimethylamino |
| 1026 | 7-dimethylamino |
| 1027 | 7-dimethylamino |
| 1028 | 7-dimethylamino |
| 1029 | 7-dimethylamino |
| 1030 | 7-dimethylamino |
| 1031 | 7-dimethylamino |
| 1032 | 7-dimethylamino |
| 1033 | 7-dimethylamino |
| 1034 | 7-dimethylamino |
| 1035 | 7-dimethylamino |
| 1036 | 7-dimethylamino |
| 1037 | 7-dimethylamino |
| 1038 | 7-dimethylamino |
| 1039 | 7-dimethylamino |
| 1040 | 7-dimethylamino |
| 1041 | 7-dimethylamino |
| 1042 | 7-dimethylamino |
| 1043 | 7-dimethylamino |
| 1044 | 7-dimethylamino |
| 1045 | 7-dimethylamino |
| 1046 | 7-dimethylamino |
| 1047 | 7-dimethylamino |
| 1048 | 7-dimethylamino |
| 1049 | 7-dimethylamino |
| 1050 | 7-dimethylamino |
| 1051 | 7-dimethylamino |
| 1052 | 7-dimethylamino |
| 1053 | 7-dimethylamino |
| 1054 | 7-dimethylamino |
| 1055 | 7-dimethylamino |
| 1056 | 7-dimethylamino |
| 1057 | 7-dimethylamino |
| 1058 | 7-dimethylamino |
| 1059 | 7-dimethylamino |
| 1060 | 7-methylamino |
| 1061 | 7-methylamino |
| 1062 | 7-methylamino |
| 1063 | 7-methylamino |
| 1064 | 7-methylamino |
| 1065 | 7-dimethylamino |
| 1066 | 7-dimethylamino |
| 1067 | 9-dimethylamino |
| 1068 | 7-dimethylamino |
| 1069 | 7-dimethylamino; |
| | 9-dimethylamino |
| 1070 | 7-dimethylamino |
| 1071 | 7-dimethylamino |
| 1072 | 7-dimethylamino |
| 1073 | 7-dimethylamino |
| 1074 | 7-dimethylamino |
| 1075 | 7-dimethylamino; 9-dimethylamino |
| 1076 | 7-dimethylamino |
| 1077 | 7-dimethylamino |
| 1078 | 7-dimethylamino |
| 1079 | 7-dimethylamino |
| 1080 | 7-dimethylamino |
| 1081 | 7-dimethylamino |
| 1082 | 7-dimethylamino |
| 1083 | 7-dimethylamino |
| 1084 | 7-dimethylamino |
| 1085 | 7-dimethylamino |
| 1086 | 7-dimethylamino |
| 1087 | 7-dimethylamino |
| 1088 | 7-dimethylamino |
| 1089 | 7-dimethylamino |
| 1090 | 7-dimethylamino |
| 1091 | 7-dimethylamino |
| 1092 | 7-dimethylamino |
| 1093 | 7-dimethylamino |
| 1094 | 7-dimethylamino |
| 1095 | 7-dimethylamino |
| 1096 | 7-dimethylamino |
| 1097 | 7-dimethylamino |
| 1098 | 7-dimethylamino |
| 1099 | 7-dimethylamino |
| 1100 | 7-dimethylamino |
| 1101 | 7-dimethylamino |
| 1102 | 7-dimethylamino |
| 1103 | 7-dimethylamino |
| 1104 | 7-dimethylamino |
| 1105 | 7-dimethylamino |
| 1106 | 7-dimethylamino |
| 1107 | 7-dimethylamino |
| 1108 | 7-dimethylamino |
| 1109 | 7-dimethylamino |
| 1110 | 7-dimethylamino |
| 1111 | 7-dimethylamino |
| 1112 | 7-dimethylamino |
| 1113 | 7-dimethylamino |
| 1114 | 7-methylamino |
| 1115 | 7-dimethylamino |
| 1116 | 7-dimethylamino |
| 1117 | 7-dimethylamino |
| 1118 | 7-dimethylamino |
| 1119 | 7-dimethylamino |
| 1120 | 7-dimethylamino |
| 1121 | 7-dimethylamino |
| 1122 | 7-dimethylamino |
| 1123 | 7-dimethylamino |
| 1124 | 7-dimethylamino |
| 1125 | 7-dimethylamino |
| 1126 | 7-dimethylamino |
| 1127 | 7-dimethylamino |
| 1128 | 7-dimethylamino |
| 1129 | 9-dimethylamino |
| 1130 | 7-dimethylamino |
| 1131 | 7-dimethylamino |
| 1132 | 7-dimethylamino |
| 1133 | 7-dimethylamino |
| 1134 | 7-dimethylamino |
| 1135 | 7-dimethylamino |
| 1136 | 7-dimethylamino |
| 1137 | 9-(2′,2′-dimethylhydrazino) |
| 1138 | 7-dimethylamino |
| 1139 | 7-dimethylamino |
| 1140 | 7-(2′,2′-dimethylhydrazino) |
| 1141 | 7-ethylmethylamino |
| 1142 | 7-dimethylamino |
| 1143 | 7-dimethylamino |
| 1144 | 7-dimethylamino |
| 1145 | 9-dimethylamino |
| 1146 | 7-dimethylammo |
| 1147 | 7-diethylamino |
| 1148 | 7-dimethylsulfonium, fluoride salt |
| 1149 | 7-ethylamino |
| 1150 | 7-ethylmethylamino |
| 1151 | 7-dimethylamino |
| 1152 | 7-(ethoxymethyl)methylamino |
| 1153 | 7-methylamino |
| 1154 | 9-methoxy |
| 1155 | 7-methyl |
| 1156 | 7-methylmercapto |
| 1157 | 7-fluoro; |
| | 9-dimethylamino |
| 1158 | 7-methoxy |
| 1159 | 7-dimethylamino |
| 1160 | 7-diethylamino |
| 1161 | 7-dimethylamino |
| 1162 | 7-dimethylamino |
| 1163 | 7-methoxy |
| 1164 | 7-methoxy |
| 1165 | 7-trimethylammonium iodide |
| 1166 | 7-trimethylammonium iodide |
| 1167 | 7-dimethylamino |
| 1168 | 7-trimethylammonium iodide |
| 1169 | 8-dimethylamino |
| 1170 | 7-ethylpropylamino |
| 1171 | 7-dimethylamino |
| 1172 | 7-methoxy |
| 1173 | 7-ethylpropylamino |
| 1174 | 7-phenyl |
| 1175 | 7-methylsulfonyl |
| 1176 | 9-fluoro |
| 1177 | 7-butylmethylamino |
| 1178 | 7-dimethylamino |
| 1179 | 8-methoxy |
| 1180 | 7-trimethylammonium iodide |
| 1181 | 7-butylmethylamino |
| 1182 | 7-methoxy |
| 1183 | 7-fluoro |
| 1184 | 7-fluoro; 9-fluoro |
| 1185 | 7-fluoro |
| 1186 | 7-fluoro; 9-fluoro |
| 1187 | 7-methyl |
| 1188 | 7-trimethylammonium iodide |
| 1189 | 7-trimethylammonium iodide |
| 1190 | 7-bromo |
| 1191 | 7-hydroxy |
| 1192 | 7-hydroxy |
| 1193 | 7-dimethylamino |
| 1194 | 7-dimethylamino |
| 1195 | 7-(4′-methylpiperazin-1-yl) |
| 1196 | 7-methoxy |
| 1197 | 7-(N-methylformamido) |
| 1198 | 7-methoxy |
| 1199 | 7-dimethylamino |
| 1200 | 7-dimethylamino |
| 1201 | 7-methyl |
| 1202 | 7-methoxy |
| 1203 | 7-(4′-tert-butylphenyl) |
| 1204 | 7-methoxy |
| 1205 | 7-dimethylamino |
| 1206 | 7-dimethylamino |
| 1207 | 7-dimethylamino |
| 1208 | 7-dimethylamino |
| 1209 | 7-dimethylphenyl |
| 1210 | 7-dimethylamino |
| 1211 | 7-dimethylammo |
| 1212 | 9-(4′-morpholino) |
| 1213 | 7-dimethylamino |
| 1214 | 7-(N-methylformamido) |
| 1215 | 9-methylmercapto |
| 1216 | 7-bromo |
| 1217 | 7-dimethylammo |
| 1218 | 9-methylsulfonyl |
| 1219 | 7-dimethylamino |
| 1220 | 7-isopropylamino |
| 1221 | 7-dimethylamino |
| 1222 | 7-ethylamino |
| 1223 | 8-bromo; |
| | 7-dimethylamino |
| 1224 | 7-fluoro |
| 1225 | 7-dimethylamino |
| 1226 | 7-bromo |
| 1227 | 7-(tert-butylamino |
| 1228 | 8-bromo; |
| | 7-dimethylamino |
| 1229 | 7-dimethylamino |
| 1230 | 9-dimethylamino; |
| | 7-fluoro |
| 1231 | 7-dimethylamino |
| 1232 | 9-dimethylamino |
| 1233 | 7-dimethylamino |
| 1234 | 7-dimethylamino |
| 1235 | 7-dimethylamino |
| 1236 | 7-dimethylamino |
| 1237 | 7-dimethylamino |
| 1238 | 7-dimethylamino |
| 1239 | 7-dimethylamino |
| 1240 | 7-dimethylamino |
| 1241 | 7-dimethylamino |
| 1242 | 7-dimethylamino |
| 1243 | 7-dimethylamino |
| 1244 | 7-(1′-methylhydrazido) |
| 1245 | 7-dimethylamino |
| 1246 | 7-dimethylamino |
| 1247 | 7-dimethylamino |
| 1248 | 7-dimethylamino |
| 1249 | 7-dimethylamino |
| 1250 | 7-dimethylamino |
| 1251 | 7-dimethylamino |
| 1252 | 7-dimethylamino |
| 1253 | 7-dimethylamino |
| 1254 | 7-dimethylamino |
| 1255 | 7-dimethylamino |
| 1256 | 7-dimethylamino |
| 1257 | 8-bromo; 7-dimethylamino |
| 1258 | 9-(tert-butylamino) |
| 1259 | 7-dimethylamino |
| 1260 | 7-dimethylamino |
| 1261 | 7-dimethylamino |
| 1262 | 7-dimethylamino |
| 1263 | 7-bromo |
| 1264 | 7-isopropylamino |
| 1265 | 9-isopropylamino |
| 1266 | 7-dimethylamino |
| 1267 | 7-carboxy, methyl ester |
| 1268 | 7-dimethylamino |
| 1269 | 7-dimethylamino |
| 1270 | 7-dimethylamino |
| 1271 | 7-dimethylamino |
| 1272 | 7-dimethylamino |
| 1273 | 7-dimethylamino |
| 1274 | 7-dimethylamino |
| 1275 | 7-dimethylamino |
| 1276 | 7-dimethylamino |
| 1277 | 7-dimethylamino |
| 1278 | 7-dimethylamino |
| 1279 | 7-dimethylamino |
| 1280 | 7-dimethylamino |
| 1281 | 7-dimethylamino |
| 1282 | 7-trimethylammonium iodide |
| 1283 | 7-dimethylamino |
| 1284 | 9-ethylamino |
| 1285 | 7-dimethylamino |
| 1286 | 7-dimethylamino |
| 1287 | 7-dimethylamino |
| 1288 | 7-dimethylamino |
| 1289 | 7-dimethylamino |
| 1290 | 7-dimethylamino |
| 1291 | 7-dimethylamino |
| 1292 | 7-dimethylamino |
| 1293 | 7-dimethylamino |
| 1294 | 7-dimethylamino |
| 1295 | 7-dimethylamino |
| 1296 | 7-dimethylamino |
| 1297 | 7-dimethylamino |
| 1298 | 7-dimethylamino |
| 1299 | 7-dimethylamino |
| 1300 | 7-dimethylamino |
| 1301 | 7-trimethylammonium iodide |
| 1302 | 9-hydroxy |
| 1303 | 7-dimethylamino |
| 1304 | 7-tert-butylamino |
| 1305 | 9-methylamino |
| 1306 | 7-dimethylamino |
| 1307 | 9-(4′-morpholino) |
| 1308 | 7-dimethylamino |
| 1309 | 9-fluoro |
| 1310 | 7-amino |
| 1311 | 7-(hydroxylamino) |
| 1312 | 8-hexyloxy |
| 1313 | 8-ethoxy |
| 1314 | 7-(hydroxylamino) |
| 1315 | 7-(hexyloxy) |
| 1316 | 8-hydroxy |
| 1317 | 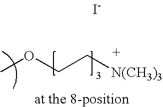 |
| 1318 | 7-dimethylamino |
| 1319 | 7-fluoro |
| 1320 | 7-amino |
| 1321 | 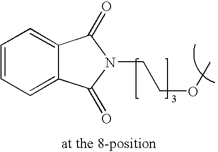 |
| 1322 | 7-dimethylamino |
| 1323 | 7-dimethylamino |
| 1324 | 7-dimethylamino |
| 1325 | 7-dimethylamino |
| 1326 | 7-dimethylamino |
| 1327 | 7-dimethylamino |
| 1328 | 7-dimethylamino |
| 1329 | 7-dimethylamino |
| 1330 | 7-dimethylamino |
| 1331 | 7-dimethylamino |
| 1332 | 7-dimethylamino |
| 1333 | 7-dimethylamino |
| 1334 | 7-dimethylamino |
| 1335 | 7-dimethylamino |
| 1336 | 7-dimethylamino |
| 1337 | 7-dimethylamino |
| 1338 | 7-(4′-methylpiperazinyl) |
| 1339 | 7-dimethylamino |
| 1340 | 7-methyl |
| 1341 | 7-dimethylamino |
| 1342 | 7-(4′-fluorophenyl) |
| 1343 | 7-amino |
| 1344 | 7-dimethylamino |
| 1345 | 7-trimethylammonium iodide |
| 1346 | 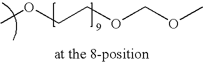 |
| 1347 | 7-dimethylamino |
| 1348 | 7-dimethylamino |
| 1349 | 7-dimethylamino |
| 1350 | 7-trimethylammonium iodide |
| 1351 | 7-dimethylamino |
| 1352 | 7-dimethylamino |
| 1353 | 7-dimethylamino |
| 1354 | 7-dimethylamino |
| 1355 | 7-dimethylamino |
| 1356 | 7-dimethylamino |
| 1357 | 7-dimethylamino |
| 1358 | 7-dimethylamino |
| 1359 | 7-dimethylamino |
| 1360 | 7-dimethylamino |
| 1361 | 7-dimethylamino |
| 1362 | 7-dimethylamino |
| 1363 | 7-dimethylamino |
| 1364 | 7-dimethylamino |
| 1365 | 7-dimethylamino |
| 1366 | 7-dimethylamino |
| 1367 | 7-dimethylamino |
| 1368 | 7-dimethylamino |
| 1369 | 7-dimethylamino |
| 1370 | 7-dimethylamino |
| 1371 | 7-dimethylamino |
| 1372 | 7-dimethylamino |
| 1373 | 7-dimethylamino |
| 1374 | 7-dimethylamino |
| 1375 | 7-dimethylamino |
| 1376 | 7-dimethylamino |
| 1377 | 7-dimethylamino |
| 1378 | 7-dimethylamino |
| 1379 | 7-dimethylanimo |
| 1380 | 7-dimethylamino |
| 1381 | 7-dimethylamino |
| 1382 | 7-dimethylamino |
| 1383 | 7-dimethylamino |
| 1384 | 7-dimethylamino |
| 1385 | 7-dimethylamino |
| 1386 | 7-dimethylamino |
| 1387 | 7-dimethylamino |
| 1388 | 7-dimethylamino |
| 1389 | 7-dimethylamino |
| 1390 | 7-dimethylamino |
| 1391 | 7-dimethylamino |
| 1392 | 7-dimethylamino |
| 1393 | 7-dimethylamino |
| 1394 | 7-dimethylamino |
| 1395 | 7-dimethylamino |
| 1396 | 7-dimethylamino |
| 1397 | 7-dimethylamino |
| 1398 | 7-dimethylamino |
| 1399 | 7-dimethylamino |
| 1400 | 7-dimethylamino |
| 1401 | 7-dimethylamino |
| 1402 | 7-dimethylamino |
| 1403 | 7-dimethylamino |
| 1404 | 7-dimethylamino |
| 1405 | 7-dimethylamino |
| 1406 | 7-dimethylamino |
| 1407 | 7-dimethylamino |
| 1408 | 7-dimethylamino |
| 1409 | 7-dimethylamino |
| 1410 | 7-dimethylamino |
| 1411 | 7-dimethylamino |
| 1412 | 7-dimethylamino |
| 1413 | 7-dimethylamino |
| 1414 | 7-dimethylamino |
| 1415 | 7-dimethylamino |
| 1416 | 7-dimethylamino |
| 1417 | 7-dimethylamino |
| 1418 | 7-dimethylamino |
| 1419 | 7-dimethylamino |
| 1420 | 7-dimethylamino |
| 1421 | 7-dimethylamino |
| 1422 | 7-dimethylamino |
| 1423 | 7-dimethylamino |
| 1424 | 7-dimethylamino |
| 1425 | 7-dimethylamino |
| 1426 | 7-dimethylamino |
| 1427 | 7-dimethylamino |
| 1428 | 7-dimethylamino |
| 1429 | 7-dimethylamino |
| 1430 | 7-dimethylamino |
| 1431 | 7-dimethylamino |
| 1432 | 7-dimethylamino |
| 1433 | 7-dimethylamino |
| 1434 | 7-dimethylamino |
| 1435 | 7-dimethylamino |
| 1436 | 7-dimethylamino |
| 1437 | 7-dimethylamino |
| 1438 | 7-dimethylamino |
| 1439 | 7-dimethylamino |
| 1440 | 7-dimethylamino |
| 1441 | 7-dimethylamino |
| 1442 | 7-dimethylamino |
| 1443 | 7-dimethylamino |
| 1444 | 7-dimethylamino |
| 1445 | 7-dimethylamino |
| 1446 | 7-methoxy; 8-methoxy |
| 1447 | 7-dimethylamino |
| 1448 | 7-dimethylamino |
| 1449 | 7-dimethylamino |
| 1450 | 7-dimethylamino |
| 1451 | 7-dimethylamino |

TABLE 11

| | |
|---|---|
| 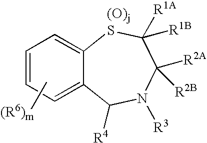 | IA |
| 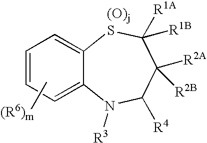 | IB |

The comments below refer to the structures denoted in Table 11 above. According to additional embodiments of the invention, in the above-noted structures IA and IB, R^{1A} and R^{1B} can be independently selected from the group consisting of: ethyl, n-propyl, n-butyl, n-pentyl, n-hexyl, iso-propyl, iso-butyl, iso-pentyl, CH₂(C═O)C₂H₅, CH₂OCH₅, CH₂CH(OH)C₂H₅, and CH₂O-(4-picoline). 1901] Additionally, R³ and R⁴ can independently be selected from the group consisting of groups (1)-(70) of Table 1 as well as the following: para-methoxy-phenyl, meta-methoxy-phenyl, m-(CH₃)₂N-Ph-, p-(CH₃)₂N-Ph-, I⁻p-((CH₃)₃—N⁺-Ph-, I⁻m-((CH₃)₃—N⁺-Ph-, I⁻p-((CH₃)₃—N⁺-CH₂CH₂—(OCH₂CH₂)₂—O-Ph-, I⁻, p-(N,N-dimethylpiperazine)-(N′)′, CH₂—(OCH₂CH₂)₂—O-Ph-, p-CH₃O-Ph-, 3,4, dioxymethylene-Ph, m-CH₃O—, p-F-Ph-, 4-pyridine, N-methyl-4-pyridinium, I⁻, 3-pyridine, N-methyl-3-pyridinium, I⁻, 2-pyridine, p-CH₃O₂C-Ph-, thienyl-2-yl, 5-Cl-thienyl-2-yl, p-F-Ph-, and m-CH₃O-Ph.

Also, R⁶ can be independently selected from the group consisting of: 7-methyl, 7-ethyl, 7-iso-propyl, 7-tert-butyl, 7-OH, 7-OCH₃, 7-O(iso-propyl), 7-SCH₃, 7-SOCH₃, 7-SO₂CH₃, 7-SCH₂CH₃, 7-NH₂,7-NHOH, 7-NHCH₃, 7-N(CH₃)₂, 7-N(CH₃)₃, I⁻7-NHC(═O)CH₃, 7-N(CH₂CH₃)₂, 7-NMeCH₂CO₂H, 7-N⁺(Me)₂CH₂CO₂H, I⁻, 7-(N)-morpholine, 7-(N)-azetidine, 7-(N)-N-methylazetidinium, I⁻, 7-(N)-pyrrolidine, 7-(N)-N-methyl-pyrrolidinium, I⁻, 7-(N)-N′-methylpiperazine, 7-(N)-N′-dimethylpiperazinium, I⁻, 7-NH-CBZ, 7-NHC(O)C₅H₁₁, 7-NHC(O)CH₂Br, 7-NH—C(NH)NH₂, 7-(2)-thiophene, 8-methyl, 8-ethyl, 8-iso-propyl, 8-tert-butyl, 8-OH, 8-OCH₃, 8-O(iso-propyl), 8-SCH₃, 8-SOCH₃, 8-SO₂CH₃, 8-SCH₂CH₃, 8-NH_{2,8}-NHOH, 8-NHCH₃, 8-N(CH₃)₂,8-N(CH₃)₃, I⁻8-NHC(═O)CH₃, 8-N(CH₂CH₃)₂, 8-NMeCH₂CO₂H, 8-N⁺(Me)₂CH₂CO₂H, I⁻, 8-(N)-morpholine, 8-(N)-azetidine, 8-(N)-N-methylazetidinium, I⁻, 8-(N)-pyrrolidine, 8-(N)-N-methyl-pyrrolidinium, I⁻, 8-(N)-N′-methylpiperazine, 8-(N)-N′-dimethylpiperazinium, I⁻, 8-NH-CBZ, 8-NHC(O)C₅H₁₁, 8-NHC(O)CH₂Br, 8-NH—C(NH)NH₂, 8-(2)-thiophene, 9-methyl, 9-ethyl, 9-iso-propyl, 9-tert-butyl, 9-OH, 9-OCH₃, 9-O(iso-propyl), 9-SCH₃, 9-SOCH₃, 9-SO₂CH₃, 9-SCH₂CH₃, 9-NH_{2,9}-NHOH, 9-NHCH₃, 9-N(CH₃)₂, 9-N⁺(CH₃)₃, I⁻9-NHC(═O)CH₃, 9-N(CH₂CH₃)₂, 9-NMeCH₂CO₂H, 9-N⁺(Me)₂CH₂CO₂H, I⁻, 9-(N)-morpholine, 9-(N)-azetidine, 9-(N)-N-methylazetidinium, I⁻, 9-(N)-pyrrolidine, 9-(N)-N-methyl-pyrrolidinium, I⁻, 9-(N)-N′-methylpiperazine, 9-(N)-N′-dimethylpiperazinium, I⁻, 9-NH-CBZ, 9-NHC(O)C₅H₁₁, 9-NHC(O)CH₂Br, 9-NH—C(NH)NH₂, and 9-(2)-thiophene.

Furthermore, R⁶ may also be selected from the group consisting of: 7-(1-aziridine), 7-EtS-, 7-CH₃S(O)—, 7-CH₃S(O)₂—, 7-PhS-, 7CH₃S—, 9-CH₃S—, 7-CH₃O—, 9-CH₃O—, 7-Et-7-iPr-, 7-t-Bu-, 7-(1-pyrazole)-, 7-(1-azetidine), 6-CH₃O—, 8-CH₃O—, 9-CH₃—, 7-CH₃, 7-(1-pyrrole), 7-(N)N′-methylpiperazine, 7-CH₃C(═CH₂)—, 7-cyclpropyl, 7-(CH₃)₂NHN—, 7-(N)-azetidine, 7-(N-pyrrolidine), 7-(CH₃)₂N—, 9-CH₃S—, 7-(CH₃)₂N—, 6-CH₃O—, 7-CH₃O—, 8-CH₃O—, 7-Me—, 9-CH₃, 7-(CH₃)₂N—, 7-cyclopropyl, 8-methyl, 8-ethyl, 8-iso-propyl, 8-tert-butyl, 8-OH, 8-OCH₃, 8-O(iso-propyl), 8-SCH₃, 8-SOCH₃, 8-SO₂CH₃, 8-SCH₂CH₃, 8-NH_{2,8}-NHOH, 8-NHCH₃, 8-N(CH₃)₂, 8-N⁺(CH₃)₃, I⁻, 8-NHC(═O)CH₃, 8-N(CH₂CH₃)₂, 8-NMeCH₂CO₂H, 8-N⁺(Me)₂CH₂CO₂H, I⁻, 8-(N)-morpholine, 8-(N)-azetidine, 8-(N)-N-methylazetidinium, I⁻, 8-(N)-pyrrolidine, 8-(N)-N-methyl-pyrrolidinium, I⁻, 8-(N)-N-methyl-morpholinium, I⁻, 8-(N)-N′-methylpiperazine, 8-(N)-N′-dimethylpiperazinium, I⁻, 8-NH-CBZ, 8-NHC(O)C₅H₁₁, 8-NHC(O)CH₂Br, 8-NH—C(NH)NH₂, 8-(2)-thiophene, 9-methyl, 9-ethyl, 9-iso-propyl, 9-tert-butyl, 9-OH, 9-OCH₃, 9-O(iso-propyl), 9-SCH₃, 9-SOCH₃, 9-SO₂CH₃, 9-SCH₂CH₃, 9-NH_{2,9}-NHOH, 9-NHCH₃, 9-N(CH₃)₂, 9-N⁺(CH₃)₃, I⁻, 9-NHC(═O)CH₃, 9-N(CH₂CH₃)₂, 9-NMeCh₂CO₂H, 9-N⁺(Me)₂CH₂CO₂H, I⁻, 9-(N)-morpholine, 9-(N)-azetidine, 9-(N)-N-methylazetidinium, I⁻, 9-(N)-pyrrolidine, 9-(N)-N-methyl-pyrrolidinium, I⁻, 9-(N)-N-methyl-morpholinium, I⁻, 9-(N)-N-methylpiperazine, 9-(N)-N′-dimethylpiperazinium, I⁻, 9-NH-CBZ, 9-NHC(O)C₅H₁₁, 9-NHC(O)CH₂Br, 9-NH—C(NH)NH₂, 9-(2)-thiophene, 7-OHC₃, 8-OCH₃, 7-SCH₃, 8-SCH₃, and 6-OCH₃.

R^{2A} and R^{2B} an be selected from among substituted and unsubstituted C₁ to C₁₀ alkyl wherein the substituent(s) can be selected from among alkylcarbonyl, alkoxy, hydroxy, and nitrogen-containing heterocycles joined to the C₁ to C₁₀ alkyl through an ether linkage. Substituents at the 3-carbon can include ethyl, n-propyl, n-butyl, n-pentyl, isobutyl, isopropyl, —CH₂C(═O)C₂H₅, —CH₂OC₂H₅ and —CH₂O-(4-picoline). Ethyl, n-propyl, n-butyl, and isobutyl are preferred. In certain particularly preferred compounds of the present invention, substituents R^{2A} and R^{2B} are identical, for example n-butyl/n-butyl, so that the compound is achiral at the 3-carbon. Eliminating optical isomerism at the 3-carbon simplifies the selection, synthesis, separation, and quality control of the compound used as an ileal bile acid transport inhibitor. In both compounds having a chiral 3-carbon and those having an achiral 3-carbon, substituents (R⁶) on the benzo ring can include hydrogen, aryl, alkyl, hydroxy, halo, alkoxy, alkylthio, alkylsulfonyl, haloalkyl, haloalkoxy, (N)-hydroxy-carbonylalkyl amine, haloalkylthio, haloalkylsufinyl, haloalkylsufonyl, amino, N-alkylamino, N,N-dialkylamino, (N)-alkoxycarbamoyl, (N)-aryloxycarbamoyl, (N)-aralkyloxycarbamyoyl, trialkylammonium (especially with a halide counterion), (N)-amido, (N)-alkylamido, -N-alkylamido, -N,N-dialkylamido, (N)-haloalkylamido, (N)-sulfonamido, (N)-alkylsulfonamido, (N)-haloalkylsulfonamido, carboxyalkyl-amino, trialkylammonium salt, (N)-carbamic acid, alkyl or benzyl estr, N-acylamine, hydroxylamine, haloaylamine, carbohydrate, thiophene a trialkyl ammonium salt having a carboxylic acid or hydroxy substiuent on one or more of the alkyl substituents, an alkylene bridge having a quaternary ammonium salt substituted thereon, —[O(CH₂)_{w}]ₓ—X where x is 2 to 12, w is 2 or 3 and X is a halo or quaternary ammonium salt, and (N)-nitrogen containing heterocycle wherein the nitrogen of said heterocycle is optional quaternized. Among the preferred species which may constitute R⁶ are methyl, ethyl isopropyl, t-butyl, hydroxy, methoxy, ethoxy, isopropoxy, methylthio, iodo, bromo, fluoro, methylsulfinyl, methylsulfonyl, ethylthio, amino, hydroxylamine, N-methylamino, N,N-dimethylamino, N,N-diethylamino, (N)-benzyloxycarbamoyl, trimethylammonium, A, —NHC(═O)C₅H₁₁, —NHC(═O)C₆H₁₃, carboxyethylamino, (N)-morpholinyl, (N)-azetidinyl, (N)-N-methylazetidinium A, (N)-pyrrolidinyl, pyrrolyl, (N)-N-methylpyridinium A, (N)-N-methylmorpholinium A, and N-N′-methylpiperazinyl, (N)-bromomethylamido, (N)-N-hexylamino, thiophene, —N+(CH₃)₂CO₂H I⁻, —NCH₃CH₂CO₂H, —(N)-N′-dimethylpiperazinium I—, (N)-t-butyloxycarbamoyl, (N)-methylsulfonamido, (N)N′-methylpyrrolidinium, and —(OCH₂CH₂)₃I, where A is a pharmaceutically acceptable anion. The benzo ring is/can be mono-substituted at the 6, 7 or 8 position, or disubstituted at the 7- and -8 positions. Also included are the 6,7,8-trialkoxy compounds, for example the 6,7,8-trimethoxy compounds. A variety of other substituents can be advantageously present on the 6, 7, 8 and/or 9-positions of the benzo ring, includes, for example, guanidinyl, cycloalkyl, carbohydrate (e.g, a 5 or 6 carbon monosaccharide), peptide, and quaternary ammonium salts linked to the ring via poly(oxyalkylene) linkages, e.g., —(OCH₂CH₂)ₙ—N⁺R¹³R¹⁴R¹⁵A, where x is 2 to 10.

As various changes could be made in the above methods and apparatus without departing from the scope of the invention, it is intended that all matter contained in the above description be interpreted as illustrative and not in a limiting sense. All documents mentioned in this application are expressly incorporated by reference as if fully set forth at length.

All patents, publication or other references mentioned in this application are incorporated herein by reference in their entirety. When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

What we claim is:

1. A compound of Formula I: 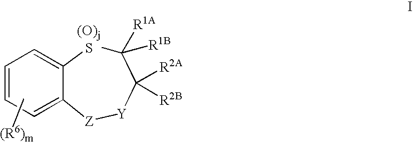
wherein:
j is 0, 1 or 2; and
m is 0, 1, 2, 3 or 4; and
R^{1A} and R^{1B} are independently selected from hydrogen and alkyl; and
R^{2A} and R^{2B} are independently selected from hydrogen, alkyl, alkenyl, alkynyl, cycloalkyl, cycloalkylalkyl, aryl and aralkyl; or
R^{2A} and R^{2B} together with the carbon atom to which they are attached form a C₃₋₁₀ cycloalkyl group; and
one of Z and Y is NR³ and the other of Z and Y is CHR⁴;
wherein R³ and R⁴ are independently selected from the group consisting of hydrogen, oxo, acyl, thioacyl, and R⁵; and
wherein R⁵ is selected from the group consisting of alkyl; cycloalkyl; alkenyl; alkynyl; aryl; heterocyclyl; quaternary heterocyclyl; —OR⁹; —SR⁹; —S(O)R⁹; —SO₂R⁹; and —SO₃R⁹;
wherein the R⁵ alkyl; cycloalkyl; alkenyl; alkynyl; aryl; heterocyclyl; and quaternary heterocyclyl radical is substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³OR¹⁴; —NR¹³NR¹⁴R¹⁵; —CO₂R¹³—OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ radical optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A-; —P(O)R⁷R⁸; —PR⁷R⁸; —P⁺E⁷R⁸R⁹A⁻; and —P(O)(OR⁷)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ radical optionally may have one or more carbons replaced by —O—; —NR⁷—; —N⁺R⁷R⁸A⁻; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A⁻; —S⁺R⁹R¹⁰A⁻; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable cation and M is a pharmaceutically acceptable cation; and
one or more R⁶ radicals are independently selected from the group consisting of R⁵, hydrogen; halogen; —CN; —NO₂; alkyl; cycloalkyl; polyalkyl; haloalkyl; hydroxyalkyl; alkenyl; alkynyl; aryl; heterocyclyl; quaternary heterocyclyl; arylalkyl; heterocyclylalkyl; polyether; acyloxy; —OR¹³; —NR¹³R¹⁴; —SR¹³; —S(O)R¹³; —S(O)₂R¹³; —SO₃R¹³; —S⁺R¹³R¹⁴A⁻; —NR¹³OR¹⁴; —NR¹³NR¹⁴R¹⁵; —CO₂R¹³; —OM; —SO₂OM; —SO₂NR¹³R¹⁴; —NR¹⁴C(O)R¹³; —C(O)NR¹³R¹⁴; —C(O)OM; —COR¹³; OR¹⁸; —S(O)NR¹³R¹⁴; —NR¹³R¹⁸; —NR¹⁸OR¹⁴; —N⁺R¹³R¹⁴R¹⁵A⁻; PR¹³R¹⁴; —P⁺R¹³R¹⁴R¹⁵A⁻; amino acid residue; peptide residue; polypeptide residue; and carbohydrate residue;
wherein the R⁶ alkyl; cycloalkyl; polyalkyl; haloalkyl; hydroxyalkyl; alkenyl; alkynyl; aryl; heterocyclyl; arylalkyl; heterocyclylalkyl; polyether; acyloxy radicals optionally may be further substituted with one or more radicals selected from the group consisting of halogen; —CN; oxo; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹¹R¹²A⁻; —S⁺R⁹R¹¹R¹⁰A⁻; and carbohydrate residue; and
wherein the R⁶ quaternary heterocyclyl radical optionally may be substituted with one or more radicals selected from the group consisting of halogen; —CN; —NO₂; oxo; alkyl; cycloalkyl; polyalkyl; haloalkyl; hydroxyalkyl; alkenyl; alkynyl; aryl; heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R¹³ OM; —SO₂OM; —SO₂NR¹³R¹⁴; —C(O)NR¹³R¹⁴; —C(O)OM; —COR¹³; —P(O)R¹³R¹⁴; —P¹³R¹⁴; —P⁺R¹³ R¹⁴R¹⁵A⁻; —P(OR¹³)OR¹⁴; —S⁺R¹³R¹⁴A⁻; —N⁺R¹³R¹⁴R¹⁵A⁻; and carbohydrate residue; and
wherein the R⁶ radicals comprising carbon optionally may have one or more carbons replaced by —O—; —NR¹³—; —N⁺R¹³R¹⁴A⁻-; —S—; —SO—; —SO₂—; —S⁺R¹³A⁻-; —PR¹³—; —P(O)R¹³—; —PR¹³R¹⁴; —P⁺R¹³R¹⁴A⁻-; phenylene; amino acid residue; peptide residue; polypeptide residue; carbohydrate residue; polyether; or polyalkyl; wherein said phenylene; amino acid residue; peptide residue; polypeptide residue; carbohydrate residue; and polyalkyl optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; or —P(O)R⁹—; and
wherein R¹⁸ is selected from the group consisting of alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl; and
wherein the R¹⁸ alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl radicals optionally may be substituted with one or more radicals selected from the group consisting of halogen; —CN; NO₂; oxo; —OR⁹; —NR⁹R¹⁰; —N⁺R⁹R¹¹R¹²A⁻; —SR⁹; —S(O)R⁹; —SO₂R⁹; —SO₃R⁹; —CO₂R⁹; —CONR⁹R¹⁰; —SO₂OM; —SO₂NR⁹R¹⁰; —PR⁹R¹⁰; —P(OR¹³)OR¹⁴; —PO(OR¹⁶)OR¹⁷; and —C(O)OM; or
a pharmaceutically acceptable salt, solvate, or prodrug thereof;
provided that at least one of R³, R⁴ and R⁶ is R⁵; and
provided that at least one of the following conditions is satisfied:
(a) the R⁵ moiety possesses an overall positive charge;
(b) the R⁵ moiety comprises a quaternary ammonium group or a quaternary amine salt;
(c) the R⁵ moiety comprises a phosphonic acid group or at least two carboxyl groups; or
(d) the R⁵ moiety comprises a polyethylene glycol group having a molecular weight of at least 1000.

2. A compound of claim 1 wherein R is aryl substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³ CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ aryl optionally may be further substituted with one or more radicals selected from the group consisting of-CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸—N⁺R⁷R⁸R⁹A⁻; —P(O)R⁷R⁸; —PR⁷R⁸; —P⁺R⁷R⁸R⁹A⁻; and —P(O)(OR⁷)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ aryl optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸ A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO²R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A⁻; —S⁺R⁹R¹⁰A-; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable anion and M is a pharmaceutically acceptable cation.

3. A compound of claim 2 wherein R⁵ is: 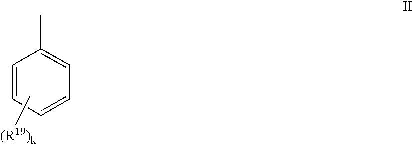
wherein
k is 0, 1, 2, 3 or 4; and
one or more R¹⁹ are independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —SO²R¹³; —SO₃R¹³; —NR¹³OR¹⁴; —NR¹³NR¹⁴R¹⁵—; —CO₂R¹³; —OM; —SO₂OM; —SO₂NR¹³R¹⁴—C(O)NR¹³R¹⁴; —C(O)OM; —COR¹³; —NR¹³C(O)R¹⁴; —NR¹³C(O)NR¹⁴R¹⁵, NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and
wherein the R¹⁹ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A-; P(O)R⁷R⁸; PR⁷R⁸; —P⁺R⁷R⁸R⁹A⁻; and —P(O)(OR⁷)OR⁸; and
wherein the R¹⁹ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A-; —S⁺R⁹R¹⁰A-; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable anion and M is a pharmaceutically acceptable cation.

4. A compound of claim 3 wherein R⁵ is: 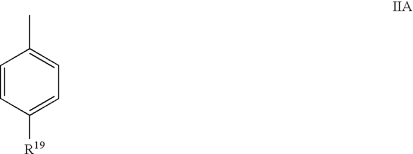
wherein R¹⁹ is as defined in claim 3.

5. A compound of claim 3 wherein R⁵ is: 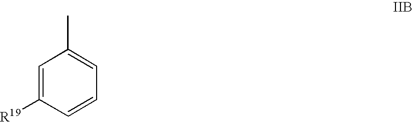
wherein R¹⁹ is as defined in claim 3.

6. A compound of claim 3 wherein:
R³ is R⁵; and
R⁴ is selected from the group consisting of hydrogen and alkyl.

7. A compound of claim 3 wherein:
R³ is selected from the group consisting of hydrogen and alkyl; and
R⁴ is R⁵.

8. A compound of claim 3 wherein:
R³ is R⁵; and
R⁴ is selected from the group consisting of hydrogen; oxo; alkyl; cycloalkyl; aryl; heterocyclyl; acyl, thioacyl, and —OR⁹;
wherein the R⁴ alkyl; cycloalkyl; aryl; heterocyclyl radical is substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³ R¹⁴; —SR¹³; —S(O)R¹³; —SO₂R¹³; —SO₃R¹³; —NR¹³OR¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁴ radical optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A-; —P(O)R⁷R⁸; —PR⁷R⁸; —P⁺R⁷R⁸R⁹A⁻; and —P(O)(OR⁷)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁴ radical optionally may have one or more carbons replaced by —O—; —NR⁷; —N⁺R⁷R⁸A⁻-; —S—; —SO—; —SO₂—; —S⁺R⁷A⁻; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹ R¹⁰R¹¹A-; —S⁺R⁹R¹⁰A-; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable cation and M is a pharmaceutically acceptable cation.

9. A compound of claim 3 wherein:
R³ is selected from the group consisting of hydrogen; oxo; alkyl; cycloalkyl; aryl; heterocyclyl; acyl, thioacyl, and —OR⁹;
wherein the R³ alkyl; cycloalkyl; aryl; heterocyclyl radical is substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R¹³; —OM, —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³ radical optionally may be further substituted with one or more radicals selected from the group consisting of —CN; halogen; hydroxy; oxo; alkyl; cycloalkyl; alkenyl; alknyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷—CONR⁷R⁸; —N⁺R⁷R⁸R⁹A-; —P(O)R⁷R⁸; —PR⁷R⁸; —P⁺R⁷R⁸R⁹A⁻; and —P(O)(OR⁷)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³ radical optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —PR⁹R¹⁰R¹¹A-; —S⁺R⁹R¹⁰A⁻; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable cation and M is a pharmaceutically acceptable cation; and
R⁴ is R⁵.

10. A compound of claim 3 wherein:
R¹⁹ is independently selected from the group consisting of —OR¹³, —NR¹³R¹⁴, —NR¹³C(O)R¹⁴, —OC(O)NR¹³R¹⁴, and —NR¹³SO₂R¹⁴, and
wherein R¹³, R¹⁴, and R¹⁵ are independently selected from the group consisting of alkyl, polyether, aryl, quaternary heterocycle, arylalkyl, heterocyclylalkyl, quaternary heterocyclylalkyl, alkylheterocyclylalkyl, and alkylammoniumalkyl,
wherein alkyl optionally has one or more carbons replaced by O or N⁺R⁹R¹⁰A-, and
wherein R¹³, R¹⁴, and R¹⁵ are optionally substituted with one or more groups selected from the group consisting of hydroxy, carboxy, alkyl, quaternary heterocyclylalkyl, —SR⁹, —S(O)R⁹, —S(O)₂R⁹, —S(O)₃R⁹, —NR⁹R¹⁰, —N⁺R⁹R¹¹R¹²A⁻, —CONR⁹R¹⁰, and —PO(OR¹⁶)OR¹⁷, and
wherein R⁹ and R¹⁰ are independently selected from the group consisting of hydrogen, alkyl, heterocyclylalkyl, carboxyalkyl, carboalkoxyalkyl, and carboxyalkylheterocycle; and
wherein R¹¹ and R¹² are independently alkyl; and
wherein A⁻ is a pharmaceutically acceptable anion and M is a pharmaceutically acceptable cation.

11. A compound of claim 3 wherein:
R¹⁹ is independently selected from the group consisting of —OR¹³, —NR¹³R¹⁴, —NR¹³C(O)R¹⁴, —OC(O)NR¹³R¹⁴, and —NR¹³ SO₂R¹⁴, and
wherein R¹³, R¹⁴, and R¹⁵ are independently selected from the group consisting of polyether, aryl, quaternary heterocycle, arylalkyl, heterocyclylalkyl, quaternary heterocyclylalkyl, and alkylheterocyclylalkyl,
wherein R¹³, R¹⁴, and R¹⁵ are optionally substituted with one or more groups selected from the group consisting of hydroxy, carboxy, alkyl, quaternary heterocyclylalkyl, —SR⁹, —S(O)R⁹, —S(O)₂R⁹, —S(O)₃R⁹, —NR⁹R¹⁰, —N⁺R⁹R¹¹R¹²A⁻, —CONR⁹R¹⁰, and —PO(OR¹⁶)OR¹⁷ and
wherein R⁹ and R¹⁰ are independently selected from the group consisting of hydrogen, alkyl, heterocyclylalkyl, carboxyalkyl, carboalkoxyalkyl, and carboxyalkylheterocycle; and
wherein R¹¹ and R¹² are independently alkyl; and
wherein A⁻ is a pharmaceutically acceptable anion and M is a pharmaceutically acceptable cation.

12. A compound of claim 10 wherein R⁵ is: 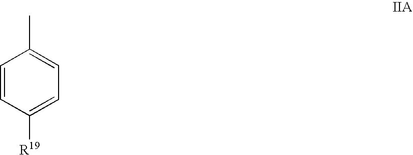
wherein R¹⁹ is as defined in claim 10.

13. A compound of claim 10 wherein R⁵ is: 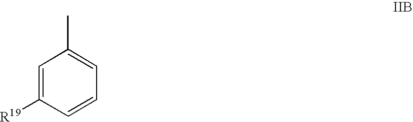
wherein R¹⁹ is as defined in claim 10.

14. A compound of claim 10 wherein R¹⁹ is selected from the group consisting of:

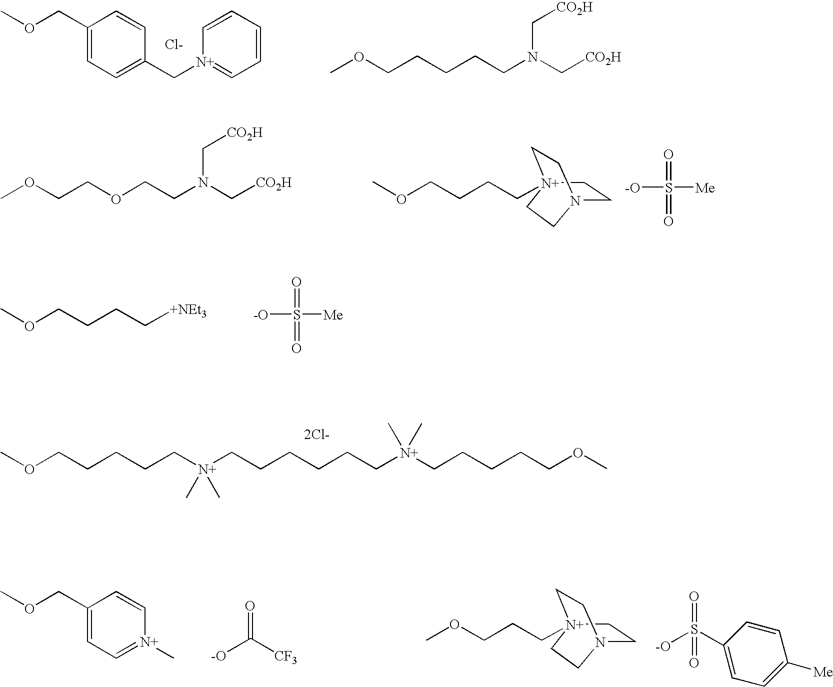

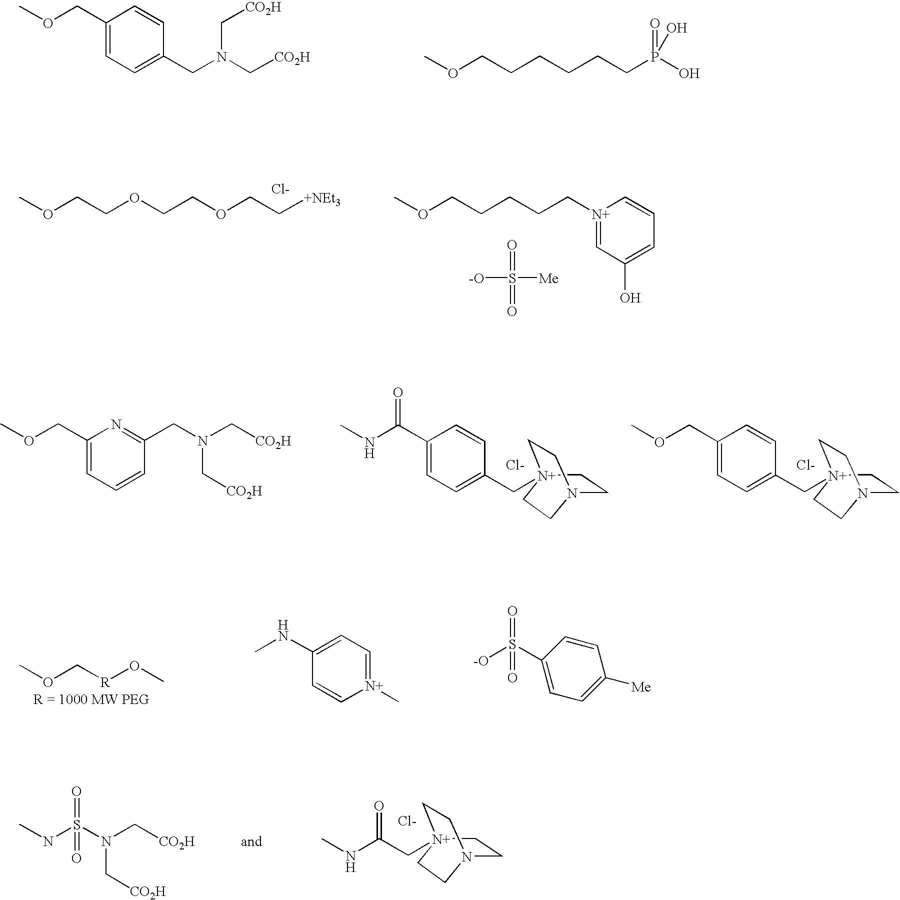

15. A compound of claim 3 wherein:
j is 2;
R^{1A} and R^{1B} are independently selected from hydrogen and alkyl; and
R^{2A} and R^{2B} are independently selected from hydrogen and alkyl.

16. A compound of claim 3 wherein:
j is 2;
R^{1A} and R^{1B} are hydrogen; and
R^{2A} and R^{2B} are independently selected from alkyl.

17. A compound of claim 3 wherein:
j is 2;
R^{1A} and R^{1B} are hydrogen; and
R^{2A} and R^{2B} are independently selected from ethyl, propyl and butyl.

18. A compound of claim 3 wherein j is 1 or 2.

19. A compound of claim 3 wherein j is 2.

20. A compound of claim 3 wherein R^{1A} and R^{1B} are hydrogen.

21. A compound of claim 3 wherein R^{2A} and R^{2B} are independently selected from the group consisting of hydrogen and C₁₋₆alkyl.

22. A compound of claim 3 wherein R^{2A} and R^{2B} are independently selected from the group consisting C₁₋₆alkyl.

23. A compound of claim 3 wherein R^{2A} and R^{2B} are the same alkyl.

24. A compound of claim 3 wherein R^{2A} and R^{2B} are each n-butyl.

25. A compound of claim 3 wherein one of R^{2A} and R^{2B} is ethyl and the other of
R^{2A} and R^{2B} is n-butyl.

26. A compound of claim 3 wherein one or more R⁶ are independently selected from methoxy and dimethylamino.

27. A compound of claim 3 wherein
j is 1 or 2;
R^{1A} and R^{1B} are hydrogen;
R^{2A} and R^{2B} are n-butyl; and
one or more R⁶ are independently selected from methoxy and dimethylamino.

28. A compound of claim 3 wherein
j is 1 or 2;
R^{1A} and R^{1B} are hydrogen;
one of R^{2A} and R^{2B} is ethyl and the other of R^{2A} and R^{2B} is n-butyl; and
one or more R⁶ are independently selected from methoxy and dimethylamino.

29. A compound of claim 1 corresponding to Formula IA: 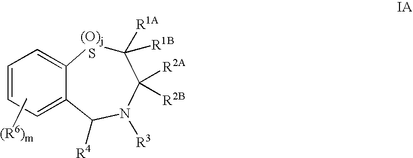
wherein:
j is 0, 1 or 2; and
m is 0, 1, 2, 3 or 4; and
R^{1A} and R^{1B} are independently selected from hydrogen and alkyl; and
R^{2A} and R^{2B} are independently selected from hydrogen, alkyl, alkenyl, alkynyl, cycloalkyl, cycloalkylalkyl, aryl, and aralkyl; or
R^{2A} and R^{2B} together with the carbon atom to which they are attached form a C₃₋₇ cycloalkyl group; and
R³ and R⁴ are independently selected from the group consisting of hydrogen, oxo, acyl, thioacyl, and R⁵; and
wherein R⁵ is selected from the group consisting of alkyl; cycloalkyl; alkenyl; alkynyl; aryl; heterocyclyl; quaternary heterocyclyl; —OR⁹; —SR⁹; —S(O)R⁹; —SO₂R⁹; and —SO₃R⁹;
wherein the R⁵ alkyl; cycloalkyl; alkenyl; alkynyl; aryl; heterocyclyl; and quaternary heterocyclyl radical is substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ radical optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A-; —P(O)R⁷R⁸; —PR⁷R⁸; —P⁺R⁷R⁸R⁹A⁻; and —P(O)(OR⁷)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ radical optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A⁻; —S⁺R⁹R¹⁰A-; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable anion and M is a pharmaceutically acceptable cation; and
one or more R⁶ radicals are independently selected from the group consisting of R⁵, hydrogen; halogen; —CN; —NO₂; alkyl; cycloalkyl; polyalkyl; haloalkyl; hydroxyalkyl; alkenyl; alkynyl; aryl; heterocyclyl; quaternary heterocyclyl; arylalkyl; heterocyclylalkyl; polyether; acyloxy; —OR¹³; —NR¹³R¹⁴; —SR¹³; —S(O)R¹³; —S(O)₂R¹³; —SO₃R¹³; —S⁺R¹³R¹⁴A⁻; —NR¹³OR¹⁴; —NR¹³NR¹⁴R¹⁵; —CO₂R¹³; —OM; —SO₂OM; —SO₂NR¹³R¹⁴; —NR¹⁴C(O)R¹³; —C(O)NR¹³R¹⁴; —C(O)OM; —COR¹³; —OR¹⁸; —S(O)NR¹³R¹⁴; —NR¹³R¹⁸; —NR¹⁸; R¹⁴; —N⁺R¹³R¹⁴R¹⁵A⁻; —PR¹³R¹⁴; —P(O)R¹³R¹⁴; —P⁺R¹³R¹⁴R¹⁵A⁻; amino acid residue; peptide residue; polypeptide residue; and carbohydrate residue;
wherein the R⁶ alkyl; cycloalkyl; polyalkyl; haloalkyl; hydroxyalkyl; alkenyl; alkynyl; aryl; heterocyclyl; arylalkyl; heterocyclylalkyl; polyether; acyloxy radicals optionally may be further substituted with one or more radicals selected from the group consisting of halogen; —CN; oxo; —OR⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹¹R¹²A⁻; —S⁺R⁹R¹⁰A⁻; and carbohydrate residue; and
wherein the R⁶ quaternary heterocyclyl radical optionally may be substituted with one or more radicals selected from the group consisting of halogen; —CN; —NO₂; oxo; alkyl; cycloalkyl; polyalkyl; haloalkyl; hydroxyalkyl; alkenyl; alkynyl; aryl; heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR⁴; —NR¹³NR¹⁴R¹⁵; —CO₂R¹³; OM; —SO₂OM; —SO₂NR¹³R¹⁴; —C(O)NR¹³R¹⁴; —C(O)OM; —COR¹³; —P(O)R¹³R¹⁴; —P¹³R¹⁴ P⁺R¹³ R¹⁴R¹⁵A⁻; —P(OR¹³)OR¹⁴; —S⁺R¹³R¹⁴A⁻; —N⁺R¹³R¹⁴R¹⁵A⁻; d carbohydrate residue; and
wherein the R⁶ radicals comprising carbon optionally may have one or more carbons replaced by —O—; —NR¹³—; —N¹³R¹⁴A⁻-; —S—; —SO—; —SO₂—; —S⁺R¹³A⁻-; —PR¹³—; —P(O)R¹³—; —PR¹³R¹⁴; —P⁺R¹³R¹⁴A⁻-; phenylene; amino acid residue; peptide residue; polypeptide residue; carbohydrate residue; polyether; or polyalkyl; wherein said phenylene; amino acid residue; peptide residue; polypeptide residue; carbohydrate residue; and polyalkyl optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; or —P(O)R⁹—; and
wherein R¹⁸ is selected from the group consisting of alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl; and
wherein the R¹⁸ alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl radicals optionally may be substituted with one or more radicals selected from the group consisting of halogen; —CN; NO₂; oxo; —OR⁹; —NR⁹R¹⁰; —N⁺R⁹R¹¹R¹²A⁻; —SR⁹; —S(O)R⁹; —SO₂R⁹; —SO₃R⁹; —CO₂R⁹; —CONR⁹R¹⁰; —SO₂OM; —SO₂NR⁹R¹⁰; —PR⁹R¹⁰; —P(OR¹³)OR¹⁴; —PO(OR¹⁶)OR¹⁷; and —C(O)OM; or
a pharmaceutically acceptable salt, solvate, or prodrug thereof;
provided that at least one of R³, R⁴ and R⁶ is R⁵; and
provided that the R⁵ alkyl, cycloalkyl, aryl, heterocyclyl, and —OR⁹ radicals are not substituted with —O(CH₂)₁₋₄NR′R″R′″ wherein R′, R″ and R′″ are independently selected from hydrogen and alkyl; and
provided that at least one of the following conditions is satisfied:
(a) the R⁵ moiety possesses an overall positive charge; and/or
(b) the R⁵ moiety comprises a quaternary ammonium group or a quaternary amine salt; and/or
(c) the R⁵ moiety comprises at least two carboxy groups.

30. A compound of claim 29 wherein R⁵ is aryl substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³; —SR¹⁴; —S(O)R¹³; —SO₂R¹³; —SO₃ R¹³; —NR¹³OR¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³ SO₂R¹⁴; —NR¹³SONR¹⁴R¹⁵; —NR¹³SO₂NR¹⁴R¹⁵; —PR¹³R¹⁴; —P(O)R¹³ R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ aryl optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A⁻; —P(O)R⁷R⁸; —PR⁷R⁸; —P⁺R⁷R⁸R⁹A⁻; and —P(O)(R 7)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ aryl optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸ A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; OR¹⁶; —NR⁹R¹⁰; —N⁺R⁹R¹⁰R^{w}A⁻; —SR¹⁶; —S(O)R⁹; —SO₂R⁹; —SO₃R¹⁶—CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A-; —S⁺R⁹R¹⁰A-; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable anion and M is a pharmaceutically acceptable cation.

31. A compound of claim 30 wherein R⁵ is: 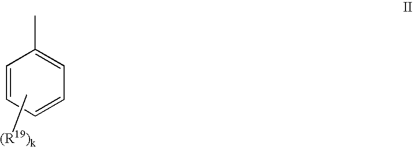
wherein
k is 0, 1, 2, 3 or 4; and
one or more R¹⁹ are independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; SR¹³; —O₂R¹³; —SO₃R¹³; —NR¹³OR¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and
wherein the R¹⁹ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may be further substituted with one or more radicals selected from the group consisting of-CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷, R⁸R⁹A-; —P(O)R⁷R⁸; —PR⁷R⁸; —P⁺R⁷R⁸R⁹A⁻; and —P(O)(OR⁷)OR⁸; and
wherein the R¹⁹ alkyl, polyalkyl, haloalkyl, -hydroxyalkyl, cycloalkyl, alkenyl, alkynyl, aryl, heterocyclyl, quaternary heterocyclyl, arylalkyl, heterocyclylalkyl, and polyether radicals optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and.
wherein R and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; CONR⁹R¹⁰; —SO₂NR⁹R¹⁰; —PO(OR¹⁶)OR¹⁷; —P⁹R¹⁰; —P⁺R⁹R¹⁰R¹¹A-; —S⁺R⁹R¹⁰A⁻; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable anion and M is a pharmaceutically acceptable cation.

32. A compound of claim 31 wherein R⁵ is: 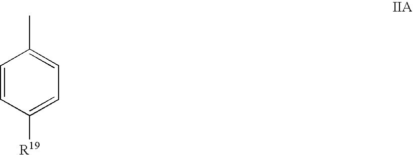
wherein R¹⁹ is as defined in claim 31.

33. A compound of claim 31 wherein R⁵ is: 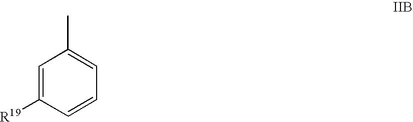
wherein R⁹ is as defined in claim 31.

34. A compound of claim 31 wherein:
R³ is R¹³ and
R⁴ is selected from the group consisting of hydrogen and alkyl.

35. A compound of claim 31 wherein:
R³ is selected from the group consisting of hydrogen and alkyl; and
R⁴ is R⁵.

36. A compound of claim 31 wherein:
R³ is R⁵; and
R⁴ is selected from the group consisting of hydrogen; oxo; alkyl; cycloalkyl; aryl; heterocyclyl; acyl, thioacyl, and —OR⁹
wherein the R⁴ alkyl; cycloalkyl; aryl; heterocyclyl radical is substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; NR¹³OR¹⁴ NR¹³NR¹⁴R¹⁵; —CO₂R¹³; —OM; —SO₂OM; —SO₂NR¹³R¹⁴; —C(O)NR¹³R¹⁴; —C(O)OM; —CO¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R₁₅A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁴ radical optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R; —SO₂R; —SO₃R⁷; —CO₂R⁷; —CONR⁷R⁸; —N⁺R⁷R⁸R⁹A-; —P(O)R⁷R⁸; —PR⁷R⁸; —P⁺R⁷R⁸R⁹A⁻; and —P(O)(R 7)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁴ radical optionally may have one or more carbons replaced by —O—; —NR⁷—; —N R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³ R¹⁴ and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A-; —S⁺R⁹R¹⁰A-; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable cation and M is a pharmaceutically acceptable cation.

37. A compound of claim 31 wherein:
R³ is selected from the group consisting of hydrogen; oxo; alkyl; cycloalkyl; aryl; heterocyclyl; acyl, thioacyl, and —OR⁹
wherein the R³ alkyl; cycloalkyl; aryl; heterocyclyl radical is substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³ radical optionally may be further substituted with one or more radicals selected from the group consisting of-CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A-; —P(O)R⁷R⁸; —PR⁷R⁸; —P⁺R⁷R⁸R⁹A⁻; and —P(O)(OR⁷)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³ radical optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹;—SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A⁻; —S⁺R⁹R¹⁰A⁻; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable cation and M is a pharmaceutically acceptable cation; and
R⁴ is R⁵.

38. A compound of claim 31 wherein:
R¹⁹ is independently selected from the group consisting of —OR¹³, —NR¹³R¹⁴, —NR¹³C(O)R¹⁴, —OC(O)NR¹³R¹⁴, and —NR¹³ SO₂R¹⁴, and
wherein R¹³, R¹⁴, and R¹⁵ are independently selected from the group consisting of alkyl, polyether, aryl, quaternary heterocycle, arylalkyl, heterocyclylalkyl, quaternary heterocyclylalkyl, alkylheterocyclylalkyl, and alkylammoniumalkyl,
wherein alkyl optionally has one or more carbons replaced by O or N⁺R⁹R¹⁰A-and
wherein R¹³, R¹⁴, and R¹⁵ are optionally substituted with one or more groups selected from the group consisting of hydroxy, carboxy, alkyl, quaternary heterocyclylalkyl, —SR⁹, —S(O)R⁹, —S(O)₂R⁹, —S(O)₃R⁹, —NR⁹R¹⁰, —N⁺R⁹R¹¹ R¹²A⁻, —CONR⁹R¹⁰, and —PO(OR¹⁶)OR¹⁷ and
wherein R⁹ and R¹⁰ are independently selected from the group consisting of hydrogen, alkyl, heterocyclylalkyl, carboxyalkyl, carboalkoxyalkyl, and carboxyalkylheterocycle; and
wherein R¹¹ and R¹² are independently alkyl; and
wherein A⁻ is a pharmaceutically acceptable anion and M is a pharmaceutically acceptable cation.

39. A compound of claim 31 wherein:
R¹⁹ is independently selected from the group consisting of —OR¹³—NR¹³R¹⁴, —NR¹³C(O)R¹⁴, —OC(O)NR¹³R¹⁴, and —NR¹³SO₂R¹⁴ and
wherein R¹³, R¹⁴, and R¹⁵ are independently selected from the group consisting of polyether, aryl, quaternary heterocycle, arylalkyl, heterocyclylalkyl, quaternary heterocyclylalkyl, and alkylheterocyclylalkyl,
wherein alkyl optionally has one or more carbons replaced by O or N⁺R⁹R¹⁰A-, and
wherein R¹³, R¹⁴, and R¹⁵ are optionally substituted with one or more groups selected from the group consisting of hydroxy, carboxy, alkyl, quaternary heterocyclylalkyl, —SR⁹, —S(O)R⁹, —S(O)₂R⁹, —S(O)₃R⁹, —NR⁹R¹⁰, —N⁺R⁹R¹¹R¹²A⁻, —CONR⁹R¹⁰ and —PO(OR¹⁶)OR¹⁷, and
wherein R⁹ and R¹⁰ are independently selected from the group consisting of hydrogen, alkyl, heterocyclylalkyl, carboxyalkyl, carboalkoxyalkyl, and carboxyalkylheterocycle; and
wherein R¹¹ and R¹² are independently alkyl; and
wherein A⁻ is a pharmaceutically acceptable anion and M is a pharmaceutically acceptable cation.

40. A compound of claim 38 wherein R⁵ is: 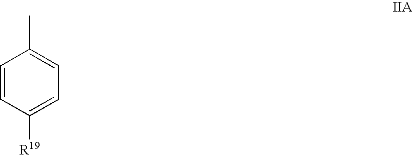
wherein R¹⁹ is as defined in claim 38.

41. A compound of claim 38 wherein R⁵ is: 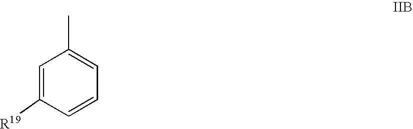
wherein R¹⁹ is as defined in claim 38.

42. A compound of claim 38 wherein R¹⁹ is selected from the group consisting of:

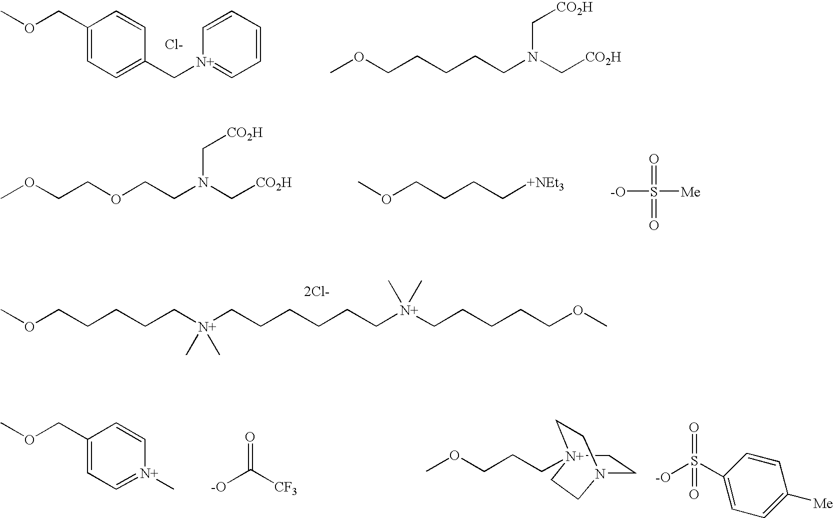

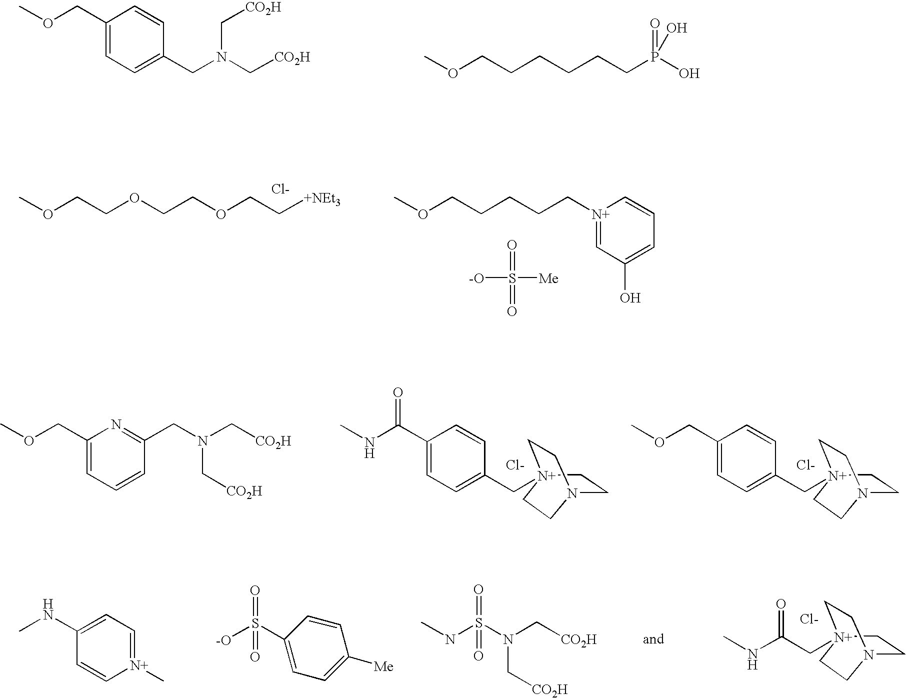

43. A compound of claim 38 wherein:
j is 2;
R^{1A} and R^{1B} are independently selected from hydrogen and alkyl; and
R^{2A} and R^{2B} are independently selected from hydrogen and alkyl.

44. A compound of claim 38 wherein:
j is 2;
R^{1A} and R^{2A} are hydrogen; and
R^{2A} and R^{2B} are independently selected from alkyl.

45. A compound of claim 38 wherein:
j is 2;
R^{1A} and R^{1B} are hydrogen; and
R^{2A} and R^{2B} are independently selected from ethyl, propyl and butyl.

46. A compound of claim 38 wherein:
j is 2;
R^{1A} and R^{1B} are hydrogen;
R^{2A} and R^{2B} are independently selected from ethyl, propyl and butyl;
R³ is R⁵; and
R⁴ is selected from hydrogen and alkyl.

47. A compound of claim 38 wherein:
j is 2;
R^{1A} and R^{1B} are hydrogen;
R^{2A} and R^{2B} are independently selected from ethyl, propyl and butyl;
R³ is selected from from hydrogen and alkyl; and
R⁴ is R⁵.

48. A compound of claim 38 wherein j is 1 or 2.

49. A compound of claim 38 wherein j is 2.

50. A compound of claim 38 wherein R^{1A} and R^{1B} are hydrogen.

51. A compound of claim 38 wherein R^{2A} and R^{2B} are independently selected from the group consisting of hydrogen and C₁₋₆alkyl.

52. A compound of claim 38 wherein R^{2A} and R^{2B} are independently selected from the group consisting C₁₋₆ alkyl.

53. A compound of claim 38 wherein R^{2A} and R^{2B} are the same alkyl.

54. A compound of claim 38 wherein R^{2A} and R^{2B} are each n-butyl.

55. A compound of claim 38 wherein one of R^{2A} and R^{2B} is ethyl and the other of R^{2A} and R^{2B} is n-butyl.

56. A compound of claim 38 wherein one or more R⁶ are independently selected from methoxy and dimethylamino.

57. A compound of claim 38 wherein
j is 1 or 2;
R^{1A} and R^{1B} are hydrogen;
R^{2A} and R^{2B} are n-butyl; and
one or more R⁶ are independently selected from methoxy and dimethylamino.

58. A compound of claim 38 wherein
j is 1 or 2;
R^{1A} and R^{1B} are hydrogen;
one of R^{2A} and R^{2B} is ethyl and the other of R^{2A} and R^{2B} is n-butyl; and
one or more R⁶ are independently selected from methoxy and dimethylamino.

59. A compound of claim 42 wherein:
j is 2;
R^{1A} and R^{1B} are independently selected from hydrogen and alkyl; and
R^{2A} and R^{2B} are independently selected from hydrogen and alkyl.

60. A compound of claim 42 wherein:
j is 2;
R^{1A} and R^{1B} are hydrogen; and
R^{2A} and R^{2B} are independently selected from alkyl.

61. A compound of claim 42 wherein:
j is 2;
R^{1A} and R^{1B} are hydrogen; and
R^{2A} and R^{2B} are independently selected from ethyl, propyl and butyl.

62. A compound of claim 42 wherein:
j is 2;
R^{1A} and R^{1B} are hydrogen;
R^{2A} and R^{2B} are independently selected from ethyl, propyl and butyl;
R³ is R⁵; and
R⁴ is selected from hydrogen and alkyl.

63. A compound of claim 42 wherein:
j is 2;
R^{1A} and R^{1B} are hydrogen;
R^{2A} and R^{2B} are independently selected from ethyl, propyl and butyl;
R³ is selected from from hydrogen and alkyl; and
R⁴ is R⁵.

64. A compound of claim 42 wherein j is 1 or 2.

65. A compound of claim 42 wherein j is 2.

66. A compound of claim 42 wherein R^{1A} and R^{1B} are hydrogen.

67. A compound of claim 42 wherein R^{2A} and R^{2B} are independently selected from the group consisting of hydrogen and C₁₋₄alkyl.

68. A compound of claim 42 wherein R^{2A} and RB are independently selected from the group consisting C₁₋₆alkyl.

69. A compound of claim 42 wherein R^{2A} and R^{2B} are the same alkyl.

70. A compound of claim 42 wherein R^{2A} and R^{2B} are each n-butyl.

71. A compound of claim 42 wherein one of R^{2A} and R^{2B} is ethyl and the other of R^{2A} and R^{2B} is n-butyl.

72. A compound of claim 42 wherein one or more R⁶ are independently selected from methoxy and dimethylamino.

73. A compound of claim 42 wherein
j is 1 or 2;
R^{1A} and R^{1B} are hydrogen;
R^{2A} and R^{2B} are n-butyl; and
one or more R⁶ are independently selected from methoxy and dimethylamino.

74. A compound of claim 42 wherein
j is 1 or 2;
R^{1A} and R^{1B} are hydrogen;
one of R^{2A} and R^{2B} is ethyl and the other of R^{2A} and R^{2B} is n-butyl; and
one or more R⁶ are independently selected from methoxy and dimethylamino.

75. A compound of claim 1 corresponding to Formula IB: 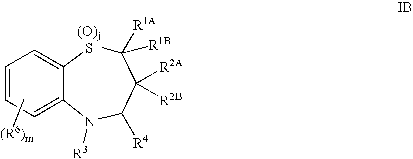
wherein:
j is 0, 1 or 2; and
m is 0, 1, 2, 3 or 4; and
R^{1A} and R^{1B} are independently selected from hydrogen and alkyl; and
R^{2A} and R^{2B} are independently selected from hydrogen, alkyl, alkenyl, alkynyl, cycloalkyl, cycloalkylalkyl, aryl, and aralkyl; or
R^{2A} and R^{2B} together with the carbon atom to which they are attached form a C₃₋₇ cycloalkyl group; and
R³ and R⁴ are independently selected from the group consisting of hydrogen, oxo, acyl, thioacyl and R⁵; and
wherein R⁵ is selected from the group consisting of alkyl; cycloalkyl; alkenyl; alkynyl; aryl; heterocyclyl; quaternary heterocyclyl; —OR⁹; —SR⁹; —S(O)R⁹; —SO₂R⁹; and —SO₃R⁹;
wherein the R alkyl; cycloalkyl; alkenyl; alkynyl; aryl; heterocyclyl; and quaternary heterocyclyl radical is substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R³; —SO₂R¹³; —SO₃R¹³; —NR¹³OR¹⁴; —NR¹³NR¹⁴R¹⁵; —CO₂R¹³—OM SO OM; —SO₂NR¹³ R⁴; —C(O)NR¹³R¹⁴; —C(O)OM; —COR¹³; —NR¹³C(O)R¹⁴; —NR³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A; —P(R¹³)OR¹⁴; —S⁺R¹³R¹⁴A⁻; and —N⁺R¹³R¹⁴R¹⁵ d
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ radical optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A-; —P(O)R⁷R⁸; —P⁺R⁷R⁸R⁹A⁻; and —P(O)(OR⁷)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ radical optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹)OR¹⁷; —P⁹R¹⁰; —S⁺R⁹R¹⁰A⁻; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P R⁹R¹A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable anion and M is a pharmaceutically acceptable cation; and
one or more R⁶ radicals are independently selected from the group consisting of R⁵, hydrogen; halogen; —CN; —NO₂; alkyl; cycloalkyl; polyalkyl; haloalkyl; hydroxyalkyl; alkenyl; alkynyl; aryl; heterocyclyl; quaternary heterocyclyl; arylalkyl; heterocyclylalkyl; polyether; acyloxy; —OR¹³; —NR¹³R¹⁴; —SR¹³; —S(O)R¹³; —S(O)₂R¹³; —SO₃R¹³; —S⁺R¹³R¹⁴A⁻; —NR¹³OR¹⁴; —NR¹³NR¹⁴R¹⁵; —CO₂R³; —OM; —SO₂OM; —SO₂NR¹³R¹⁴; —NR¹⁴C(O)R¹³; —C(O)NR R¹⁴; —C(O)OM; —COR¹³; OR¹⁴; —S(O)NR¹³R¹⁴; —NR¹³R¹⁴; —NR¹³OR¹⁴; —N⁺R¹³R¹⁴R¹⁵A⁻-; —PR¹³R¹⁴; —P(O)R¹³R¹⁴; —P⁺R¹³R¹⁴R¹⁵A⁻; amino acid residue; peptide residue; polypeptide residue; and carbohydrate residue;
wherein the R⁶ alkyl; cycloalkyl; polyalkyl; haloalkyl; hydroxyalkyl; alkenyl; alkynyl; aryl; heterocyclyl; arylalkyl; heterocyclylalkyl; polyether; acyloxy radicals optionally may be further substituted with one or more radicals selected from the group consisting of halogen; —CN; oxo; OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹¹ R¹²A⁻; —S⁺R⁹R¹⁰A⁻; and carbohydrate residue; and
wherein the R⁶ quaternary heterocyclyl radical optionally may be substituted with one or more radicals selected from the group consisting of halogen; —CN; —NO₂; oxo; alkyl; cycloalkyl; polyalkyl; haloalkyl; hydroxyalkyl; alkenyl; alkynyl; aryl; heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R³; OM; —SO₂OM; —SO₂NR¹³R¹⁴; —C(O)NR¹³R¹⁴; —C(O)OM; COR¹³; —P(O)R¹³R¹⁴; —P¹³R¹⁴; —PR¹³R¹⁴R¹⁵A⁻; —P(OR¹³)OR¹⁴; —S⁺R¹³R¹⁴A⁻; —N⁺R¹³R¹⁴R¹⁵A⁻; and carbohydrate residue; and
wherein the R⁶ radicals comprising carbon optionally may have one or more carbons replaced by —O—; —NR¹³—; —N⁺R¹³R¹⁴A⁻-; —S—; —SO—; —SO₂—; —S⁺R¹³A⁻-; —PR¹³; —P(O)R¹³—; —PR¹³R¹⁴; —P⁺R¹³R¹⁴A⁻-; phenylene; amino acid residue; peptide residue; polypeptide residue; carbohydrate residue; polyether; or polyalkyl; wherein said phenylene; amino acid residue; peptide residue; polypeptide residue; carbohydrate residue; and polyalkyl optionally may have one or more carbons replaced by —O—; —NR⁹—; —N⁺R⁹R¹⁰A⁻; —S—; —SO—; —SO₂—; —S⁺R⁹A⁻-; —PR⁹—; —P⁺R⁹R¹⁰A⁻-; or —P(O)R⁹—; and
wherein R¹⁸ is selected from the group consisting of alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl; and
wherein the R¹⁸ alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl radicals optionally may be substituted with one or more radicals selected from the group consisting of halogen; —CN; NO₂; oxo; —OR⁹; —NR⁹R¹⁰; —N⁺R⁹R¹¹R¹²A⁻; —SR⁹; —S(O)R⁹; —SO₂R⁹; —SO₃R⁹; —CO₂R⁹; —CONR⁹R¹⁰; —SO₂OM; —SO₂NR⁹R¹⁰; —PR⁹R¹⁰; P(OR 3)OR 14; —PO(OR¹⁶)OR¹⁷; and —C(O)OM; or
a pharmaceutically acceptable salt, solvate, or prodrug thereof;
provided that at least one of R³, R⁴ and R⁶ is R⁵; and
provided that the R⁵ alkyl, cycloalkyl, aryl, and heterocyclyl, and —OR⁹ radicals are not substituted with —O(CH₂)₁₋₄NR′R″R′″ wherein R′, R″ and R′″ are independently selected from hydrogen and alkyl; and
provided that at least one of the following conditions is satisfied:
(a) the R⁵ moiety possesses an overall positive charge;
(b) the R⁵ moiety comprises a quaternary ammonium group or a quaternary amine salt; and
(c) the R⁵ moiety comprises at least two carboxy groups.

76. A compound of claim 75 wherein R⁵ is aryl substituted with one or more radicals independently selected from the group consisting of halogen; —CN; —NO₂, oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³R¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴—P⁺R¹³R¹⁴R¹⁵ A; —P(OR¹³)OR¹⁴; —S⁺R¹³R¹⁴A; and —N⁺R¹³R¹⁴R¹⁵A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ aryl optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A⁻; —P(O)R⁷R⁸; —PR⁷R⁸; —P⁺R⁷R⁸R⁹A⁻; and —P(O)(OR⁷)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁵ aryl optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸ A⁻-; or phenylene;
wherein R⁷ and R⁸ are independently selected from the group consisting of hydrogen; and alkyl;
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkyl; heterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; N⁺R⁹R¹⁰R^{w}A⁻; —SR¹⁶; —S(O)R⁹; —SO₂R?; —SO₃R¹⁶; —CO₂R¹⁶; —CONR⁹R¹⁰; —SO₂NR⁹R¹⁰; —PO(OR¹⁶)OR¹⁷; —P⁹R¹⁰—P⁺R⁹R¹⁰R¹¹A⁻; —S⁺R⁹R¹⁰A⁻; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable anion and M is a pharmaceutically acceptable cation.

77. A compound of claim 76 wherein R⁵ is: 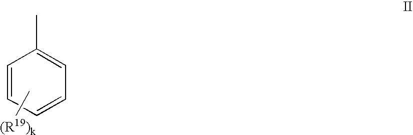
wherein
k is 0, 1, 2, 3 or 4; and
one or more R¹⁹ are independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴—NR¹³NR¹⁵; —CO₂R¹³; —OM; —SO₂OM; —SO₂NR¹³R¹; C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR 13R¹⁴; —P(O)R¹³R¹⁴; —P⁺R¹³R¹⁴R¹⁵A-; —P(OR¹³)OR¹⁴; —S⁺R¹³R¹⁴A⁻; and —N⁺R¹³R¹⁴R¹⁵A-; and
wherein the R¹⁹ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may be further substituted with one or more radicals selected from the group consisting of-CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸—; —N⁺R⁷R⁸R⁹A-; —P(O)R⁷R⁸; —PR⁷R⁸; —P⁺R⁷R⁸R⁹A⁻; and —P(O)(OR⁷)OR⁸; and
wherein the R¹⁹ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and-CONR⁹R¹⁰; or R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹ 7; —P⁹R¹⁰; —P⁺R⁹R¹⁰R¹¹A⁻; —S⁺R⁹R¹⁰A⁻; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable anion and M is a pharmaceutically acceptable cation.

78. A compound of claim 77 wherein R⁵ is: 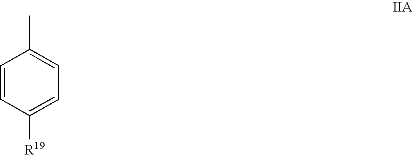
wherein R¹⁹ is as defined in claim 77.

79. A compound of claim 77 wherein R⁵ is: 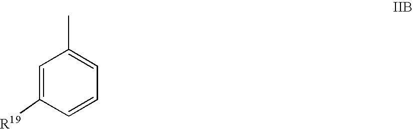
wherein R¹⁹ is as defined in claim 77.

80. A compound of claim 77 wherein:
R³ is R⁵: and
R⁴ is selected from the group consisting of hydrogen and alkyl.

81. A compound of claim 77 wherein:
R³ is selected from the group consisting of hydrogen and alkyl; and
R⁴ is R⁵.

82. A compound of claim 77 wherein:
R³ is R⁵ and
R⁴ is selected from the group consisting of hydrogen; oxo; alkyl; cycloalkyl; aryl; heterocyclyl; acyl, thioacyl, and —OR⁹;
wherein the R⁴ alkyl; cycloalkyl; aryl; heterocyclyl radical is substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR 13; —NR¹³ R¹⁴; SR¹³; S(O)R³; —SO₂R¹³; —SO₃R¹³; —NR¹³ 0R⁴; —NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³R¹⁴CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A; —P(OR¹³)OR¹⁴; —S⁺R¹³R¹⁴A⁻; and —N⁺R¹³R¹⁴R¹⁵A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁴ radical optionally may be further substituted with one or more radicals selected from the group consisting of-CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A⁻; —P(O)R⁷R⁸; —PR⁷R⁸; —P⁺R⁷R⁸R⁹A⁻; and —P(O)(OR⁷)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R⁴ radical optionally may have one or more carbons replaced by —O—; —NR⁷—; —S—; —SO—; —SO₂—; —S⁺R⁷A⁻-; —PR⁷—; —P(O)R⁷; P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰—P⁺R⁹R¹⁰ R¹¹A⁻; —SR⁹R¹⁰A⁻; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable cation and M is a pharmaceutically acceptable cation.

83. A compound of claim 77 wherein:
R³ is selected from the group consisting of hydrogen; oxo; alkyl; cycloalkyl; aryl; heterocyclyl; acyl, thioacyl, and —OR⁹;
wherein the R³ alkyl; cycloalkyl; aryl; heterocyclyl radical is substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³ radical optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷—SO₂R⁷; —SO₃R⁷; —CO₂R⁷; —CONR⁷R⁸; —N⁺R⁷R⁸R⁹A-; —P(O)R⁷R⁸; —PR⁷R⁸; —P⁺R⁷R⁸R⁹A⁻; and —P(O)(OR⁷)OR⁸ and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³ radical optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alknyl; aryl; heterocyclyl; quaternary heterocyclyl; quaternary heterocyclylalkyl; carboxy; carboxyalkyl; guanidinyl; —OR⁶; —NR⁹R¹⁰; —N⁺R⁹R¹⁰R^{w}A⁻; —SR⁶; —S(O)R⁹; —SO₂R⁹; —SO₃¹⁶; —CO₂R¹⁶; —CONR⁹R¹⁰; —SO₂NR⁹R¹⁰; —PO(OR¹⁶)OR¹⁷; —P⁺R⁹R¹⁰R¹¹A⁻; —S⁺R⁹R¹⁰A-; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable cation and M is a pharmaceutically acceptable cation; and
R⁴ is R⁵.

84. A compound of claim 77 wherein:
R¹⁹ is independently selected from the group consisting of —OR¹³, —NR¹³R¹⁴, NR¹³C(O)R¹⁴, —OC(O)NR¹³R¹⁴, and —NR¹³SO₂R¹⁴ and
wherein R¹³ R¹⁴ and R¹⁵ are independently selected from the group consisting of alkyl, polyether, aryl, quaternary heterocycle, arylalkyl, heterocyclylalkyl, quaternary heterocyclylalkyl, alkylheterocyclylalkyl, and alkylammoniumalkyl,
wherein alkyl optionally has one or more carbons replaced by O or N⁺R⁹R¹⁰A-, and
wherein R¹³, R¹⁴, and R¹⁵ are optionally substituted with one or more groups selected from the group consisting of hydroxy, carboxy, alkyl, quaternary heterocyclylalkyl, —SR⁹, —S(O)R⁹, —S(O)₂R⁹, —S(O)₃R⁹, —NR⁹R¹⁰, —N⁺R⁹R¹¹R¹²A⁻, —CONR⁹R¹⁰, and —PO(OR¹⁶)OR¹⁷, and
wherein R⁹ and R¹⁰ are independently selected from the group consisting of hydrogen, alkyl, heterocyclylalkyl, carboxyalkyl, carboalkoxyalkyl, and carboxyalkylheterocycle; and
wherein R¹¹ and R¹² are independently alkyl; and
wherein A⁻ is a pharmaceutically acceptable anion and M is a pharmaceutically acceptable cation.

85. A compound of claim 77 wherein:
R¹⁹ is independently selected from the group consisting of, —NR¹³R¹⁴, —NR¹³C(O)R¹⁴, —OC(O)NR¹³R¹⁴, and —NR¹³SO²R⁴ and
wherein R¹³, R¹⁴, and R¹⁵ are independently selected from the group consisting of polyether, aryl, quaternary heterocycle, arylalkyl, heterocyclylalkyl, quaternary heterocyclylalkyl, and alkylheterocyclylalkyl,
wherein alkyl optionally has one or more carbons replaced by O or N⁺R⁹R¹⁰A-, and
wherein R¹³, R¹⁴, and R¹⁵ are optionally substituted with one or more groups selected from the group consisting of hydroxy, carboxy, alkyl, quaternary heterocyclylalkyl, —SR⁹, —S(O)R⁹, —S(O)₂R⁹, —S(O)₃R⁹, —NR⁹R¹⁰, —N⁺R⁹R¹¹R¹²A⁻, —CONR⁹R¹⁰, and —PO(OR¹⁶)OR¹⁷, and
wherein R⁹ and R¹⁰ are independently selected from the group consisting of hydrogen, alkyl, heterocyclylalkyl, carboxyalkyl, carboalkoxyalkyl, and carboxyalkylheterocycle; and
wherein R¹¹ and R¹² are independently alkyl; and
wherein A⁻ is a pharmaceutically acceptable anion and M is a pharmaceutically acceptable cation.

86. A compound of claim 84 wherein R⁵ is: 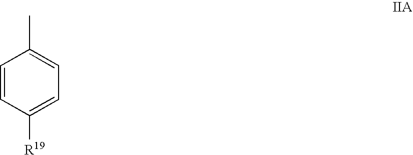
wherein R¹⁹ is as defined in claim 84.

87. A compound of claim 84 wherein R⁵ is: 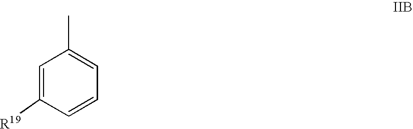
wherein R¹⁹ is as defined in claim 84.

88. A compound of claim 84 wherein R¹⁹ is selected from the group consisting

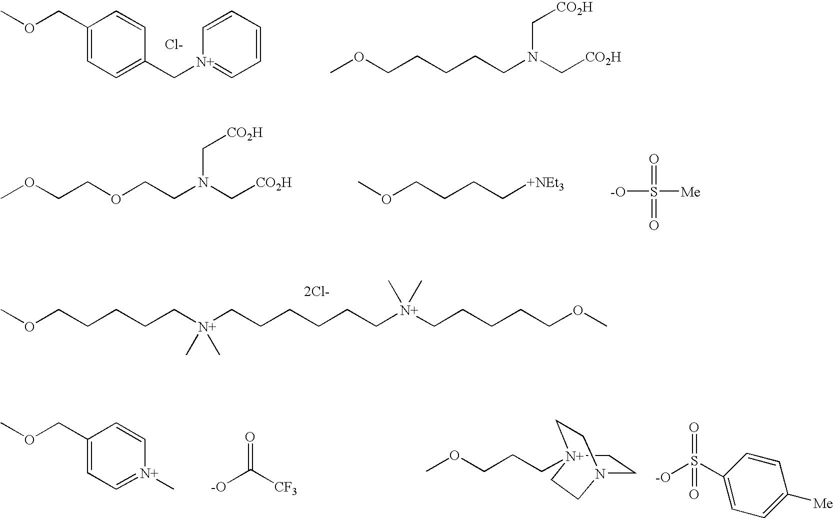

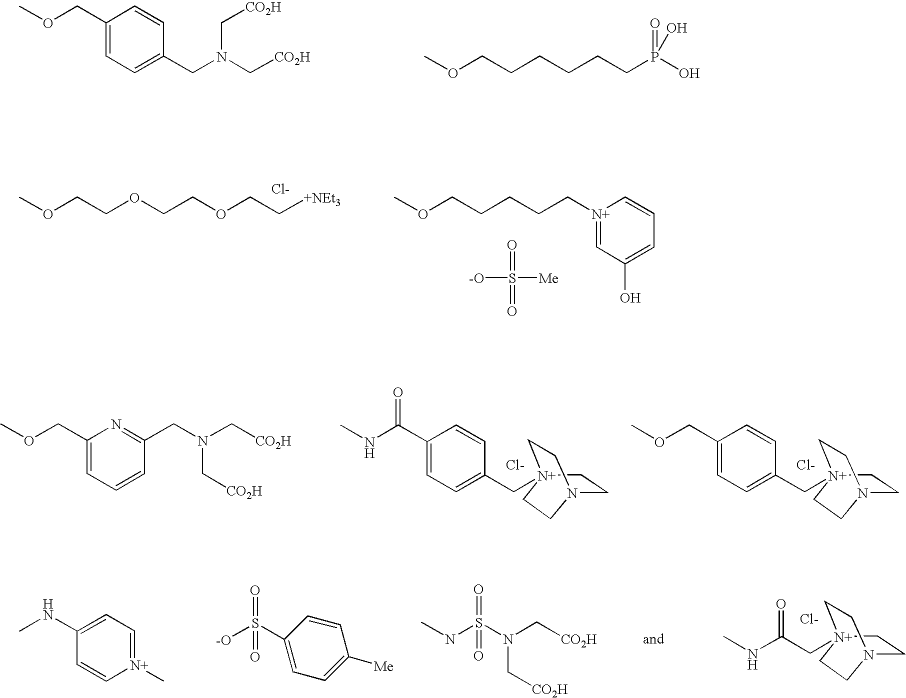

89. A compound of claim 84 wherein:
j is 2; —
R^{1A} and R^{1B} are independently selected from hydrogen and alkyl; and
R^{2A} and R^{2B} are independently selected from hydrogen and alkyl.

90. A compound of claim 84 wherein:
j is 2;
R^{1A} and R^{1B} are hydrogen; and
R^{2A} and R^{2B} are independently selected from alkyl.

91. A compound of claim 84 wherein:
j is 2;
R^{1A} and R^{1B} are hydrogen; and
R^{2A} and R^{2B} are independently selected from ethyl, propyl and butyl.

92. A compound of claim 84 wherein:
j is 2;
R^{1A} and R^{1B} are hydrogen;
R^{2A} and R^{2B} are independently selected from ethyl, propyl and butyl;
R³ is R⁵; and
R⁴ is selected from hydrogen and alkyl.

93. A compound of claim 84 wherein:
j is 2;
R^{1A} and R^{1B} are hydrogen;
R^{2A} and R^{2B} are independently selected from ethyl, propyl and butyl;
R³ is selected from from hydrogen and alkyl; and
R⁴ is R⁵.

94. A compound of claim 84 wherein j is 1 or 2.

95. A compound of claim 84 wherein j is 2.

96. A compound of claim 84 wherein R^{1A} and R^{1B} are hydrogen.

97. A compound of claim 84 wherein R^{2A} and R^{2B} are independently selected from the group consisting of hydrogen and C₁₋₆alkyl.

98. A compound of claim 84 wherein R^{2A} and R^{2B} are independently selected from the group consisting C₁₋₆alkyl.

99. A compound of claim 84 wherein R^{2A} and R^{2B} are the same alkyl.

100. A compound of claim 84 wherein R^{2A} and R^{2B} are each n-butyl.

101. A compound of claim 84 wherein one of R^{2A} and R^{2B} is ethyl and the other of R^{2A} and R^{2B} is n-butyl.

102. A compound of claim 84 wherein one or more R⁶ are independently selected from methoxy and dimethylamino.

103. A compound of claim 84 wherein
j is 1 or 2;
R^{1B} and R^{1B} are hydrogen;
R^{2A} and R^{2B} are n-butyl; and
one or more R⁶ are independently selected from methoxy and dimethylamino.

104. A compound of claim 84 wherein
j is 1 or 2;
R^{1A} and R^{1B} are hydrogen;
one of R^{2A} and R^{2B} is ethyl and the other of R^{2A} and R^{2B} is n-butyl; and
one or more R⁶ are independently selected from methoxy and dimethylamino.

105. A compound of claim 88 wherein:
j is 2;
R^{1A} and R^{1B} are independently selected from hydrogen and alkyl; and
R^{2A} and R^{2B} are independently selected from hydrogen and alkyl.

106. A compound of claim 88 wherein:
j is 2;
R^{1A} and R^{1B} are hydrogen; and
R^{2A} and R^{2B} are independently selected from alkyl.

107. A compound of claim 88 wherein:
j is 2;
R^{1A} and R^{1B} are hydrogen; and
R^{2A} and R^{2B} are independently selected from ethyl, propyl and butyl.

108. A compound of claim 88 wherein:
j is 2;
R^{1A} and R^{1B} are hydrogen;
R^{2A} and R^{2B} are independently selected from ethyl, propyl and butyl;
R³ is R⁵; and
R⁴ is selected from hydrogen and alkyl.

109. A compound of claim 88 wherein:
j is 2;
R^{1A} and R^{1B} are hydrogen;
R^{2A} and R^{2B} are independently selected from ethyl, propyl and butyl;
R³ is selected from from hydrogen and alkyl; and
R⁴ is R⁵.

110. A compound of claim 88 wherein j is 1 or 2.

111. A compound of claim 88 wherein j is 2.

112. A compound of claim 88 wherein R^{1A} and R^{1B} are hydrogen.

113. A compound of claim 88 wherein R^{2A} and R^{2B} are independently selected from the group consisting of hydrogen and C₁₋₆alkyl.

114. A compound of claim 88 wherein R^{2A} and R^{2B} are independently selected from the group consisting C₁₋₆alkyl.

115. A compound of claim 88 wherein R^{2A} and R^{2B} are the same alkyl.

116. A compound of claim 88 wherein R^{2A} and R^{2B} are each n-butyl.

117. A compound of claim 88 wherein one of R^{2A} and R^{2B} is ethyl and the other of R^{1A} and R^{2B} is n-butyl.

118. A compound of claim 88 wherein one or more R⁶ are independently selected from methoxy and dimethylamino.

119. A compound of claim 88 wherein
j is 1 or 2;
R^{1A} and R^{1B} are hydrogen;
R^{2A} and R^{2B} are n-butyl; and
one or more R⁶ are independently selected from methoxy and dimethylamino.

120. A compound of claim 88 wherein
j is 1 or 2;
R^{1A} and R^{1B} are hydrogen;
one of R^{2A} and R^{2B} is ethyl and the other of R^{2A} and R^{2B} is n-butyl; and
one or more R⁶ are independently selected from methoxy and dimethylamino.

121. A compound of Formula III: 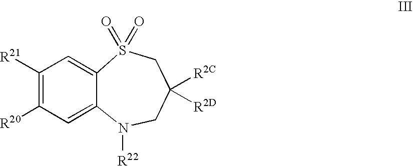
wherein:
R^{2C} and R^{2D} are independently selected from C₁₋₆alkyl; and
R²⁰ is selected from the group consisting of halogen and R²³;
R²¹ is selected from the group consisting of hydroxy, alkoxy, and R²³; and
wherein R²³ is aryl substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³ CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R 13_{R}14_{R}15_{A}_{⁻}; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R²³ aryl optionally may be further substituted with one or more radicals selected from the group consisting of-CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷—CONR⁷R⁸; —N⁺R⁷R⁸R⁹A-; —P(O)R⁷R⁸; —PR⁷R⁸; —P⁺R⁷R⁸R⁹A⁻; and —P(O)(OR⁷)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R²³ aryl optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸ A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹RO; —N⁺R⁹R¹⁰R^{w}A⁻; —SR¹⁶; S(O)R⁹ SO²R⁹ SO³R¹⁶; —CO₂R¹⁶; —CONR⁹R¹⁰; —SO₂NR⁹R¹⁰; —PO(OR¹⁶)OR¹⁷; —P⁹R¹⁰—P⁺R⁹R¹⁰R¹¹A⁻; —S⁺R⁹R¹⁰A-; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable anion and M is a pharmaceutically acceptable cation; and
R²² is unsubstituted phenyl or R²³; or
a pharmaceutically acceptable salt, solvate, or prodrug thereof;
provided that at least one of R²⁰, R²¹ and R²² is R²³.

122. A compound of claim 121 wherein R²³ is: 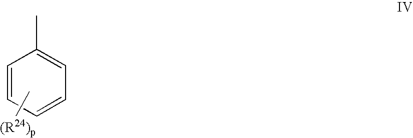
wherein
p is 0, 1, 2, 3 or 4; and
one or more R²⁴ are independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; SO₂R¹³-SO₃R 3 NR¹³OR¹⁴; —NR¹³NR¹¹⁵; —CO₂R³; —OM; —SO₂OM; —SO₂NR R¹⁴—C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R⁴; —NR³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴—S⁺R¹³R¹⁴A⁻; and —N⁺R¹³R¹⁴R¹⁵A⁻; and
wherein the R²⁴ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may be further substituted with one or more radicals selected from the group consisting of-CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A-; —P(O)R⁷R⁸; —PR⁷R⁸; —P⁺R⁷R⁸R⁹A⁻; and —P(O)(OR⁷)OR⁸; and
wherein the R²⁴ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may have one or more carbons replaced by —O—; —NR⁷—; —N⁺R⁷R⁸A⁻; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R 14 and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A-; S⁺R⁹R¹⁰A-; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl-arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable anion and M is a pharmaceutically acceptable cation.

123. A compound of claim 122 wherein R²³ is: 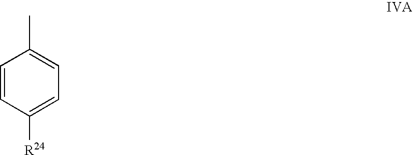
wherein R²⁴ is as defined in claim 122.

124. A compound of claim 122 wherein R²³ is: 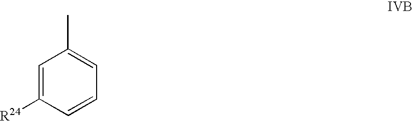
wherein R²⁴ is as defined in claim 122.

125. A compound of claim 122 wherein:
R²⁴ is independently selected from the group consisting of —OR¹³, —NR¹³R¹⁴, —NR¹³C(O)R¹⁴, —OC(O)NR¹³R¹⁴, and —NR¹³SO₂R¹⁴, and
wherein R¹³, R¹⁴, and R¹⁵ are independently selected from the group consisting of alkyl, polyether, aryl, quaternary heterocycle, arylalkyl, heterocyclylalkyl, quaternary heterocyclylalkyl, alkylheterocyclylalkyl, and alkylammoniumalkyl,
wherein alkyl optionally has one or more carbons replaced by O or N⁺R⁹R¹⁰A-, and
wherein R¹³, R¹⁴, and R¹⁵ are optionally substituted with one or more groups selected from the group consisting of hydroxy, carboxy, alkyl, quaternary heterocyclylalkyl, —SR⁹, —S(O)R⁹, —S(O)₂R⁹, —S(O)₃R⁹, —NR⁹R¹⁰, —N⁺R⁹R¹¹R¹²A⁻, —CONR⁹R¹⁰, and —PO(OR¹⁶)OR¹⁷, and
wherein R⁹ and R¹⁰ are independently selected from the group consisting of hydrogen, alkyl, heterocyclylalkyl, carboxyalkyl, carboalkoxyalkyl, and carboxyalkylheterocycle; and
wherein R¹¹ and R¹² are independently alkyl; and
wherein A⁻ is a pharmaceutically acceptable anion and M is a pharmaceutically acceptable cation.

126. A compound of claim 125 wherein R²³ is: 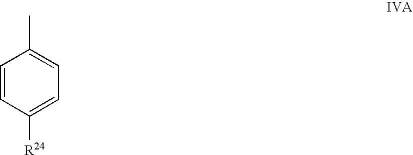
wherein R is as defined in claim 125.

127. A compound of claim 125 wherein R²³ is: 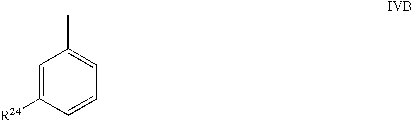
wherein R²⁴ is as defined in claim 125.

128. A compound of claim 125 wherein R²⁴ is selected from the group consisting of:

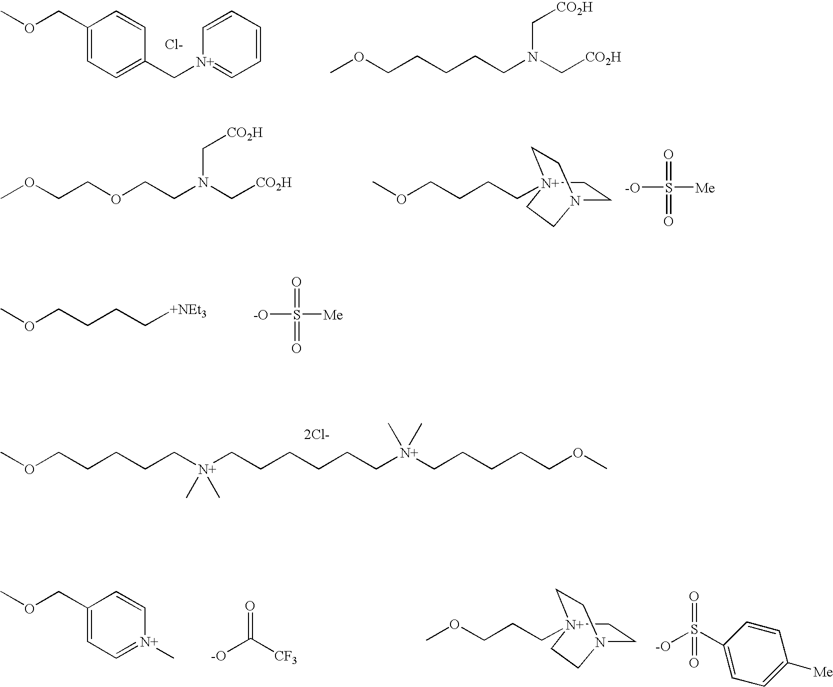

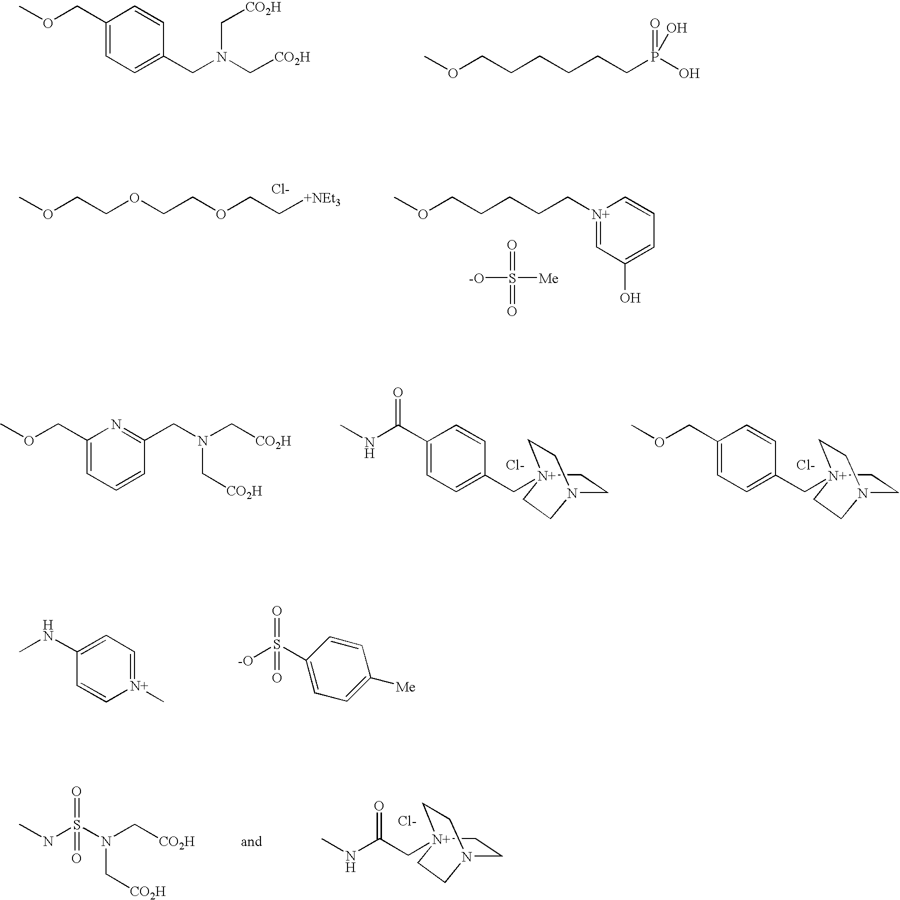

129. A compound of claim 122 wherein:
R^{2C} and R^{2D} are independently selected from ethyl and n-butyl;
R²⁰ is chloro; and
R²¹ is selected from the group consisting of hydroxy and methoxy.

130. A compound of claim 122 wherein:
R^{2C} and R^{2D} are n-butyl;
R²⁰ is chloro; and
R²¹ is selected from the group consisting of hydroxy and methoxy.

131. A compound of claim 122 wherein:
one of R^{2C} and R^{2D} is ethyl and the other of R^{2C} and R^{2D} is n-butyl;
R²⁰ is chloro; and
R²¹ is selected from the group consisting of hydroxy and methoxy.

132. A compound of claim 122 wherein R^{2C} and R^{2D} are the same alkyl.

133. A compound of claim 122 wherein R^{2C} and R^{2D} are each n-butyl.

134. A compound of claim 122 wherein one of R^{2C} and R^{2D} is ethyl and the other of R^{2C} and R^{2D} is n-butyl.

135. A compound of claim 125 wherein:
R^{2C} and R^{2D} are independently selected from ethyl and n-butyl;
R²⁰ is chloro; and
R²¹ is selected from the group consisting of hydroxy and methoxy.

136. A compound of claim 125 wherein:
R^{2C} and R^{2D} are n-butyl;
R²⁰ is chloro; and
R²¹ is selected from the group consisting of hydroxy and methoxy.

137. A compound of claim 125 wherein:
one of R^{2C} and R^{2D} is ethyl and the other of R^{2C} and R^{2D} is n-butyl;
R²⁰ is chloro; and
R²¹ is selected from the group consisting of hydroxy and methoxy.

138. A compound of claim 125 wherein R^{2C} and R^{2D} are the same alkyl.

139. A compound of claim 125 wherein R^{2C} and R^{2D} are each n-butyl.

140. A compound of claim 125 wherein one of R^{2C} and R^{2D} is ethyl and the other of R^{2C} and R^{2D} is n-butyl.

141. A compound of Formula V: 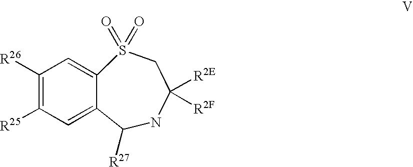
wherein:
R^{2E} and R^{2F} are independently selected from C₁₋₆ alkyl; and
R²⁵ and R²⁶ are independently selected from the group consisting of hydrogen, alkoxy, and R²⁸;
wherein R²⁸ is aryl substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR⁴R⁵; —CO₂R³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R²⁸ aryl optionally may be further substituted with one or more radicals selected from the group consisting of-CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁷; —N⁺R⁷R⁸R⁹A-; —P(O)R⁷R⁸; —P⁺R⁷R⁸; —P⁺R⁷R⁸R⁹A⁻; —P(O)(OR⁷)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R²⁸ aryl optionally may have one or more carbons replaced by —O—; —NR⁷—; —N⁺R⁷R⁸A⁻; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and-CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A-; —S⁺R⁹R¹⁰A⁻; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁹R¹⁰A⁻-; —P(O)₉—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable anion and M is a pharmaceutically acceptable cation; and
R²⁷ is unsubstituted phenyl or R²⁸; or
a pharmaceutically acceptable salt, solvate, or prodrug thereof;
provided that at least one of R²⁵, R²⁶ and R²⁷ is R²⁸.

142. A compound of claim 141 wherein R²⁸ is: 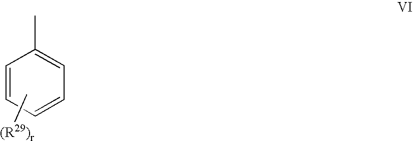
wherein
r is 0, 1, 2, 3 or 4; and
one or more R²⁹ are independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴R¹⁵A⁻; —P(OR¹³)OR¹⁴; —S⁺R¹³R¹⁴A⁻; and —N⁺R¹³R¹⁴R¹⁵A⁻; and
wherein the R²⁹ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may be further substituted with one or more radicals selected from the group consisting of-CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸—N⁺R⁷ R⁸R⁹A⁻; —P(O)R⁷R⁸; —PR⁷R⁸; —P⁺R⁷R⁸R⁹A⁻; and —P(O)(OR⁷)OR⁸; and
wherein the R²⁹ alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether radicals optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹, —SO₂R⁹; —SO₃R⁹; —CO₂R⁹; and —CONR⁹R¹⁰; or R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkyl heterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁺R⁹R¹⁰R¹¹A-; —S⁺R⁹R¹⁰A⁻; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable anion and M is a pharmaceutically acceptable cation.

143. A compound of claim 142 wherein R²⁸ is: 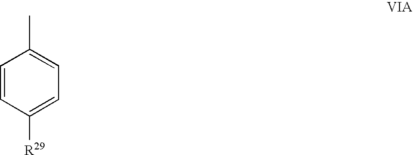
wherein R²⁹ is as defined in claim 142.

144. A compound of claim 142 wherein R is: 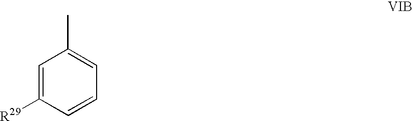
wherein R²⁹ is as defined in claim 142.

145. A compound of claim 142 wherein:
R²⁹ is independently selected from the group consisting of —OR¹³, —NR¹³R¹⁴ NR¹³C(O)R¹⁴, —OC(O)NR¹³R¹⁴, and —NR³SO₂R¹⁴, and
wherein R¹³, R¹⁴ and R¹⁵ are independently selected from the group consisting of alkyl, polyether, aryl, quaternary heterocycle, arylalkyl, heterocyclylalkyl, quaternary heterocyclylalkyl, alkylheterocyclylalkyl, and alkylammoniumalkyl,
wherein alkyl optionally has one or more carbons replaced by O or N⁺R⁹R¹⁰A-, and
wherein R¹³, R¹⁴, and R¹⁵ are optionally substituted with one or more groups selected from the group consisting of hydroxy, carboxy, alkyl, quaternary heterocyclylalkyl, —SR⁹, —S(O)R⁹, —S(O)₂R⁹, —S(O)₃R⁹, —NR⁹R¹⁰, —N⁺R⁹R¹¹R¹²A⁻, CONR⁹R¹⁰, and —PO(OR¹⁶)OR¹⁷, and
wherein R⁹ and R¹⁰ are independently selected from the group consisting of hydrogen, alkyl, heterocyclylalkyl, carboxyalkyl, carboalkoxyalkyl, and carboxyalkylheterocycle; and
wherein R¹¹ and R¹² are independently alkyl; and
wherein A⁻ is a pharmaceutically acceptable anion and M is a pharmaceutically acceptable cation.

146. A compound of claim 145 wherein R²⁸ is: 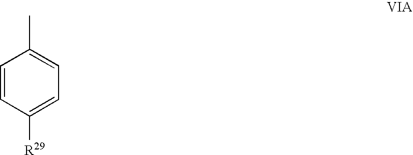
wherein R²⁹ is as defined in claim 145.

147. A compound of claim 145 wherein R²⁸ is: 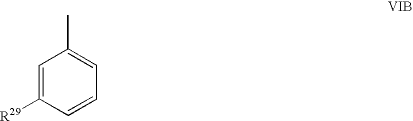
wherein R²⁹ is as defined in claim 145.

148. A compound of claim 145 wherein R²⁹ is selected from the group consisting of:

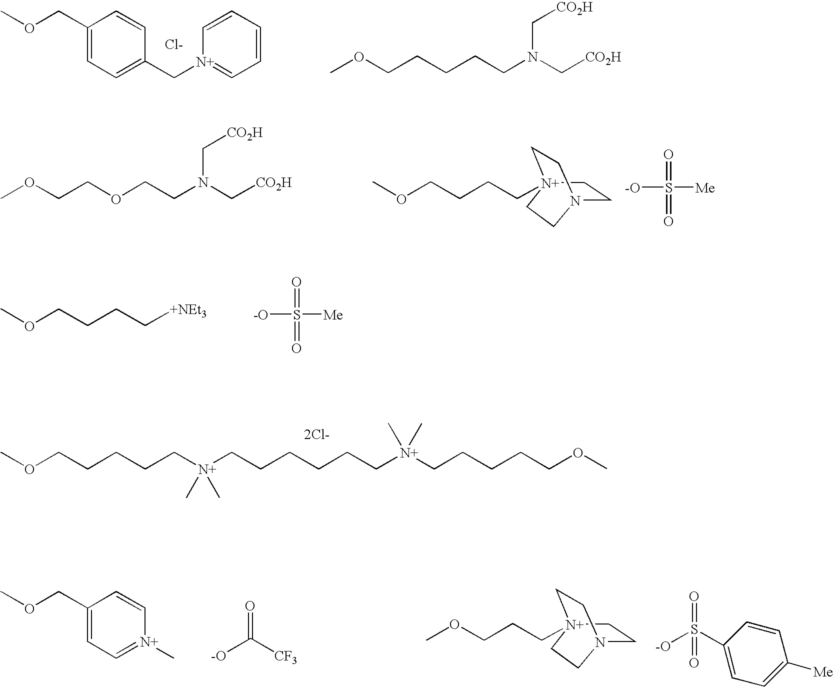

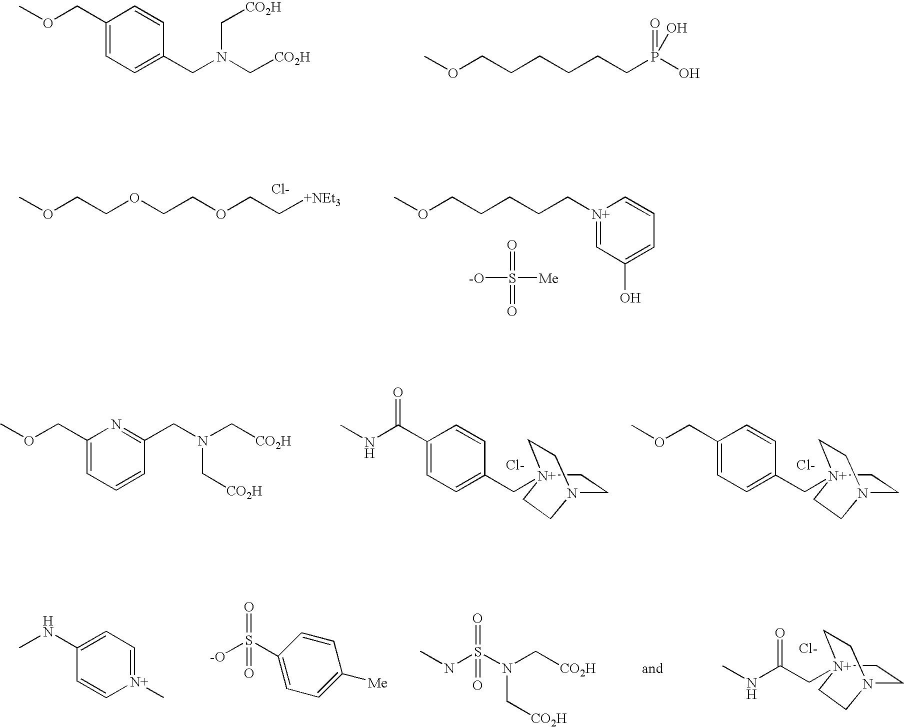

149. A compound of claim 142 wherein:
R^{2E} and R^{2F} are independently selected from ethyl and n-butyl; and
R²⁵ and R²⁶ are independently selected from hydrogen and methoxy.

150. A compound of claim 142 wherein:
R^{2E} and R^{2F} are n-butyl; and
R²⁵ and R²⁶ are independently selected from hydrogen and methoxy.

151. A compound of claim 142 wherein:
one of R^{2E} and R^{2F} is ethyl and the other of R^{2E} and R^{2F} is n-butyl; and
R²⁵ and R²⁶ are independently selected from hydrogen and methoxy.

152. A compound of claim 142 wherein R²E. and R^{2F} are the same alkyl.

153. A compound of claim 142 wherein R^{2E} and R^{2F} are each n-butyl.

154. A compound of claim 142 wherein one of R^{2E} and R^{2F} is ethyl and the other of R^{2E} and R^{2F} is n-butyl.

155. A compound of claim 145 wherein:
R^{2E} and R^{2F} are independently selected from ethyl and n-butyl; and
R²⁵ and R²⁶ are independently selected from hydrogen and methoxy.

156. A compound of claim 145 wherein:
R^{2E} and R^{2F} are n-butyl; and
R²⁵ and R²⁶ are independently selected from hydrogen and methoxy.

157. A compound of claim 145 wherein:
one of R^{2E} and R^{2F} is ethyl and the other of R^{2E} and R^{2F} is n-butyl; and
R²⁵ and R²⁶ are independently selected from hydrogen and methoxy.

158. A compound of claim 145 wherein R^{2E} and R^{2F} are the same alkyl.

159. A compound of claim 145 wherein R^{2E} and R^{2F} are each n-butyl.

160. A compound of claim 145 wherein one of R^{2E} and R^{2F} is ethyl and the other of R^{2E} and R^{2F} is n-butyl.

161. A compound of claim 142 wherein:
one of R^{2E} and R^{2F} is ethyl and the other of R^{2E} and R^{2F} is n-butyl;
R²⁵ and R²⁶ are hydrogen; and
R²⁷ is: 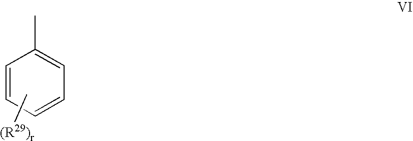
wherein r is 1 and R²⁹ is as defined in claim 142.

162. A compound of claim 142 wherein:
one of R^{2E} and R^{2F} is ethyl and the other of R^{2E} and R^{2F} is n-butyl; and
R²⁵ and R²⁶ are methoxy; and
R²⁷ is: 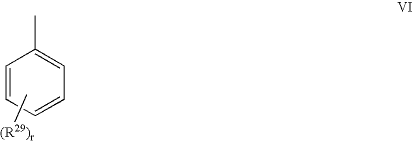
wherein r is 1 and R²⁹ is as defined in claim 142.

163. A compound of Formula VII: 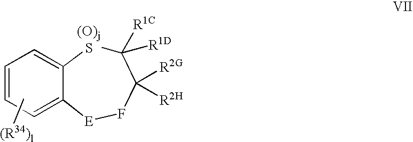
wherein:
i is 0, 1 or 2; and
1 is 0, 1, 2, 3 or 4; and
R^{1C} and R^{1D} are independently selected from hydrogen and alkyl; and
R^{2G} and R^{2H} are independently selected from hydrogen, alkyl, alkenyl, alkynyl, cycloalkyl, cycloalkylalkyl, aryl and aralkyl; or
R^{2G} and R^{2H} together with the carbon atom to which they are attached form a C₃₋₁₀ cycloalkyl group; and
one of E and F is NR³⁰ and the other of E and F is CHR³¹;
wherein R³⁰ and R³¹ are independently selected from the group consisting of hydrogen; oxo; alkyl; cycloalkyl; aryl; heterocyclyl; acyl, thioacyl, —OR⁹, and R³²;
wherein the R³⁰ and R³¹ alkyl; cycloalkyl; aryl; heterocyclyl radicals are independently substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³⁰ and R³¹ radicals optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A⁻; —P(O)R⁷R⁸; —PR⁷R⁸; —P⁺R⁷R⁸R⁹A⁻; and —P(O)(OR⁷)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³⁰ and R³¹ radicals optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹ A-; —S⁺R⁹R¹⁰A-; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹-; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable cation and M is a pharmaceutically acceptable cation; and
R³² is selected from the group consisting of cycloalkyl, aryl and heterocyclyl,
wherein said cycloalkyl, aryl and heterocyclyl are substituted with —N(H)—X—R³³ or —O—X—R³³ and wherein:
X is selected from the group consisting of:
—(C═O)₅-alkyl-;
—(C═O)ₛ-alkyl-NH—;
—(C═O)ₛ-alkyl-O—;
—(C═O)ₛ-alkyl-(C═O)ₜ; and
a covalent bond;
R₃₃ is selected from selected from the group consisting of monosaccharides, disaccharides, and polysaccharides, wherein said monosaccharides, disaccharides, and polysaccharides may be protected with one or more sugar protecting groups;
s and t are independently 0 or 1; and
one or more R³⁴ radicals are independently selected from the group consisting of R³², hydrogen; halogen; —CN; —NO₂; alkyl; cycloalkyl; polyalkyl; haloalkyl; hydroxyalkyl; alkenyl; alkynyl; aryl; heterocyclyl; quaternary heterocyclyl; arylalkyl; heterocyclylalkyl; polyether; acyloxy; —OR¹³; —NR¹³R¹⁴; —SR¹³; —S(O)R¹³; —S(O)₂R¹³; —SO₃R¹³; —S⁺R¹³R¹⁴A⁻; —NR¹³OR¹⁴; —NR¹³NR¹⁴R¹⁵; —CO₂R¹³; —OM; —SO₂OM; —SO₂NR¹³R¹⁴; —NR¹⁴C(O)R¹³; —C(O)NR¹³R¹⁴; —C(O)OM; —COR¹³; —OR¹⁴; —S(O)ₙNR¹³R¹⁴; —NR¹³R¹⁸; —NR¹⁸OR¹⁴; —N⁺R¹³R¹⁴R¹⁵A⁻; —PR¹³R¹⁴; —P(O)R¹³ R¹⁴; —P⁺R¹³ R⁴R⁵A⁻; amino acid residue; peptide residue; polypeptide residue; and carbohydrate residue;
wherein the R³⁴ alkyl; cycloalkyl; polyalkyl; haloalkyl; hydroxyalkyl; alkenyl; alkynyl; aryl; heterocyclyl; arylalkyl; heterocyclylalkyl; polyether; acyloxy radicals optionally may be further substituted with one or more radicals selected from the group consisting of halogen; —CN; oxo; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹¹R¹²A⁻; —S⁺R⁹R¹⁰A⁻; and carbohydrate residue; and
wherein the R³⁴ quaternary heterocyclyl radical optionally may be substituted with one or more radicals selected from the group consisting of halogen; —CN; —NO₂; oxo; alkyl; cycloalkyl; polyalkyl; haloalkyl; hydroxyalkyl; alkenyl; alkynyl; aryl; heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R¹³; OM; —SO₂OM; —SO₂NR¹³R¹⁴; —C(O)NR¹³R¹⁴; —C(O)OM; —COR¹³; P(O)R¹³R¹⁴; —P³ R¹⁴; —P⁺R¹³R¹⁴R¹⁵A⁻; —P(OR¹³)OR¹⁴; —S⁺R¹³R¹⁴A⁻; —N⁺R¹³R¹⁴R¹⁵A⁻; and carbohydrate residue; and
wherein the R³⁴ radicals comprising carbon optionally may have one or more carbons replaced by —O—; —NR¹³—; —N R¹³R¹⁴A⁻; —S—; —SO—; —SO₂—; —S⁺R¹³A⁻; —PR¹³—; P(O)R¹³—; —PR¹³R¹⁴; —P⁺R¹³R¹⁴A⁻-; phenylene; amino acid residue; peptide residue; polypeptide residue; carbohydrate residue; polyether; or polyalkyl; wherein said phenylene; amino acid residue; peptide residue; polypeptide residue; carbohydrate residue; and polyalkyl optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; or —P(O)R⁹—; and
wherein R¹⁸ is selected from the group consisting of alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl; and
wherein the R¹⁸ alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl radicals optionally may be substituted with one or more radicals selected from the group consisting of halogen; —CN; NO₂; oxo; —OR⁹; —NR⁹R¹⁰; —N⁺R⁹R¹¹ R¹²A⁻; —SR⁹; —S(O)R⁹; —SO₂R⁹; —SO₃R⁹; —CO₂R⁹; —CONR⁹R¹⁰; —SO₂OM; —SO₂NR⁹R¹⁰; —PR⁹R¹⁰; —P(OR¹³)OR¹⁴; PO(OR¹⁶)OR¹⁷; and —C(O)OM; or
a pharmaceutically acceptable salt, solvate, or prodrug thereof;
provided that at least one of R³⁰, R³¹ and R³⁴ is R³².

164. A compound of claim 163 wherein R³² is phenyl substituted with —N(H)—X—R³³ or —O—X—R³³ wherein:
X is selected from the group consisting of:
—(C═O)ₛ-alkyl-;
—(C═O)ₛ-alkyl-NH—;
—(C═O)ₛ-alkyl-O—;
—(C═O)ₛ-alkyl-(C═O)ₜ; and
a covalent bond;
R₃₃ is selected from selected from the group consisting of monosaccharides, disaccharides, and polysaccharides; and
s and t are independently 0 or 1.

165. A compound of claim 164 wherein R³² is phenyl substituted at the para-position with —N(H)—X—R³³ or —O—X—R³³ wherein:
X is selected from the group consisting of:
—(C═O)ₛ-alkyl-;
—(C═O)ₛ-alkyl-NH—;
—(C═O)ₛ-alkyl-O—;
—(C═O)ₛ,-alkyl-(C═O)ₜ; and
a covalent bond; and
R₃₃ is selected from selected from the group consisting of monosaccharides, disaccharides, and polysaccharides; and
s and t are independently 0 or 1.

166. A compound of claim 164 wherein R³² is phenyl substituted at the meta-position with —N(H)—X—R³³ or —O—X—R³³ wherein:
X is selected from the group consisting of:
—(C═O)ₛ-alkyl-;
—(C═O)ₛ-alkyl-NH—;
—(C═O)ₛ-alkyl-O—;
—(C═O)ₛ-alkyl-(C═O)ₜ; and
a covalent bond; and
R₃₃ is selected from selected from the group consisting of monosaccharides, disaccharides, and polysaccharides; and
s and t are independently 0 or 1;

167. A compound of claim 164 wherein:
R³⁰ is R³²; and
R³¹ is selected from the group consisting of hydrogen and alkyl.

168. A compound of claim 165 wherein:
R³⁰ is selected from the group consisting of hydrogen and alkyl; and
R³¹ is R³².

169. A compound of claim 164 wherein R³² is phenyl substituted with a radical selected from the group consisting of:

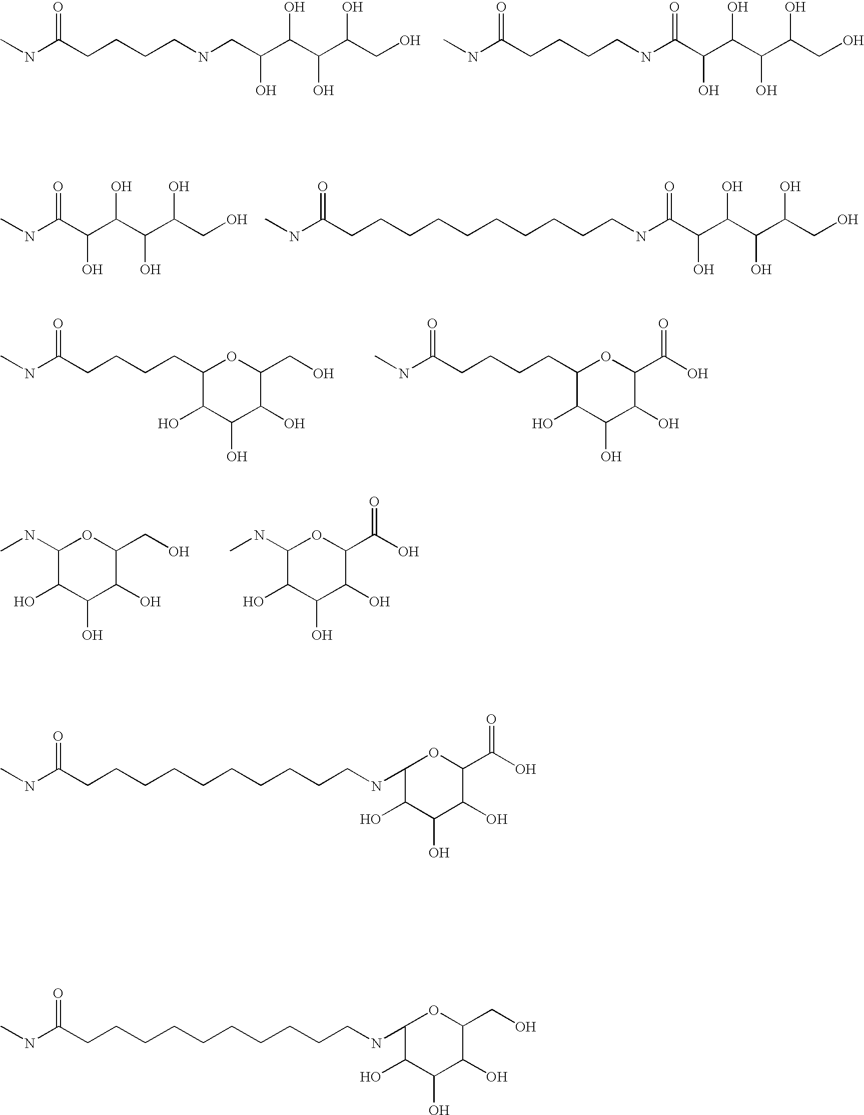

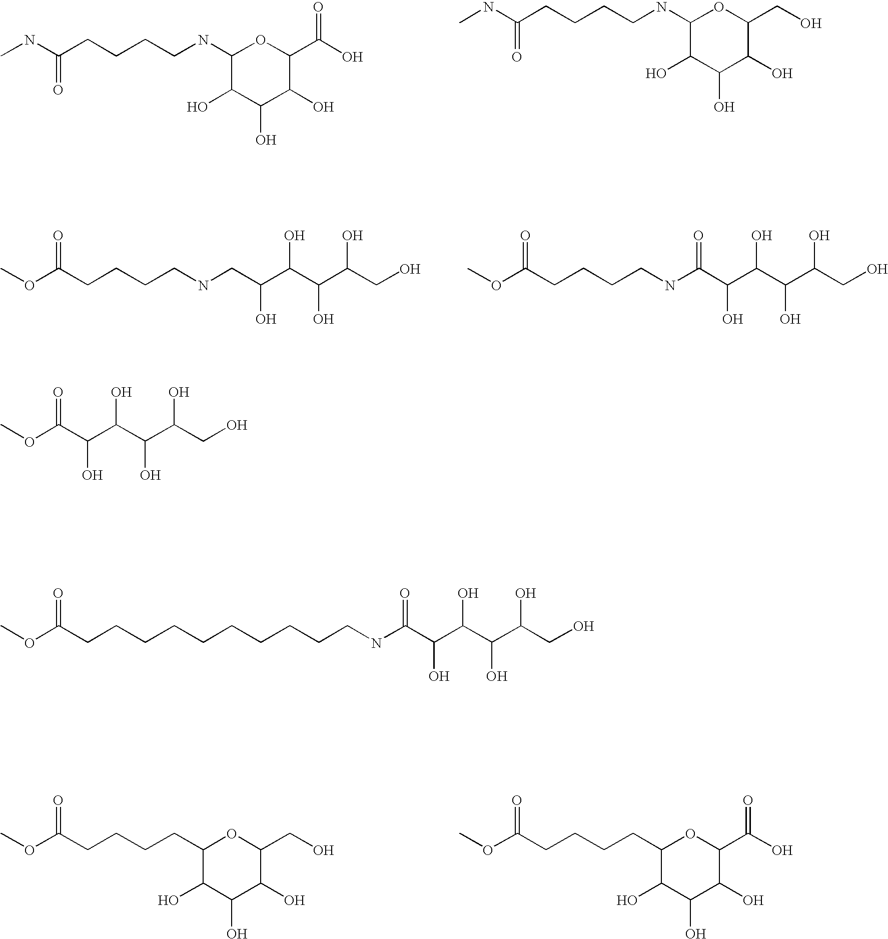

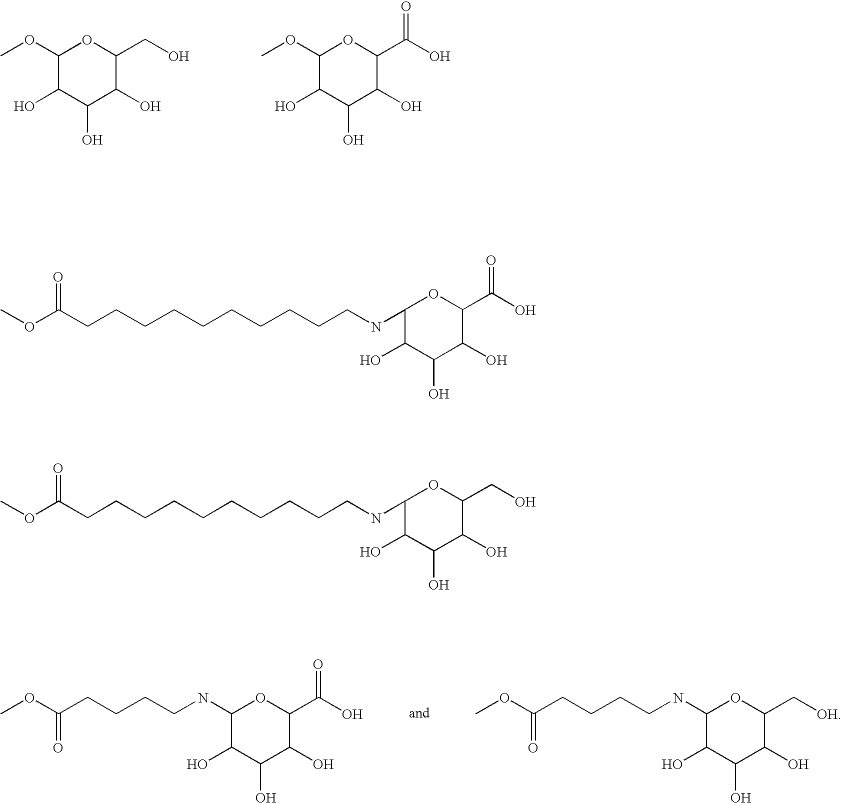

170. A compound of claim 164 wherein:
i is 2;
R^{1C} and R^{1D} are independently selected from hydrogen and alkyl; and
R^{2G} and R^{2H} are independently selected from hydrogen and alkyl.

171. A compound of claim 164 wherein:
i is 2; R^{1C} and R^{1D} are hydrogen; and R^{2G} and R^{2H} are independently selected from alkyl.

172. A compound of claim 164 wherein:
i is 2;
R^{1C} and R^{1D} are hydrogen; and
R^{2G} and R^{2H} are independently selected from ethyl, propyl and butyl.

173. A compound of claim 164 wherein i is 1 or 2.

174. A compound of claim 164 wherein i is 2.

175. A compound of claim 164 wherein R^{1C} and R^{1D} are hydrogen.

176. A compound of claim 164 wherein R^{2G} and R^{2H} are independently selected from the group consisting of hydrogen and C₁₋₆alkyl.

177. A compound of claim 164 wherein R^{2G} and R^{2H} are independently selected from the group consisting C₁₋₄alkyl.

178. A compound of claim 164 wherein R^{2G} and R^{2H} are the same alkyl.

179. A compound of claim 164 wherein R^{2G} and R^{2H} are each n-butyl.

180. A compound of claim 164 wherein one of R^{2G} and R^{2H} is ethyl and the other of R^{2G} and R^{2H} is n-butyl.

181. A compound of claim 164 wherein one or more R³⁴ are independently selected from methoxy and dimethylamino.

182. A compound of claim 164 wherein
i is or 2;
R^{1C} and R^{1D} are hydrogen;
R^{2G} and R^{2H} are n-butyl; and
one or more R³⁴ are independently selected from methoxy and dimethylamino.

183. A compound of claim 164 wherein
i is or 2;
R^{1C} and R_{1D} are hydrogen;
one of R^{2G} and R^{2H} is ethyl and the other of R^{2G} and R^{2H} is n-butyl; and
one or more R³⁴ are independently selected from methoxy and dimethylamino.

184. A compound of claim 163 corresponding to Formula VIIA: 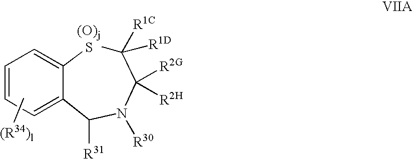
wherein:
i is 0, 1 or 2; and
1 is 0, 1, 2, 3 or 4; and
R^{1C} and R^{1D} are independently selected from hydrogen and alkyl; and
R^{2G} and R^{2H} are independently selected from hydrogen, alkyl, alkenyl, alkynyl, cycloalkyl, cycloalkylalkyl, aryl and aralkyl; or
R^{2G} and R^{2H} together with the carbon atom to which they are attached form a C₃₋₇ cycloalkyl group; and
R³⁰ and R³¹ are independently selected from the group consisting of hydrogen; oxo; alkyl; cycloalkyl; aryl; heterocyclyl; acyl, thioacyl, —OR⁹, and R³²;
wherein the R³⁰ and R³¹ alkyl; cycloalkyl; aryl; heterocyclyl radicals are independently substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R₁₃; —SO₂R¹³; —SO₃R¹³; —NR¹³OR¹⁴; —NR¹⁴R¹⁵; CO₂R³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; P(OR¹³)OR¹⁴; —S⁺R¹³R¹⁴A⁻; and —N⁺R¹³R¹⁴R¹⁵A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³⁰ and R³¹ radicals optionally may be further substituted with one or more radicals selected from the group consisting of —CN; halogen; hydroxy; oxo; alkyl; cycloalkyl; alkenyl; alkynyl; aryl; heterocyclyl; arylalkyl; heterocyclylalkyl; quaternary heterocyclyl; —OR⁷; —NR⁷R⁸; —SR⁷; —S(O)R⁷; —SO₂R⁷; —SO₃R⁷; —CO₂R⁷; —CONR⁷R⁸; —N⁺R⁷R⁸R⁹A⁻; —P(O)R⁷ R⁸; —PR⁷R⁸; —P⁺R⁷R⁸R⁹A; and —P(O)(OR⁷)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³⁰ and R³¹ radicals optionally may have one or more carbons replaced by —O—; —NR⁷; —N⁺R⁷R⁸A⁻-; —S—; —SO—; —SO₂—; —S⁺R⁷A⁻; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰—SR⁹; —S(O)R⁹; —SO₂R⁹; —SO₃R⁹; —CO₂R⁹; and —CONR⁹R¹⁰; or
R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR 6; —NR⁹RO; —N⁺R⁹R¹⁰R^{w}A⁻; —SR¹⁶; —S(O)R⁹ SO²R⁹ SO³R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A⁻; —S⁺R⁹R¹⁰A⁻; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable cation and M is a pharmaceutically acceptable cation; and
R³² is selected from the group consisting of cycloalkyl, aryl and heterocyclyl,
wherein said cycloalkyl, aryl and heterocyclyl are substituted with —N(H)—X—R³³ or —O—X—R³³ and wherein:
X is selected from the group consisting of:
—(C═O)ₛ-alkyl-;
—(C═O)ₛ-alkyl-NH—;
—(C═O)ₛ-alkyl-O—;
—(C═O)ₛ-alkyl-(C═O)ₜ; and
a covalent bond; and
R₃₃ is selected from selected from the group consisting of monosaccharides, disaccharides, and polysaccharides, wherein said monosaccharides, disaccharides, and polysaccharides may be protected with one or more sugar protecting groups; and
s and t are independently 0 or 1; and
one or more R³⁴ radicals are independently selected from the group consisting of R³², hydrogen; halogen; —CN; —NO₂; alkyl; cycloalkyl; polyalkyl; haloalkyl; hydroxyalkyl; alkenyl; alkynyl; aryl; heterocyclyl; quaternary heterocyclyl; arylalkyl; heterocyclylalkyl; polyether; acyloxy; —OR¹³; —NR¹³ R¹⁴; —SR¹³; —S(O)R¹³ ; —S(O)₂R¹³; —SO₃R¹³; —S⁺R¹³R¹⁴A⁻; —NR¹³OR¹⁴; —NR¹³NR¹⁴R¹⁵; —CO₂R¹³; —OM; —SO₂OM; —SO₂NR¹³R¹⁴; —NR¹⁴C(O)R¹³; —C(O)NR¹³R¹⁴; —C(O)OM; —COR¹³; —R¹⁸; —S(O)ₙNR¹³R¹⁴; —NR¹³R¹⁸; —NR¹⁸OR¹⁴; —N⁺R¹³R¹⁴R¹⁵A⁻; —PR¹³R¹⁴; —P(O)R¹³R¹⁴; —P⁺R¹³R¹⁴R¹⁵A⁻; amino acid residue; peptide residue; polypeptide residue; and carbohydrate residue;
wherein the R³⁴ alkyl; cycloalkyl; polyalkyl; haloalkyl; hydroxyalkyl; alkenyl; alkynyl; aryl; heterocyclyl; arylalkyl; heterocyclylalkyl; polyether; acyloxy radicals optionally may be further substituted with one or more radicals selected from the group consisting of halogen; —CN; oxo; OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹¹R¹²A⁻; —S⁺R⁹R¹⁰A⁻; and carbohydrate residue; and
wherein the R³⁴ quaternary heterocyclyl radical optionally may be substituted with one or more radicals selected from the group consisting of halogen; —CN; —NO₂; oxo; alkyl; cycloalkyl; polyalkyl; haloalkyl; hydroxyalkyl; alkenyl; alkynyl; aryl; heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR³OR¹⁴; —NR¹³NR⁴R⁵; —CO₂R¹³; OM; —SO₂OM; —SO₂NR¹³R¹⁴; —C(O)NR¹³R¹⁴; —C(O)OM; —COR¹³; —P(O)R¹³R¹⁴; —P¹³R¹⁴ P⁺R R¹⁴R¹⁵A⁻; —P(OR¹³)OR¹⁴; —S⁺R¹³R¹⁴A⁻; —N⁺R¹³R¹⁴R¹⁵A⁻; and carbohydrate residue; and
wherein the R³⁴ radicals comprising carbon optionally may have one or more carbons replaced by —O—; —NR¹³ —; —N⁺R¹³R¹⁴A⁻-; —S—; —SO—; —SO₂—; —S⁺R¹³A⁻-; —PR¹³—; —P(O)R¹³—; —PR¹³R¹⁴; —P⁺R¹³R¹⁴A⁻-; phenylene; amino acid residue; peptide residue; polypeptide residue; carbohydrate residue; polyether; or polyalkyl; wherein said phenylene; amino acid residue; peptide residue; polypeptide residue; carbohydrate residue; and polyalkyl optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; or —P(O)R⁹—; and
wherein R¹⁸ is selected from the group consisting of alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl; and
wherein the R¹⁸ alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl radicals optionally may be substituted with one or more radicals selected from the group consisting of halogen; —CN; NO₂; oxo; —OR⁹; —NR⁹R¹⁰; —N⁺R⁹R¹¹R¹²A⁻; —SR⁹; —S(O)R⁹; —SO₂R⁹; —SO₃R⁹; —CO₂R⁹; —CONR⁹R 10; —SO₂OM; —SO₂NR⁹R¹⁰; —PR⁹R¹¹; —P(OR¹³)OR¹⁴; —PO(OR¹⁶)OR¹⁷; and —C(O)OM; or
a pharmaceutically acceptable salt, solvate, or prodrug thereof;
provided that at least one of R³⁰, R³¹ and R³⁴ is R³².

185. A compound of claim 184 wherein R³² is phenyl substituted with —N(H)—X—R³³ or —O—X—R³³ wherein:
X is selected from the group consisting of:
—(C═O)ₛ-alkyl-;
—(C═O)ₛ-alkyl-NH—;
—(C═O)ₛ-alkyl-O—;
—(C═O)ₛ-alkyl-(C═O)ₜ; and
a covalent bond; and
R₃₃ is selected from selected from the group consisting of monosaccharides, disaccharides, and polysaccharides; and
s and t are independently 0 or 1.

186. A compound of claim 185 wherein R³² is phenyl substituted at the para-position with —N(H)—X—R³³ or —O—X—R³³ wherein:
X is selected from the group consisting of:
—(C═O)ₛ-alkyl-;
—(C═O)ₛ-alkyl-NH—;
—(C═O)ₛ-alkyl-O—;
—(C═O)ₛ-alkyl-(C═O)ₜ; and
a covalent bond; and
R₃₃ is selected from selected from the group consisting of monosaccharides, disaccharides, and polysaccharides; and
s and t are independently 0 or 1.

187. A compound of claim 185 wherein R³² is phenyl substituted at the meta-position with —N(H)—X—R³³ or —O—X—R³³ wherein:
X is selected from the group consisting of:
—(C═O)ₛ-alkyl-;
—(C═O)ₛ-alkyl-NH—;
—(C═O)ₛ-alkyl-O—;
—(C═O)ₛ-alkyl-(C═O)ₜ; and
a covalent bond; and
R₃₃ is selected from selected from the group consisting of monosaccharides, disaccharides, and polysaccharides; and
s and t are independently 0 or 1.

188. A compound of claim 185 wherein:
R³⁰ is R³²; and
R³¹ is selected from the group consisting of hydrogen and alkyl.

189. A compound of claim 185 wherein:
R³⁰ is selected from the group consisting of hydrogen and alkyl; and
R³¹ is R³².

190. A compound of claim 185 wherein R³² is phenyl substituted with a radical selected from the group consisting of:

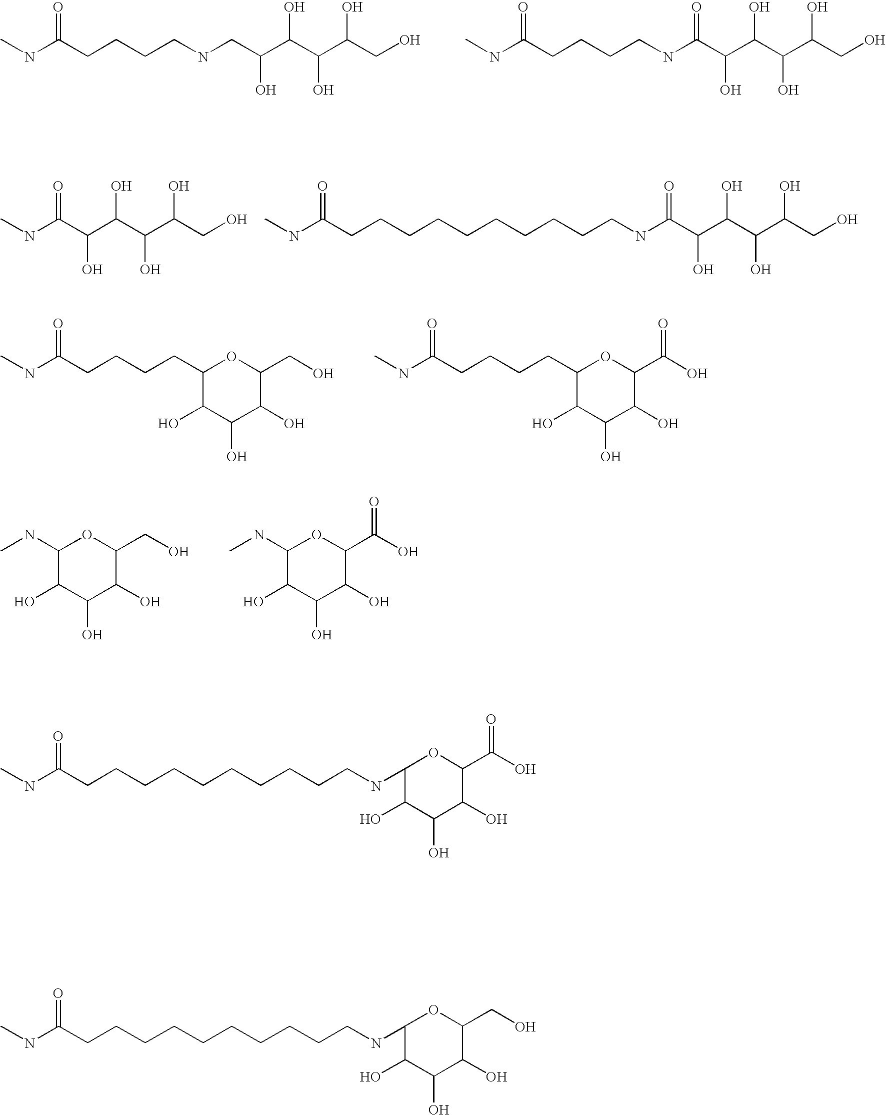

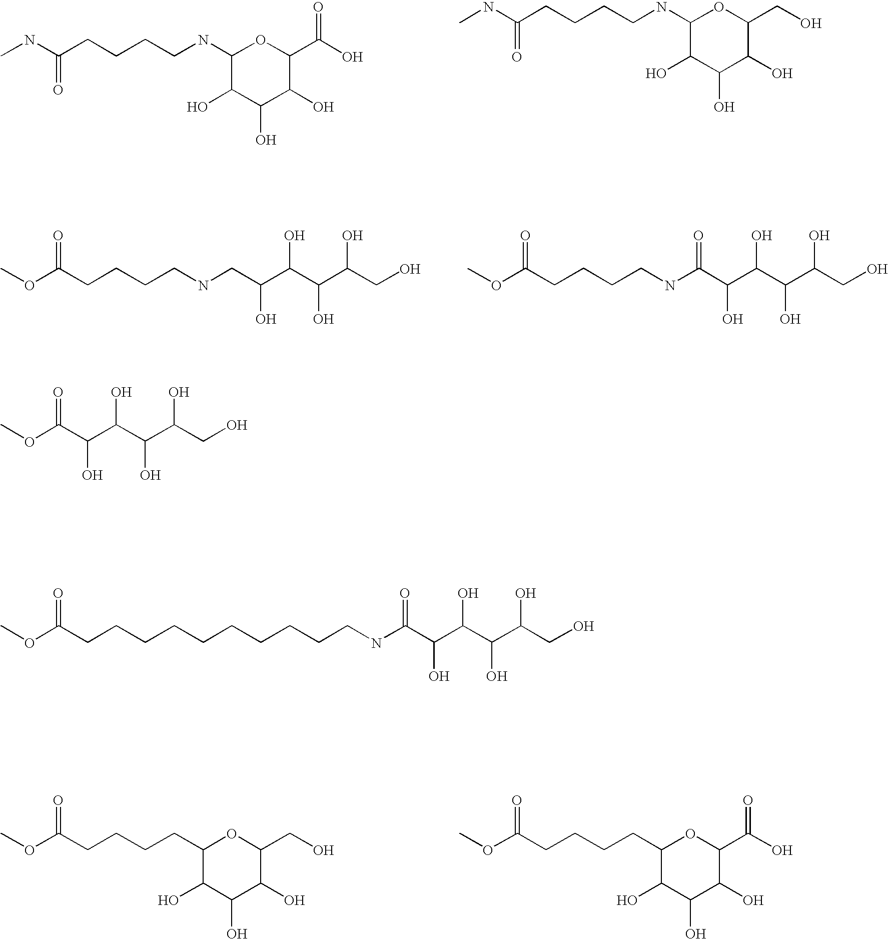

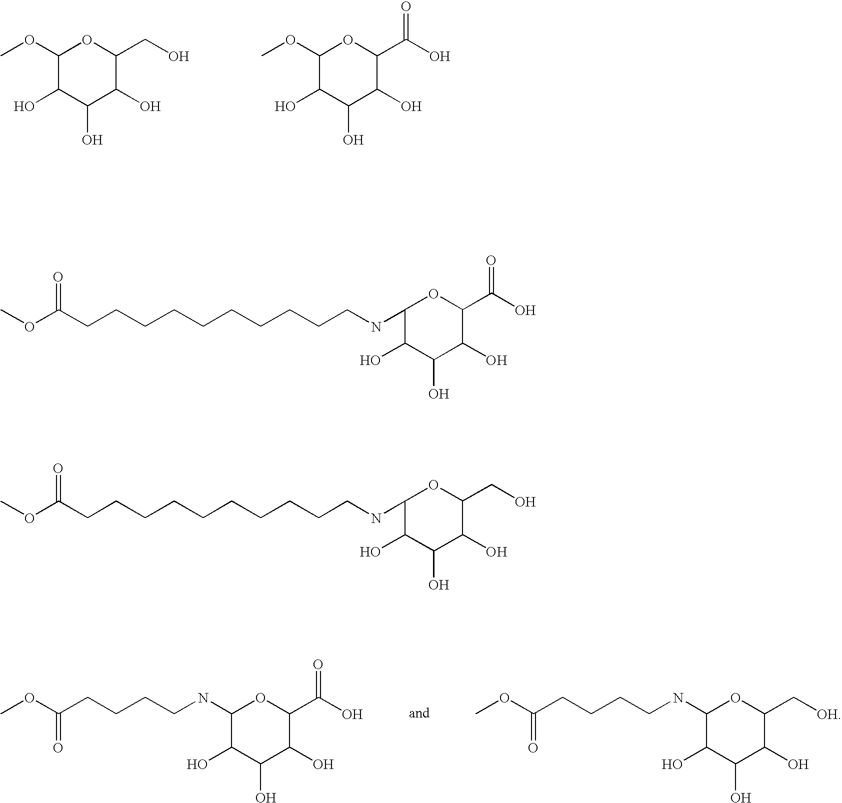

191. A compound of claim 185 wherein:
i is 2;
R^{1C} and R^{1D} are independently selected from hydrogen and alkyl; and
R^{2G} and R^{2H} are independently selected from hydrogen and alkyl.

192. A compound of claim 185 wherein:
i is 2;
R^{1C} and R^{1D} are hydrogen; and
R^{2G} and R^{2H} are independently selected from alkyl.

193. A compound of claim 185 wherein:
i is 2;
R^{1C} and R^{1D} are hydrogen; and
R^{2G} and R^{2H} are independently selected from ethyl, propyl and butyl.

194. A compound of claim 185 wherein i is 1 or 2.

195. A compound of claim 185 wherein i is 2.

196. A compound of claim 185 wherein R^{1C} and R^{1D} are hydrogen.

197. A compound of claim 185 wherein R^{2G} and R^{2H} are independently selected from the group consisting of hydrogen and C₁₋₆alkyl.

198. A compound of claim 185 wherein R^{2G} and R^{2H} are independently selected from the group consisting C₁₋₆alkyl.

199. A compound of claim 185 wherein R^{2G} and R^{2H} are the same alkyl.

200. A compound of claim 185 wherein R^{2G} and R^{2H} are each n-butyl.

201. A compound of claim 185 wherein one of R^{2G} and R^{2H} is ethyl and the other of R^{2G} and R^{2H} is n-butyl.

202. A compound of claim 185 wherein one or more R³⁴ are independently selected from methoxy and dimethylamino.

203. A compound of claim 185 wherein
i is 1 or 2;
R^{1C} and R^{1D} are hydrogen;
R^{2G} and R^{2H} are n-butyl; and
one or more R³⁴ are independently selected from methoxy and dimethylamino.

204. A compound of claim 185 wherein
i is 1 or 2;
R^{1C} and R^{1D} are hydrogen;
one of R^{2G} and R^{2H} is ethyl and the other of R^{2G} and R^{2H} is n-butyl; and
one or more R³⁴ are independently selected from methoxy and dimethylamino.

205. A compound of claim 163 corresponding to Formula VIIB: 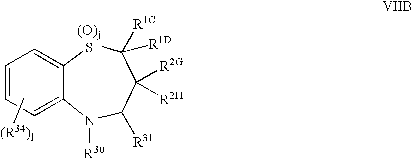
wherein:
i is 0, 1 or 2; and
1 is 0, 1, 2, 3 or 4; and
R^{1C} and R^{1D} are independently selected from hydrogen and alkyl; and
R^{2G} and R^{2H} are independently selected from hydrogen, alkyl, alkenyl, alkynyl, cycloalkyl, cycloalkylalkyl, aryl and aralkyl; or
R^{2G} and R^{2H} together with the carbon atom to which they are attached form a C₃₋₇ cycloalkyl group; and
R³⁰ and R³¹ are independently selected from the group consisting of hydrogen; oxo; alkyl; cycloalkyl; aryl; heterocyclyl; acyl, thioacyl, —OR⁹, and R³²;
wherein the R³⁰ and R³¹ alkyl; cycloalkyl; aryl; heterocyclyl radicals are independently substituted with one or more radicals independently selected from the group consisting of halogen; —CN; —NO₂; oxo; alkyl; polyalkyl; haloalkyl; hydroxyalkyl; cycloalkyl; alkenyl; alkynyl; aryl; heterocyclyl; quaternary heterocyclyl; arylalkyl; heterocyclylalkyl; polyether; —OR¹³; —NR¹³R¹⁴; —SR¹³; —S(O)R¹³; —SO₂R¹³; SO₃R¹³; —NR¹³OR¹⁴; —NR¹³NR¹⁴R¹⁵; —CO₂R¹³; —OM; —SO₂OM; —SO₂NR¹³R¹⁴; —C(O)NR¹³R¹⁴; —C(O)OM; —COR¹³; —NR¹³C(O)R¹⁴; —NR¹³C(O)NR¹⁴R¹⁵; —NR¹³CO₂R¹⁴; —OC(O)R¹³; —OC(O)NR¹³R¹⁴; —NR¹³SOR¹⁴; —NR¹³SO₂R¹⁴; —NR¹³SONR¹⁴R¹⁵; —NR¹³SO₂NR¹⁴R¹⁵; —PR¹³R¹⁴; —P(O)R¹³R¹⁴; —P⁺R¹³R¹⁴R¹⁵A⁻; —P(OR¹³)OR¹⁴; —S⁺R¹³R¹⁴A⁻; and —N⁺R¹³R¹⁴R¹⁵A⁻; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³⁰ and R³¹ radicals optionally may be further substituted with one or more radicals selected from the group consisting of —CN; halogen; hydroxy; oxo; alkyl; cycloalkyl; alkenyl, alkynyl; aryl; heterocyclyl; arylalkyl; heterocyclylalkyl; quaternary heterocyclyl; —OR⁷; —NR⁷R⁸—SR⁷; S(O)R; —SO₂R⁷; —SO₃R⁷; —CO₂R⁷; —CONR⁷R⁸; —N⁺R⁷R⁸R⁹A⁻; —P(O)R⁷R⁸; —PR⁷R⁸; —P⁺R⁷R⁸R⁹A⁻; and —P(O)(OR⁷)OR⁸; and
wherein the alkyl, polyalkyl, haloalkyl, hydroxyalkyl, cycloalkyl, alkenyl, alkynyl, aryl, heterocyclyl, quaternary heterocyclyl, arylalkyl, heterocyclylalkyl, and polyether substituents of the R³⁰ and R³¹ radicals optionally may have one or more carbons replaced by —O—; —NR⁷—; —N⁺R⁷R⁸A⁻-; —S—; —SO—; —SO₂—; —S⁺R⁷A⁻-; —PR⁷—; —P(O)R⁷—; —P⁺R⁷R⁸A⁻-; or phenylene; and
wherein R⁷ and R⁸ are independently selected from the group consisting of hydrogen; and alkyl; and
wherein R⁹, R¹⁰, and R^{w} are independently selected from the group consisting of hydrogen; alkyl; cycloalkyl; alkenyl; alkynyl; aryl; heterocyclyl; alkylammoniumalkyl; arylalkyl; heterocyclylalkyl; carboxyalkyl; alkoxyalkyl; carboalkoxyalkyl; carboxyaryl; carboxyheterocyclyl; amino; alkylamino; carboxyalkylamino; alkoxyalkylamino; and acyl; and
wherein R¹¹ and R¹² are independently selected from the group consisting of hydrogen; —CN; halogen; oxo; alkyl; alkenyl; alkynyl; aryl; heterocyclyl; arylalkyl; heterocyclylalkyl; carboxyalkyl; alkoxyalkyl; carboalkoxyalkyl; cycloalkyl; cycloalkenyl; haloalkyl; hydroxyalkyl; cyanoalkyl; —OR⁹; —NR⁹R¹⁰; —SR⁹; —S(O)R⁹; —SO₂R⁹; —SO₃R⁹; —CO₂R⁹; and —CONR⁹R¹⁰; or R¹¹ and R¹² together with the carbon atom to which they are attached form a cyclic ring; and
wherein R¹³, R¹⁴, and R¹⁵ are independently selected from the group consisting of hydrogen; alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether; or
wherein R¹³ and R¹⁴ together with the nitrogen atom to which they are attached form a mono- or polycyclic heterocyclyl that is optionally substituted with one or more radicals selected from the group consisting of oxo, carboxy, and quaternary salts; or
wherein R¹⁴ and R¹⁵ together with the nitrogen atom to which they are attached form a cyclic ring; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may be substituted with one or more radicals selected from the group consisting of halogen; —CN; sulfo; oxo; alkyl; haloalkyl; hydroxyalkyl; sulfoalkyl; alkenyl; alkynyl; aryl; heterocyclyl; quaternary heterocyclyl; quaternary heterocyclylalkyl; carboxy; carboxyalkyl; guanidinyl; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹⁰R¹¹A-; —S⁺R⁹R¹⁰A-; and carbohydrate residue; and
wherein the R¹³, R¹⁴, and R¹⁵ alkyl; haloalkyl; cycloalkyl; polyalkyl; alkenyl; alkynyl; aryl; heterocyclyl; quaternary heterocyclyl; arylalkyl; heterocyclylalkyl; quaternary heterocyclylalkyl; alkylarylalkyl; alkylheterocyclylalkyl; alkylammoniumalkyl; aminoalkyl; aminocarbonylalkyl; alkylaminocarbonylalkyl; carboxyalkylaminocarbonylalkyl; and polyether radicals optionally may have one or more carbons replaced by —O—; —NR⁹—; —N⁺R⁹R¹⁰A⁻-; —S—; —SO—; —SO₂—; —S⁺R⁹A⁻-; —PR⁹—; —P⁺R⁹R¹⁰A⁻-; —P(O)R⁹—; phenylene; carbohydrate residue; amino acid residue; peptide residue; or polypeptide residue; and
wherein R¹⁶ and R¹⁷ are independently selected from the group consisting of R⁹ and M; and
wherein A⁻ is a pharmaceutically acceptable cation and M is a pharmaceutically acceptable cation; and
R³² is selected from the group consisting of cycloalkyl, aryl and heterocyclyl, wherein said cycloalkyl, aryl and heterocyclyl are substituted with —N(H)—X—R³³ or —O—X—R³³ and wherein:
X is selected from the group consisting of:
—(C═O)ₛ-alkyl-;
—(C═O)ₛ-alkyl-NH—;
—(C═O)ₛ-alkyl-O—;
—(C═O)ₛ-alkyl-(C═O)ₜ; and
a covalent bond; and
R₃₃ is selected from selected from the group consisting of monosaccharides, disaccharides, and polysaccharides, wherein said monosaccharides, disaccharides, and polysaccharides may be protected with one or more sugar protecting groups; and
s and t are independently 0 or 1; and
one or more R³⁴ radicals are independently selected from the group consisting of R³², hydrogen; halogen; —CN; —NO₂; alkyl; cycloalkyl; polyalkyl; haloalkyl; hydroxyalkyl; alkenyl; alkynyl; aryl; heterocyclyl; quaternary heterocyclyl; arylalkyl; heterocyclylalkyl; polyether; acyloxy; —OR¹³; —NR¹³R¹⁴; —SR¹³; —S(O)R¹³; —S(O)₂R¹³; —SO₃R¹³; —S⁺R¹³R¹⁴A⁻; —NR¹³OR¹⁴; —NR¹³NR¹⁴NR¹⁵; —CO₂R¹³; —OM; —SO₂OM; —SO₂NR¹³R¹⁴; —NR¹⁴C(O)R¹³; —C(O)NR¹³ R¹⁴; —C(O)OM; —COR¹³; —OR¹⁸; —S(O)ₙNR¹³R¹⁴; —NR¹³R¹⁴; —NR¹⁸OR¹⁴; —N⁺R¹³R¹⁴R¹⁵A⁻; —PR¹³R¹⁴; —P(O)R¹³R¹⁴—P⁺R¹³R¹⁴R¹⁵A⁻; amino acid residue; peptide residue; polypeptide residue; and carbohydrate residue;
wherein the R³⁴ alkyl; cycloalkyl; polyalkyl; haloalkyl; hydroxyalkyl; alkenyl; alkynyl; aryl; heterocyclyl; arylalkyl; heterocyclylalkyl; polyether; acyloxy radicals optionally may be further substituted with one or more radicals selected from the group consisting of halogen; —CN; oxo; —OR¹⁶; —NR⁹R¹⁰; —N⁺R⁹R¹⁰R^{w}A⁻; —SR¹⁶; —S(O)R⁹; —SO₂R⁹; —SO₃R¹⁶; —CO₂R¹⁶; —CONR⁹R¹⁰; —SO₂NR⁹R¹⁰; —PO(OR¹⁶)OR¹⁷; —P⁹R¹⁰; —P⁺R⁹R¹¹ R¹²A⁻; —S⁺R⁹R¹⁰A⁻; and carbohydrate residue; and
wherein the R³⁴ quaternary heterocyclyl radical optionally may be substituted with one or more radicals selected from the group consisting of halogen; —CN; —NO₂; oxo; alkyl; cycloalkyl; polyalkyl; haloalkyl; hydroxyalkyl; alkenyl; alkynyl; aryl; heterocyclyl; arylalkyl; heterocyclylalkyl; polyether; —OR¹³; —NR¹³R¹⁴; —SR¹³; —S(O)R¹³; —SO₂R¹³; —SO₃R¹³; —NR¹³OR¹⁴; —NR¹³NR¹⁴R¹⁵; —CO₂R¹³; OM; —SO₂OM; —SO₂NR¹³R¹⁴; —C(O)NR¹³R¹⁴; —C(O)OM; —COR¹³; —P(O)R¹³R¹⁴; —P¹³R¹⁴; —P⁺R¹³R¹⁴R¹⁵A⁻; —P(OR¹³)OR¹⁴; —S⁺R¹³R¹⁴A⁻; —N⁺R¹³R¹⁴R¹⁵A⁻; and carbohydrate residue; and
wherein the R³⁴ radicals comprising carbon optionally may have one or more carbons replaced by —O—; —NR¹³—; —N⁺R¹³R¹⁴A⁻-; —S—; —SO—; —SO₂—; —S⁺R¹³A⁻-; —PR¹³—; —P(O)R¹³—; —PR¹³R¹⁴; —P⁺R¹³R¹⁴A⁻-; phenylene; amino acid residue; peptide residue; polypeptide residue; carbohydrate residue; polyether; or polyalkyl; wherein said phenylene; amino acid residue; peptide residue; polypeptide residue; carbohydrate residue; and polyalkyl optionally may have one or more carbons replaced by —O—; —NR⁹—; —N⁺R⁹R¹⁰A⁻-; —S—; —SO—; —SO₂—; —SR 9A⁻-; —PR⁹—; —P⁺R⁹R¹⁰A⁻-; or —P(O)R⁹-; and
wherein R¹⁸ is selected from the group consisting of alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl; and
wherein the R¹⁸ alkyl; alkenyl; alkynyl; aryl; heterocyclyl; quaternary heterocyclyl; arylalkyl; heterocyclylalkyl; acyl; alkoxycarbonyl; arylalkoxycarbonyl; and heterocyclylalkoxycarbonyl radicals optionally may be substituted with one or more radicals selected from the group consisting of halogen; —CN; NO₂; oxo; —OR⁹; —NR⁹R¹⁰; —N⁺R⁹R¹¹ R¹²A⁻; —SR⁹; —S(O)R⁹; —SO₂R⁹; —SO₃R⁹; —CO₂R⁹; —CONR⁹R¹⁰; —SO₂OM; —SO₂NR⁹R¹⁰; —PR⁹R¹⁰; —P(OR¹³)OR¹⁴; —PO(OR¹⁶)OR¹⁷; and —C(O)OM; or
a pharmaceutically acceptable salt, solvate, or prodrug thereof;
provided that at least one of R³⁰, R³¹ and R³⁴ is R³².

206. A compound of claim 205 wherein R³² is phenyl substituted with —N(H)—X—R³³ or —O—X—R³³ wherein:
X is selected from the group consisting of:
—(C═O)ₛ-alkyl-;
—(C═O)ₛ-alkyl-NH—;
—(C═O)ₛ-alkyl-O—;
—(C═O)ₛ-alkyl-(C═O)ₜ; and
a covalent bond; and
R₃₃ is selected from selected from the group consisting of monosaccharides, disaccharides, and polysaccharides; and
s and t are independently 0 or 1.

207. A compound of claim 206 wherein R³² is phenyl substituted at the para-position with —N(H)—X—R³³ or —O—X—R³³ wherein:
X is selected from the group consisting of:
—(C═O)ₛ-alkyl-;
—(C═O)ₛ-alkyl-NH—;
—(C═O)ₛ-alkyl-O—;
—(C═O)ₛ-alkyl-(C═O)ₜ; and
a covalent bond; and
R³³ is selected from selected from the group consisting of monosaccharides, disaccharides, and polysaccharides; and
s and t are independently 0 or 1.

208. A compound of claim 206 wherein R³² is phenyl substituted at the meta-position with —N(H)—X—R³³ or —O—X—R³³ wherein:
X is selected from the group consisting of:
—(C═O)ₛ-alkyl-;
—(C═O)ₛ-alkyl-NH—;
—(C═O)ₛ-alkyl-O—;
—(C═O)ₛ-alkyl-(C═O)ₜ; and
a covalent bond; and
R₃₃ is selected from selected from the group consisting of monosaccharides, disaccharides, and polysaccharides; and
s and t are independently 0 or 1.

209. A compound of claim 206 wherein:
R³⁰ is R³² and
R³¹ is selected from the group consisting of hydrogen and alkyl.

210. A compound of claim 206 wherein:
R³⁰ is selected from the group consisting of hydrogen and alkyl; and
R³¹ is R³².

211. A compound of claim 206 wherein R³² is phenyl substituted with a radical selected from the group consisting of:

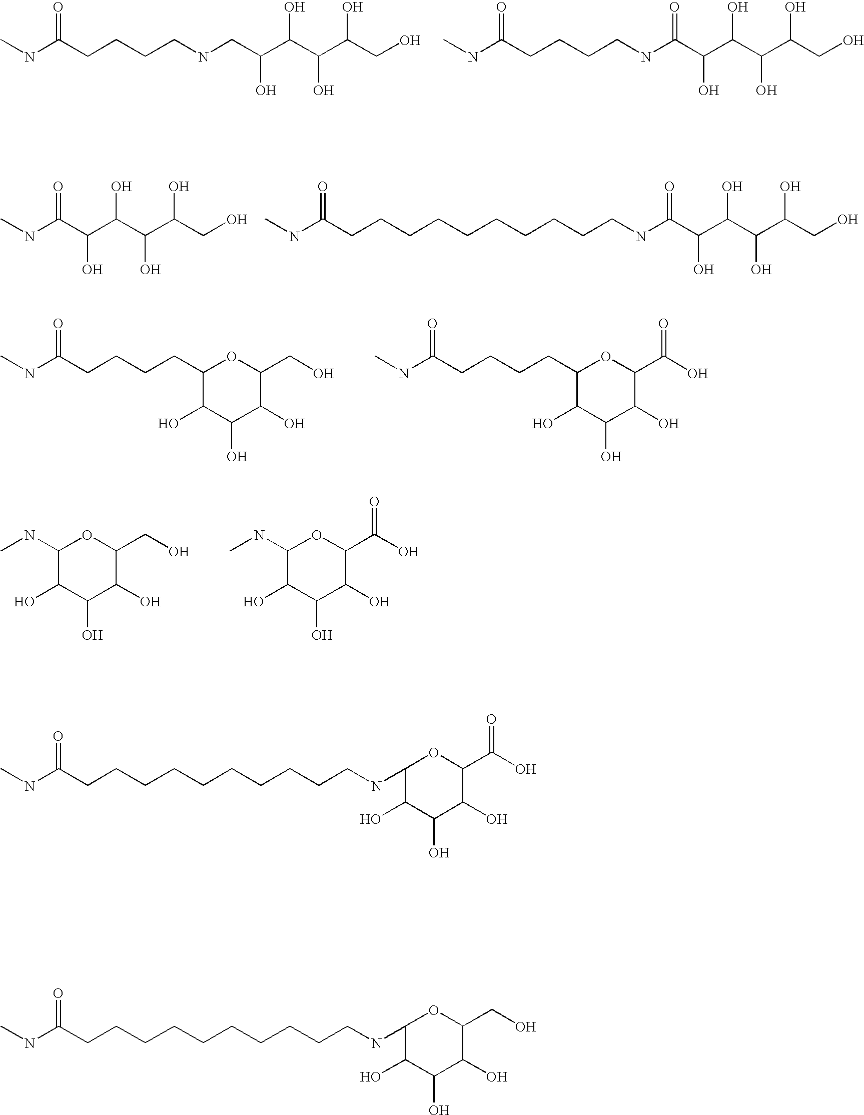

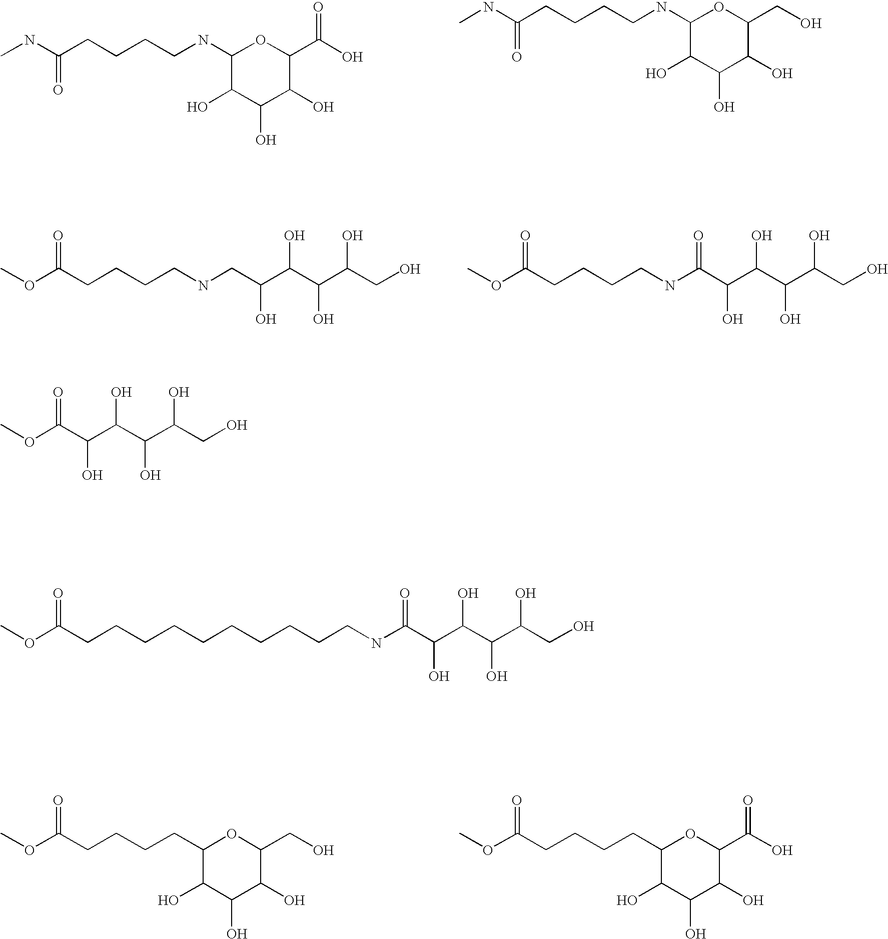

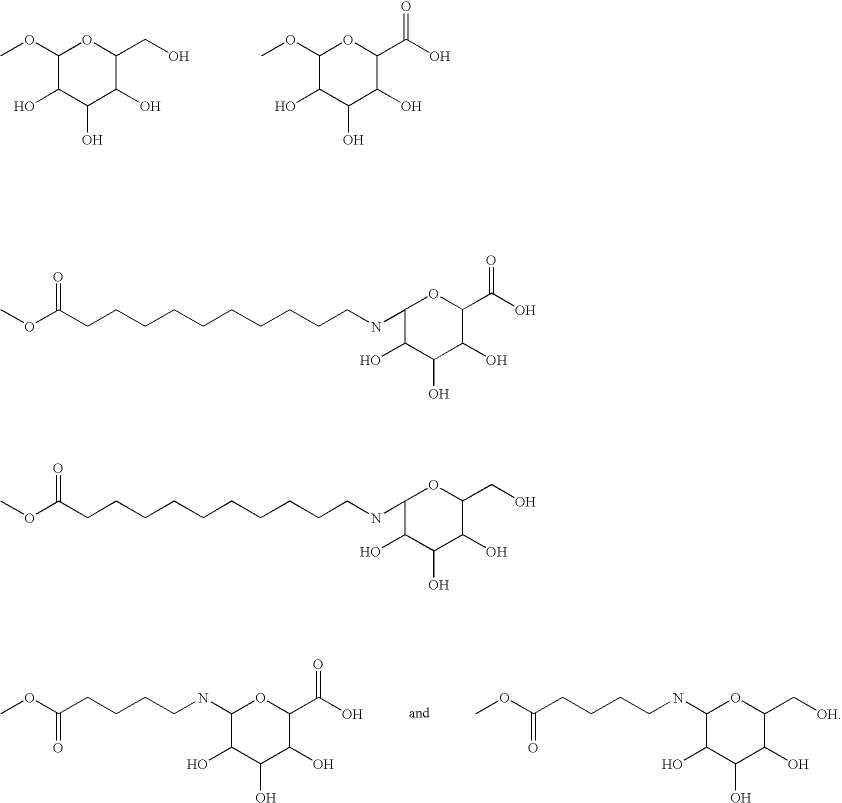

212. A compound of claim 206 wherein:
i is 2;
R^{1C} and R^{1D} are independently selected from hydrogen and alkyl; and
R^{2G} and R^{2H} are independently selected from hydrogen and alkyl.

213. A compound of claim 206 wherein:
i is 2;
R^{1C} and R^{1D} are hydrogen; and
R^{2G} and R^{2H} are independently selected from alkyl.

214. A compound of claim 206 wherein:
i is 2;
R^{1C} and R^{1D} are hydrogen; and
R ^{2G} and R^{2H} are independently selected from ethyl, propyl and butyl.

215. A compound of claim 206 wherein i is 1 or 2.

216. A compound of claim 206 wherein i is 2.

217. A compound of claim 206 wherein R^{1C} and R^{1D} are hydrogen.

218. A compound of claim 206 wherein R^{2G} and R^{2H} are independently selected from the group consisting of hydrogen and C₁₋₆alkyl.

219. A compound of claim 206 wherein R^{2G} and R^{2H} are independently selected from the group consisting C₁₋₆alkyl.

220. A compound of claim 206 wherein R^{2G} and R^{2H} are the same alkyl.

221. A compound of claim 206 wherein R^{2G} and R^{2H} are each n-butyl.

222. A compound of claim 206 wherein one of R^{2G} and R^{2H} is ethyl and the other of R^{2G} and R^{2H} is n-butyl.

223. A compound of claim 206 wherein one or more R³⁴ are independently selected from methoxy and dimethylamino.

224. A compound of claim 206 wherein
i is 1 or 2;
R^{1C} and R^{1D} are hydrogen;
R^{1G} and R^{2H} are n-butyl; and
one or more R³⁴ are independently selected from methoxy and dimethylamino.

225. A compound of claim 206 wherein
i is 1 or 2;
R^{1C} and R^{1D} are hydrogen;
one of R^{2G} and R^{2H} is ethyl and the other of R^{2G} and R^{2H} is n-butyl; and
one or more R³⁴ are independently selected from methoxy and dimethylamino.

226. A compound of Formula VIII: 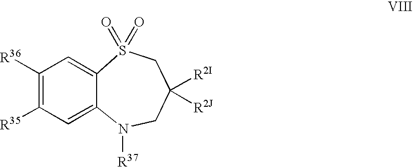
wherein:
R^{2I} and R^{2J} are independently selected from C₁₋₆alkyl; and
R³⁵ is selected from the group consisting of halogen and R³⁸;
R³⁶ is selected from the group consisting of hydroxy, alkoxy, and R³⁸;
wherein R³⁸ is selected from the group consisting of cycloalkyl, aryl and heterocyclyl, wherein said cycloalkyl, aryl and heterocyclyl are substituted with —N(H)—X—R³⁹ or —O—X—R³⁹ and wherein:
X is selected from the group consisting of:
—(C═O)ᵤ-alkyl-;
—(C═O)ᵤ-alkyl-NH—;
—(C═O)ᵤ-alkyl-O—;
—(C═O)ᵤ-alkyl-(C═O)ᵥ; and
a covalent bond; and
R³⁹ is selected from selected from the group consisting of monosaccharides, disaccharides, and polysaccharides, wherein said monosaccharides, disaccharides, and polysaccharides may be protected with one or more sugar protecting groups; and
u and v are independently 0 or 1; and
R³⁷ is unsubstituted phenyl or R³⁸; or
a pharmaceutically acceptable salt, solvate, or prodrug thereof;
provided that at least one of R³⁵, R³⁶ and R³⁷ is R³⁸.

227. A compound of claim 226 wherein R³⁸ is phenyl substituted with —N(H)—X—R³⁹ or —O—X—R³⁹ wherein:
X is selected from the group consisting of:
—(C═O)ᵤ-alkyl-;
—(C═O)ᵤ-alkyl-NH—;
—(C═O)ᵤ-alkyl-O—;
—(C═O)ᵤ-alkyl-(C═O)ᵥ; and
a covalent bond; and
R³⁹ is selected from selected from the group consisting of monosaccharides, disaccharides, and polysaccharides; and
u and v are independently 0 or 1.

228. A compound of claim 227 wherein R³⁸ is phenyl substituted at the para-position with —N(H)—X—R³⁹ or —O—X—R³⁹ wherein:
X is selected from the group consisting of:
—(C═O)ᵤ-alkyl-;
—(C═O)ᵤ-alkyl-NH—;
—(C═O)ᵤ-alkyl-O—;
—(C═O)ᵤ-alkyl-(C═O)ᵥ; and
a covalent bond; and
R³⁹ is selected from selected from the group consisting of monosaccharides, disaccharides, and polysaccharides; and
u and v are independently 0 or 1.

229. A compound of claim 227 wherein R³⁸ is phenyl substituted at the meta-position with —N(H)—X—R³⁹ or —O—X—R³⁹ wherein:
X is selected from the group consisting of:
—(C═O)ᵤ-alkyl-;
—(C═O)ᵤ-alkyl-NH—;
—(C═O)ᵤ-alkyl-O—;
—(C═O)ᵤ-alkyl-(C═O)ᵥ; and
a covalent bond; and
R³⁹ is selected from selected from the group consisting of monosaccharides, disaccharides, and polysaccharides; and
u and v are independently 0 or 1.

230. A compound of claim 227 wherein R³⁸ is phenyl substituted with a radical selected from the group consisting of:

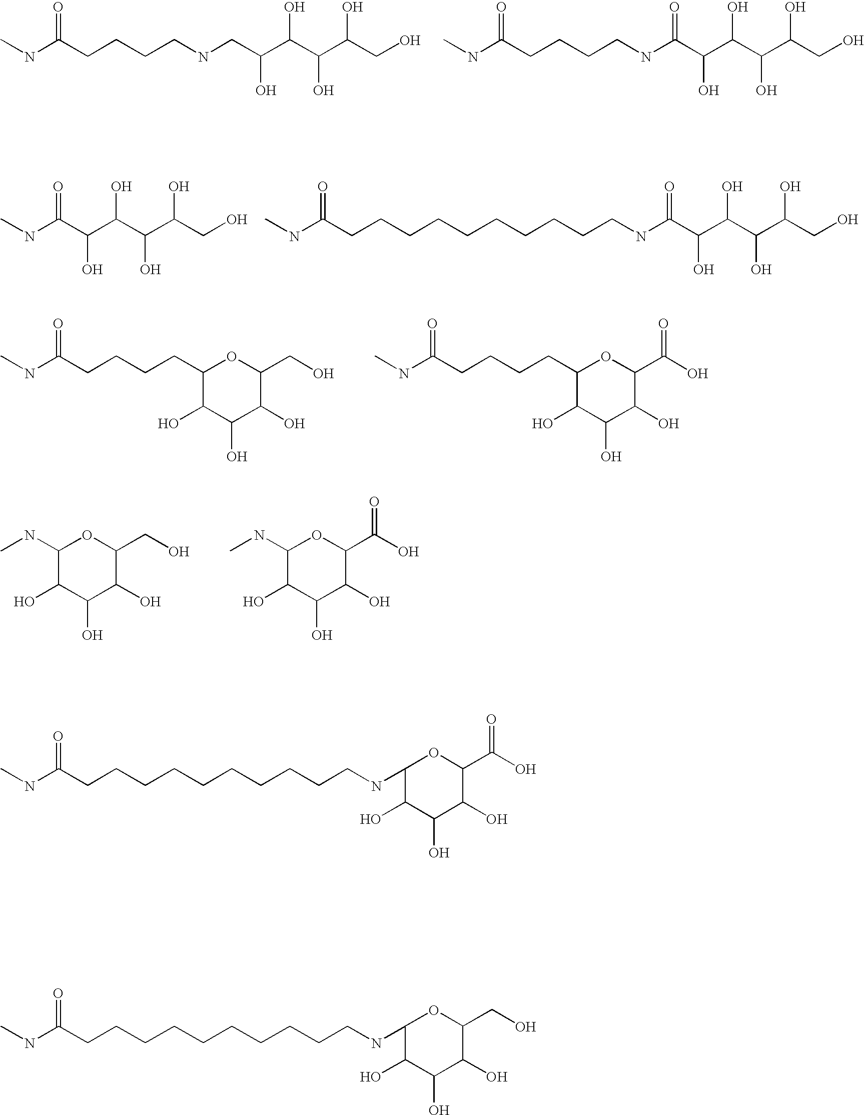

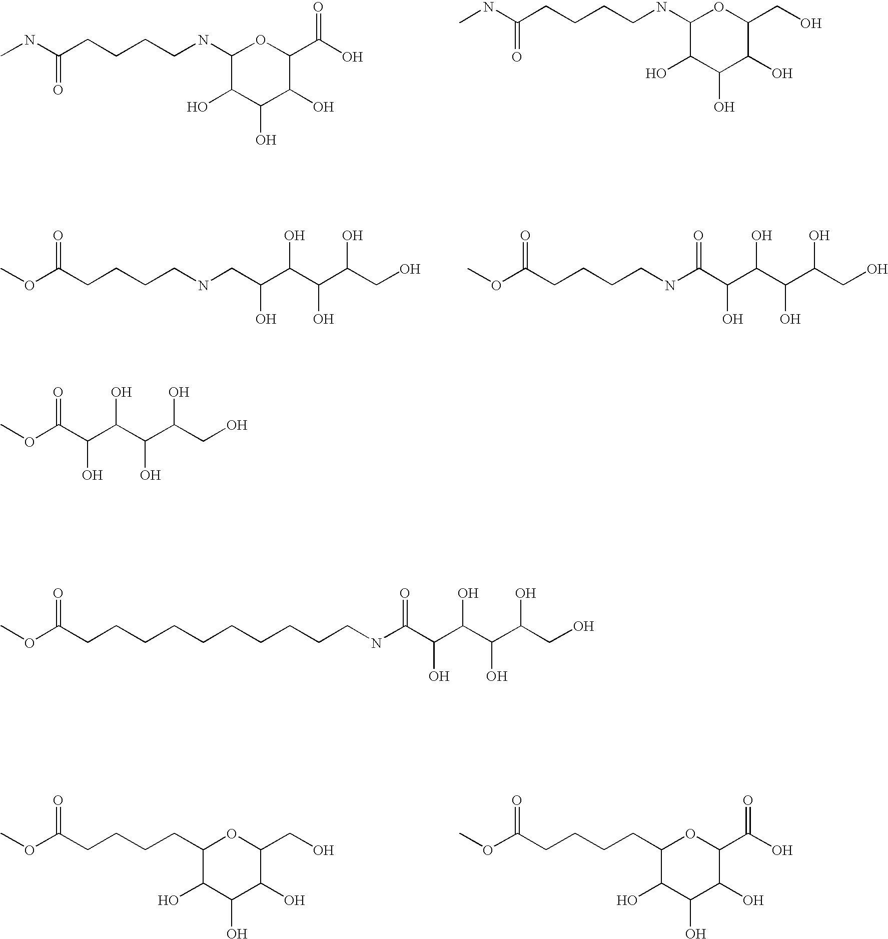

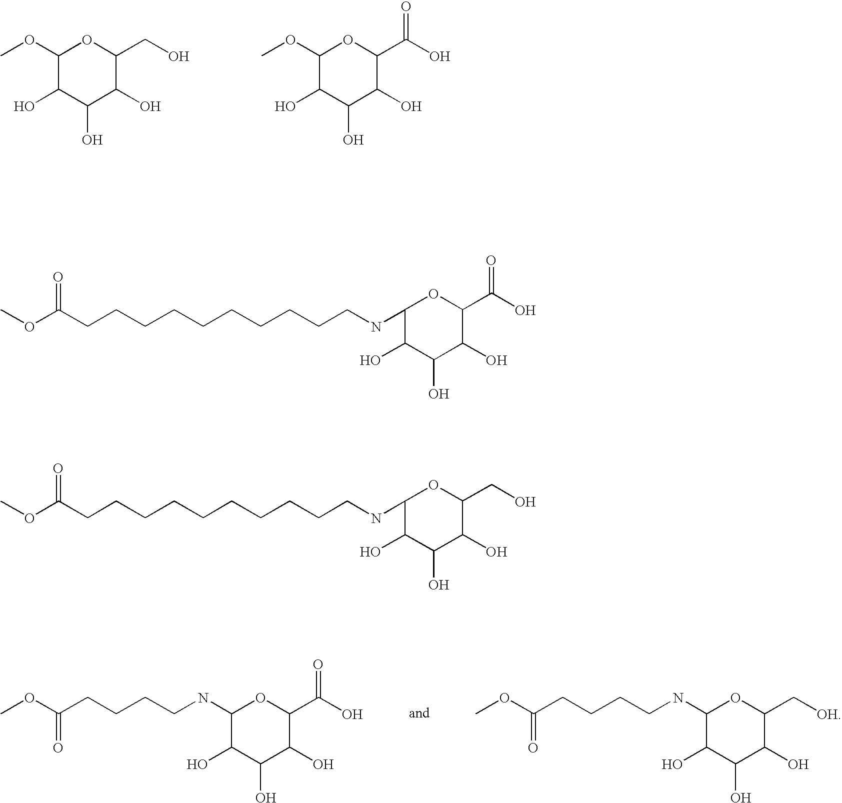

231. A compound of claim 227 wherein:
R^{2I} and R^{2J} are independently selected from ethyl and n-butyl;
R³⁵ is chloro; and
R³⁶ is selected from the group consisting of hydroxy and methoxy.

232. A compound of claim 227 wherein:
R^{2I} and R^{2J} are n-butyl;
R³⁵ is chloro; and
R³⁶ is selected from the group consisting of hydroxy and methoxy.

233. A compound of claim 227 wherein:
one of R^{2I} and R^{2J} is ethyl and the other of R^{2I} and R^{2J} is n-butyl;
R³⁵ is chloro; and
R³⁶ is selected from the group consisting of hydroxy and methoxy.

234. A compound of claim 227 wherein R^{2I} and R^{2J} are the same alkyl.

235. A compound of claim 227 wherein R^{2I} and R^{2J} are each n-butyl.

236. A compound of claim 227 wherein one of R^{2I} and R^{2J} is ethyl and the other of R^{2I} and R^{2J} is n-butyl.

237. A compound of Formula IX: 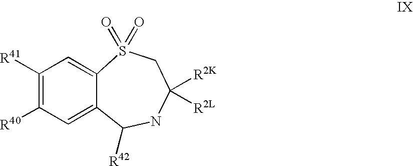
wherein:
R^{2K} and R^{2L} are independently selected from C₁₋₆ alkyl; and
R⁴⁰ and R⁴¹ are independently selected from the group consisting of hydrogen, alkoxy, and R⁴³;
wherein R⁴³ is selected from the group consisting of cycloalkyl, aryl and heterocyclyl, wherein said cycloalkyl, aryl and heterocyclyl are substituted with —N(H)—X—R⁴⁴ or —O—X—R⁴⁴ and wherein:
X is selected from the group consisting of:
—(C═O)ₐ-alkyl-;
—(C═O)ₐ-alkyl-NH—;
—(C═O)ₐ-alkyl-O—;
—(C═O)ₐ-alkyl-(C═O)_{b}; and
a covalent bond; and
R⁴⁴ is selected from selected from the group consisting of monosaccharides, disaccharides, and polysaccharides, wherein said monosaccharides, disaccharides, and polysaccharides may be protected with one or more sugar protecting groups; and
a and b are independently 0 or 1; and
R⁴² is unsubstituted phenyl or R⁴³; or
a pharmaceutically acceptable salt, solvate, or prodrug thereof;
provided that at least one of R⁴⁰, R⁴¹ and R⁴² is R⁴³.

238. A compound of claim 237 wherein R⁴³ is phenyl substituted with —N(H)—X—R⁴⁴ or —O—X—R⁴⁴ wherein:
X is selected from the group consisting of:
—(C═O)ₐ-alkyl-;
—(C═O)ₐ-alkyl-NH—;
—(C═O)ₐ-alkyl-O—;
—(C═O)ₐ-alkyl-(C═O)_{b}; and
a covalent bond; and
R⁴⁴ is selected from selected from the group consisting of monosaccharides, disaccharides, and polysaccharides; and
a and b are independently 0 or 1.

239. A compound of claim 238 wherein R⁴³ is phenyl substituted at the para-position with —N(H)—X—R⁴⁴ or —O—X—R⁴⁴ wherein:
X is selected from the group consisting of:
—(C═O)ₐ-alkyl-;
—(C═O)ₐ-alkyl-NH—;
—(C═O)ₐ-alkyl-O—;
—(C═O)ₐ-alkyl-(C═O)_{b}; and
a covalent bond; and
R⁴⁴ is selected from selected from the group consisting of monosaccharides, disaccharides, and polysaccharides; and
a and b are independently 0 or 1.

240. A compound of claim 238 wherein R⁴³ is phenyl substituted at the meta-position with —N(H)—X—R⁴⁴ or —O—X—R⁴⁴ wherein:
X is selected from the group consisting of:
—(C═O)ₐ-alkyl-;
—(C═O)₂-alkyl-NH—;
—(C═O)ₐ-alkyl-O-;
—(C═O)ₐ-alkyl-(C═O)_{b}; and
a covalent bond; and
R⁴⁴ is selected from selected from the group consisting of monosaccharides, disaccharides, and polysaccharides; and
a and b are independently 0 or 1.

241. A compound of claim 238 wherein R⁴³ is phenyl substituted with a radical selected from the group consisting of:

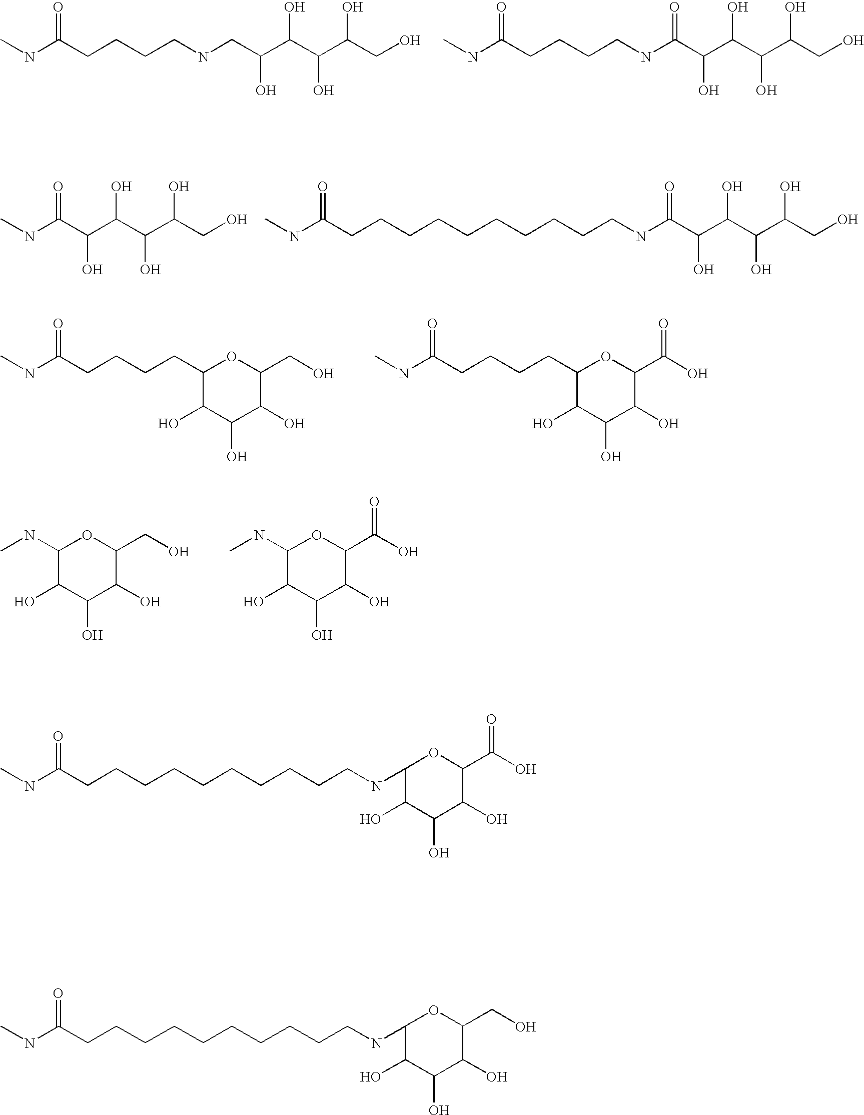

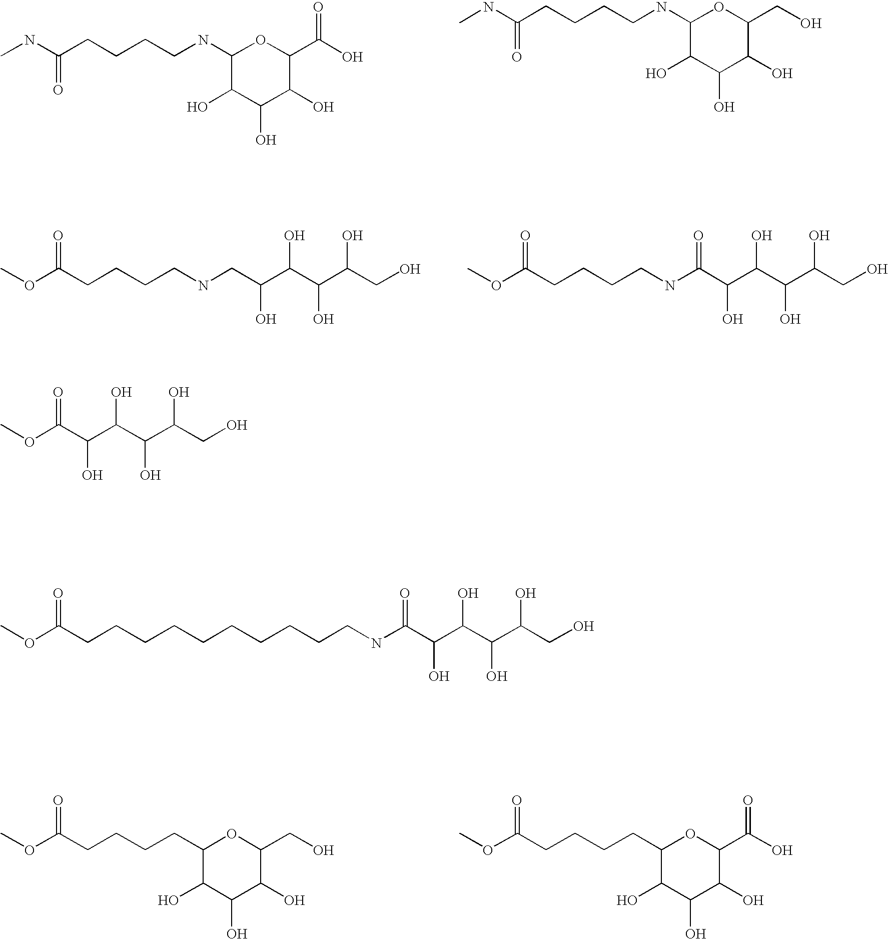

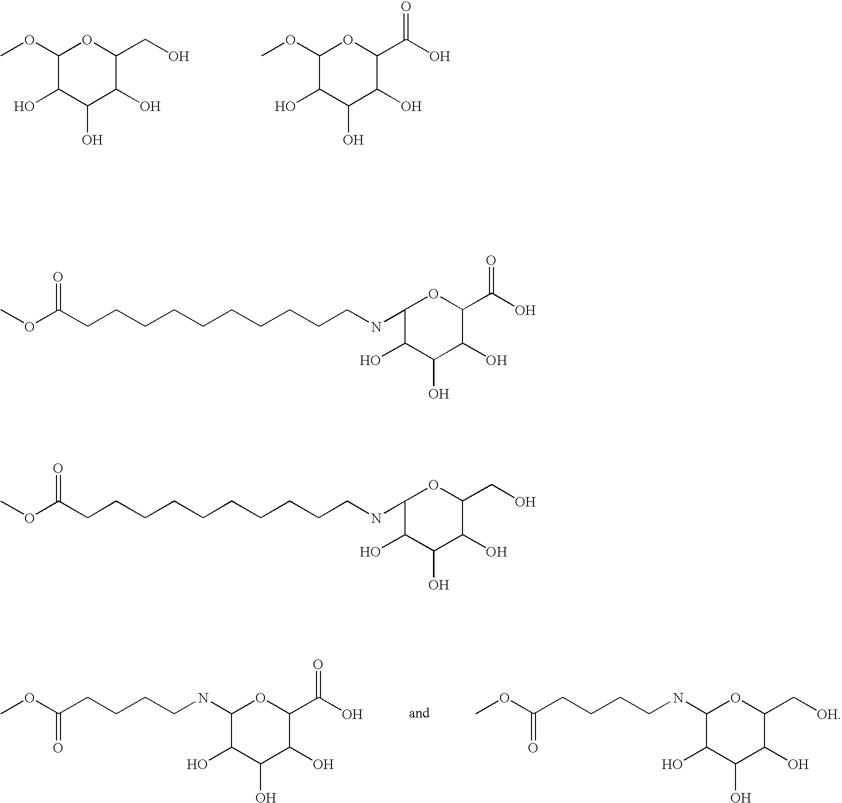

242. A compound of claim 238 wherein:
R^{2K} and R^{2L} are independently selected from ethyl and n-butyl; and
R⁴⁰ and R⁴¹ are independently selected from hydrogen and methoxy.

243. A compound of claim 238 wherein:
R^{2K} and R^{2L} are n-butyl; and
R⁴⁰ and R⁴¹ are independently selected from hydrogen and methoxy.

244. A compound of claim 238 wherein:
one of R^{2K} and R^{2L} is ethyl and the other of R^{2K} and R^{2L} is n-butyl; and
R⁴⁰ and R⁴¹ are independently selected from hydrogen and methoxy.

245. A compound of claim 238 wherein R^{2K} and R^{2L} are the same alkyl.

246. A compound of claim 238 wherein R^{2K} and R^{2L} are each n-butyl.

247. A compound of claim 238 wherein one of R^{2K} and R^{2L} is ethyl and the other of R^{2K} and R^{2L} is n-butyl.

249. A compound of claim 238 wherein:
one of R^{2K} and R^{2L} is ethyl and the other of R^{2K} and R^{2L} is n-butyl; and
R⁴⁰ and R⁴¹ are hydrogen.

250. A compound of claim 238 wherein:
one of R^{2K} and R^{2L} is ethyl and the other of R^{2K} and R^{2L} is n-butyl; and
R⁴⁰ and R⁴¹ are methoxy.

251. A method of treating a hyperlipidemic condition in a subject comprising administering to the subject a therapeutically effective amount of a compound of Formula I according to any one of claims 1 to 120, or a pharmaceutically acceptable salt, solvate or prodrug thereof.

252. A method of treating a hyperlipidemic condition in a subject comprising administering to the subject a therapeutically effective amount of a compound of Formula III according to any one of claims 121 to 140, or a pharmaceutically acceptable salt, solvate or prodrug thereof.

253. A method of treating a hyperlipidemic condition in a subject comprising administering to the subject a therapeutically effective amount of a compound of Formula V according to any one of claims 141 to 162, or a pharmaceutically acceptable salt, solvate or prodrug thereof.

254. A method of treating a hyperlipidemic condition in a subject comprising administering to the subject a therapeutically effective amount of a compound of Formula VII according to any one of claims 163 to 225, or a pharmaceutically acceptable salt, solvate or prodrug thereof.

255. A method of treating a hyperlipidemic condition in a subject comprising administering to the subject a therapeutically effective amount of a compound of Formula VIII according to any one of claims 226 to 236, or a pharmaceutically acceptable salt, solvate or prodrug thereof.

256. A method of treating a hyperlipidemic condition in a subject comprising administering to the subject a therapeutically effective amount of a compound of Formula IX according to any one of claims 237 to 250, or a pharmaceutically acceptable salt, solvate or prodrug thereof.

257. The method of claim 251 wherein the hyperlipidemic condition is atherosclerosis.

258. A pharmaceutical composition comprising a compound of Formula I according to any one of claims 1 to 120 or a pharmaceutically acceptable salt, solvate or prodrug thereof, and a pharmceutically acceptable carrier.

259. A pharmaceutical composition comprising a compound of Formula III according to any one of claims 121 to 140 or a pharmaceutically acceptable salt, solvate or prodrug thereof, and a pharmceutically acceptable carrier.

260. A pharmaceutical composition comprising a compound of Formula V according to any one of claims 141 to 162 or a pharmaceutically acceptable salt, solvate or prodrug thereof, and a pharmceutically acceptable carrier.

261. A pharmaceutical composition comprising a compound of Formula VII according to any one of claims 163 to 225 or a pharmaceutically acceptable salt, solvate or prodrug thereof, and a pharmceutically acceptable carrier.

262. A pharmaceutical composition comprising a compound of Formula VIII according to any one of claims 226 to 236 or a pharmaceutically acceptable salt, solvate or prodrug thereof, and a pharmceutically acceptable carrier.

263. A pharmaceutical composition comprising a compound of Formula IX according to any one of claims 237 to 250 or a pharmaceutically acceptable salt, solvate or prodrug thereof, and a pharmceutically acceptable carrier.
